SEQUENCE LISTING (478 sequences)

SEQ ID NO: 1 - protein, 1307 aa, synthetic construct
MADLSQFTHKYQVPKTLRFELIPQGKTLENLSAYGMVADDKQRSENYKKLKPVIDRIYKYFIEESLKNTNLDWNPLYEAIREYRKEKTTATITNLKEQQDICRRAIASRFEGKVPDKGDKSVKDFNKKQSKLFKELFGKELFTDSVLEQLPGVSLSDEDKALLKSFDKFTTYFVGFYDNRKNVFSSDDISTGIPHRLVQENFPKFIDNCDDYKRLVLVAPELKEKLEKAAEATKIFEDVSLDEIFSIKFYNRLLQQNQIDQFNQLLGGIAGAPGTPKIQGLNETLNLSMQQDKTLEQKLKSVPHRFSPLYKQILSDRSSLSFIPESFSCDAEVLLAVQEYLDNLKTEHVIEDLKEVFNRLTTLDLKHIYVNSTKVTAFSQALFGDWNLCREQLRVYKMSNGNEKITKKALGELESWLKNSDIAFTELQEALADEALPAKVNLKVQEAISGLNEQMAKSLPKELKIPEEKEELKALLDAIQEVYHTLEWFIVSDDVETDTDFYVPLKETLQIIQPIIPLYNKVRNFATQKPYSVEKFKLNFANPTLADGWDENKEQQNCAVLFQKGNNYYLGILNPKNKPDFDNVDTEKQGNCYQKMVYKQFPDFSKMMPKCTTQLKEVKQHFEGKDSDYILNNKNFIKPLTITREVYDLNNVLYDGKKKFQIDYLRKTKDEDGYYTALHTWIDFAKKFVASYKSTSIYDTSTILPPEKYEKLNEFYGALDNLFYQIKFENIPEEIIDTYVEDGKLFLFQIYNKDFAAGATGAPNLHTIYWKAVFDPENVKDVVVKLNGQAELFYRPKSNMDVIRHKVGEKLVNRTLKDGSILTDELHKELYLYANGSLKKGLSEDAKIILDKNLAVIYDVHHEIVKDRRFTTDKFFFHVPLTLNYKCDKNPVKFNAEVQEYLKENPDTYVIGIDRGERNLIYAVVIDPKGRIVEQKSFNVINGFDYHGKLDQREKERVKARQAWTAVGKIKELKQGYLSLVVHEISKMMVRYQAVVVLENLNVGFKRVRSGIAEKAVYQQFEKMLINKLNYLMFKDAGGTEPGSVLNAYQLTDRFESFAKMGLQTGFLFYIPAAFTSKIDPATGFVDPFRWGAIKTLADKREFLSGFESLKFDSTTGNFILHFDVSKNKNFQKKLEGFVPDWDIIIEANKMKTGKGATYIAGKRIEFVRDNNSQGHYEDYLPCNALAETLRQCDIPYEEGKDILPLILEKNDSKLLHSVFKVVRLTLQMRNSNAETGEDYISSPVEDVSGSCFDSRMENEKLPKDADANGAYHIALKGMLALERLRKDEKMAISNNDWLNYIQEKRA

SEQ ID NO: 2 - protein, 492 aa, synthetic construct
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 3 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 4 - protein, 1297 aa, synthetic construct
MATLVSFTKQYQVQKTLRFELIPQGKTQANIDAKGFINDDLKRDENYMKVKGVIDELHKNFIEQTLVNVDYDWRSLATAIKNYRKDRSDTNKKNLEKTQEAARKEIIAWFEGKRGNSAFKNNQKSFYGKLFKKELFSEILRSDDLEYDEETQDAIACFDKFTTYFVGFHENRKNMYSTEAKSTSVAYRVVNENFSKFLSNCEAFSVLEAVCPNVLVEAEQELHLHKAFSDLKLSDVFKVEAYNKYLSQTGIDYYNQIIGGISSAEGVRKIRGVNEVVNNAIQQNDELKVALRNKQFTMVQLFKQILSDRSTLSFVSEQFTSDQEVITVVKQFNDDIVNNKVLAVVKTLFENFNSYDLEKIYINSKELASVSNALLKDWSKIRNAVLENKIIELGANPPKTKISAVEKEVKNKDFSIAELASYNDKYLDKEGNDKEICSIANVVLEAVGALEIMLAESLPADLKTLENKNKVKGILDAYENLLHLLNYFKVSAVNDVDLAFYGAFEKVYVDISGVMPLYNKVRNYATKKPYSVEKFKLNFAMPTLADGWDKNKERDNGSIILLKDGQYYLGVMNPQNKPVIDNAVCNDAKGYQKMVYKMFPEISKMVTKCSTQLNAVKAHFEDNTNDFVLDDTDKFISDLTITKEIYDLNNVLYDGKKKFQIDYLRNTGDFAGYHKALETWIDFVKEFLSKYRSTAIYDLTTLLPTNYYEKLDVFYSDVNNLCYKIDYENISVEQVNEWVEEGNLYLFKIYNKDFATGSTGKPNLHTMYWNAVFAEENLHDVVVKLNGGAELFYRPKSNMPKVEHRVGEKLVNRKNVNGEPIADSVHKEIYAYANGKISKSELSENAQEELPLAIIKDVKHNITKDKRYLSDKYFFHVPITLNYKANGNPSAFNTKVQAFLKNNPDVNIIGIDRGERNLLYVVVIDQQGNIIDKKQVSYNKVNGYDYYEKLNQREKERIEARQSWGAVGKIKELKEGYLSLVVREIADMMVKYNAIVVMENLNAGFKRVRGGIAEKAVYQKFEKMLIDKLNYLVFKDVEAKEAGGVLNAYQLTDKFDSFEKMGNQSGFLFYVPAAYTSKIDPVTGFANVFSTKHITNTEAKKEFICSFNSLRYDEAKDKFVLECDLNKFKIVANSHIKNWKFIIGGKRIVYNSKNKTYMEKYPCEDLKATLNASGIDFSSSEIINLLKNVPANREYGKLFDETYWAIMNTLQMRNSNALTGEDYIISAVADDNEKVFDSRTCGAELPKDADANGAYHIALKGLYLLQRIDISEEGEKVDLSIKNEEWFKFVQQKEYAR

SEQ ID NO: 5 - protein, 1145 aa, synthetic construct
MCMKITKIDGISHKKYKEKGKLIKNNDTAKDIIEERFNDIEKKTKELFQKTLDFYVKNYEKCKEQNKERREKAKNYFSKVKILVDNKKITICNENTEKMEIEDFNEYDVRSGKYFNVLNKILNGENYTEEDLEVFENDLQKRTGRIKSIKNSLEENKAHFKKESINNNIIYDRVKGNNKKSLFYEYYRISSKHQEYVNNIFEAFDKLYSNSHEAMNNLFSEITKDSKDRNIRKIREAYHEILNKNKTEFGEELYKKIQDNRNNFDKLLEIEPEIKELTKSQIFYKYYIDKVNLDETSIKHCFCHLVEIEVNQLLKNYVYSKRNINKEKLENIFEYCKLKNLIKNKLVNKLNNYIRNCGKYNAYISNNDVVVNSEKISEIRTKEAFLRSIIGVSSSAYFSLRNILNTDNTQDITNKVDKEVDKLYQENKKIELEERLKLFFGNYFDINNQQEIKVFLMNIDKIISSIRHEIIHFKMETNAQNIFDFNNVNLGNTAKNIFSNEINEEKIKFKIFKQLNSANVFDYLSNKDITEYMDKVVFSFTNRNVSFVPSFTKIYNRVQDLANSLEIKKWKIPDKSEGKDAQIYLLKNIYYGKFLDEFLNEENGIFISIKDKIIELNRNQNKRTGFYKLEKFEKIEETNPKKYLEIIQSLYMINIEEIDSEGKNIFLDFIQKIFLKGFFEFIKNNYNYLLELKKIQDKKNIFDSEMSEYIAGEKTLEDIGEINEIIQDIKITEIDKILNQTDKINCFYLLLKLLNYKEITELKGNLEKYQILSKTNVYEKELMLLNIVNLDNNKVKIENFKILAEEIGKFIEKINIEEINKNKKIKTFEELRNFEKGENTGEYYNIYSDDKNIKNIRNLYNIKKYGMLDLLEKISEKTNYCIKKKDLEEYSELRKQLEDEKTNFYKIQEYLHSKYQQKPKKILLKNNKNDYEKYKKSIENIEKYVHLKNKIEFNELNLLQSLLLKILHRLVGFTSIWERDLRFRLIGEFPDELDVEDIFDHRKRYKGTGKGICKKYDRFINTHTEYKNNNKMENVKFADNNPVRNYIAHFNYLPNPKYSILKMMEKLRKLLDYDRKLKNAVMKSIKDILEEYGFKAEFIINSDKEIILNLVKSVEIIHLGKEDLKSRRNSEDLCKLVKAMLEYSK

SEQ ID NO: 6 - protein, 1271 aa, synthetic construct
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKRLEKIQEKKRKEIYQYLSDDAVFSEMFKEKMISGILPDFIRCNEGYSEEEKEEKLKTVALFHRFTSSFNDFFLNRKNVFTKEAIVTAIGYRVVHENAEIFLENMVAFQNIQKSAESQISIIERKNEHYFMEWKLSHIFTADYYMMLMTQKAIEHYNEMCGVVNQQMREYCQKEKKNWNLYRMKRLHKQILSNASTSFKIPEKYENDAEVYESVNSFLQNVMEKTVMERIAVLKNSTDNFDLSKIYITAPYYEKISNYLCGSWNTITDCLTHYYEQQIAGKGARKDQKVKAAVKADKWKSLSEIEQLLKEYARAEEVKRKPEEYIAEIENIVSLKEAHLLEYHPEVNLIENEKYATEIKDVLDNYMELFHWMKWFYIEEAVEKEVNFYGELDDLYEEIKDIVPLYNKVRNYVTQKPYSDTKIKLNFGTPTLANGWSKSKEYDYNAILLQKDGKYYMGIFNPIQKPEKEIIEGHSQPLEGNEYKKMVYYYLPSANKMLPKVLLSKKGMEIYQPSEYIINGYKERRHIKSEEKFDLQFCHDLIDYFKSGIERNSDWKVFGFDFSDTDTYQDISGFYREVEDQGYKIDWTYIKEADIDRLNEEGKLYLFQIYNKDFSEKSTGRENLHTMYLKNLFSEENVREQVLKLNGEAEIFFRKSSVKKPIIHKKGTMLVNRTYMEEVNGNSVRRNIPEKEYQEIYNYKNHRLKGELSTEAKKYLEKAVCHETKKDIVKDYRYSVDKFFIHLPITINYRASGKETLNSVAQRYIAHQNDMHVIGIDRGERNLIYVSVINMQGEIKEQKSFNIINEFNYKEKLKEREQSRGAARRNWKEIGQIKDLKEGYLSGVIHEIAKMMIKYHAIIAMEDLNYGFKRGRFKVERQVYQKFENMLIQKLNYLVFKDRPADEDGGVLRGYQLAYIPDSVKKMGRQCGMIFYVPAAFTSKIDPTTGFVDIFKHKVYTTEQAKREFILSFDEICYDVERQLFRFTFDYANFVTQNVTLARNNWTIYTNGTRAQKEFGNGRMRDKEDYNPKDKMVELLESEGIEFKSGKNLLPALKKVSNAKVFEELQKIVRFTVQLRNSKSEENDVDYDHVISPVLNEEGNFFDSSKYKNKEEKKESLLPVDADANGAYCIALKGLYIMQAIQKNWSEEKALSPDVLRLNNNDWFDYIQNKRYR

SEQ ID NO: 7 - protein, 1229 aa, synthetic construct
MEEKKMSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 8 - protein, 835 aa, synthetic construct
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 9 - protein, 1264 aa, synthetic construct
MENYGGFTGLYPLQKTLKFELRPQGRTMEHLVSSNFFEEDRDRAEKYKIVKKVIDNYHKDFINECLSKRSFDWTPLMKTSEKYYASKEKNGKKKQDLDQKIIPTIENLSEKDRKELELEQKRMRKEIVSVFKEDKRFKYLFSEKLFSILLKDEDYSKEKLTEKEILALKSFNKFSGYFIGLHKNRANFYSEGDESTAIAYRIVNENFPKFLSNLKKYREVCEKYPEIIQDAEQSLAGLNIKMDDIFPMENFNKVMTQDGIDLYNLAIGGKAQALGEKQKGLNEFLNEVNQSYKKGNDRIRMTPLFKQILSERTSYSYILDAFDDNSQLITSINGFFTEVEKDKEGNTFDRAVGLIASYMKYDLSRVYIRKADLNKVSMEIFGSWERLGGLLRIFKSELYGDVNAEKTSKKVDKWLNSGEFSLSDVINAIAGSKSAETFDEYILKMRVARGEIDNALEKIKCINGNFSEDENSKMIIKAILDSVQRLFHLFSSFQVRADFSQDGDFYAEYNEIYEKLFAIVPLYNRVRNYLTKNNLSMKKIKLNFKNPALANGWDLNKEYDNTAVIFLREGKYYLGIMNPSKKKNIKFEEGSGTGPFYKKMAYKLLPDPNKMLPKVFFAKKNINYYNPSDEIVKGYKAGKYKKGENFDIDFCHKLIDFFKESIQKNEDWRAFNYLFSATESYKDISDFYSEVEDQGYRMYFLNVPVANIDEYVEKGDLFLFQIYNKDFASGAKGNKDMHTIYWNAAFSDENLRNVVVKLNGEAELFYRDKSIIEPICHKKGEMLVNRTCFDKTPVPDKIHKELFDYHNGRAKTLSIEAKGYLDRVGVFQASYEIIKDRRYSENKMYFHVPLKLNFKADGKKNLNKMVIEKFLSDKDVHIIGIDRGERNLLYYSVIDRRGNIIDQDSLNIIDGFDYQKKLGQREIERREARQSWNSIGKIKDLKEGYLSKAVHKVSKMVLEYNAIVVLEDLNFGFKRGRFKVEKQVYQKFEKMLIDKLNYLVFKEVLDSRDAGGVLNAYQLTTQLESFNKLGKQSGILFYVPAAYTSKIDPTTGFVSLFNTSRIESDSEKKDFLSGFDSIVYSAKDGGIFAFKFDYRNRNFQREKTDHKNIWTVYTNGDRIKYKGRMKGYEITSPTKRIKDVLSSSGIRYDDGQELRDSIIQSGNKVLINEVYNSFIDTLQMRNSDGEQDYIISPVKNRNGEFFRTDPDRRELPVDADANGAYHIALRGELLMQKIAEDFDPKSDKFTMPKMEHKDWFEFMQTRGD

SEQ ID NO: 10 - protein, 500 aa, synthetic construct
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 11 - protein, 1261 aa, synthetic construct
MIIHNCYIGGSFMKKIDSFTNCYSLSKTLRFKLIPIGATQSNFDLNKMLDEDKKRAENYSKAKSIIDKYHRFFIDKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQPTAISYRCINDNLPKFLDNVRTFKNSDVASILNDNLKILNEDFDGIYGTSAEDVFNVDYFPFVLSQKGIEAYNSILGGYTNSDGSKIKGLNEYINLYNQKNENIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSIESFSLADIQKYADVSESSETNAEVTEWLRNEIKEKCNLAVQGYESSKDLISKPYTESKKLFNNDNAVELIKNALDSVKELENVLRLLLGTGKEESKDENFYGEFLPCYERICEVDSLYDKVRNYMTQKLYKTDKIKLNFQNPQFLGGWDRNKEADYSAVLLRRNSLYYIAIMPSGYKRVFEKIPAPKADETVYEKVIYKLLPGPNKMLPKVFFSKKGIETFNPPKEILEKYELGTHKTGDGFNLDDCHALIDYFKSALDVHSDWSNFGFRFSDTSTYKNIADFYNEVKNQGYKITFCDVPQSYINELVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNLGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 12 - protein, 478 aa, synthetic construct
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 13 - protein, 1229 aa, synthetic construct
MKEQFINRYPLSKTLRFSLIPVGETENNFNKNLLLKKDKQRAENYEKVKCYIDRFHKEYIESVLSKARIEKVNEYANLYWKSNKDDSDIKAMESLENDMRKQISKQLTSTEIYKKRLFGKELICEDLPSFLTDKDERETVECFRSFTTYFKGFNTNRENMYSSDGKSTAIAYRCINDNLPRFLDNVKSFQKVFDNLSDETITKLNTDLYNIFGRNIEDIFSVDYFEFVLTQSGIEIYNSMIGGYTCSDKTKIQGLNECINLYNQQVAKNEKSKKLPLMKPLYKQILSEKDSVSFIPEKFNSDNEVLHAIDDYYTGHIGDFDLLTELLQSLNTYNANGIFVKSGVAITDISNGAFNSWNVLRSAWNEKYEALHPVTSKTKIDKYIEKQDKIYKAIKSFSLFELQSLGNENGNEITDWYISSINESNSKIKEAYLQAQKLLNSDYEKSYNKRLYKNEKATELVKNLLDAIKEFQKLIKPLNGTGKEENKDELFYGKFTSYYDSIADIDRLYDKVRNYITQKPYSKDKIKLNFDNPQLLGGWDKNKESDYRTVLLHKDGLYYLAVMDKSHSKAFVDAPEITSDDKDYYEKMEYKLLPGPNKMLPKVFFASKNIDTFQPSDRILDIRKRESFKKGATFNKAECHEFIDYFKDSIKKHDDWSQFGFKFSPTESYNDISEFYREISDQGYSVRFNKISKNYIDGLVNNGYIYLFQIYNKDFSKYSKGTPNLHTLYFKMLFDERNLSNVVYKLNGEAEMFYREASIGDKEKITHYANQPIKNKNPDNEKKESVFEYDIVKDKRFTKRQFSLHLPITINFKAHGQEFLNYDVRKAVKYKDDNYVIGIDRGERNLIYISVINSNGEIVEQMSLNEIISDNGHKVDYQKLLDTKEKERDKARKNWTSVENIKELKEGYISQVVHKICELVIKYDAVIAMEDLNFGFKRGRFPVEKQVYQKFENMLISKLNLLIDKKAEPTEDGGLLRAYQLTNKFDGVNKAKQNGIIFYVPAWDTSKIDPATGFVNLLKPKCNTSVPEAKKLFETIDDIKYNANTDMFEFYIDYSKFPRCNSDFKKSWTVCTNSSRILTFRNKEKNNKWDNKQIVLTDEFKSLFNEFGIDYKGNLKDSILSISNADFYRRLIKLLSLTLQMRNSITGSTLPEDDYLISPVANKSGEFYDSRNYKGTNAALPCDADANGAYNIARKALWAINVLKDTPDDMLNKAKLSITNAEWLEYTQK

SEQ ID NO: 14 - protein, 1242 aa, synthetic construct
MKEQFVNQYPISKTLRFSLIPIGKTEENFNKNLLLKEDEKKAEEYQKVKGYIDRYHKFFIETALCNINFEGFEEYSLLYYKCSKDDNDLKTMEDIEIKLRKQISKTMTSHKLYKDLFGENMIKTILPNFLDSDEEKNSLEMFRGFYTYFSGFNTNRKNMYTEEAKSTSIAYRCINDNLPKFLDNSKSFEKIKCALNKEELKAKNEEFYEIFQIYATDIFNIDFFNFVLTQPGIDKYNGIIGGYTCSDGTKVQGLNEIINLYNQQIAKDDKSKRLPLLKMLYKQILSDRETVSFIPEKFSSDNEVLESINNYFSKNVSNAIKSLKELFQGFEAYNMNGIFISSGVAITDLSNAVFGDWNAISTAWEKAYFETNPPKKNKSQEKYEEELKANYKKIKSFSLDEIQRLGSIAKSPDSIGSVAEYYKITVTEKIDNITELYDGSKELLNCNYSESYDKKLIKNDTVIEKVKTLLDAVKSLEKLIKPLVGTGKEDKDELFYGTFLPLYTSLSAVDRLYDKVRNYATQKPYSKDKIKLNFNCSSFLSGWATDYSSNGGLIFEKDGLYYLGIVNKKFTTEEIDYLQQNADENPAQRIVYDFQKPDNKNTPRLFIRSKGTNYSPSVKEYNLPVEEIVELYDKRYFTTEYRNKNPELYKASLVKLIDYFKLGFTRHESYRHYDFKWKKSEEYNDISEFYKDVEISCYSLKQEKINYNTLLNFVAENRIYLFQIYNKDFSKYSKGTPNLHTRYFKALFDENNLSDVVFKLNGGSEMFFRKASIKDNEKVVHPANQPIDNKNPDNSKKQSTFDYELIKDKRFTKHQFSIHIPITMNFKARGRDFINNDIRKAIKSEYKPYVIGIDRGERNLIYISVINNNGEIVEQMSLNDIISDNGYKVDYQRLLDRKEKERDNARKSWGTIENIKELKEGYISQVIHKICELVIKYDAVIAMEDLNFGFKRGRFNVEKQVYQKFENMLISKLNYLCDKKSEANSEGGLLKAYQLTNKFDGVNKGKQNGIIFYVPAWLTSKIDPVTGFVDLLHPKYISVEETHSLFEKLDDIRYNFEKDMFEFDIDYSKLPKCNADFKQKWTVCTNADRIMTFRNSEKNNEWDNKRILLSDEFKRLFEEFGIDYCHNLKNKILSISNKDFCYRFIKLFALTMQMRNSITGSTNPEDDYLISPVRDENGVFYDSRNFIGSKAGLPIDADANGAYNIARKGLWAINAIKSTADDMLDKVDLSISNAKWLEYVQK

SEQ ID NO: 15 - protein, 1168 aa, synthetic construct
MKITKIDGILHKKYIKEGKLVKSTSEENKTDERLSELLTIRLDTYIKNPDNASEEENRIRRETLKEFFSNKVLYLKDSILYLKDRREKNQLQNKNYSEEDISEYDLKNKNSFLVLKKILLNEDINSEELEIFRNDFEKKLDKINSLKYSLEENKANYQKINENNIKKVEGKSKRNIFYNYYKDSAKRNDYINNIQEAFDKLYKKEDIENLFFLIENSKKHEKYKIRECYHKIIGRKNDKENFATIIYEEIQNVNNMKELIEKVPNVSELKKSQVFYKYYLNKEKLNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSTAYFSLRNILETENENDITGRIKGKTVKNKKGEEKYISGEIDKLYDNNKQNEVKKNLKMFYSYDFNMNRKKEIEDFFSNIDEAISSIRHGIVHFNLELEGKDIFTFKNIVPSQISKKMFQNEINEKKLKLKIFRQLNSANVFRYLEKYKILNYLNRTRFEFVNKNIPFVPSFTKLYSRIDDLKNSLCIYWKIPKANDNNKTKEITDAQIYLLKNIYYGEFLNYFMSNNGNFFEIIKEIIELNKNDKRNLKTGFYKLQKFENLQEKTPKEYLANIQSFYMIDAGNKDEEEKDAYIDFIQKIFLKGFMTYLANNGRLSLMYIGNDEQINTSLAGKKQEFDKFLKKYEQNNNIEIPHEINEFVREIKLGKILKYTESLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGILNLLEKISDEAKYKISIEELKNYSNKKIEIEKNHTTQENLHRKYARPRKDEKFNDEDYKKYEKTIRNIQQYTHLKNKVEFNELNLLQSLLLRILHRLVGYTSIWERDLRFRLKGEFPENQYIEEIFNFDNSKNVKYKNGQIVEKYISFYKELYKDDMEKISIYSDKKVKELKKEKKDLYIRNYIAHFNYIPNAEVSLLEVLENLRKLLSYDRKLKNAIMKSIVDILKEYGFVVTFKIEKDKKIRIESLKSEEVVHLKKLKLKDNDKKKEPIKTYRNSKELCKLVKVMFEYKMKEKKSEN

SEQ ID NO: 16 - protein, 1168 aa, synthetic construct
MKITKIDGISHKKYIKEGKLVKSTSEENKTDERLSELLTIRLDTYIKNPDNASEEENRIRRENLKEFFSNKVLYLKDGILYLKDRREKNQLQNKNYSEEDISEYDLKNKNSFLVLKKILLNEDINSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIRECYHKIIGRKNDKENFAKIIYEEIQNVNNIKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIAGNRQNEAFLRNIIGVSSVAYFSLRNILETENKDDITGKMRGKTRIDSKTGEEKYIPGEVDQIYYENKQNEVKNKLKMFYGYDFDMDNKKEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISREIIELNKNDKRNLKTGFYKLQKFEDIQEKTPKKYLANIQSLYMINAGNQDEEEKDTYIDFIQKIFLKGFMTYLANNGRLSLMYIGNDEQINTSLAGKKQEFDKFLKKYEQNNNIEIPHEINEFLREIKLGKILKYTESLNMFYLILKLLNHKELTNLKGSLEKYQSANKEETFSDELELINLLNLDNNRVTEDFELEANEIGKFLDFNGNKIKDRKELKKFDTKKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENQYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSVVDILKEYGFVATFKIGADKKIGIQTLESEKIVHLKNLKKKKLMTDRNSEELCKLVKVMFEYKMEEKNLKTKKCKVI

SEQ ID NO: 17 - protein, 1224 aa, synthetic construct
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 18 - protein, 1225 aa, synthetic construct
MKKIDSFVNYYPLSKTLRFSLIPVGKTEDNFNAKLLLEEDEKRAIEYEKVKRYIDRYHKHFIETVLANFHLDDVNEYAELYYKAGKDDKDLKYMEKLEGKMRKSISAAFTKDKKYKEIFGQEIIKNILPEFLENEDEKESVKMFQGFFTYFTGFNDNRKNMYTHEAQTTAISYRCINENLPKFLDNVQSFAKIKESISSDIMNKLDEVCMDLYGVYAQDMFCTDYFSFVLSQSGIDRYNNIIGGYVDDKGVKIQGINEYINLYNQQVDEKNKRLPLMKKLYKQILIEKESISFIPEKFESDNIVINAISDYYHNNVENLFDDFNKLFNEFSEYDDNGIFVTSGLAVTDISNAVFGSWNIISDSWNEEYKDSHPMKKTTNAEKYYEDMKKEYKKNLSFTIAELQRLGEAGCNDECKGDIKEYYKTTVAEKIENIKNAYEISKDLLASDYEKSNDKKLCKNDSAISLLKNLLDSIKDLEKTIKPLLGTGKEENKDDVFYGKFTNLYEMISEIDRLYDKVRNYVTQKPYSKDKIKLNFENPQHLGGWDKNKERDYRSVLLKKEDKYYLAIMDKSNNKAFIDFPDDGECYEKIEYKLLPGPNKMLPKVFFASSNIEYFAPSKKILEIRSRESFKKGDMFNLKDCHEFIDFFKESIKKHEDWSQFGFEFSPTEKYNDISEFYNEVKIQGYSLKYKNVSKKYIDELIECGQLYLFQIYNKDFSVYAKGNPNLHTMYFKMLFDERNLANVVYQLNGGAEMFYRKASIKDSEKIVHHANQPIKNKNADNVKKESVFEYDIIKDKRFTKRQFSIHIPITLNFKAKGQNFINNDVRMALKKADENYVIGIDRGERNLLYICVINSKGEIVEQKSLNEIIGDNGYRVDYHKLLDKKEAERDEARKSWGTIENIKELKEGYLSQIVHEISKLVIKYDAVIAIEDLNSGFKKGRFKVEKQVYQKFENMLCTKLNYLVDKNADANECGGLLKAYQLTNKEDGANRGRQNGIIFSVPAWLTSKIDPVTGFADLLRPKYKSVSESVEFISKIDNIRYNSKEDYFEFDIDYSKFPNSTASYKKKWTVCTYGERIINVRNKEKNNMWDNKTIVLTDEFKKLFADFGVDVSKNIKESVLAIDSKDFYYRFINLLANTLQLRNSEVGNVDVDYLISPVKGVDGSFYDSRLVKEKTLPENADANGAYNIARKALWAIDVLKQTKDEELKNANLSIKNAEWLEYVQK

SEQ ID NO: 19 - protein, 1165 aa, synthetic construct
MKNQNTLPSNPTDILKDKPFWAAFFNLARHNVYLTVNHINKLLDLEKLYNKDKHKEIFEHEDIFNISDDVMNDVNSNGKKRKLDIKKIWANLDTDLTRKYQLRELILKHFPFIQPAIIGAQTKERTTIDKDKRSTSTSNDSLKPTGEGDINDPLSLSNVKSIFFRLLQMLEQLRNYYSHVKHSKSATMPNFDEGLLKSMYNIFIDSVNKVKEDYSSNSVIDPNTSFSHLISKDEQGEIKPCRYSFTSKDGSINASGLLFFVSLFLEKQDSIWMQKKIPGFKKTSENYMKMTNEVFCRNHILLPKMRLETVYDKDWMLLDMLNEVVRCPLSLYKRLAPADQNKFKVPEKSSDNANRQEDDNPFSRILVRHQNRFPYFALRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLERLNKQPEQKPNVAAKNTGTLLRNGQIADWLVKDMMRFQPVKRDKEGNPINCSKANSTEYQMLQRAFAFYTTDSYRLPRYFEQLHLINCDNSHLFLSRFEYDKQPNLIAFYAAYLEAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYTYNFNVGNVSKITEANYLSKEKRENLFKSYQNKFKNNIPAEKTKEYREYKNFSSWKKFERELRLIKNQDILTWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDINLEKYPLTKYQVDSELDLYQKYRIDIFKQTLDLEAQLLDKYSDLNTDNFNQMLSGWSEKEGIPRNIKQDVAFLIGVRNGFSHNQYPDSKRIAFSRIKKFNPKTSSLQESEGLNIAKQMYEEAQQVVNKIKNIESFD

SEQ ID NO: 20 - protein, 1194 aa, synthetic construct
MKVTKIDGISHKKFEDEGKLVKFTGHFNIKNEMKERLEKLKELKLSNYIKNPENVKNKDKNKEKETKSRRENLKKYFSEIILRKKEEKYLLKKTRKFKNITEEINYDDIKKRENQQKIFDVLKELLEQRINENDKEEILNFDSVKLKEAFEEDFIKKELKIKAIEESLEKNRADYRKDYVELENEKYEDVKGQNKRSLVFEYYKNPENREKFKENIKYAFENLYTEENIKNLYSEIKEIFEKVHLKSKVRYFYQNEIIGESEFSEKDEEGISILYKQIINSVEKKEKFIEFLQKVKIKDLTRSQIFYKYFLENEELNDENIKYVFSYFVEIEVNKLLKENVYKTKKFNEGNKYRVKNIFNYDKLKNLVVYKLENKLNNYVRNCGKYNYHMENGDIATSDINMKNRQTEAFLRSILGVSSFGYFSLRNILGVNDDDFYKIEKDERKNENFILKKAKEDFTSKNIFEKVVDKSFEKKGIYQIKENLKMFYGNSFDKVDKDELKKFFVNMLEAITSVRHRIVHYNINTNSENIFDFSNIEVSKLLKNIFEKEIDTRELKLKIFRQLNSAGVFDYWESWVIKKYLENVKFEFVNKNVPFVPSFKKLYDRIDNLKGWNALKLGNNINIPKRKEAKDSQIYLLKNIYYGEFVEKFVNDNKNFEKIVKEIIEINRGAGTNKKTGFYKLEKFETLKANTPTKYLEKLQSLHKISYDKEKIEEDKDVYVDFVQKIFLKGFVNYLKKLDSLKSLNLLNLRKDETITDKKSVHDEKLKLWENSGSNLSKMPEEIYEYVKKIKISNINYNDRMSIFYLLLKLIDYRELTNLRGNLEKYESMNKNKIYSEELTIINLVNLDNNKVRTNFSLEAEDIGKFLKSSITIKNIAQLNNFSKIFADGENVIKHRSFYNIKKYGILDLLEKIVAKADLKITKEEIKKYENLQNELKRNDFYKIQEQIHRNYNQKPFSIKKIENKKDFEKYKKVIEKIQDYTQLKNKIEFNDLNLLQSLIFRILHRLAGYTSLWERDLQFKLKGEFPEDKYIDEIFNSDGNNNQKYKHGGIADKYANFLIEKKEEKSGEILNKKQRKKKIKEDLEIRNYIAHFNYLPNAEKSILEILEELRELLKHDRKLKNAVMKSIKDIFREYGFIVEFTISHTKNGKKIKVCSVKSEKIKHLKNNELITTRNSEDLCELVKIMLEHKELQK

SEQ ID NO: 21 - protein, 1159 aa, synthetic construct
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 22 - protein, 1134 aa, synthetic construct
MNELVKNRCKQTKTICQKLIPIGKTRETIEKYNLMEIDRKIAANKELMNKLFSLIAGKHINDTLSKCTDLDFEPLLTSLSSLNNAKENDRDNLREYYDSVFEEKKTLAEEISSRLTAVKFAGKDFFTKNIPDFLETYEGDDKNEMSELVSLVIENTVTAGYVKKLEKIDRSMEYRLVSGTVVKRVLTDNADIYEKNIEKAKDFDYGVLNIDEASQFTTLVAKDYANYLTADGIAIYNVGIGKINLALNEYCQKNKEYSYNKLALLPLQKMLYGEKLSLFEKLEDFTSDEELINSYNKFAKTVNESGLAEIIKKAVPSYDEIVIKPNKISNYSNSITGHWSLVNRIMKDYLENNGIKNADKYMEKGLTLSEIGDALENKNIKHSDFISNLINDLGHTYTEIKENKESLKKDESVNALIIKKELDMLLSILQNLKVFDIDNEMFDTGFGIEVSKAIEILGYGVPLYNKIRNYITKKPDPKKKFMTKFGSATIGTGITTSVEGSKKATFLKDGDAVFLLLYNTAGCKANNVSVSNLADLINSSLEIENSGKCYQKMIYQTPGDIKKQIPRVFVYKSEDDDLIKDFKAGLHKTDLSFLNGRLIPYLKEAFATHETYKNYTFSYRNSYESYDEFCEHMSEQAYILEWKWIDKKLIDDLVEDGSLLMFRVWNRFMKKKEGKISKHAKIVNELFSDENASNAAIKLLSVFDIFYRDKQIDNPIVHKAGTTLYNKRTKDGEVIVDYTTMVKNKEKRPNVYTTTKKYDIIKDRRYTEEQFEIHLHVNIGKEENKEKLETSKVINEKKNTLVVTRSNEHLLYVVIFDENDNILLKKSLNTVKGMNFKSKLEVVEIQKKENMQSWKTVGSNQALMEGYLSFAIKEIADLVKEYDAILVLEQNSVGKNILNERVYTRFKEMLITNLSLDVDYENKDFYSYTELGGKVASWRDCVTNGICIQVPSAYKYKDPTTSFSTISMYAKTTAEKSKKLKQIKSFKYNRERGLFELVIAKGVGLENNIVCDSFGSRSIIENDISKEVSCTLKIEKYLIDAGIEYNDEKEVLKDLDTAAKTDAVHKAVTLLLKCFNESPDGRYYISPCGEHFTLCDAPEVLSAINYYIRSRYIREQIVEGVKKMEYKKTILLAK

SEQ ID NO: 23 - protein, 1282 aa, synthetic construct
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 24 - protein, 1399 aa, synthetic construct
MNKDIRKNFTDFVGISEIQKTLRFILIPIGKTAQNIDKYNMFEDDEIRHEYYPILKEACDDFYRNHIDQQFENLELDWSKLDEALASEDRDLINETRATYRQVLFNRLKNSVDIKGDSKKNKTLSLESSDKNLGKKKTKNTFQYNFNDLFKAKLIKAILPLYIEYIYEGEKLENAKKALKMYNRFTSRLSNFWQARANIFTDDEISTGSPYRLVNDNFTIFRINNSIYTKNKPFIEEDILEFEKKLKSKKIIKDFESVDDYFTVNAFNKLCTQNGIDKYNSILGGFTTKEREKVKGLNELFNLAQQSINKGKKGEYRKNIRLGKLTKLKKQILAISDSTSFLIEQIEDDQDLYNKIKDFFELLLKEEIENENIFTQYANLQKLIEQADLSKIYINAKHLNKISHQVTGKWDSLNKGIALLLENININEESKEKSEVISNGQTKDISSEAYKRYLQIQSEEKDIERLRTQIYFSLEDLEKALDLVLIDENMDRSDKSILSYVQSPDLNVNFERDLTDLYSRIMKLEENNEKLLANHSAIDLIKEFLDLIMLRYSRWQILFCDSNYELDQTFYPIYDAVMEILSNIIRLYNLARNYLSRKPDRMKKKKINFNNPTLADGWSESKIPDNSSMLFIKDGMYYLGIIKNRAAYSELLEAESLQSSEKKKSENSSYERMNYHFLPDAFRSIPKSSIAMKAVKEHFEINQKTADLLLDTDKFSKPLRITKEIFDMQYVDLHKNKKKYQVDYLRDTGDKKGYRKALNTWLNFCKDFISKYKGRNLFDYSKIKDADHYETVNEFYNDVDKYSYHIFFTSVAETTVEKFISEGKLYLFQLYNKDFSPHSTGKPNLHTIYWRALFSEENLTSKNIKLNGQAEIFFRPKQIETPFTHKKGSILVNRFDVNGNPIPINVYQEIKGFKNNVIKWDDLNKTTQEGLENDQYLYFESEFEIIKDRRYTEDQLFFHVPISFNWDIGSNPKINDLATQYIVNSNDIHIIGIDRGENHLIYYSVIDLQGAIVEQGSLNTITEYTENKFLNNKTNNLRKIPYKDILQQREDERADARIKWHAIDKIKDLKDGYLGQIVHFLAKLIIKYNAIVILEDLNYGFKRGRFKVERQVYQKFEMALMKKLNVLVFKDYDIDEIGGPLKPWQLTRPIDSYERMGRQNGILFYVPAAYTSAVDPVTGFANLFYLNNVKNSEKFHFFSKFESIKYHSDQDMFSFAFDYNNFGTTTRINDLSKSKWQVFTNHERSVWNNKEKNYVTQNLTDLIKKLLRTYNIEFKNNQNVLDSILKIENNTDKENFARELFRLFRLTIQLRNTTVNENNTEITENELDYIISPVKDKNGNFFDSRDELKNLPDNGDANGAYNIARKGLLYIEQLQESIKTGKLPTLSISTLDWFNYIMK

SEQ ID NO: 25 - protein, 1313 aa, synthetic construct
MNKGGYVIMEKMTEKNRWENQFRITKTIKEEIIPTGYTKVNLQRVNMLKREMERNEDLKKMKEICDEYYRNMIDVSLRLEQVRTLGWESLIHKYRMLNKDEKEIKALEKEQEDLRKKISKGFGEKKAWTGEQFIKKILPQYLMDHYTGEELEEKLRIVKKFKGCTMFLSTFFKNRENIFTDKPIHTAVGHRITSENAMLFAANINTYEKMESNVTLEIERLQREFWRRGINISEIFTDAYYVNVLTQKQIEAYNKICGDINQHMNEYCQKQKLKFSEFRMRELKKQILAVVGEHFEIPEKIESTKEVYRELNEYYESLKELHGQFEELKSVQLKYSQIYVQKKGYDRISRYIGGQWDLIQECMKKDCASGMKGTKKNHDAKIEEEVAKVKYQSIEHIQKLVCTYEEDRGHKVTDYVDEFIVSVCDLLGADHIITRDGERIELPLQYEPGTDLLKNDTINQRRLSDIKTILDWHMDMLEWLKTFLVNDLVIKDEEFYMAIEELNERMQCVISVYNRIRNYVTQKGYEPEKIRICFDKGTILTGWTTGDNYQYSGFLLMRNDKYYLGIINTNEKSVRKILDGNEECKDENDYIRVGYHLINDASKQLPRIFVMPKAGKKSEILMKDEQCDYIWDGYCHNKHNESKEFMRELIDYYKRSIMNYDKWEGYCFKFSSTESYDNMQDFYKEVREQSYNISFSYINENVLEQLDKDGKIYLFQVYNKDFAAGSTGTPNLHTMYLQNLFSSQNLELKRLRLGGNAELFYRPGTEKDVTHRKGSILVDRTYVREEKDGIEVRDTVPEKEYLEIYRYLNGKQKGDLSESAKQWLDKVHYREAPCDIIKDKRYAQEKYFLHFSVEINPNAEGQTALNDNVRRWLSEEEDIHVIGIDRGERNLIYVSLMDGKGRIKDQKSYNIVNSGNKEPVDYLAKLKVREKERDEARRNWKAIGKIKDIKTGYLSYVVHEIVEMAVREKAIIVMEDLNYGFKRGRFKVERQVYQKFEEMLINKLNYVVDKQLSVDEPGGLLRGYQLAFIPKDKKSSMRQNGIVFYVPAGYTSKIDPTTGFVNIFKFPQFGKGDDDGNGKDYDKIRAFFGKFDEIRYECDEKVTADNTREVKERYRFDFDYSKFETHLVHMKKTKWTVYAEGERIKRKKVGNYWTSEVISDIALRMSNTLNIAGIEYKDGHNLVNEICALRGKQAGIILNELLEIVRLTVQLRNSTTEGDVDERDEIISPVLNEKYGCFYHSTEYKQQNGDVLPKDADANGAYCIGLKGIYEIRQIKNKWKEDMTKGEGKALNEGMRISHDQWFEFIQNMNKGE

SEQ ID NO: 26 - protein, 1470 aa, synthetic construct
MNNPRGAFGGFTNLYSLSKTLRFELKPYLEIPEGEKGKLFGDDKEYYKNCKTYTEYYLKKANKEYYDNEKVKNTDLQLVNFLHDERIEDAYQVLKPVFDTLHEEFITDSLESAEAKKIDFGNYYGLYEKQKSEQNKDEKKKIDKPLETERGKLRKAFTPIYEAEGKNLKNKAGKEKKDKDILKESGFKVLIEAGILKYIKNNIDEFADKKLKNNEGKEITKKDIETALGAENIEGIFDGFFTYFSGFNQNRENYYSTEEKATAVASRIVDENLSKFCDNILLYRKNENDYLKIFNFLKNKGKDLKLKNSKFGKENEPEFIPAYDMKNDEKSFSVADFVNCLSQGEIEKYNAKIANANYLINLYNQNKDGNSSKLSMFKILYKQIGCGEKKDFIKTIKDNAELKQILEKACEAGKKYFIRGKSEDGGVSNIFDFTDYIQSHENYKGVYWSDKAINTISGKYFANWDTLKNKLGDAKVFNKNTGEDKADVKYKVPQAVMLSELFAVLDDNAGEDWREKGIFFKASLFEGDQNKSEIIKNANRPSQALLKMICDDMESLAKNFIDSGDKILKISDRDYQKDENKQKIKNWLDNALWINQILKYFKVKANKIKGDSIDARIDSGLDMLVFSSDNPAEDYDMIRNYLTQKPQDEINKLKLNFENSSLAGGWDENKEKDNSCIILKDEQDKQYLAVMKYENTKVFEQKNSQLYIADNAAWKKMIYKLVPGASKTLPKVFFSKKWTANRPTPSDIVEIYQKGSFKKENVDFNDKKEKDESRKEKNREKIIAELQKTCWMDIRYNIDGKIESAKYVNKEKLAKLIDFYKENLKKYPSEEESWDRLFAFGFSDTKSYKSIDQFYIEVDKQGYKLEFVTINKARLDEYVRDGKIYLFEIRSRDNNLVNGEEKTSAKNLQTIYWNAAFGGDDNKPKLNGEAEIFYRPAIAENKLNKKKDKNGKEIIDGYRFSKEKFIFHCPITLNFCLKETKINDKLNAALAKPENGQGVYFLGIDRGEKHLAYYSLVNQKGEILEQGTLNLPFLDKNGKSRSIKVEKKSFEKDSNGGIIKDKDGNDKIKIEFVECWNYNDLLDARAGDRDYARKNWTTIGTIKELKDGYISQVVRKIVDLSIYKNTETKEFREMPAFIVLEDLNIGFKRGRQKIEKQVYQKLELALAKKLNFLVDKKADIGEIGSVTKAIQLTPPVNNFGDMENRKQFGNMLYIRADYTSQTDPATGWRKSIYLKSGSESNVKEQIEKSFFDIRYESGDYCFEYRDRHGKMWQLYSSKNGVSLDRFHGERNNSKNVWESEKQPLNEMLDILFDEKRFDKSKSLYEQMFKGGVALTRLPKEINKKDKPAWESLRFVIILIQQIRNTGKNGDDRNGDFIQSPVRDEKTGEHFDSRIYLDKEQKGEKADLPTSGDANGAYNIARKGIVVAEHIKRGFDKLYISDEEWDTWLAGDEIWDKWLKENRESLTKTRK

SEQ ID NO: 27 - protein, 1259 aa, synthetic construct
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKKIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 28 - protein, 477 aa, synthetic construct
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 29 - protein, 1293 aa, synthetic construct
MRTMVTFEDFTKQYQVSKTLRFELIPQGKTLENMKRDGIISVDRQRNEDYQKAKGILDKLYKYILDFTMETVVIDWEALATATEEFRKSKDKKTYEKVQSKIRTALLEHVKKQKVGTEDLFKGMFSSKIITGEVLAAFPEIRLSDEENLILEKFKDFTTYFTGFFENRKNVFTDEALSTSFTYRLVNDNFIKFFDNCIVFKNVVNISPHMAKSLETCASDLGIFPGVSLEEVFSISFYNRLLTQTGIDQFNQLLGGISGKEGEHKQQGLNEIINLAMQQNLEVKEVLKNKAHRFTPLFKQILSDRSTMSFIPDAFADDDEVLSAVDAYRKYLSEKNIGDRAFQLISDMEAYSPELMRIGGKYVSVLSQLLFYSWSEIRDGVKAYKESLITGKKTKKELENIDKEIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAILLRKEGEYFLGIFNSKNKPKLVSDGGAGIGYEKMIYKQFPDFKKMLPKCTISLKDTKAHFQKSDEDFTLQTDKFEKSIVITKQIYDLGTQTVNGKKKFQVDYPRLTGDMEGYRAALKEWIDFGKEFIQAYTSTAIYDTSLFRDSSDYPDLPSFYKDVDNICYKLTFEWIPDAVIDDCIDDGSLYLFKLHNKDFSSGSIGKPNLHTLYWKALFEEENLSDVVVKLNGQAELFYRPKSLTRPVVHEEGEVIINKTTSTGLPVPDDVYVELSKFVRNGKKGNLTDKAKNWLDKVTVRKMPHAITKDRRFTVDKFFFHVPITLNYKADSSPYRFNDFVRQYIKDCSDVKIIGIDRGERNLIYAVVIDGKGNIIEQRSFNTVGTYNYQEKLEQKEKERQTARQDWATVTKIKDLKKGYLSAVVHELSKMIVKYKAIVALENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPYDADANGAFHIAMKGLLLTERIRNDDKLAISNEEWLNYIQEMRG

SEQ ID NO: 30 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 31 - protein, 1286 aa, synthetic construct
MTNFDNFTKKYVNSKTIRLEAIPVGKTLKNIEKMGFIAADRQRDEDYQKAKSVIDHIYKAFMDDCLKDLFLDWDPLYEAVVACWRERSPEGRQALQIMQADYRKKIADRFRNHELYGSLFTKKIFDGSVAQRLPDLEQSAEEKSLLSNFNKFTSYFRDFFDKRKRLFSDDEKHSAIAYRLINENFLKFVANCEAFRRMTERVPELREKLQNTGSLQVYNGLALDEVFSADFYNQLIVQKQIDLYNQLIGGIAGEPGTPNIQGLNATINLALQGDSSLHEKLAGIPHRFNPLYKQILSDVSTLSFVPSAFQSDGEMLAAVRGFKVQLESGRVLQNVRRLFNGLETEADLSRVYVNNSKLAAFSSMFFGRWNLCSDALFAWKKGKQKKITNKKLTEIKKWLKNSDIAIAEIQEAFGEDFPRGKINEKIQAQADALHSQLALPIPENLKALCAKDGLKSMLDTVLGLYRMLQWFIVGDDNEKDSDFYFGLGKILGSLDPVLVLYNRVRNYITKKPYSLTKFRLNFDNSQLLNGWDENNLDTNCASIFIKDGKYYLGISNKNNRPQFDTVATSGKSGYQRMVYKQFANWGRDLPHSTTQMKKVKKHFSASDADYVLDGDKFIRPLIITKEIFDLNNVKFNGKKKLQVDYLRNTGDREGYTHALHTWINFAKDFCACYKSTSIYDISCLRPTDQYDNLMDFYADLGNLSHRIVWQTIPEEAIDNYVEQGQLFLFQLYNKDFAPGADGKPNLHTLYWKAVFNPENLEDVVVKLNGKAELFYRPRSNMDVVRHKVGEKLVNRKLKNGLTLPSRLHEEIYRYVNGTLNKDLSADARSVLPLAVVRDVQHEIIKDRRFTADKFFFHASLTFNFKSSDKPVGFNEDVREYLREHPDTYVVGVDRGERNLIYIVVIDPQGNIVEQRSFNMINGIDYWSLLDQKEKERVEAKQAWETVGKIKDLKCGYLSFLIHEITKIIIKYHAVVILENLSLGFKRVRTGIAEKAVYQQFERMLVTKLGYVVFKDRAGKAPGGVLNAYQLTDNTRTAENTGIQNGFLFYVPAAFTSRVDPATGFFDFYDWGKIKTATDKKNFIAGFNSVRYERSTGDFIVHVGAKNLAVRRVAEDVRTEWDIVIEANVRKMGIDGNSYISGKRIRYRSGEQGHGQYENHLPCQELIRALQQYGIQYETGKDILPAILQQDDAKLTDTVFDVFRLALQMRNTSAETGEDYFNSVVRDRSGRCFDTRRAEAAMPKEADANDAYHIALKGLFVLEKLRKGESIGIKNTEWLRYVQQRHS

SEQ ID NO: 32 - protein, 1272 aa, synthetic construct
MTPIFCNFVVYQIMLFNNNININVKTMNKKHLSDFTNLFPVSKTLRFRLEPQGKTMENIVKAQTIETDEERSHDYEKTKEYIDDYHRQFIDDTLDKFAFKVESTGNNDSLQDYLDAYLSANDNRTKQTEEIQTNLRKAIVSAFKMQPQFNLLFKKEMVKHLLPQFVDTDDKKRIVAKFNDFTTYFTGFFTNRENMYSDEAKSTSIAYRIVNQNLIKFVENMLTFKSHILPILPQEQLATLYDDFKEYLNVASIAEMFELDHFSIVLTQRQIEVYNSVIGGRKDENNKQIKPGLNQYINQHNQAVKDKSARLPLLKPLFNQILSEKAGVSFLPKQFKSASEVVKSLNEAYAELSPVLAAIQDVVTNITDYDCNGIFIKNDLGLTDIAQRFYGNYDAVKRGLRNQYELETPMHNGQKAEKYEEQVAKHLKSIESVSLAQINQVVTDGGDICDYFKAFGATDDGDIQRENLLASINNAHTAISPVLNKENANDNELRKNTMLIKDLLDAIKRLQWFAKPLLGAGDETNKDQVFYGKFEPLYNQLDETISPLYDKVRSYLTKKPYSLDKFKINFEKSNLLGGWDPGADRKYQYNAVILRKDNDFYLGIMRDEATSKRKCIQVLDCNDEGLDENFEKVEYKQIKPSQNMPRCAFAKKECEENADIMELKRKKNAKSYNTNKDDKNALIRHYQRYLDRTYPEFGFVYKDADEYDTVKAFTDSMDSQDYKLSFLQVSETGLNKLVDEGDLYLFKITNKDFSSYAKGRPNLHTIYWRMLFDPKNLANVVYKLEGKAEVFFRRKSLASTTTHKAKQAIKNKSRYNEAVKPQSTFDYDIIKDRRFTADKFEFHVPIKMNFKAAGWNSTRLTNEVREFIKSQGVRHIIGIDRGERHLLYLTMIDMDGNIVKQCSLNAPAQDNARASEVDYHQLLDSKEADRLAARRNWGTIENIKELKQGYLSQVVHLLATMMVDNDAILVLENLNAGFMRGRQKVEKSVYQKFEKMLIDKLNYIVDKGQSPDKPTGALHAVQLTGLYSDFNKSNMKRANVRQCGFVFYIPAWNTSKIDPVTGFVNLFDTHLSSMGEIKAFFSKFDSIRYNQDKGWFEFKFDYSRFTTRAEGCRTQWTVCTYGERIWTHRSKNQNNQFVNDTVNVTQQMLQLLQDCGIDPNGNLKEAIANIDSKKSLETLLHLFKLTVQMRNSVTGSEVDYMISPVADERGHFFDSRESDEHLPANADANGAFNIARKGLMVVRQIMATDDVSKIKFAVSNKDWLRFAQHIDD

SEQ ID NO: 33 - protein, 477 aa, synthetic construct
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 34 - protein, 1281 aa, synthetic construct
MLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 35 - protein, 1367 aa, synthetic construct
MEKSLNDFIGLYSVSKTLRFELKPVSETLENIKKFHFLEEDKKKANDYKDVKKIIDNYHKYFIDDVLKNASFNWKKLEEAIREYNKNKSDDSALVAEQKKLGDAILKLFTSDKRYKALTAATPKELFESILPDWFGEQCNQDLNKAALKTFQKFTSYFTGFQENRKNVYSAEAIPTAVPYRIVNDNFPKFLQNVLIFKTIQEKCPQIIDEVEKELSSYLGKEKLAGIFTLESFNKYLGQGGKENQRGIDFYNQIIGGVVEKEGGINLRGVNQFLNLYWQQHPDFTKEDRRIKMVPLYKQILSDRSSLSFKIESIENDEELKNALLECADKLELKNDEKKSIFEEVCDLFSSVKNLDLSGIYINRKDINSVSRILTGDWSWLQSRMNVYAEEKFTTKAEKARWQKSLDDEGENKSKGFYSLTDLNEVLEYSSENVAETDIRITDYFEHRCRYYVDKETEMFVQGSELVALSLQEMCDDILKKRKAMNTVLENLSSENKLREKTDDVAVIKEYLDAVQELLHRIKPLKVNGVGDSTFYSVYDSIYSALSEVISVYNKTRNYITKKAASPEKYKLNFDNPTLADGWDLNKEQANTSVILRKDGMFYLGIMNPKNKPKFAEKYDCGNESCYEKMIYKQFDATKQIPKCSTQKKEVQKYFLSGATEPYILNDKKSFKSELIITKDIWFMNNHVWDGEKFVPKRDNETRPKKFQIGYFKQTGDFDGYKNALSNWISFCKNFLQSYLSATVYDYNFKNSEEYEGLDEFYNYLNATCYKLNFINIPETEINKMVSEGKLYLFQIYNKDFASGSTGMPNMHTLYWKNLFSDENLKNVCLKLNGEAELFYRPAGIKEPVIHKEGSYLVNRTTEDGESIPEKIYFEIYKNANGKLEKLSDEAQNYISNHEVVIKKAGHEIIKDRHYTEPKFLFHVPLTINFKASGNSYSINENVRKFLKNNPDVNIIGLDRGERHLIYLSLINQKGEIIKQFTFNEVERNKNGRTIKVNYHEKLDQREKERDAARKSWQAIGKIAELKEGYLSAVIHQLTKLMVEYNAVVVMEDLNFGFKRGRFHVEKQVYQKFEHILIDKSNYLVFKDRGLNEPGGVLNGYQIAGQFESFQKLGKQSGMLFYVPAGYTSKIDPKTGFVSMMNFKDLTNVHKKRDFFSKFDNIHYDEANGSFVFTFDYKKFDGKAKEEMKLTKWSVYSRDKRIVYFAKTKSYEDVLPTEKLQKIFESNGIDYKSGNNIQDSVMAIGADLKEGAKPSKEISDFWDGLLSNFKLILQMRNSNARTGEDYIISPVMADDGTFFDSREEFKKGEDAKLPLDADANGAYHIALKGLSLINKINLSKDEELKKFDMKISNADWFKFAQEKNYAK

SEQ ID NO: 36 - protein, 809 aa, synthetic construct
MIKNPSNRHSLPKVIISEVDHEKILEFKIKYEKLARLDRFEVKAMHYEGKEIVFDEVLVNGGLIEVEYQDDNKTLFVKVGEKSYSIRGKKVGGKQRLLEDRVSKTKVQLELSDGVVDNKGNLRKSRTERELIVADNIKLYSQIVGREVTTTKEIYLVKRFLAYRSDLLFYYSFVDNFFKVAGNEKELWKINFDDATSAQFMGYIPFMVNDNLKNDNAYLKDYVRNDVQIKDDLKKVQTIFSALRHTLLHFNYEFFEKLFNGEDVGFDFDIGFLNLLIENIDKLNIDAKKEFIDNEKIRLFGENLSLAKVYRLYSDICVNRVGFNKFINSMLIKDGVENQVLKAEFNRKFGGNAYTIDIHSNQEYKRIYNEHKKLVIKVSTLKDGQAIRRGNKKISELKEQMKSMTKKNSLARLECKMRLAFGFLYGEYNNYKAFKNNFDTNIKNSQFDVNDVEKSKAYFLSTYERRKPRTREKLEKVAKDIESLELKTVIANDTLLKFILLMFVFMPQELKGDFLGFVKKYYHDVHSIDDDTKEQEEDVVEAMSTSLKLKILGRNIRSLTLFKYALSSQVNYNSTDNIFYVEGNRYGKIYKKLGISHNQEEFDKTLVVPLLRYYSSLFKLMNDFEIYSLAKANPTAVSLQELVDDETSPYKQGNYFNFNKMLRDIYGLTSDEIKSGQVVFMRNKIAHFDTEVLLSKPLLGQTKMNLQRKDIVSFIEARGDIKELLGYDAINDFRMKVIHLRTKMRVYSDKLQTMMDLLRNAKTPNDFYNVYKVKGVESINKHLLEVLAQTAEERTVEKQIRDGNEKYDL

SEQ ID NO: 37 - protein, 839 aa, synthetic construct
LNSIEKIKKPSNRNSIPSIIISDYDENKIKEIKVKYLKLARLDKITIQDMEIRDNIVEFKKILLNGIEHTIKDNQKIEFDNYEITAYVRASKQRRDGKITQAKYVVTITDKYLRDNEKEKRFKSTERELPNDTLLMRYKQISGFDTLTSKDIYKIKRYIDFKNEMLFYFQFIEEFFSPLLPKGTNFYSLNIEQNKDKVVKYIVYRLNDDFKNQSLNQFIKKTDTIKYDFLKIQKILSDFRHALAHFDFDFIQKFFDDELDKNRFDISTISLIKTMLQEKEEKYYQEKNNYIEDSDTLTLFDEKESNFSKIHNFYIKISQKKPAFNKLINSFLSKDGVPNEELKSYLATKKIDFFEDIHSNKEYKKIYIKHKNLVVEKQKEESQEKPNGQKLKNYNDELQKLKDEMNKITKQNSLNRLEVKLRLAFGFIANEYNYNFKNFNDKFTLDVKKEQKIKVFKNSSNEKLKEYFESTFIEKRFFHFCVKFFNKKTKKEETKQKNIFNLIENETLEELVKESPLLQIITLLYLFIPKELQGEFVGFILKIYHHTKNITNDTKEDEKSIEDTQNSFSLKLKILAKNLRGLQLFNYSLSHNTLYNTKEHFFYEKGNRWQSVYKSLEISHNQDEFDIHLVIPVIKYYINLNKLIGDFEIYALLTYADKNSITEKLSDITKRDDLKFRGYYNFSTLLFKTFMINTNYEQNQKSTQYIKQTRNDIAHQNIENMLKAFENNEIFAQREEIVNYLQKEHKMQEILHYNPINDFTMKTVQYLKSLNIHSQKESKIADIHKKESLVPNDYYLIYKLKVIELLKQKVIEAIGETKDEEKIKNAIAKEEQIKKGYNK

SEQ ID NO: 38 - protein, 840 aa, synthetic construct
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRNKQTSKISRIKKIQDTYYRGKDVFILDNNIEILDKKQTKDKFIVTLNDITNNKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYNYANDRKKVLNDLRNIQYVFKEFRHKLAHFDYNFLDNFFSNSVEEKYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGKKINSLNQKINKLKIDMKNITKPNALNRLIYRLRVAFGFIYKEYATINNFNKSFLQDTKTKRFENISQQDIKSYLDISYQDKGKFFVKSKKTFKNKTTVKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKNILNMKNIQKINRYILDIL

SEQ ID NO: 39 - protein, 840 aa, synthetic construct
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRDKQTSKISRIKKIQDTYYRGKDVFILDNNIEILDKKQTKDKFIVTLNDITNDKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYDYADDREKVLNDLKNIQYVFTEFRHKLAHFDYNFLDNFFSNSVTDQYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGQKINSLNQKINKLKIEMKNITKPNALNRLIYRLRVAFGFIYKEYATINNFNKSFLQDTKIKRFENISQQDIKNYLDISYQDKGKFFVKSKKTFKNKTTIKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKKILNMKNIQKINRYILDIL

SEQ ID NO: 40 - protein, 849 aa, synthetic construct
MSQLKNPSNKNSLPRIIISDFNEIKINEIKIKYHKLDRLDKIIVKEMEIINNKIFFKKILFNNQIKDINSENIELENYILAGEVKPSNTKIILNRDGKEKSFIVYDGFTFKYKPNDKRISETKTNAKYILTIKDKTRHRESSTQRDILKSSIIETYKQISGFENITSKDIYTIKRYIDFKNEMMFYYTFIDDFFFPITGKNKQDKKNNFYNYKIKENAKKFISLINYRINDDFKNKNGILYDYLSNKEEIIINDFIHIQTILKDVRHAIAHFNFDFIQKLFDNEQAFNSKFDGIEILNILFNQKQEKYFEAQTNYIEEETIKILDEKELSFKKLHSFYSQICQKKPAFNKLINSFIIQDGIENKELKDYISQKYNSKFDYYLDIHTCKIYKDIYNQHKKFVADKQFLENQKTDGQKIKKLNDQINQLKTKMNNLTKKNSLKRLEIKFRLAFGFIFTEYQTFKNFNERFIEDIKANKYSTKIELLDYGKIKEYISITHEEKRFFNYKTFNKKTNKNINKTIFQSLEKETFENLVKNDNLIKMMFLFQLLLPRELKGEFLGFILKIYHDLKNIDNDTKPDEKSLSELNISTALKLKILVKNIRQINLFNYTISNNTKYEEKEKRFYEEGNQWKDIYKKLYISHDFDIFDIHLIIPIIKYNINLYKLIGDFEVYLLLKYLERNTNYKTLDKLIEAEELKYKGYYNFTTLLSKAINIALNDKEYHNITHLRNNTSHQDIQNIISSFKNNKLLEQRENIIELISKESLKKKLHFDPINDFTMKTLQLLKSLEVHSDKSEKIENLLKKEPLLPNDVYLLYKLKGIEFIKKELISNIGITKYEEKIQEKIAKGVEK

SEQ ID NO: 41 - protein, 822 aa, synthetic construct
MVKNPANRHALPKVIISEVDNNNILEFKIKYEKLARLDKVEVKSMHFDNNKQVVFDEVVINGGLIEPTYEDKHKKLVVTAGEKSYSIVGQKVGGKPRLLEDRVSKTKVQLELTNYVEDKEGKKRVSKTERELIVADNIELYSQIVGREVKTTKEIYLIKRFLEYRSDLLFYYGFVDNFFKVAGNGKELWKIDFTNSDSLHLIEYFKFSINDNLKNDENYLKNYVSDNTKIENDLVKCQNNFNSLRHALMHFDYDFFEKLFNGEDVGFDFDIEFLNIMIDKVDKLNIDTKKEFIDDEEVTLFGEALSLKKLYGLFSHIAINRVAFNKLINSFIIEDGIENKELKDFFNNKKESQAYEIDIHSNAEYKALYVQHKKLVMATSAMTDGDEIAKKNQEISDLKEKMKVITKENSLARLEHKLRLAFGFIYTEYKDYKTFKKHFDQDIKGAKYKGLNVEKLKEYYETTLKNSKPKTDEKLEDVAKKIDKLSLKELIDDDTLLKFVLLLFIFMPQELKGDFLGFIKKYYHDKKHIDQDTKDKDTEIEELSTGLKLKVLDKNIRSLSILKHSFSFQVKYNRKDKNFYEDGNLHGKFYKKLSISHNQEEFNKSVYAPLFRYYSALYKLINDFEIYALAQHVENHETLADQVNKSQFIQKSYFNFRKLLDNTDSISQSSSYNTLIVMRNDISHLSYEPLFNYPLDERKSYKKKTQKGVKTFHVELLYISRAKIIELISLQTDMKKLLGYDAVNDFNMKVVHLRKRLSVYANKEESIRKMQADAKTPNDFYNIYKVKGVESINQHLLKVIGVTEAEKSIEKQINEGNKKHNT

SEQ ID NO: 42 - protein, 806 aa, synthetic construct
MTKKPSNRNSLPKVIINKVDESSILEFKIKYEKLARLDRFEVRSMRYDGDGRIIFDEVVANAGLLDVDYEDDNRTIVVKIENKAYNIYGKKVGGEKRLNGKISKAKVQLILTDSIRKNANDTHRHSLTERELINKNEVDLYSKIAEREISTTKDIYLVKRFLAYRSDLLLYYAFINHYVRVNGNKKEFWKTEIDDKIIDYFIYTINDTLKNKEGYLEKYIVDRDQIKKDLEKIKQIFSHLRHKLMHYDFRFFTDLFDGKDVDIKVDNSIQKISELLDIEFLNIVIDKLEKLNIDAKKEFIDDEKITLFGQEIELKKLYSLYAHTSINRVAFNKLINSFLIKDGVENKELKEYFNAHNQGKESYYIDIHQNQEYKKLYIEHKNLVAKLSATTDGKEIAKINRELADKKEQMKQITKANSLKRLEYKLRLAFGFIYTEYKDYERFKNSFDTDTKKKKFDAIDNAKIIEYFEATNKAKKIEKLEEILKGIDKLSLKTLIQDDILLKFLLLFFTFLPQEIKGEFLGFIKKYYHDITSLDEDTKDKDDEITELPRSLKLKIFSKNIRKLSILKHSLSYQIKYNKKESSYYEAGNVFNKMFKKQAISHNLEEFGKSIYLPMLKYYSALYKLINDFEIYALYKDMDTSETLSQQVDKQEYKRNEYFNFETLLRKKFGNDIEKVLVTYRNKIAHLDFNFLYDKPINKFISLYKSREKIVNYIKNHDIQAVLKYDAVNDFVMKVIQLRTKLKVYADKEQTIESMIQNTQNPNGFYNIYKVKAVENINRHLLKVIGYTESEKAVEEKIRAGNTSKS

SEQ ID NO: 43 - protein, 832 aa, synthetic construct
MIKNPSNRYALPKVIISKIDNQNILEFKIKYKKLSKLDIVKVKSMHYDDRAIIFDEVIVNDGLIDVEYRDNHKTIFVKVGNKSYSISGQKVGGKERLLENRVSKTKVQLELKDKATNRVSKTERELIVDDNIKIYSQIVGRDVKTTKDIYLIKRFLAYRSDLLFYYGFVNNFFHVANNRSEFWKIDFNDSNNSKLIEYFKFTINDHLKNDENYLKDYISDNEKLKNDLIKVKNSFEKIRHALMHFDYDFFVKLFNGEDVGLELDIEFLDIMIDKLDKLNIDTKKEFIDDEKITIFGEELSLAKLYRFYAHTAINRVAFNKLINSFIIENGVENQSLKEYFNQQAGGIAYEIDIHQNREYKNLYNEHKKLVSRVLSISDGQEIAILNQKIAKLKDQMKQITKANSIKRLEYKLRLALGFIYTEYENYEEFKNNFDTDIKNGRFTPKDNDGNKRAFDSRELEQLKGYYEATIQTQKPKTDEKIEEVSKKIDRLSLKSLIADDILLKFILLMFTFMPQELKGEFLGFIKKYYHDTKHIDQDTISDSDDTIETLSIGLKLKILDKNIRSLSILKHSLSFQTKYNKKDRNYYEDGNIHGKFFKKLGISHNQEEFNKSVYAPLFRYYSALYKLINDFEIYTLSLHIVGSETLTDQVNKSQFLSGRYFNFRKLLTQSYHINNNSTHSTIFNAVINMRNDISHLSYEPLFDCPLNGKKSYKRKIRNQFKTINIKPLVESRKIIIDFITLQTDMQKVLGYDAVNDFTMKIVQLRTRLKAYANKEQTIQKMITEAKTPNDFYNIYKVQGVEEINKYLLEVIGETQAEKEIREKIERGNIANF

SEQ ID NO: 44 - protein, 1281 aa, synthetic construct
MKKSIFDQFVNQYALSKTLRFELKPVGETGRMLEEAKVFAKDETIKKKYEATKPFFNKLHREFVEEALNEVELAGLPEYFEIFKYWKRYKKKFEKDLQKKEKELRKSVVGFFNAQAKEWAKKYETLGVKKKDVGLLFEENVFAILKERYGNEEGSQIVDESTGKDVSIFDSWKGFTGYFIKFQETRKNFYKDDGTATALATRIIDQNLKRFCDNLLIFESIRDKIDFSEVEQTMGNSIDKVFSVIFYSSCLLQEGIDFYNCVLGGETLPNGEKRQGINELINLYRQKTSEKVPFLKLLDKQILSEKEKFMDEIENDEALLDTLKIFRKSAEEKTTLLKNIFGDFVMNQGKYDLAQIYISRESLNTISRKWTSETDIFEDSLYEVLKKSKIVSASVKKKDGGYAFPEFIALIYVKSALEQIPTEKFWKERYYKNIGDVLNKGFLNGKEGVWLQFLLIFDFEFNSLFEREIIDENGDKKVAGYNLFAKGFDDLLNNFKYDQKAKVVIKDFADEVLHIYQMGKYFAIEKKRSWLADYDIDSFYTDPEKGYLKFYENAYEEIIQVYNKLRNYLTKKPYSEDKWKLNFENPTLADGWDKNKEADNSTVILKKDGRYYLGLMARGRNKLFDDRNLPKILEGVENGKYEKVVYKYFPDQAKMFPKVCFSTKGLEFFQPSEEVITIYKNSEFKKGYTFNVRSMQRLIDFYKDCLVRYEGWQCYDFRNLRKTEDYRKNIEEFFSDVAMDGYKISFQDVSESYIKEKNQNGDLYLFEIKNKDWNEGANGKKNLHTIYFESLFSADNIAMNFPVKLNGQAEIFYRPRTEGLEKERIITKKGNVLEKGDKAFHKRRYTENKVFFHVPITLNRTKKNPFQFNAKINDFLAKNSDINVIGVDRGEKQLAYFSVISQRGKILDRGSLNVINGVNYAEKLEEKARGREQARKDWQQIEGIKDLKKGYISQVVRKLADLAIQYNAIIVFEDLNMRFKQIRGGIEKSVYQQLEKALIDKLTFLVEKEEKDVEKAGHLLKAYQLAAPFETFQKMGKQTGIVFYTQAAYTSRIDPVTGWRPHLYLKYSSAEKAKADLLKFKKIKFVDGRFEFTYDIKSFREQKEHPKATVWTVCSCVERFRWNRYLNSNKGGYDHYSDVTKFLVELFQEYGIDFERGDIVGQIEVLETKGNEKFFKNFVFFFNLICQIRNTNASELAKKDGKDDFILSPVEPFFDSRNSEKFGEDLPKNGDDNGAFNIARKGLVIMDKITKFADENGGCEKMKWGDLYVSNVEWDNFVANK

SEQ ID NO: 45 - protein, 1280 aa, synthetic construct
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPFSKDKIKINFDAPTLLNGWDLNKESDNKSIILRKDGNFYLAIMHPKHTKVFDCYSASEAAGKCYEKMNYKLLSGANKMLPKVFFSKKGIETFSPPQEILDLYKNNEHKKGATFKLESCHKLIDFFKRNIPKYKVHPTDNFGWDVFGFHFSPTSSYGDLSGFYREVEAQGYKLWFSDVSEAYINKCVEEGKLFLFQIYNKDFSPNSTGKPNLHTLYWKGLFEPENLKDVVLKLNGEAEIFYRKHSIKHEDKTIHRAKDPIANKNADNPKKQSVFDYDIIKDKRYTQDKFFFHVPISLNFKSQGVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 46 - protein, 832 aa, synthetic construct; identical to SEQ ID NO: 43

SEQ ID NO: 47 - protein, 809 aa, synthetic construct; identical to SEQ ID NO: 36

SEQ ID NO: 48 - protein, 1285 aa, synthetic construct
MEEKMLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 49 - protein, 1367 aa, synthetic construct; identical to SEQ ID NO: 35

SEQ ID NO: 50 - protein, 1366 aa, synthetic construct
MNTQKKEFNPKSFKDFTNLYSLNKTLRFSLTPNKKTAEILEFNKQKEVKCFSNDRKIAGAYQEIKKYLNKLHQEFIQEAMKFFAFSEEELKGFEKEYLNLLNFTDKDNFKKKNKIRNEYEQERKILTIKIATYFSKFKSEKYQSFNLANITGKKVFSILEQKYKEDKKTLKIIHIFKYKPTKDEKKEGEAVNFSTYLTGFNENRKNFYKSEDKAGQFATRTIDNLAQFIKNKKLFEDKYQKNYSKIGILDEQIKIFNLDYFNNLFLQEGLDEYNGILGNNKGEENKSNEGINQKINIFKQKEKARLKKEKENFNKSDFPLFKELYKQIGSIRKENDVYVEIKTDKELVEELNNFPKNVENYLKDIQSFYKTFFEKLQNEEYELDKIYLPKSVGTYFSYIAFSDWNKLAFIYNKRYKNEKIKIVEGGDVNVQYRSLEVLKNRIDELKDEDNLNFNKFFIDKLKFNEAKKENNWQNFWFCIEYYINSQFIGGEKNILNKEKNEYEILPFGSLKELKEKYFEAVKKYKEKMVDTESGLTDDEEKEIKETLKNYLDRIKEIERIAKYFDLKKSFEEIKQEDLDSNFYGEYQKVVDKTNELKIYQYYSEFRNYLTQNNSVEEKIKLNFNSGLLLDGWDLNKEKVKFSIIFQENGKYYLGIINKEKDKTILDKDKHPEIFTKNSDFRKMEYKLFPSPSKMLPKISFSETAKKGDEDVGWSEEIQKIKDEFAEFQEYKKKSKDNWKDEFNRGKLNKLIDYYKQVLEKHSEGYMNTYNFELKDSSKYKNLGEFNDDIARQNYKVKFVGIDKNYIDEKVANGELFLFQIYNKDFSEDKKEGSTNNLETIYFKELFSKENLENPVFKLSGGAEMFFRNKIEKKKEKKKLDKDGKPMISKKGEKVVDKRRFSENKILFHLPIEINYGKGKMPNFNKKINEYISKNPENIKIIGIDRGEKHLLYYSIIDQNGNNIESMSLNAVDEFGNFVNPEKLEEYEIDNNGKKERRWKYIVNDKEIKVTNYQRKLDELEKERQKSRQSWQNINKIKNLKKGYISFVVKKIVDLAIENNAIIILEDLNFGFKSFRQKIEKNVYQQFEKALIDKLGFVVDKQKQNQRFAPQLSAPFESFQKIGKQTGIVYYVLANNTSKVCPSCQWIKNFYLKYEKKNTIFNLQKNQKLKVFFEQEKNRFRFEYQMSKEYISVYSDVDRQRYDKTKNQNKGGYLEYKNSNQKEIIDKDGVIQKQSITLQLKELFKENHIDLEKEILKQLDNKKEKNSGYTGVYNKFIYLFNLILQIRNAISFREKDYIQCPSCHFDTRKENYLKINDGDGNGAYNIALRGLYLLKGKNGIINNLEKIKLIFSNNDYFQWAKKLKNKK

SEQ ID NO: 51 - protein, 1275 aa, synthetic construct
MQNKQSFADFTNLYSLSKTLRFELKPIGQTQAMLDENKIFEVDENRKKAYDKTKPYFDRLHREFINESLSNAQLKGISEYFETFKQFRSNQNNKDLKELINKQQKFLRHQIVTLFDENGKHWATTKYAHLKIKKKNLDILFDEQVFYILKERYGSEKETQLVDKETGAVTSIFDNWKGFTGYFTKFFETRKNFYKSDGTSTALATRIIDQNLNRFFDNLETFHKIKDKIDVKEVEIFFKLKADNVFSIDFYNQCLLQNGIDKYNDFLGGQTLENGEKQKGINEIINKYRQDNKDQKLPFLKKLDKQILSEKDRFINEIESKEEFFQVLTEFYQSATVKVTIIKTLLNDFVHNTDKYKLEKIYLTKEAFNTIANKWTDETQIFEDNLDLVLKNKKITAKQDFIPLAYIKEALEVIEKDRKFFKDRYYNDPQIGFFPDQSYWEQFLAILNFEFMTHFQRVAKDKITGKKIELGYFVFEKRIKELLDSDPSLNSQSKIIIKEFADEVLHIFQMAKYFALEKKREWKGDYYQLDDQFYNHIDYGFKDQFYENAYEKIVQPYNKIRNYLTKKPYSDVKWKLNFGNPTLANGWDKNKEADNTAVILKKDGNYYLGVMKKGKNKIFSDQNKEKYKAYNSAYYEKLVYKLFPDPSKMFPKVCFSKKGLNFFQPSEEILRIYKNNEFKKGNTFSISSMQKLIAFYIDCLGLYEGWKHYEFKNIKDVRQYKENIGEFYADVAESGYKLWFEKISEEYITQKNQLGELFLFQIYNKDFAKKTTGRKNLHTIYFEELFSQTNIDNNFPFKLNGQAELFYRPKSLEKIEEKRNFKRSIVNKKRYTQNKIFFHVPITLNRTSENIGRFNVRVNNFLANNSNVNIVGVDRGEKNLAYYSIIKQNGEVLKSGSLNIINGVDYHALLTDRAQRREQERRNWQDVESIKDLKRGYISQVVHELVSLAIKYNAIIVMEDLNMRFKQIRGGIEKSTYQQLEKALIEKLNFLVNKEETDSNQAGNLLNAYQLTAPFKTFKDMGKQTGIIFYTQASYTSKIDPLTGWRPNIYLRYSNAKQAKADILMFTNIYFSEKKDRFEFTYDLEKIDDKRKDLPIKTEWTVCSNVERFSWEKSLNNNKGGYVHYPIQDSNGEESITSKLKKLFMDFGIDLTDIKTQIESLDTNKKDNANFFRKFIFYFQLICQIRNTQVNKSDDGNDFIFSPVEPFFDSRFADKFRKNLPKNGDENGAYNIARKGLIILHKISDYFVKEGSTDKISWKDLSISQTEWDNFTTDK

SEQ ID NO: 52 - protein, 1368 aa, synthetic construct
MKKEKEFKSFGDFTNLYEISKTLRFELKPVENTQTMLDEADVFGKDKVIKDKYTKTKPFIDKLHREFVDESLKDVSLSGLKKYSEVLENWKKNKKDKDIVKELKKEEERLRKEVVEFFDNTAKKWANEKYKELGLKKKDIGILFEESVFDLLKEKYGEEQDSFLKEEKGDFLKNEKGEKVSIFDEWKGFVGYFTKFQETRKNFYKNDGTETALATRIIDQNLKRFCDNIDDFKKIKNKIDFSEVEKNFNKTADVFSLDFYNQCLLQKGIDSYNEFIGGKTLENGKKLKGVNELVNEYRQKNKNEKVSFLKLLDKQILSEKEKLSFGIENDEQLLVVLNSFYETAEEKTKILRTLFGDFVEHNENYDLDKTYISKVAFNTISHKWTNETHKFEELLYGAMKEDKPIGLNYDKKEDSYKFPDFIALGYLKKCLNNLDCDTKFWKEKYYENNADKKDKDKGFLTGGQNAWDQFLQIFIFEFNQLFNSEAFDNKGKEIKIGYDNFRKDFEEIINQKDFKNDENLKIAIKNFADSVLWIYQMAKYFAIEKKRGWDDDFELSEFYTNPSNGYSLFYDRAYEEIVQKYNDLRNYLTKKPYKEDKWKLNFENPTLANGFDKNKESDNSTVILRKKRKYYLGLMKKGNNKIFEDRNKAEFIRNIESGAYEKMAYKYLPDVAKMIPKCSTQLNEAKNHFRNSADDLEIKKSFSNPLKITKRIFDLNNIQYDKTNVSKKISGDNKGIKIFQKEYYKISGDFDVYKSALNDWIDFCKDFLSKYDSTKDFDFSILRKTKDYKSLDEFYVDVAKITYKISFTPVSESYIDQKNKNGELYLFEIYNQDFAKGKMGAKNLHTLYFENVFSPENISKNFPIKLNGNAELFFRPKSIESKKEKRNFVREIVNKKRYSEDKIFFHCPITLNRETGSIYRFNNYVNNFLSENNINIIGVDRGEKHLAYYSVIDKNGVKIGGGSFNEINKVDYAKKLEERAGEREQSRKDWQVVEGIKDLKKGYISQVVRELADLAIKHNAIIVLEDLNMRFKQIRGGIEKSIYQQLEKALIDKLSFLVEKGEKDPNQAGHILKAYQLAAPFTSFKDMGKQTGIVFYTQASYTSKTCPNCGFRKNNNKFYFENNIGKAQDALKKLKTFEYDSENKCFGLSYCLSDFANKEEVEKNKNKKRNNAPYSDIEKKDCFELSTKDAVRYRWHDKNTERGKTFFEGESVYEEKEEKEIGQTKRGLVKEYDISKCLIGLFEKTGLDYKQNLLDKINSGKFDGTFYKNLFNYLNLLFEIRNSISGTEIDYISCPECQFHTDKSKTIKNGDDNGSYNIARKGMIILDKIKQFKKENGSLDKMGWGELFIDLEEWDKFAQKKNNNIIDK

SEQ ID NO: 53 - protein, 1257 aa, synthetic construct
MKYTDFTGIYSVSKTLRFELIPQGSTVENMKREGILNNDMHRADSYKEMKKLIDEYHKAFIERCLSDFSLKYDDTGKHDSLEEYFFYYEQKRNDKTKKIFEDIQVALRKQISKRFTGDTAFKRLFKKELIKEDLPSFVKNDPVKTELIKEFSDFTTYFQEFHKNRKNMYTSDAKSTAIAYRIINENLPKFIDNINAFDIVAKVPEMQEHFKTIADELRSHLQVGNDIDKMFNLQFFNKVLTQSQLDVYNAVIGGKSEGNKKIQGINEYINLYNQQHKKARLPMLKLLYKQILSDRVAISWLQDEFDNDQDMLDTIEAFYNKLNSNETGVLGEGKLKQILMGLDGYNLDGVFMRNDLQLSEVSQRLCGGWNIIKDAMTSDLKRSVQKKKKETDADFEERVSKLFSAQNSFSIAYINQCLGQAGIRCKIQDYFACLGAKEGENEAETTPDIFDQIAEAYHGAAPILNARPSSHNLAQDIEKVKAIKALLDALKRLQRFVKPLLGRGDEGDKDNFFYGDFMPIWEVLDQLTPLYNKVRNRMTRKPYSQEKIKLNFENSTLLNGWDLNKEHDNTSVILRREGLYYLGIMNKNYNKIFDANNVETIGDCYEKMIYKLLPGPNKMLPKVFFSKSRVQEFSPSKKILEIWESKSFKKGDNFNLDDCHALIDFYKDSIAKHPDWNKFNFKFSDTQSYTNISDFYRDVNQQGYSLSFTKVSVDYVNRMVDEGKLYLFQIYNKDFSPQSKGTPNMHTLYWRMLFDERNLHNVIYKLNGEAEVFYRKASLRCDRPTHPAHQPITCKNENDSKRVCVFDYDIIKNRRYTVDKFMFHVPITINYKCTGSDNINQQVCDYLRSAGDDTHIIGIDRGERNLLYLVIIDQHGTIKEQFSLNEIVNEYKGNTYCTNYHSLLEEKEAGNKKARQDWQTIESIKELKEGYLSQVIHKISMLMQRYHAIVVLEDLNGSFMRSRQKVEKQVYQKFEHMLINKLNYLVNKQYDATEPGGLLHALQLTSRMDSFKKLGKQSGFLFYIPAWNTSKIDPVTGFVNLFDTRYCNEAKAKEFFEKFDDISYNDERDWFEFSFDYRHFTNKPTGTRTQWTLCTQGTRVRTFRNPEKSNHWDNEEFDLTQAFKDLFNKYGIDIASGLKARIVNGQLTKETSAVKDFYESLLKLLKLTLQMRNSVTGTDIDYLVSPVADKDGIFFDSRTCGSLLPANADANGAFNIARKGLMLLRQIQQSSIDAEKIQLAPIKNEDWLEFAQEKPYL

SEQ ID NO: 54 - protein, 766 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 55 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 56 - protein, 766 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 57 - protein, 718 aa, synthetic construct
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 58 - protein, 757 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 59 - protein, 761 aa, synthetic construct
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 60 - protein, 708 aa, synthetic construct
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 61 - protein, 661 aa, synthetic construct
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 62 - protein, 845 aa, synthetic construct
MIKKPSNRHALPKVIISKVDNQNILEFKIKYKKLSRLDRVEIKTMHYDDRAIVFDEVIINGGLIDVEYRDNHKTIFVKVGDKSYSISGQKVGGKERLLENRISQTKVQLELKDEATNRVSKTERELIVDDNIKLYSQIVGRDVKTTKDIYLIKRFLGYRSDLLFYYGFVNNFFHVANNRPEFWKIDFNDNRNSKLIEYFIFTINDHLKNDENYLKDYISDRGQIVDDLENIKHIFSALRHGLMHFDYDFFEALFNGEDIDIKMDNQGNTQPLSSLNIKFLDIMIDKLDKLNIDTKKEFIDAEKITIFGEELSLAKLYRFYAHTAINRVAFNKLINSFIIENGVENQSLKEYFNQQAGGIAYEIDIHQNREYKNLYNEHKKLVSRVLSISDGQEIATLNQKIVELKEQMKQITKINSIKRLEYKLRLAFGFIYTEYKNYEEFKNSFDTDIKNGRFTPKDEDGNKRAFDSRELEHLKGYYKATLQTQKPQTDEKMEEVSKRVDRLSLKSLIGDDTLLKFILLMFTFMPQELKGEFLGFIKKYYHDTKHIDQDTISDSDDTIEEGLSIGLKLKILDKNIRSLSILKHSLSFQTKYNKKDRSYYEDGNIHGKFFKKLGISHNQEEFNKSVYAPLFRYYSALYKLINDFEIYTLSLHIVGNETLSDQVNKPQFLSGRYFNFRKLLTQSYNISNNSTHSVIFNAVINMRNDISHLSYEPLLDCPLNGKKSYKRKIRNQFRTINIKPLVESRKMIIDFITLQTDMQKVLGCDAVNDFTMKIVQLRTRLKAYANKEQTIEKMITEAKTPNDFYNIYKVKGVEAINKYLLEVIGETQVEKEIREEIERGNIANS

SEQ ID NO: 63 - protein, 802 aa, synthetic construct
MLKKPSNRYALPKVILSTVDHEKILEFKVKYEKLARLDRLVVERMHFDGESVVFDEVIANSGDLEIAYQDDHRKLLIQAAGKSYTITGKKVGGKKRKLEERISRAKIQLTLTDGQEDQHRRIRATVTEKALLEPKEDRDIYSKISDRKIKTSKEIYLVKRFLSYRSDLLFYYFFVDNFFKVGNNKQELWKIKFQNQPELIEYFRFIINDRFKNAKNDKFDNYLKNDKAIQEDLEKIQKVFEKLRHALMHYDYGFFEKLFGGEDQGFDLDIAFLDNFVKKIDKLNIDTKKEFVDDEKIKIFGEDLNLADLYKLYASISINRVGFNRVVNEMIIKDGIEKSELKRAFEKKLDKTYALDIHSDPSYKKLYNEHKRLVTEVSTYTDGNKIKEGNQKIAKLKYEMKEITKKNALVRLECKMRLAFGLIYGRYDTHEAFKNGFDTDLKRGEFAQIGSEEAIGYFNTTFEKSKPKSKEEIKKIARQIDNLSLSTLIEDDPLMKFIVLMFLFVPRELKGEFLGFWRKYYHDIHSIDSDAKSDEMPDEVSLSLKLKILTRNIRRLNLFEYSLSEKIKYSPKNTQFYTDKSPYQKVYKRLKISHNKEEFDKTLLVPLFRYYSILFKLINDFEIYSLAKANPDASSLSELTKTKHGFRGHYNFTTLMMDAHKVSQGDSKKHFGIRGEIAHINTKDLIYDPLFRKSKMAQQRNDVIDFVLKYEKEIKAVLGYDAINDFRMKVVQLRTKLKVYSDKTQTIEKLLNEVEAPDDFYVLYKVKGVEAINKYLLEIVSVTQAEEEIERKIITGNKRYNT

SEQ ID NO: 64 - protein, 1281 aa, synthetic construct
MIKSYDDFTKLYSLQKTLRFELKPIGKTLEHIKKSEIIESDETLEEQYAIVKNIIDRFHRKHIDEALSLVDFTKHLDTFKTIQELYLKRGKTDREKKELEELSADLRKLIVSYLKGNVKQKTQHNLNPIKERFEILFGKELFTNEEFFTLAENKEEKKAIQAFKGFTTYFKGFQENRKNMYSEEDKSTAIAYRIINENMPLFIENIARFQKVLDVIEKTKLTELKQNLKTELKGHSVSDIFRIEYFNNVLTQEGISRYNTIIGGKTTTEGVKIQGLNEIINLHNQQSKDVKLPILKPLHKQILSEEYSTSFKIKAFENDNEVLKAIDTFWNEHIEKSIHPVTGKRFNILLKIENLCKKLEKYKDKEIEKLFIERKNLSTVSHQVYGQWNIIRDALRMHLEMNNKNIKEKDIDKYLDNDAFAWKEIKDSIKIYKEHVEDAKELDENGIVKYFSSMSINEEDDEKEYSISLIKNINEKYNNVKSILEEDRTGKSDLHQDKEKVAIIKEFLDSLKQLQWFLKLLYVTVPLDEKDYEFYNELEVYYEALLPLNSLYNKVRNYMTRKPYSVEKFKLNFYSPTLLDGWDKNKETANLSIILKKNGKYYLGIMNKENNTIFENFPKSKSNDYYEKMIYKLLPGPNKMLPKVFFSKKGLEYYKPSKEILRIYEKGEFKKDKSGNFKKESLHTLIDFYKEAIAKNEDWKIFKFKFKNTREYEDISQFYRDVEEQGYLIIFEKVDANYVDKLVEEGELYLFQIYNKDFSENKKSKGNPNLHTIYWESLFDNQNLKDVVYKLNGEAEVFYRKKSIDYPEEIYNNGHHKEELNGKFNYPIIKDRRYTQDKFLFHVPITMNFISKEEKRVNQLACEYLSTTKEDVHIIGIDRGERHLLYLSLIDKEGNIKKQLSLNTIKNENYDKEIDYRVKLDEKEKKRDEARKNWDVIENIKELKEGYMSQVIHIIAKMMAEEKAILIMEDLNIGFKRGRFKVEKQVYQKFEKMMIDKLNYLVFKNKEPLEPGGSLNAYQLTSKFDSFKKLGKQSGFIFYVPSAYTSKIDPTTGFYNFIRVDVPNLEKGKEFFSKFEKIIYNTKEDYFEFHCKYGKFVPEPKNKDNDRKTKESLTYYNAIKDTVWVVCSTHHERYKIVRNKAGYYESQPVDVTKNLRDIFSEANINYSDGKDIKPIIIESNNAKLLKSIAEQLKLILAMRYNNGKHDDDEKDYILSPVKNKQGKFFCSLDGDQSLPINADANGAYNIALKGLLLIEKIKKQQGKAKDLYISNLEWFMFMMSR

SEQ ID NO: 65 - protein, 1332 aa, synthetic construct
MKSIFDNFTGLYSLSKTLRFELRPVGQTLENIKNGHFLESDKKMADDYQDVKKIIDNYHKFFIDDVLKGASFDWALLEKELTDFNKNKTDDSKVEAEQKKLREQIAKTLAGDKRFKSLTASTPNDLFNKDKDFIGWLEQSSVKEIRKDALDTFKKFSSYFKGFQENRKNVYSADDIPTAVPYRIVNDNFPKFLQNISIFKTIQEKCPQVIADVENELASYLGKEKLADIFTVQAFNKYLCQGGKENQRGIDFYNQVIGGIAEKEGGVNLRGINQFLNLYWQQHPDFAKENRRIKMVPLYKQILSDRSSLSFKIETIDTDEELKTAISEYADKLESKSNDEKKSVLDVCVELFDSIKEQNLQEIYVNRKDINNISRILTGDWSWLQSRMNLYADEVFTTKAEKTRWQKSVDGDEGENKSKGAYSLAELNRVLEYASENVAETDIRITDYFCHRNRFYYEKESGLFKQGEELVALSIKESCEDILSKRKAMNEAFANISESNSLRDNSEDIAKTKTYLDSVQDLLHRIKPLKVNGLGDPSFYAVFDSIYSALSEVISIYNKTRNYITRKAESPEKYKLNFDNPTLANGWDLNKEKDNTCVLLRKNGMYYLGIMNPKDKPKFAEKYDCGTESCYEKMIYKLLPGPNKMLPKVFFSTKGKKQYNPPENILHGYEQGKHKKGVAFDINFCHELIDWFKSAINQHEDWKKFGFKFSDTKSYKDISDFYREVTEQGYKLTFINIPESEISKMVSEGKLYLFQIYNMDFAPGANGMPNMHTLYWKNLFSEENLKDVVLKLNGEAELFYRPAGIKEPVVHAKGSYLVNRITKDGEPIPEKIHDEIYRNANGNLNNLSKEASEYKESHKVVIKQASHEIIKDRHYTEPKFLFHIPLTINFKAPSMKPTVCTAIQNENVRRFLKNNPDVNIIGLDRGERNLIYLSLINQKGEIIKQFSFNDVEREQNGQTVKVNYHEKLDQREKERDAARKSWQAIGKIAELKEGYLSAVIHQLTKLMVEYNAIVVMEDLNFGFKRGRFHVEKQVYQKFEHMLIDKLNYLVFKDRGLNEPGGALNGYQLAGQFESFQKLGKQSGMLFYVPAAYTSKIDPKTGFVSMMNFKDLTNVHKKRDFFSKFEDIHFDEATCSFVFTFDYKNFNGKAKEEMKQTKWAVYSREKRIVYFSKTKSSEDIMPTEKLRALFESDGIEYKSGNNIHDSVMAVGADLKEGAKPSKEIADFWDGLLYNFKLILQMRNSNAKTGEDYIISPVMASDGTFFDSRVEAKKGKDAKLPLDADANGAYHIALKGLSLINKINLAGEDELKKFDMKISNEDWFNFAQEKKYAE

SEQ ID NO: 66 - protein, 1345 aa, synthetic construct
MTKQNKSVTQNTKRKNFGEFTNLYSLSKTLRFELKPVKATKTILEVERKDRENFKKDRKIAKNYQKLKGILNELHQEFIQDVMREFSFQEKEIKEFEERYLEALNFKEKDNYKKRTQLKNAYEKVAKKLAGKIATAFGKYNQEKYGVKFTKKNLTGENVFDILEGKYKGDKKILGIIHTFKFKPTKEEKKQGKEAVNFSTYLGGFNQNRENFYKGEMKAGQFATRTIENLIQFLKNKKLFIDKYKDNYQKIGFSQEQVEIFNLNYFNNLFLQEGLDVYNGILGAKKGEKNTENDGLNQKINLFKQKEKTRCKANGEKFNKSDYPIFKELYKQIGSIKKDNDVYVEIKSDEELVNVLQSLPEKTANTLREVQKFYENFFDKIFNDEFDLDKIYLPKSVGTHFSHLAFSDWSKLAFVFNKRWRNEKVKIKEGEDVNVQSRSLADIKKRMEEILEMDGGVSFGKTYCQKVGLEKEARTIEDVWSGFWKIIQYHINSQFIGGEKEVFDKEKKDDKTEKIQTIDDLQEEYLQATEMYRERMVESEEGLNDGEEKEIKTKLKNYLDRIKDIERIARYFDLRKHFDDIDEASKDGDFYFIYQELLQDISEAKINDHYNEIRNYLTKANVVDDKFKLNFNDGQTLSGWDLNKETEKFSLIFKRKVDGGVEYYLGIINKEKNKTIFDKKKHPEIFTENSEFEKMEYKLFPSPSKMLPKIAFTKNKEGERIKPVFLDENAGKEIAQIKKEFALFQDAKKEDKNKWSDEFDRKKLNKLIDYYKLVLEKHPEKYMQTFNFVFKSSAKYKNLGEFNDDVARQNYVTKFVSVDKDYIDQKVESGELYLFKIHNKDWNLTKAGDTKKQSKKNLHTIYFEELFSEKNIAEPVFKLSGGAEVFFRDAIEKKKQKKKKDKKGKEILEKFRFTKNKILFHVPITINYGKPSINQGQFNQKINEFIADNSRSVNILGIDRGEKHLLYYSLVDNNGKIIKSGSLNEINGVDYHEKLDKAEKERQEARKSWQKINQIKNLKAGYISQVIKKIVDLAIENNAIIVLEDLNFGFKSFRQKIEKNVYQQFEKALIDKLGFVTDKEKLNHRQAPQLSAPFESFEKMGKQTGIVFYVLATNTSKVCPQCQWKKNIFPHYSTKKSIAENLQKQYKMKMYWRENENRFEFEYKGDGDKEFSSIFSNVDRVRYDKRANNNQGGYVIYQIDSTTKEKDGRNIKEKSITNLLKELLLEKFEIDNLEGELLVKLSEKSPDVSKETIKDFFGLLNSILNIRNSMTDTEEDYIQCPACGFDTRKENKIGIKNGDDNGAYNIALRGRFLIERIKKAKKEDKKPNLTFSNNDYFQWVREFVK

SEQ ID NO: 67 - protein, 1265 aa, synthetic construct
MRTTTSLDAFTNRYALSKTLRFELKPIGNTQMMLEQNNVFAKDRAIREKYEKTKPWIDLLHREFVAESLQNAQLGNLDDYYAALQNVQKITKDTNAEDKKRWKKGFEKQEKRLRKEVVALFDKAAHIWATQRYPQLKKKTKDFLFEEGVFEHVLFARYGSAPDTTVKIVTSNPETGEVIDEREESIFKGWKGFTGYFDKFFETRKNFYKDNGTATAIATRAINQNLRRFAENMQKLTDIKNNYPELLAHTDFGDFDIAHAQSLDFYARTCLLQEGIDAYNKKFVGVLKSAINEYQQKNKGVRISYPKTLDNQILGERERRLFDVIEDDRELHDVFRAFVDDGTVFAAEMRQLAQAFSAQNGTYDYTQIYISKKGFETISRKYTHDTRAWHDALADVFKAKAKKRIATTASGEKKFPAYIPVAYITQALTLVQESEDTECTWKERYASITENKTLEEGFFAIFADEFERLFVHMEATVQDTDYVVAEDKAKKLLSDGQITKNEQTTQIIKEYADALLRIYQMAKYFAVEKKSMWDDAVAIDDTFYETFKEIYGNTHSTIVASYNLLRNYLTKKPWEDVQKWKLNFENPTLLDGWDKNKEAANFGVILRDGDKFYLGIMRKGHNNIFANQHHSNFEGQGLQKMVYKFFPDPKKMFPKVCFSAKGMEFFAPSEEIVRIYKNAEFKSGDTFNVESMQKLIDFYKNALQKYDGWKIYDFKHLKDTAQYTSNIGEFYDDVAKGGYQLGWQNISKEYVEEKNANGELYLFQIKNKDWNDGATGRKNLHTLYFEYLFSEKNAAADFVFRLNGGAEVFYRPAAIESKTERRGNREVAAKKRYTQDKVFLHVPITLNRTAGDVKTSAFNDAVNRFLAGNPDINIMGIDRGEKHLAYYSIIDQNGNRIVSGSFNTIGSKDYHALLTERQGAREEARKNWQRVEQIKDLKKGYISLVVREIADLAIKHNAIIVLENLNMRFKQIRGGIEKSVYQQLEKALIEKLNFLVNKGEVDATKAGHLLRAYQLAAPFETFEKMGNQTGIIFYTTASYTSQVDPVTGWRPHVYLKYRNAQKTKEDILRIFDDIVFNDEKQRFEFAYRHNGVSWTVCSSVERHRWNRSNNAGKGGYDVFPVEGEGSITQRLQEACAQRGIDTTRNILAQIDELDESASATVSFLRDLCFYFRLICQIRNTDDGADDINAQDFLMSPVEPFFDTRNAQEQYPQNGDENGAYNIARKGIIILQKITAWGRSQDTQRRYPDTFVSQDEWDTFLTQHTT

SEQ ID NO: 68 - protein, 1259 aa, synthetic construct
MLKEKQFKTFGDFTNLYELSKTLRFELRPTPETKDLLDKNKIIQTDKKIAENYQEIKKYFDKLHKKFIKEALSNTQIDFSDFCKLWEQNSKDSGKIKDLSRKLRKSIKQAFDKKGADWHKRYLEKGIKLKKKNLDILFEERVLDILKEEFKDDVDVKLFESFKGFSTYFTNFHESRKNFYKDDGTASAIATRIIDENLKRFCDNIKVKKHSKKLISELNEREAKIFEADFYNRCLLQQGIDDYNQVIGDINKKINNLRQNKIENTPTLKILYKQILGDVRRQETEQDAFIEIKNNEEVFDFLQDFIKHSDENNKYFKNLFYKFIEGKHSLDKIFLAKRFVNTISGKWFASWEVFGAELIKKFGNKKDLPDFIPFAAVKDVLQNCNIPANELFKEKIKNDEDKNIYDIFINLWKEEFDSNLKKVEESKKEVENMIAEDKVYSNKKEKRKNDNGEEIEIEIQKEKIKNYADAAMNIFRMMKYFLLEKNGKTVEGMGEDNNFYNELNIVFKGGEIDGKVYEGVKTYLYYNEFRNYLTKKPFNEEKTKLNFDCGQILSGWDKNKESEKLGVILRKDNKYYLAIINKKHNKIFDVKKNSYAYIVGDNFYEKMEYKLFPDAKRMIPKIAFAKNNKEKFGWTDEIQKIKNEYAEFQEGKKNDKNLWKDKFNKNKMEKLITYYQNCLEKGGYKDIYNFRWKSPDKYSGIGEFNDEIDRQSYCLKFVKVDFNYVFEKVKSGELYLFQIYNKDFSDKADRAQKENIHTEYFKLLFDQRNLDNVVLKLSGGAEIFYRPKTEGLPKKKDNKGNEVVRHRRYADDKYFLHLPIQLNFGRGNLSGGEFNSKINQYLSEQREIKIIGIDRGEKHLAYYSVINQDGKIEEIESLNTVNGIDYRKKLDELEKKREQERKSWQSISKIKDLKKGYISHVIKKICDLAIEHNAIIVFEDLSGGFKNSRKKIEKQIYQNLELALATKLNYLTFKDKNFGESGHYLNAYQLAPKIDNYQDIKMQTGIVFYTPAGYTSSTCPQCGFRKTLKFDYTATISKAEDLIRGSKLNIVFEKEKNRFKINYLFNPIEKKKKKIKENELFADAGAKNEFTIYSDVKRIKWHNTGTKRLEEAEGERLLENKNSRGRDKEYDINKCLTRLFRENKIDVNGDIIGQITKIKSLKLYQDLFYYLFLATLIRNNVSGSDIDYIQCPSCHFHSDGGFQKQKFNGDANGAYNIARKGILILKKIKQFAAQDKDMKNFGWKHLTVDINEWDKFTQK

SEQ ID NO: 69 - protein, 1371 aa, synthetic construct
MNKNFSNFTELYTLSKTLRFELKPVAQTKENIKKGKFLESDKKKADDYKDVKKIIDNYHKFFIDDVLKNASFDWTVLEKEMSDFNKSKADDSKVEAEQKKLRDQIAKKLTSDKRFKALTASTPSDLFNKDKDFIDWFTQNSTKDINKEALETFKRFSSYFKGFQENRKNVYSAEPIPTAVPYRLVNDNFPKFLQNIALFKIIQEKCPQVISDVEKELASYLGKEKLADIFTVQAFNKYLCQGGKENQRGIDFYNNILGGIAEKEGGINLRGINQFLNLYWQQHQDFAKQNKRIKMIPLFKQILSDRSSLSFKIESINTDQELLTSITEYADKLETKSNDEKKSVLDICSDLFASIKAQNLQEIYVNRKDINSISRILTGDWSWLQSRMNVYADEVFTTKAEKTRWQKSIDGDEGENKSKGVFSLAELNSVLEYSSENVSETDVRITDFFDHRNRFYYEKESGLFKQGDELVALSIRESCEDILAKRKAMDEAFANVSENNSLRDNSEDVAKIKIYLDCVQELLHRIKPLKVNGLGDPAFYAVFDTVYNSLSEVISLYNKTRNYITKKAANPEKYKLNFDNPTLADGWDLNKEQANTSVLMRKDGMYYLGIMNPKDKPKFAEKYECGNEACYEKMIYKQFDATKQIPKCSTQVKEVKKHFQSGATDSIILNDKSKFKLDLVITKEIWFLNNHVWNGEKFVPKRESNETRPKKFQIGYYKQTGDLGGYKEALNIWISFCKTFLQSYISSSIYDYDFKESSNYDSLDEFYNYLNATCYKLSFINIPEATISQMVSEGKLYLFQIYNKDFAPGASGMPNMHTLYWKNLFSEENLKNVVLKLNGEAELFYRPAGIKEPVIHAKGSYLVNRITKDGEPIPEKIHDEIYRNANGKLESLSKEATEYKASHKVVIKEAKHDIIKDRHYTEPKFLFHVPLTINFKASGNSYINENVRRFLKNNPDVNVIGLDRGERHLIYLSLINQKGEIIKQFTFNEVERNKNGQVIKVNYHEKLDQREKVRGAARKSWQAIGKIAELKEGYLSAVIHQLTKLMVEYNAIVVMEDLNFGFKRGRFHVEKQVYQKFEHMLIDKLNYLVFKDRGLTEAGGVLNGYQLAGQFESFQKLGKQSGMLFYVPAGYTSKIDPKTGFASMSNFKDLTNVHKKRAFFSKFDDIHFDDATGSFVFTFDYKNFDGKAKEEMKRTKWSVYSKDKRIVYLSKTKSYEDVQPTEKIKASLESVGIEYMSGNNLIDSIMVIGAELKDGAKPSKEIADFWDRLLYNFKLILQMRNSNAKTGEDYIISPVMADDGTFFDSREEFKKGENAKMPVDADANGAYHIALKGLSLLKRFDAASENELKKFDMKISNVDWFKFAQEKSYAE

SEQ ID NO: 70 - protein, 1160 aa, synthetic construct
MKAKKSFYNQKRKFGKRGYRLHDERIAYSGGIGSMRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAV

SEQ ID NO: 71 - protein, 1285 aa, synthetic construct
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 72 - protein, 1344 aa, synthetic construct
MLRRDKEVKKLYNVFNQIQVGTKPKKWNNDEKLSPEENERRAQQKNIKMKNYKWREACSKYVESSQRIINDVIFYSYRKAKNKLRYMRKNEDILKKMQEAEKLSKFSGGKLEDFVAYTLRKSLVVSKYDTQEFDSLAAMVVFLECIGKNNISDHEREIVCKLLELIRKDFSKLDPNVKGSQGANIVRSVRNQNMIVQPQGDRFLFPQVYAKENETVTNKNVEKEGLNEFLLNYANLDDEKRAESLRKLRRILDVYFSAPNHYEKDMDITLSDNIEKEKFNVWEKHECGKKETGLFVDIPDVLMEAEAENIKLDAVVEKRERKVLNDRVRKQNIICYRYTRAVVEKYNSNEPLFFENNAINQYWIHHIENAVERILKNCKAGKLFKLRKGYLAEKVWKDAINLISIKYIALGKAVYNFALDDIWKDKKNKELGIVDERIRNGITSFDYEMIKAHENLQRELAVDIAFSVNNLARAVCDMSNLGNKESDFLLWKRNDIADKLKNKDDMASVSAVLQFFGGKSSWDINIFKDAYKGKKKYNYEVRFIDDLRKAIYCARNENFHFKTALVNDEKWNTELFGKIFERETEFCLNVEKDRFYSNNLYMFYQVSELRNMLDHLYSRSVSRAAQVPSYNSVIVRTAFPEYITNVLGYQKPSYDADTLGKWYSACYYLLKEIYYNSFLQSDRALQLFEKSVKTLSWDDKKQQRAVDNFKDHFSDIKSACTSLAQVCQIYMTEYNQQNNQIKKVRSSNDSIFDQPVYQHYKVLLKKAIANAFADYLKNNKDLFGFIGKPFKANEIREIDKEQFLPDWTSRKYEALCIEVSGSQELQKWYIVGKFLNARSLNLMVGSMRSYIQYVTDIKRRAASIGNELHVSVHDVEKVEKWVQVIEVCSLLASRTSNQFEDYFNDKDDYARYLKSYVDFSNVDMPSEYSALVDFSNEEQSDLYVDPKNPKVNRNIVHSKLFAADHILRDIVEPVSKDNIEEFYSQKAEIAYCKIKGKEITAEEQKAVLKYQKLKNRVELRDIVEYGEIINELLGQLINWSFMRERDLLYFQLGFHYDCLRNDSKKPEGYKNIKVDENSIKDAILYQIIGMYVNGVTVYAPEKDGDKLKEQCVKGGVGVKVSAFHRYSKYLGLNEKTLYNAGLEIFEVVAEHEDIINLRNGIDHFKYYLGDYRSMLSIYSEVFDRFFTYDIKYQKNVLNLLQNILLRHNVIVEPILESGFKTIGEQTKPGAKLSIRSIKSDTFQYKVKGGTLITDAKDERYLETIRKILYYAENEEDNLKKSVVVTNADKYEKNKESDDQNKQKEKKNKDNKGKKNEETKSDAEKNNNERLSYNPFANLNFKLSN

SEQ ID NO: 73 - RNA, 10 nt, synthetic construct
  source 1..10: mol_type = other RNA
  misc_feature 1: note = 6-Fluorescein moiety attached
  misc_feature 10: note = Iowa Black FQ moiety attached
tttttttttt

SEQ ID NO: 74 - RNA, 10 nt, synthetic construct
  source 1..10: mol_type = other RNA
  misc_feature 1: note = IRDye 700 moiety attached
  misc_feature 10: note = IRDye QC-1 Quencher moiety attached
tttttttttt

SEQ ID NO: 75 - RNA, 10 nt, synthetic construct
  source 1..10: mol_type = other RNA
  misc_feature 1: note = TYE 665 moiety attached
  misc_feature 10: note = Iowa Black RQ moiety attached
tttttttttt

SEQ ID NO: 76 - RNA, 10 nt, synthetic construct
  source 1..10: mol_type = other RNA
  misc_feature 1: note = Alexa Fluor 594 (NHS Ester) moiety attached
  misc_feature 10: note = Iowa Black RQ moiety attached
tttttttttt

SEQ ID NO: 77 - RNA, 10 nt, synthetic construct
  source 1..10: mol_type = other RNA
  misc_feature 1: note = ATTO TM 633 (NHS Ester) moiety attached
  misc_feature 10: note = Iowa Black RQ moiety attached
tttttttttt

SEQ ID NO: 78 - DNA, 10 bp, synthetic construct
  source 1..10: mol_type = other DNA
  misc_feature 1: note = 6-Fluorescein moiety attached
  misc_feature 10: note = Iowa Black FQ moiety attached
  misc_feature 1..4: note = DNA fragment
  misc_feature 5..6: note = RNA fragment
  misc_feature 7..10: note = DNA fragment
tttttttttt

SEQ ID NO: 79 - DNA, 10 bp, synthetic construct
  source 1..10: mol_type = other DNA
  misc_feature 1: note = IRDye 700 moiety attached
  misc_feature 10: note = IRDye QC-1 Quencher moiety attached
  misc_feature 1..4: note = DNA fragment
  misc_feature 5..6: note = RNA fragment
  misc_feature 7..10: note = DNA fragment
tttttttttt

SEQ ID NO: 80 - DNA, 10 bp, synthetic construct
  source 1..10: mol_type = other DNA
  misc_feature 1: note = TYE 665 moiety attached
  misc_feature 10: note = Iowa Black RQ moiety attached
  misc_feature 1..4: note = DNA fragment
  misc_feature 5..6: note = RNA fragment
  misc_feature 7..10: note = DNA fragment
tttttttttt

SEQ ID NO: 81 - DNA, 10 bp, synthetic construct
  source 1..10: mol_type = other DNA
  misc_feature 1: note = Alexa Fluor 594 (NHS Ester) moiety attached
  misc_feature 1..4: note = DNA fragment
  misc_feature 5..6: note = RNA fragment
  misc_feature 7..10: note = DNA fragment
  misc_feature 10: note = Iowa Black RQ moiety attached
tttttttttt

SEQ ID NO: 82 - DNA, 10 bp, synthetic construct
  source 1..10: mol_type = other DNA
  misc_feature 1: note = ATTO TM 633 (NHS Ester) moiety attached
  misc_feature 1..4: note = DNA fragment
  misc_feature 5..6: note = RNA fragment
  misc_feature 7..10: note = DNA fragment
  misc_feature 10: note = Iowa Black RQ moiety attached
tttttttttt

SEQ ID NO: 83 - DNA, 10 bp, synthetic construct
  source 1..10: mol_type = other DNA
  misc_feature 1: note = 6-Fluorescein moiety attached
  misc_feature 10: note = Iowa Black FQ moiety attached
tttttttttt

SEQ ID NO: 84 - DNA, 12 bp, synthetic construct
  source 1..12: mol_type = other DNA
  misc_feature 1: note = 6-Fluorescein moiety attached
  misc_feature 10: note = Iowa Black FQ moiety attached
tttttttttttt

SEQ ID NO: 85 - DNA, 14 bp, synthetic construct
  source 1..14: mol_type = other DNA
  misc_feature 1: note = 6-Fluorescein moiety attached
  misc_feature 10: note = Iowa Black FQ moiety attached
tttttttttttttt

SEQ ID NO: 86 - DNA, 12 bp, synthetic construct
  source 1..12: mol_type = other DNA
tttttttttttt

SEQ ID NO: 87 - RNA, 50 nt, synthetic construct
  source 1..50: mol_type = other RNA
gccaccccaaaaatgaaggggactaaaacaagaagaattcagatttttaa

SEQ ID NO: 88 - DNA, 12 bp, synthetic construct
  source 1..12: mol_type = other DNA
  misc_feature 1: note = Alexa Fluor 647 (NHS Ester) moiety attached
  misc_feature 10: note = Iowa Black RQ moiety attached
tttttttttttt

SEQ ID NO: 89 - protein, 1277 aa, synthetic construct
MIKSYDDFINHYAIQKTLRFELQPIGKTREHIQKNGIIEHDEALEQKYQIVKKIIDRFHRKHIDEALSLVDFTKHLDTFKTIQELYLKRGKTDREKKELEELSADLRKRVVSFLEGKVEGDARFAKVQQRYGILFDAKIFKDKEFISTACDDIEKDAIEAFKRFATYFTGFHENRKNMYSADEESTAIAYRVINENLPRFLENKARFEKILDVIEKTKLTELKQNLKTELKGHSVSDIFRIEYFNNVLTQAGITYYNTILGGKTKENGEKVQGLNEIINLFNQKNKDTMLPLLKPLYKQILSEEYSTSFKIKAFENDNEVLKAIDTFWNEHIEKSIHPVTGKRFNILLKIENLCKKLEKYKDKEIEKLFIERKNLSTVSHQVYGQWNIIRDALRMHLEMNNKNIKEKDIDKYLDNDAFAWKEIKDSIKIYKEHVEDAKELDENGIVKYFSSMSINEEDDEKEYSISLIKNINEKYNNVKSILEEDRTGKSDLHQDKEKVAIIKEFLDSLKQLQWFLKLLYVTVPLDEKDYDFYNELEMYHDTLLPLTTLYNKVRNYMTRKPYSVEKFKLTFEKSTLLDGWDKNKERANLGVILRKGNNYYLGIMNKKYNDIFDSIPGLTTTDYCEKMNYKLLPGPNKMLPKVFFSKKGVQFYKPSKEILRIYEKGEFKKDKSGNFKKESLHTLIDFYKEAIAKNEDWKIFKFKFKNTREYADISQFYKDVERQGYKISFDKIDWEYILLLVDEGKLFLFKIYNKDFSPYSKGKPNLHTIYWKNIFSHDNLNNVVYKLNGEAEVFYRKKSIDYPEEIYNNGHHKEELNGKFNYPIIKDKRYAEDKFLFHVPITMNFISKEEKRVNQLACEYLSTTKEDVHIIGIDRGERHLLYLSLIDKEGNIKKQLSLNTIKNENYDKEIDYHAKLDEKEKKREEARKNWDVIENIKELKEGYLSQVVHQIAKLMVEYKAILVMEDLNTGFKRGRFKVEKQVYQKFEKMMIDKLNYLVLKDRQATQPGGSLKAYQLASSLESFKKLGKQCGMIFYVPSAYTSKIDPTTGFYNFIRVDVPNLEKGKEFFSKFEKIIYNTKEDYFEFHCKYGKFVPEPKNKDNDRKTKESLTYYNAIKDTVWVVCSTHHERYKIVRNKAGYYESQPVDVTKNLRDIFSEANINYSDGKDIKPIIIESNNAKLLKSIAEQLKLILAMRYNNGKHDDDEKDYILSPVKNKQGKFFCSLDGDQSLPINADANGAYNIALKGLLLIEKIKKQQGKAKDLYISNLEWFMFMMSR

SEQ ID NO: 90 - protein, 1276 aa, synthetic construct
MIKSYDDFINHYAIQKTLRFELQPIGKTREHIQKNGIIEHDEALEQKYQIVKKIIDRFHRKHIDEALSLVDFTKHLDTFKTIQELYLKRGKTDREKKELEELSADLRKLIVSYLKGNVKQKTQHNLNPIKERFEILFGKELFTNEEFFTLAENKEEKKAIQAFKGFTTYFKGFQENRKNMYSEEDKSTAIAYRIINENMPLFIENIARFQKVLDVIEKTKLTELKQNLKTELKGHSVSDIFRIEYFNNVLTQEGISRYNTIIGGKTTTEGVKIQGLNEIINLHNQQSKDVKLPLLKPLYKQILSEEYSTSFTISAFEKDNDVLQAIGSFCNDCIFYAKNNVNGKAYNLLQTVQAFCNSIDTYNDNRLDGLHIERKNLATLSHQVYGEWNILRDALQIHYEAYEQKDNGNNNNYLESKTFSWKALKDALTTYKSLVEEAQDIDENGFIAYFKDMKFKEEIDGKTTSIDLIENIQTRYKSIETILQEDRNNKNNLHQEKEKVATIKGFLDSVKYLQWFLNLMYIASPVDDKDYDFYNELEMYHDTLLPLTTLYNKVRNYMTRKPYSVEKFKLTFEKSTLLDGWDKNKERANLGVILRKGNNYYLGIMNKKYNDIFDSIPGLTTTDYCEKMNYKLLPGPNKMLPKVFFSKKGVQFYKPSQEIIRLYNNKEFKKGDTFNKNSLHKLINFYKESIAKTEDWSVFQFKFKNTNDYADISQFYKDVERQGYKISFDKIDWEYILLLVDEGKLFLFKIYNKDFSPYSKGKPNLHTIYWKNIFSHDNLNNVVYKLNGEAEVFYRKKSIEYPEEILQKGHHVNELKDKFKYPIIKDKRYAEDKFLFHVPITMNFLSKEEKRVNQLACEYLSTTKEDVHIIGIDRGERHLLYLSLIDKEGNIKKQLSLNTIKNENYDKEIDYRVKLDEKEKKRDEARKNWDVIENIKELKEGYMSQVIHIIAKMMAEEKAILIMEDLNIGFKRGRFKVEKQVYQKFEKMMIDKLNYLVFKNKEPLEPGGSLNAYQLTSKFDSFKKLGKQSGFIFYVPSAYTSKIDPTTGFYNFIRVDVPNLEKGKEFFSKFEKIIYNTKEDYFEFHCKYGKFVPEPKNKDNDRKTKESLTYYNAIKDTVWVVCSTHHERYKIVRNKAGYYESQPVDVTKNLRDIFSEANINYSDGKDIKPIIIESNNAKLLKSIAEQLKLILAMRYNNGKHDDDEKDYILSPVKNKQGKFFCSLDGDQSLPINADANGAYNIALKGLLLIEKIKKQQGKAKDLYISNLEWFMFMMSR

SEQ ID NO: 91 - protein, 1278 aa, synthetic construct
MIKSYDDFINHYAIQKTLRFELQPIGKTREHIQKNGIIEHDEALEQKYQIVKKIIDRFHRKHIDEALSLVDFTKHLDTFKTIQELYLKRGKTDREKKELEELSADLRKLIVSYLKGNVKQKTQHNLNPIKERFEILFGKELFTNEEFFTLAENKEEKKAIQAFKGFTTYFKGFQENRKNMYSEEDKSTAIAYRIINENMPLFIENIARFQKVLDVIEKTKLTELKQNLKTELKGHSVSDIFRIEYFNNVLTQEGISRYNTIIGGKTTTEGVKIQGLNEIINLHNQQSKDVKLPILKPLHKQILSEEYSTSFKIKAFENDNEVLKAIDTFWNEHIEKSIHPVTGKRFNILLKIENLCKKLEKYKDKEIEKLFIERKNLSTVSHQVYGQWNIIRDALRMHLEMNNKNIKEKDIDKYLDNDAFAWKEIKDSIKIYKEHVEDAKELDENGIVKYFSSMSINEEDDEKEYSISLIKNINEKYNNVKSILEEDRTGKSDLHQDKEKVAIIKEFLDSLKQLQWFLKLLYVTVPLDEKDYEFYNELEVYYEALLPLNSLYNKVRNYMTRKPYSVEKFKLNFYSPTLLDGWDKNKETANLSIILKKNGKYYLGIMNKENNTIFENFPKSKSNDYYEKMIYKLLPGPNKMLPKVFFSKKGLEYYKPSKEILRIYEKGEFKKDKSGNFKKESLHTLIDFYKEAIAKNEDWKIFKFKFKNTREYEDISQFYRDVEEQGYLIIFEKVDANYVDKLVEEGELFLFQIYNKDFSENKKSKGNPNLHTIYWESLFDNQNLKDVVYKLNGEAEVFYRKKSIDYPEEIYNNGHHKEELNGKFNYPIIKDRRYTQDKFLFHVPITLNFLAKSDEKVNEMVKNYIAATNEKIHIIGIDRGERNLLYLSLIDSNGNIVKQQSLNIIELPKYQKQIDYHAKLNEKEKQRLAARQNWDVIENIKELKEGYLSQVIHQIARLMVDYKAILVMEDLNFGFKRGRFKVEKQVYQKFEKMLIDKLSYLVFKEKNLCEPGGSLRAYQLSAPFKSFKALGKQSGMIFYVPAQYTSKIDPTTGFYNFLNIDVSNLARSKETFSKFDKIVYNKKEDYFEFYCKMINFESANQLTKKSQNKANAELKEFQWILCSTHHDRFKVERKNNQINYCKINVNEELKKLLNSKGINYEKSNDLKSEILNIDESKFFKELGYLLKILVSLRYNNGKKGSEEQDFILSPVKNASGKFFCTLDNNNTLPLDADANGAYNIALKGLMIVQRVKAGGKLDLSISKDDWINFLIMNKKLPK

SEQ ID NO: 92 - DNA, 19 bp, synthetic construct
  source 1..19: mol_type = other DNA
tttttcatcgactagacgt

SEQ ID NO: 93 - DNA, 23 bp, synthetic construct
  source 1..23: mol_type = other DNA
cattgatgaatgtcataacttca

SEQ ID NO: 94 - DNA, 41 bp, synthetic construct
  source 1..41: mol_type = other DNA
gcaagaattcaccatgttggttaaatagcgattggtgttgt

SEQ ID NO: 95 - DNA, 44 bp, synthetic construct
  source 1..44: mol_type = other DNA
cttatggttttttaacgcaatggtcgaagatggtaagatccgtc

SEQ ID NO: 96 - DNA, 16 bp, synthetic construct
  source 1..16: mol_type = other DNA
acttactgcgaccagg

SEQ ID NO: 97 - DNA, 18 bp, synthetic construct
  source 1..18: mol_type = other DNA
atatactgaccgagtagg

SEQ ID NO: 98 - DNA, 22 bp, synthetic construct
  source 1..22: mol_type = other DNA
gtttattctcaaatagcgattg

SEQ ID NO: 99 - DNA, 23 bp, synthetic construct
  source 1..23: mol_type = other DNA
cattgatgaatgtcataacttca

SEQ ID NO: 100 - DNA, 43 bp, synthetic construct
  source 1..43: mol_type = other DNA
atcgttaatagtgtgcaagaattcagtgttgtaaggatcctgg

SEQ ID NO: 101 - DNA, 44 bp, synthetic construct; identical to SEQ ID NO: 95
  source 1..44: mol_type = other DNA

SEQ ID NO: 102 - DNA, 16 bp, synthetic construct
  source 1..16: mol_type = other DNA
tactgcgaccaggatc

SEQ ID NO: 103 - DNA, 16 bp, synthetic construct
  source 1..16: mol_type = other DNA
tatactgaccgagtag

SEQ ID NO: 104 - DNA, 19 bp, synthetic construct
  source 1..19: mol_type = other DNA
tttttcatcgactagacgt

SEQ ID NO: 105 - DNA, 23 bp, synthetic construct
  source 1..23: mol_type = other DNA
cattgatgaatgtcataacttca

SEQ ID NO: 106 - DNA, 38 bp, synthetic construct
  source 1..38: mol_type = other DNA
gcaagaattcaccatgttggaatagcgattggtgttgt

SEQ ID NO: 107 - DNA, 40 bp, synthetic construct
  source 1..40: mol_type = other DNA
tggttttttaacgcaatggtcgaagatggtaagatccgtc

SEQ ID NO: 108 - DNA, 16 bp, synthetic construct
  source 1..16: mol_type = other DNA
acttactgcgaccagg

SEQ ID NO: 109 - DNA, 18 bp, synthetic construct
  source 1..18: mol_type = other DNA
atatactgaccgagtagg

SEQ ID NO: 110 - DNA, 22 bp, synthetic construct
  source 1..22: mol_type = other DNA
gtttattctcaaatagcgattg

SEQ ID NO: 111 - DNA, 23 bp, synthetic construct
  source 1..23: mol_type = other DNA
cattgatgaatgtcataacttca

SEQ ID NO: 112 - DNA, 43 bp, synthetic construct; identical to SEQ ID NO: 100
  source 1..43: mol_type = other DNA

SEQ ID NO: 113 - DNA, 44 bp, synthetic construct; identical to SEQ ID NO: 95
  source 1..44: mol_type = other DNA

SEQ ID NO: 114 - DNA, 20 bp, synthetic construct
  source 1..20: mol_type = other DNA
catgttggttaacttactgc

SEQ ID NO: 115 - DNA, 18 bp, synthetic construct
  source 1..18: mol_type = other DNA
atatactgaccgagtagg

SEQ ID NO: 116 - DNA, 22 bp, synthetic construct
  source 1..22: mol_type = other DNA
gtttattctcaaatagcgattg

SEQ ID NO: 117 - DNA, 23 bp, synthetic construct
  source 1..23: mol_type = other DNA
cattgatgaatgtcataacttca

SEQ ID NO: 118 - DNA, 43 bp, synthetic construct; identical to SEQ ID NO: 100
  source 1..43: mol_type = other DNA

SEQ ID NO: 119 - DNA, 44 bp, synthetic construct; identical to SEQ ID NO: 95
  source 1..44: mol_type = other DNA

SEQ ID NO: 120 - DNA, 19 bp, synthetic construct
  source 1..19: mol_type = other DNA
gttggttaacttactgcga

SEQ ID NO: 121 - DNA, 16 bp, synthetic construct
  source 1..16: mol_type = other DNA
tatactgaccgagtag

SEQ ID NO: 122 - DNA, 22 bp, synthetic construct
  source 1..22: mol_type = other DNA
gtttattctcaaatagcgattg

SEQ ID NO: 123 - DNA, 23 bp, synthetic construct
  source 1..23: mol_type = other DNA
cattgatgaatgtcataacttca

SEQ ID NO: 124 - DNA, 41 bp, synthetic construct
  source 1..41: mol_type = other DNA
cgttaatagtgtgcaagaattcagtgttgtaaggatcctgg

SEQ ID NO: 125 - DNA, 40 bp, synthetic construct; identical to SEQ ID NO: 107
  source 1..40: mol_type = other DNA

SEQ ID NO: 126 - DNA, 20 bp, synthetic construct
  source 1..20: mol_type = other DNA
catgttggttaacttactgc

SEQ ID NO: 127 - DNA, 18 bp, synthetic construct
  source 1..18: mol_type = other DNA
atatactgaccgagtagg

SEQ ID NO: 128 - DNA, 20 bp, synthetic construct
  source 1..20: mol_type = other DNA
gtttacttttagtccgtatc

SEQ ID NO: 129 - DNA, 18 bp, synthetic construct
  source 1..18: mol_type = other DNA
ttactcttttgtatcgca

SEQ ID NO: 130 - DNA, 44 bp, synthetic construct
  source 1..44: mol_type = other DNA
acaattattggtgagtgtcatatgacagtcaacactatgttagc

SEQ ID NO: 131 - DNA, 38 bp, synthetic construct
  source 1..38: mol_type = other DNA
cggtaatcttgtcgatgagtctctcgggtattcggtag

SEQ ID NO: 132 - DNA, 19 bp, synthetic construct
  source 1..19: mol_type = other DNA
gtgaaagctatatcgacag

SEQ ID NO: 133 - DNA, 22 bp, synthetic construct
  source 1..22: mol_type = other DNA
gacatatagtattcttgtattc

SEQ ID NO: 134 - DNA, 18 bp, synthetic construct
  source 1..18: mol_type = other DNA
tccgtatccagtcaacac

SEQ ID NO: 135 - DNA, 18 bp, synthetic construct
  source 1..18: mol_type = other DNA
tccgtatccagtcaacac

SEQ ID NO: 136 - DNA, 18 bp, synthetic construct
  source 1..18: mol_type = other DNA
tccgtatccagtcaacac

SEQ ID NO: 137 - DNA, 18 bp, synthetic construct
  source 1..18: mol_type = other DNA
tccgtatccagtcaacac

SEQ ID NO: 138 - DNA, 19 bp, synthetic construct
  source 1..19: mol_type = other DNA
gtgaaagctatatcgacag

SEQ ID NO: 139 - DNA, 22 bp, synthetic construct
  source 1..22: mol_type = other DNA
gacatatagtattcttgtattc

SEQ ID NO: 140 - DNA, 20 bp, synthetic construct
  source 1..20: mol_type = other DNA
gtttacttttagtccgtatc

SEQ ID NO: 141 - DNA, 18 bp, synthetic construct
  source 1..18: mol_type = other DNA
ttactcttttgtatcgca

SEQ ID NO: 142 - DNA, 38 bp, synthetic construct
  source 1..38: mol_type = other DNA
attggtgagtgtcatatgacagtcaacactatgttagc

SEQ ID NO: 143 - DNA, 38 bp, synthetic construct; identical to SEQ ID NO: 131
  source 1..38: mol_type = other DNA

SEQ ID NO: 144 - DNA, 19 bp, synthetic construct
  source 1..19: mol_type = other DNA
gtgaaagctatatcgacag

SEQ ID NO: 145 - DNA, 22 bp, synthetic construct
  source 1..22: mol_type = other DNA
gacatatagtattcttgtattc

SEQ ID NO: 146 - DNA, 18 bp, synthetic construct
  source 1..18: mol_type = other DNA
tccgtatccagtcaacac

SEQ ID NO: 147 - DNA, 18 bp, synthetic construct
  source 1..18: mol_type = other DNA
ttactcttttgtatcgca

SEQ ID NO: 148 - DNA, 42 bp, synthetic construct
  source 1..42: mol_type = other DNA
aattctacaattattggtgagtgtatgttagcatttctgtcg

SEQ ID NO: 149 - DNA, 38 bp, synthetic construct; identical to SEQ ID NO: 131
  source 1..38: mol_type = other DNA

SEQ ID NO: 150 - DNA, 19 bp, synthetic construct
  source 1..19: mol_type = other DNA
gtgaaagctatatcgacag

SEQ ID NO: 151 - DNA, 22 bp, synthetic construct
  source 1..22: mol_type = other DNA
gacatatagtattcttgtattc

SEQ ID NO: 152 - DNA, 18 bp, synthetic construct
  source 1..18: mol_type = other DNA
tccgtatccagtcaacac

SEQ ID NO: 153 - DNA, 18 bp, synthetic construct
  source 1..18: mol_type = other DNA
ttactcttttgtatcgca

SEQ ID NO: 154 - DNA, 42 bp, synthetic construct; identical to SEQ ID NO: 148
  source 1..42: mol_type = other DNA

SEQ ID NO: 155 - DNA, 38 bp, synthetic construct; identical to SEQ ID NO: 131
  source 1..38: mol_type = other DNA

SEQ ID NO: 156 - DNA, 19 bp, synthetic construct
  source 1..19: mol_type = other DNA
gtgaaagctatatcgacag

SEQ ID NO: 157 - DNA, 22 bp, synthetic construct
  source 1..22: mol_type = other DNA
gacatatagtattcttgtattc

SEQ ID NO: 158 - DNA, 18 bp, synthetic construct
  source 1..18: mol_type = other DNA
tccgtatccagtcaacac

SEQ ID NO: 159 - DNA, 18 bp, synthetic construct
  source 1..18: mol_type = other DNA
ttactcttttgtatcgca

SEQ ID NO: 160 - DNA, 38 bp, synthetic construct
  source 1..38: mol_type = other DNA
ctacaattattggtgagtgtatgttagcatttctgtcg

SEQ ID NO: 161 - DNA, 38 bp, synthetic construct; identical to SEQ ID NO: 131
  source 1..38: mol_type = other DNA

SEQ ID NO: 162 - DNA, 19 bp, synthetic construct
  source 1..19: mol_type = other DNA
gtgaaagctatatcgacag

SEQ ID NO: 163 - DNA, 22 bp, synthetic construct
  source 1..22: mol_type = other DNA
gacatatagtattcttgtattc

SEQ ID NO: 164 - DNA, 18 bp, synthetic construct
  source 1..18: mol_type = other DNA
tattcgactggtgtcagg

SEQ ID NO: 165 - DNA, 18 bp, synthetic construct
  source 1..18: mol_type = other DNA
tgtattgtgtgcgcgtaa

SEQ ID NO: 166 - DNA, 46 bp, synthetic construct
  source 1..46: mol_type = other DNA
ctatcactcctattaaaggctggatacgttcgatataaacatatgc

SEQ ID NO: 167 - DNA, 43 bp, synthetic construct
  source 1..43: mol_type = other DNA
tctgtataataagatgcaaaggcaaaaataattcacatattgg

SEQ ID NO: 168 - DNA, 18 bp, synthetic construct
  source 1..18: mol_type = other DNA
tgaaccgatccactgatg

SEQ ID NO: 169 - DNA, 18 bp, synthetic construct
  source 1..18: mol_type = other DNA
gtagtaaagatgctagtg

SEQ ID NO: 170 - DNA, 18 bp, synthetic construct
  source 1..18: mol_type = other DNA
tattcgactggtgtcagg

SEQ ID NO: 171 - DNA, 18 bp, synthetic construct
  source 1..18: mol_type = other DNA
tgtattgtgtgcgcgtaa

SEQ ID NO: 172 - DNA, 39 bp, synthetic construct
  source 1..39: mol_type = other DNA
tcactcctattaaaggctgcgttcgatataaacatatgc

SEQ ID NO: 173 - DNA, 43 bp, synthetic construct; identical to SEQ ID NO: 167
  source 1..43: mol_type = other DNA

SEQ ID NO: 174 - DNA, 18 bp, synthetic construct
  source 1..18: mol_type = other DNA
tgaaccgatccactgatg

SEQ ID NO: 175 - DNA, 18 bp, synthetic construct
  source 1..18: mol_type = other DNA
gtagtaaagatgctagtg

SEQ ID NO: 176 - DNA, 20 bp, synthetic construct
  source 1..20: mol_type = other DNA
aatacatgtcttagatgttc

SEQ ID NO: 177 - DNA, 20 bp, synthetic construct
  source 1..20: mol_type = other DNA
tcttgataatcttgatgagt

SEQ ID NO: 178 - DNA, 42 bp, synthetic construct
  source 1..42: mol_type = other DNA
agaaatactttagatacgtgcgtaaaacttaatctctctcct

SEQ ID NO: 179 - DNA, 43 bp, synthetic construct
  source 1..43: mol_type = other DNA
agttaaattagatttcgaacgaaggtataccagaaaagacggt

SEQ ID NO: 180 - DNA, 21 bp, synthetic construct
  source 1..21: mol_type = other DNA
ggtatcaatttttgttaagag

SEQ ID NO: 181 - DNA, 21 bp, synthetic construct
  source 1..21: mol_type = other DNA
tcaaatgaacgccagaggcgt

SEQ ID NO: 182 - DNA, 21 bp, synthetic construct
  source 1..21: mol_type = other DNA
caatacatgtcttagatgttc

SEQ ID NO: 183 - DNA, 20 bp, synthetic construct
  source 1..20: mol_type = other DNA
tcttgataatcttgatgagt

SEQ ID NO: 184 - DNA, 38 bp, synthetic construct
  source 1..38: mol_type = other DNA
gaaatactttagatacgtgccaaacttaatctctctcc

SEQ ID NO: 185 - DNA, 42 bp, synthetic construct
  source 1..42: mol_type = other DNA
gttaaattagatttcgaacgaaggtataccagaaaagacggt

SEQ ID NO: 186 - DNA, 21 bp, synthetic construct
  source 1..21: mol_type = other DNA
ggtatcaatttttgttaagag

SEQ ID NO: 187 - DNA, 21 bp, synthetic construct
  source 1..21: mol_type = other DNA
tcaaatgaacgccagaggcgt

SEQ ID NO: 188 - DNA, 18 bp, synthetic construct
  source 1..18: mol_type = other DNA
ttcagggaaatcgcatct

SEQ ID NO: 189 - DNA, 20 bp, synthetic construct
  source 1..20: mol_type = other DNA
cccatatactttattcatgg

SEQ ID NO: 190 - DNA, 41 bp, synthetic construct
  source 1..41: mol_type = other DNA
ccattagctccataatacagttctatgaaaggaaagaatgt

SEQ ID NO: 191 - DNA, 41 bp, synthetic construct
  source 1..41: mol_type = other DNA
caaaatatgtagaaaaggaggtttgaatgagatattctgag

SEQ ID NO: 192 - DNA, 18 bp, synthetic construct
  source 1..18: mol_type = other DNA
ttgacgctggagaaatga

SEQ ID NO: 193 - DNA, 20 bp, synthetic construct
  source 1..20: mol_type = other DNA
agaacatggataaggttagc

SEQ ID NO: 194 - DNA, 18 bp, synthetic construct
  source 1..18: mol_type = other DNA
ttcagggaaatcgcatct

SEQ ID NO: 195 - DNA, 20 bp, synthetic construct
  source 1..20: mol_type = other DNA
cccatatactttattcatgg

SEQ ID NO: 196 - DNA, 39 bp, synthetic construct
  source 1..39: mol_type = other DNA
ccattagctccataatacagtctatgaaaggaaagaatg

SEQ ID NO: 197 - DNA, 39 bp, synthetic construct
  source 1..39: mol_type = other DNA
caaaatatgtagaaaaggaggtgaatgagatattctgag

SEQ ID NO: 198 - DNA, 17 bp, synthetic construct
  source 1..17: mol_type = other DNA
tgacgctggagaaatga

SEQ ID NO: 199 - DNA, 20 bp, synthetic construct
  source 1..20: mol_type = other DNA
agaacatggataaggttagc

SEQ ID NO: 200 - DNA, 18 bp, synthetic construct
  source 1..18: mol_type = other DNA
tcgcatcttctatgaaag

SEQ ID NO: 201 - DNA, 20 bp, synthetic construct
  source 1..20: mol_type = other DNA
cccatatactttattcatgg

SEQ ID NO: 202 - DNA, 44 bp, synthetic construct
  source 1..44: mol_type = other DNA
ccattagctccataatacagtgaaagaatgtattcatttctcca

SEQ ID NO: 203 - DNA, 41 bp, synthetic construct; identical to SEQ ID NO: 191
  source 1..41: mol_type = other DNA

SEQ ID NO: 204 - DNA, 17 bp, synthetic construct
  source 1..17: mol_type = other DNA
actgacgagattgacgc

SEQ ID NO: 205 - DNA, 20 bp, synthetic construct
  source 1..20: mol_type = other DNA
agaacatggataaggttagc

SEQ ID NO: 206 - DNA, 18 bp, synthetic construct
  source 1..18: mol_type = other DNA
tcgcatcttctatgaaag

SEQ ID NO: 207 - DNA, 20 bp, synthetic construct
  source 1..20: mol_type = other DNA
cccatatactttattcatgg

SEQ ID NO: 208 - DNA, 38 bp, synthetic construct
  source 1..38: mol_type = other DNA
cattagctccataatacagagaatgtattcatttctcc

SEQ ID NO: 209 - DNA, 34 bp, synthetic construct
  source 1..34: mol_type = other DNA
tatgtagaaaaggaggtgaatgagatattctgag

SEQ ID NO: 210 - DNA, 17 bp, synthetic construct
  source 1..17: mol_type = other DNA
actgacgagattgacgc

SEQ ID NO: 211 - DNA, 19 bp, synthetic construct
  source 1..19: mol_type = other DNA
gaacatggataaggttagc

SEQ ID NO: 212 - DNA, 18 bp, synthetic construct
  source 1..18: mol_type = other DNA
tgttatccaacttgacaa

SEQ ID NO: 213 - DNA, 18 bp, synthetic construct
  source 1..18: mol_type = other DNA
atactcgagtctctgctg

SEQ ID NO: 214 - DNA, 48 bp, synthetic construct
  source 1..48: mol_type = other DNA
aggttattatctggatcatctatcacttaatatcagagagatagaaga

SEQ ID NO: 215 - DNA, 39 bp, synthetic construct
  source 1..39: mol_type = other DNA
gtaaatcccacagaactaatggttcgccatctatcctct

SEQ ID NO: 216 - DNA, 17 bp, synthetic construct
  source 1..17: mol_type = other DNA
gcgtttgtctcatatgt

SEQ ID NO: 217 - DNA, 20 bp, synthetic construct
  source 1..20: mol_type = other DNA
tctaggtacttatggacaac

SEQ ID NO: 218 - DNA, 19 bp, synthetic construct
  source 1..19: mol_type = other DNA
agttccattatctaaagct

SEQ ID NO: 219 - DNA, 18 bp, synthetic construct
  source 1..18: mol_type = other DNA
atactcgagtctctgctg

SEQ ID NO: 220 - DNA, 41 bp, synthetic construct
  source 1..41: mol_type = other DNA
gtttgtctcatatgttcttctatatccatgttatccaactt

SEQ ID NO: 221 - DNA, 39 bp, synthetic construct
  source 1..39: mol_type = other DNA
gtaaatcccacagaactaatgttcgccatctatcctctg

SEQ ID NO: 222 - DNA, 20 bp, synthetic construct
  source 1..20: mol_type = other DNA
ctctgatattaagatttgtc

SEQ ID NO: 223 - DNA, 20 bp, synthetic construct
  source 1..20: mol_type = other DNA
tctaggtacttatggacaac

SEQ ID NO: 224 - DNA, 18 bp, synthetic construct
  source 1..18: mol_type = other DNA
ggaatccatgttatccaa

SEQ ID NO: 225 - DNA, 18 bp, synthetic construct
  source 1..18: mol_type = other DNA
ccaatactcgagtctctg

SEQ ID NO: 226 - DNA, 43 bp, synthetic construct
  source 1..43: mol_type = other DNA
tcatctatcagcgtttgtctcttgacaaatcttaatatcagag

SEQ ID NO: 227 - DNA, 39 bp, synthetic construct; identical to SEQ ID NO: 221
  source 1..39: mol_type = other DNA

SEQ ID NO: 228 - DNA, 17 bp, synthetic construct
  source 1..17: mol_type = other DNA
catatgttcttctatct

SEQ ID NO: 229 - DNA, 20 bp, synthetic construct
  source 1..20: mol_type = other DNA
tctaggtacttatggacaac

SEQ ID NO: 230 - DNA, 18 bp, synthetic construct
  source 1..18: mol_type = other DNA
tgttatccaacttgacaa

SEQ ID NO: 231 - DNA, 18 bp, synthetic construct
  source 1..18: mol_type = other DNA
aatactcgagtctctgct

SEQ ID NO: 232 - DNA, 43 bp, synthetic construct
  source 1..43: mol_type = other DNA
tcaggaggttattatctggagagagatagaagaacatatgaga

SEQ ID NO: 233 - DNA, 38 bp, synthetic construct
  source 1..38: mol_type = other DNA
gtaaatcccacagactaatgttcgccatctatcctctg

SEQ ID NO: 234 - DNA, 17 bp, synthetic construct
  source 1..17: mol_type = other DNA
tcatctatcagcgtttg

SEQ ID NO: 235 - DNA, 20 bp, synthetic construct
  source 1..20: mol_type = other DNA
tctaggtacttatggacaac

SEQ ID NO: 236 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaactcggctacacctatcgttaa

SEQ ID NO: 237 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaacaccaatagtgagttcggcga

SEQ ID NO: 238 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaaccgataggtgtagccgataaa

SEQ ID NO: 239 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaacttaacgattgtcgaccctct

SEQ ID NO: 240 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaactgaagatgccatgtactacg

SEQ ID NO: 241 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaacacttttacgcctctggcgtt

SEQ ID NO: 242 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaacaaaatggccaaagcgggtta

SEQ ID NO: 243 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaacgatccactgctgaacagcta

SEQ ID NO: 244 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaactttatcggctacacctatcg

SEQ ID NO: 245 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaactaacccgctttggccatttt

SEQ ID NO: 246 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaacatagctgttcagcagtggat

SEQ ID NO: 247 - RNA, 161 nt, synthetic construct
  source 1..161: mol_type = other RNA
gaccgcttcaccaagtgctgtcccttaggggattagcacttgagtgaaggtgggctgcttgcatcagcctaatgtcgagaagtgctttcttcggaaagtaaccctcgaaacaaattcattgaaagaatgaaggaatgcaactcgccgaactcactattggt

SEQ ID NO: 248 - DNA, 20 bp, Monkeypox virus
  source 1..20: mol_type = genomic DNA
tcggctacacctatcgttaa

SEQ ID NO: 249 - DNA, 20 bp, Monkeypox virus
  source 1..20: mol_type = genomic DNA
accaatagtgagttcggcga

SEQ ID NO: 250 - DNA, 20 bp, Monkeypox virus
  source 1..20: mol_type = genomic DNA
cgataggtgtagccgataaa

SEQ ID NO: 251 - DNA, 20 bp, Monkeypox virus
  source 1..20: mol_type = genomic DNA
ttaacgattgtcgaccctct

SEQ ID NO: 252 - DNA, 20 bp, Monkeypox virus
  source 1..20: mol_type = genomic DNA
tgaagatgccatgtactacg

SEQ ID NO: 253 - DNA, 20 bp, Monkeypox virus
  source 1..20: mol_type = genomic DNA
acttttacgcctctggcgtt

SEQ ID NO: 254 - DNA, 20 bp, Monkeypox virus
  source 1..20: mol_type = genomic DNA
aaaatggccaaagcgggtta

SEQ ID NO: 255 - DNA, 20 bp, Monkeypox virus
  source 1..20: mol_type = genomic DNA
gatccactgctgaacagcta

SEQ ID NO: 256 - DNA, 20 bp, Monkeypox virus
  source 1..20: mol_type = genomic DNA
tttatcggctacacctatcg

SEQ ID NO: 257 - DNA, 20 bp, Monkeypox virus
  source 1..20: mol_type = genomic DNA
taacccgctttggccatttt

SEQ ID NO: 258 - DNA, 20 bp, Monkeypox virus
  source 1..20: mol_type = genomic DNA
atagctgttcagcagtggat

SEQ ID NO: 259 - DNA, 20 bp, Monkeypox virus
  source 1..20: mol_type = genomic DNA
tcgccgaactcactattggt

SEQ ID NO: 260 - protein, 1228 aa, unidentified; identical to SEQ ID NO: 30

SEQ ID NO: 261 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 262 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 263 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 264 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 265 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 266 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 267 - protein, 1306 aa, Thiomicrospira sp.
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 268 - protein, 1214 aa, Butyrivibrio sp.
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 269 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 270 - protein, 1224 aa, synthetic construct; identical to SEQ ID NO: 17

SEQ ID NO: 271 - protein, 1297 aa, synthetic construct; identical to SEQ ID NO: 4

SEQ ID NO: 272 - protein, 1229 aa, synthetic construct; identical to SEQ ID NO: 13

SEQ ID NO: 273 - protein, 1470 aa, synthetic construct; identical to SEQ ID NO: 26

SEQ ID NO: 274 - protein, 1282 aa, synthetic construct; identical to SEQ ID NO: 23

SEQ ID NO: 275 - protein, 1225 aa, synthetic construct; identical to SEQ ID NO: 18

SEQ ID NO: 276 - protein, 1293 aa, synthetic construct; identical to SEQ ID NO: 29

SEQ ID NO: 277 - protein, 1399 aa, synthetic construct; identical to SEQ ID NO: 24

SEQ ID NO: 278 - protein, 1272 aa, synthetic construct; identical to SEQ ID NO: 32

SEQ ID NO: 279 - protein, 1313 aa, synthetic construct; identical to SEQ ID NO: 25

SEQ ID NO: 280 - protein, 1134 aa, synthetic construct; identical to SEQ ID NO: 22

SEQ ID NO: 281 - protein, 1259 aa, synthetic construct; identical to SEQ ID NO: 27

SEQ ID NO: 282 - protein, 1271 aa, synthetic construct; identical to SEQ ID NO: 6

SEQ ID NO: 283 - protein, 1367 aa, synthetic construct; identical to SEQ ID NO: 35

SEQ ID NO: 284 - protein, 1229 aa, synthetic construct; identical to SEQ ID NO: 7

SEQ ID NO: 285 - protein, 1261 aa, synthetic construct; identical to SEQ ID NO: 11

SEQ ID NO: 286 - protein, 1242 aa, synthetic construct; identical to SEQ ID NO: 14

SEQ ID NO: 287 - protein, 1307 aa, synthetic construct; identical to SEQ ID NO: 1

SEQ ID NO: 288 - protein, 1286 aa, synthetic construct; identical to SEQ ID NO: 31

SEQ ID NO: 289 - protein, 1264 aa, synthetic construct; identical to SEQ ID NO: 9

SEQ ID NO: 290 - protein, 1232 aa, synthetic construct
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 291 - protein, 1067 aa, synthetic construct
MKNGINLFKTKTTKTKGVDMEKYQITKTIRFKLLPDNAHEIVEKVKSLKTSNVDELMDEVKNVHLKGLELLFALKKYFYFDGNQCKSFKSTLEIKARWLRLYTPDQYYLKKSSKNSYQLKSLSYFKDVFNDWLFNWEESVSELAIIYEKYKICQHQRDSRADIALLIKKLSMKEYFPFISDLIDCVNDKNSNKTFLMKLSEELSVLLEKCNSRALPYQSNGIVVGKASLNYYTVSKSEKMLQNEYEDVCQSLDKNYDITEMKVILYKEKLDNLNFKDVTIANAYNLLKENKALQKRLFSEYVSQGKVLSLIKTELPLFSNINDNDFEKYKEWSNEIKKLADKKNTFCKKTQQDKIKDIQNKISELKKKRGALFQYKFTSFQKHCDNYKKVAVQYGKLKARKKAIEKDEIEANLLRYWSVILEQEDKHSLVLIPKNNAKDAKQYIETINTKGGKYIIHHLDSLTLRALNKLCFNAVDIEKGQMVRENTFYQGIKEEFERNKINCDNQGVLKIQGLYSFKTEGGQINEKEAVEFFKEVLKSNYAREVLNLPYDLESNIFQKEYTNLDQFRQDLEKCCYALHSKIGKDDLDEFTRRFEAQVFDITSIDLKSKKEKTKTTGEMKKHTQLWLEFWKGAIEQNFATRVNPELSIFWRAPKSSREKKYGKGSDLYDPNKNNRYLYEQYTLALTITENAGSHFKDIAFKDTSKIKEAIKEFNMSLSQSKYCFGIDRGNAELVSLCLIKNEKDFPFEKFPVYRLRDLTYQGDFKDKHDQMRYGVAIKNISYFIDQEDLFEKNNLSAIDMTTAKLIKNKIVLNGDVLTYLKLKEETAKHKLTQFFQGSSINKNSRVYFDEDENVFKITTNRNHNPEEIIYFYRGEYGAIKNKNDLEDILNEYLCKMETGESEIVLLNRVNHLRDAISANIVGILSYLIDLFPETIVALENLAKGTIDRHVSQSYENITRRFEWALYRKLLNKQLAPPELKENILLREGDDKIDQFGIIHFVEEKNTSKDCPNCRKTTQQTNDNKFKEKKFVCKSCGFDTSKDRKGMDSLNSPDTVAAYNVARKKFES

SEQ ID NO: 292 - protein, 1309 aa, synthetic construct
MAKETKEFKTFDDFTNLYEVQKTLRFELEAVPETEIVLENRGIWYKRDKKRADEKPIVKFYMDILHREFTDEALEKIKESGVLNLSGYFKLFEELRRLQNHGANTKEEKKLKLEEIRAKKREISNELSQIRRVFSVRGFDVVDSDWKKKYTIEGKKIKNDKSKTYLILSENILNFLENRFTSKEVERLRSIDKKHVEDYGNVVNSGGENIFATFKGFFGYFDSLIKNRENFYETDGKAGRVATRSVDENLNFFAENLHIFSTDLPKALKDDLSDTQKAIFERSYYKNCLLQKDIKSYNLIIGDINKEINKHRQQRDTKIKFLNTLFKQILSIEEKEQYKHIEINNDEDLIRAIRDFISLNESKISEGTKIFNQFIQRCLQKEDLGQIYLPKDSVNTIAHRIFKPWDEIMALFDRKYFVSLEEIKDLTESSVWKERVLEESKTKSLIFKDTHIHTIISGQEIFSNFILILEKEYKNQFSGFISETRRGKAAFVGYDESLKNLRATIKWFEGKNLKLSETEKVEWIKAIKDYADAALRIFQMTKYLWLPVVGDEEDKDYLRIKAEIDQLTKDNDFYNKINAFIDGYKPEPFIYRSSFQEYLTRRPFSTDKFKINFENSRLLDGWDKDMIDDRMGILLQRDGDYFLGILNKEDRHCLDNLVDVKSEDKNSYALMQFKQLTGLYRQLPRMAFPKKKQPVLEANAEIKKIKEDFDFLQKQKKEREVNVNVVFDNKKLNLLINHYAEFLKENYKDEKCYDFSLLNKEKVYESLSDFYADVDKITYSLSFIQVSIDQLIKTGKILLFRLKNKDLLKGSLGQNKNLHTYYFHALFERENLSQGRIRLGAQAEIFFRPASIEKEKDKNRSNALKKSPKTRYVKEILKNKRYSEDKVFLHLPIQLNADAYDLPSINQNVFEFIKNRQEKVKIIGIDRGEKNLAYYSVISQNSNGKIKIEEPPRDLNLGYLEPLDELENKRQDERKAWQSISEIKSKRDGYISYAVSKIVELMLKYQAIIVLEDLSGKFKRSRMKFEKAPYQQLELALIKKLNYLVKKNSKSGKPGHYLSAYQLTEPVGSYKEMGKQTGIIFYTQAGYTSRTCPTCGWRKRVQGLYYKDRTSAQRRFDPKTGVKIFYDSVNDRFVFQYHPVYEQKELKEWDKEIYSDVTRIRWNNEEKKNNEYRKGDITLKIKRLFRDRGIDLSRNINEQLVNVGDASFWEELINLLRLITEIRNIDNENNRDFIECPHCHFQSENGFHGVAWNGDANGAYNIARKGLLITKAVCDPEKNVGDITWSDLKVDMKDWDAATDEWAKKNPEK

SEQ ID NO: 293 - protein, 1456 aa, synthetic construct
MENEKIFSDLTNRYQVVKTLPFELKPVPRTRVLLGLDNPNKGEIFSKDRERAENFTIIKKYIDRLHSLFINESLKKADIDFSNFYKQYGKNINTKNNKNIDDDNDINDDEKEDSENDNLKKYRQEIANLFNKSKYKSWVNVGKDGDKISGMLFEKGLIDLLRTHFSDNLNEDIEIPELFSNKKIKDTRKLKEIINSFGKDGKDGQNFTTYFSVSFHNNRKNYYKSDGKMGRVSTRIVDENLERFCKNIYLYKEIIGKNEIKEIFSGNWDIYLQKKPNFSNDKTYKKLDEFKNDKYDWEMIFRDVNSYNKYFLQSDIEFYNYIRGKLNQDINEYNGKKRDSKEKINSQFENLRNQVHGEKKNYDDDFEIDEDNIIQFINEIFVRHNQNKMRFSEKLFSDFIDLLMVDNGDKLDKVYFSQKAVENAIARYYFVEETTNEGREPLLISLLLQNAGKDRKKLSNKPIKLGDIKFVLDQANNKPAEDIFKNRYVLSESNNDGIINANDKNHWANLLRLIKKDFYFHKDNLIKSQDKLALETKYNKGSDEGERQIETIKNFAESAKAILRMTKYFDLRKNGVIQNVIGGKDPIHEEVDKYFDGDVLSGEESCRISKYYDALRNFITKKAWSADKIILNFDCSEFLGGWDRSQEQKKRGIILRHRDGDEERYYLAVLGKNGKQYFENRTLFKGCESSDWQKIEYNVIQKPHMSLPKNLITPFFKKDKITNERFIDRSKKGAKALIEIDINPSDEFLNNYNLGKHTKENLDKSFLCDYFKYLMDAIAKYYKGEFNFNFPDVSNFDNTQPFYSFIEKNAYSIKYFGISSKEIEKLIADCYYKEDVYLFQIYCKDFEIDPKIGKAKYGNEFRTKAEIRKSKGEEAGNENLNTKYFKLLFDEKNLKNQNGIVYKLNGGAKMFYRPSSIKKDEKIDGKWRYKEDKYSLNITITCNFSSKKDDLSIDKDINKKIAEVNANSDFRIISIDRGEKNLAYCCVMDENANILDIKSLNRITRYDKNGKAIKEKNMFHEVKDGKLCYGEPVYDFYKDYQNLLDEREIKRLVNRRSWNVIEDIKNLKKGYVALLINYICKAVVIAINEGKYPIIVLESLDKGMLHNRVKIEKQIYRGVEEGLVRKLNYFVDKKTDNVLNAWQLLAKFETVGSSLDRKKQLGIIFYVDPGYTSITCPCCGFRQRKYIKAERAEENFKEIKIKFDGKRYSFAYDYRCIDDNGKEKSKEDIIYSNVKRLLRSGRNGRAVQIEDVTDELTNLFKKHNINIEQDINEQLAGKDNKFWKQLLWWFNAIEQIRNTQSLRRKFNTEENKLEILENNDCDFILCPHCYFDSNKDKFQNKIWNGDANGAFNIGRKGIIDIFEIKKHQRMLSDFMEQWGIDKLPKANGGNQAVIEIVKNDKKYNLCILNNKKIPYYCLRIGKEKIDSIADDRKCNQLPDLMVNWKKWDMWLDKWGK

SEQ ID NO: 294 - protein, 1486 aa, synthetic construct
MPEVKNVFQDFTNLYELSKTLRFELKPVPETEKILELNAAKTKKFPKDLYRAENFEIIKKYTDELHRTYIRETLNNVNIDYLKFLEIFRINGKKKNEMTDENEESDENNEKDDIQKIKKELRSKIGNLFNKWNNDKDNKFKDWVKIDVGKKEKEVSGDLFGKELITILKNYFKNKLDSKVNVPMLFFNEQEIKNGEAKKQRKLEAVFENFDKFTTYFTDSFYNNRKNYYKTEGRVGQVATRIIDENLPRFCSNLIAFNEVVSLYSTLLNNFDLGWKEYLNEKKINQTWVEKFELSNYDWKALFNDVNYYNQCLLQEGIDKYNYIIKKLNKDINEYTQNKYKSVEKGNNNNPDINFFQKLHKQIHGERDFKLIEIDIDENNIFTKILPEFILHSDMKLMTKIDEEVGVEEIVGAERIIKIFIKQELKDLEKIYLSRRAIETISAKWFHSWETLKDLILGYLNKDLLESKKRKKVPDFVDFNIIKIVLENNKDDYKDLFKRKYFEADKNEFVDWIDSSGGTKKLEFGGENWINFLNVFEYEFGTLLTEYKKNKNALLYLIDKKIDYDKNNEVGQTAAIKNFADSALGIFRMVSYFALRKKGVMVEPKNGKDEIFYAFVDRYLDGDDNDREEQNKIVQYYNTLRNFVTQKAWSIDKVRLCFDCGEFLKGWDKDKIHERLGIILRNNNKFYLGILNKNHKQIFIKIKSHDNNNFYYVIYDYKQLNNVYRQIPRLAFPSRSVKKGDAYMLRAIQERKKKFFLEDEEFIELQEIKNEYDKIGNDLSKEKLTKLIEYYKKVVISNYSSLYNVSNLNNKKFNSINEFNQYVENLMYSLIPTRISPDFIKEKISKGELYLFQIYNKDFELDESIGKEKFGEDFAPVIMDGKNNLHTEYFKLLFNDSNLKNPNGVVFKLSGGAKMFYRPATENLPIKKDRDGNIIKNKKGENVIVGQRYKEDKYFLHLPIILNFVNKGKNYSINDMVNKAITNASDDQDKFRIIGLDRGEKHLVYYSVINERQEIIEIGSLNNISRKDNKGEIIEEKNWYHDKFGNIEKEPTKEYHKDYHNLLDQREIERLKSRQSWEKIENIKELKEGYISAVINKICNLVIKAIKENKIPIVALENLNSGMKRGRIKIDKQIYQKLELKLAKKLNFLVDKKEKNYLSAWQFTPKIETFSGDIEKKNQVGIIFYVDPAFTSATCPNCGFRKRIKMDPQNAKKKIKDMEITYENGIYKFDYPIENGENDVVYSDVERLKWDNEKKKVIKTKNVSDDFGKLFEDIKDKNNLKKELLSIGEENKEFWKEFSRCFNLLLRIRNSKLIKRKLNDDTGKVEIIADDDLADRDRDFIYCPQCHFHSEGGDVFGEFVKKKYLGKDNFEFNGDANGAYNIARKTIIAVNKIKDYQLGLNHFIEKYRISELPNNGKDKKNIFYNNNSYILSFFEVQDEKFRKVKVYGLKKDGDRQIIQKKEMWYRRYPDIFVNNKEWDKFVQNKS

SEQ ID NO: 295 - protein, 1403 aa, synthetic construct
MLFFMSTDITNKPREKGVFDNFTNLYEFSKTLTFGLIPLKWDDNKKMIVEDEDFSVLRKYGVIEEDKRIAESIKIAKFYLNILHRELIGKVLGSLKFEKKNLENYDRLLGEIEKNNKNENISEDKKKEIRKNFKKELSIAQDILLKKVGEVFESNGSGILSSKNCLDELTKRFTRQEVDKLRRENKDIGVEYPDVAYREKDGKEETKSFFAMDVGYLDDFHKNRKQLYSVKGKKNSLGRRILDNFEIFCKNKKLYEKYKNLDIDFSEIERNFNLTLEKVFDFDNYNERLTQEGLDEYAKILGGESNKQERTANIHGLNQIINLYIQKKQSEQKAEQKETGKKKIKFNKKDYPTFTCLQKQILSQVFRKEIIIESDRDLIRELKFFVEESKEKVDKARGIIEFLLNHEENDIDLAMVYLPKSKINSFVYKVFKEPQDFLSVFQDGASNLDFVSFDKIKTHLENNKLTYKIFFKTLIKENHDFESFLILLQQEIDLLIDGGETVTLGGKKESITSLDEKKNRLKEKLGWFEGKVRENEKMKDEEEGEFCSTVLAYSQAVLNITKRAEIFWLNEKQDAKVGEDNKDMIFYKKFDEFADDGFAPFFYFDKFGNYLKRRSRNTTKEIKLHFGNDDLLEGWDMNKEPEYWSFILRDRNQYYLGIGKKDGEIFHKKLGNSVEAVKEAYELENEADFYEKIDYKQLNIDRFEGIAFPKKTKTEEAFRQVCKKRADEFLGGDTYEFKILLAIKKEYDDFKARRQKEKDWDSKFSKEKMSKLIEYYITCLGKRDDWKRFNLNFRQPKEYEDRSDFVRHIQRQAYWIDPRKVSKDYVDKKVAEGEMFLFKVHNKDFYDFERKSEDKKNHTANLFTQYLLELFSCENIKNIKSKDLIESIFELDGKAEIRFRPKTDDVKLKIYQKKGKDVTYADKRDGNKEKEVIQHRRFAKDALTLHLKIRLNFGKHVNLFDFNKLVNTELFAKVPVKILGMDRGENNLIYYCFLDEHGEIENGKCGSLNRVGEQIITLEDDKKVKEPVDYFQLLVDREGQRDWEQKNWQKMTRIKDLKKAYLGNVVSWISKEMLSGIKEGVVTIGVLEDLNSNFKRTRFFRERQVYQGFEKALVNKLGYLVDKKYDNYRNVYQFAPIVDSVEEMEKNKQIGTLVYVPASYTSKICPHPKCGWRERLYMKNSASKEKIVGLLKSDGIKISYDQKNDRFYFEYQWEQEHKSDGKKKKYSGVDKVFSNVSRMRWDVEQKKSIDFVDGTDGSITNKLKSLLKGKGIELDNINQQIVNQQKELGVEFFQSIIFYFNLIMQIRNYDKEKSGSEADYIQCPSCLFDSRKPEMNGKLSAITNGDANGAYNIARKGFMQLCRIRENPQEPMKLITNREWDEAVREWDIYSAAQKIPVLSEEN

SEQ ID NO: 296 - protein, 1356 aa, synthetic construct
MTIKKHKPFTNFECLTPVQKTLRFRLIPVGRTTEFVKCRNIIEADRKRSEMYPLLKELADRFYREFMTDQLSNLLFDWSPLVEALLLARNNTDPRENQRIASLVRDEQKKYRTLLLKRLSGQVDRNGTPLPKNTASVNKKYYDDLFKARFVTETLPAYLEHLKNKPDGRISDELFDAYKDALDSYQKFTSRLTNFWQARKNIFTDEDIATGFAYRIVHEIVPDYLFNRRVYEQHKLDFPEPLDLLETELKKKNLIANDESLDALFTIPAINRLLTQKGVDLHNAVIGGFFTDDHTKVQGFNELANLKNQTLKNVSDNSEIKPVGKMTRLKKHILSISESTSFLFEQIESDDDLLARIIEFNNTLSEPDIDGLSIADINDQLYNIMTGVDPSTILVHARNLNKLSHEASLSWNRLRDGLYQMATESPYREDERFKRYIDASEEERDLSKLKNDIYFSLQELQFALDQSIDLEEEATPTEDIFLPFEFPGMDLKSELTVLFRSIEQLISSETKLIGNPDAIATIKKYLDAIMARYSIWNLLSCEAVELQDDLFYPEYDRVMGSLSNIILLYNLARNYLSRKPSSKEKFRLNFDKPTLADGWSESKVPDNFSVLLRKDDLFYLGILKDRKAYRVLSYENCDETAKNIKGYYERMIYHFSPDAYRMIPKCSTARKDVKKHFGEQGETTGYTLYPGASNFVKPFTIPYEIYRLQTELVNDKKRYQADYLKQTEDEEGYRQAVTAWIDFCKSYLESYEGTSTFDYSHLLKSEDYEDVNQFYADVDRASYSIYFEKVSVDLIHTMVDRGDLYLFQLYNKDFSPHSTGKPNLHTMYWRALFSNDNLQNNTIKLNGQAELFYRPKQVEQPTVHLQGSYLLNRFDKHGDVIPAGLYCEIYNHINERHPEGYTLSEEATQGLLDGRFVYREAPFELVKDKRYTEDQLFLHVPLEFNWTASANVPFENLANEYIKKDSDLHIIGIDRGERNLLYYSVINLQGDIVKQGSLNTLIQQTTLKGETVERQIPYQSMLKQREDERAEARQNWQSIDRIKDLKEGYLSHVIYKLSRLIIKYHAIVVMENLNVGFKRGRFKVERQVYQKFEVALINKLNALSFKEYEPNELGGVMRPWQLARRVVSPEDTRSQNGIVFYVPASYTSIVDPVTGFANLFYLNRIRNKDLNSFYGHFQEIRYDHEFDRFIFRFNYADFGVFCRIKNVPSRTWNLVSGERKAFNPKRRMIEKRDTTDEIKKALEAHGIAYQNEQNLLPLLLENENLLARIHRSFRLVLQLRNSDSDRDDIVSPALDKENNTFDSGQQPYESSLPINADANGAYNIARKGLLLVDKVKNDKRAVLSNREWFEYLMAEE

SEQ ID NO: 297 - protein, 1296 aa, synthetic construct
MENKDYSLSRFTKQYQNSKTVRFALTPIGRTEEYIIQNQYIEAARRKNQAYKIVKPIIDEKFRSMIDDVLTHCEKQDWVTLDKLILQYQNNKCRENMDALAEQQEEIRKNISEEFTKSDEYKNFFGKEDSKKLFKIFLPEYLNQINASESDKEAVNEFQKFKTYFSNFLIVRADIFKADNKHNTIPYRIVNENFMIFAGNKRTFSNIIRLIPNALEEIAKDGMKKEEWSFYNIQNVDSWFEPDSFQMCMSQKGIQKYNFIIGLVNSYINLYTQQNPQATEVKRSRLKLRMLHKQILSDRVNPSWLPEQFKEGEEGEKQIYEAILALENDLIKNCFDKKYDLWIQSIDIQNPRIYIAASEMARVSSALHMGWNGLNDVRKTILLKSDKKQAKVEKILKQDVSLKDLSDTLNRYADIYKEEQIPSLYQYIEYGSELLQDCAITRKEYHDLLNGNSNTLSLNQNEKLIEGLKAYLDSYQAIVHFLNVFIVGDELDKDTDFYAELDGLVESLSEIVPLYNKVRNYITRKVYSLDKMRIMFERSDFLGGWGQSFDTKEALLFQKDNLYYIGIIEKKYTNMDVEYLHEGIKEGNRAIRFIYNFQKADNKNIPRTFIRSKGTNYAPAVRKYNLPIESIIDIYDVGKFKTNYKKINEKEYYESLEKLIDYFKDGILKNENYKKFHFNWKPSNEYENINEFYNDTNNACFLLEKEEINYDHLKEQANQGKIYLFQISSKDFNEGSKGTPNLQTMYWRELFSNQNCKDGVIKLCGGASIYMRDASIKQPVVHRKNAWLINKWYKVNGQNVVIPDNTYVKFTKIAQERMNEDELTPQERQLWNSGLIQKKKATHDIMKDRRFTKKQYMLHAPLTINYKQQDSPRYFNEKVRSFLKDNPDINIIGIDRGEKNLIYITIIDQKGNILKGMQKSFNQIEEKGKEGRTIDYYSKLESVEARHDAARKNWKQIGTIRELKEGYLSQVVHEITQLMIQYNAVIVMENLNMGFKKGRMKVEKSVYQKFEKMLIDKMNYLAFKRDMQGNAIDPYEVGGVMNGYQLTDRFTSFADMGSQNGFIFYVPAAYTSVIDPVTGFVNVFQKTEFKTNDFLHRFDSISWNDKEQSFVFTFDYQNFKCNGTCYQNKWSLYADVDRIETIIKNNQVDRIEPCNPNQKLIDFFDKKGIIYRDGHNIVDDLEKYDSKTISEIIHNFKLILQLRNSMRNPDTGEIIDYIASPVMHNEERFDSRKRNPELPQDADANGAYHIALKGLMFLQKINEYADSDGNMDNRKLKITNEEWFKYMQTRKEHTYF

SEQ ID NO: 298 - protein, 1250 aa, synthetic construct
MSNKTSSITTTNKLSYTGFHNNGKQSKTLMFELKPIGRTTEHLDRKGYLADDIDRAESYKTFKEIADNFHKNLIEESLATFTFSDTLKDYFDLWLSPVRTNEDTPKLRKMEAKLRKELSSALKQHPSFAATSSGKRLIDEALYPNASDKERQCLDRFKGRSSYLDSYTEVRSFIYTDLCKHNTIAYRVVNENLKIYLENILAYEKLMQTAVNGKLETVKEMFHDLYPTFSMDISIFFTSYGFDYCLSQNAITRYNILLGGWSDDNGIHHKGLNNYINEYNQTVPRNKRLPKLNKLQKMILSEENSMSFIIDKFENDVDLANAIRYWLKNCQFDALNLLIWTLDVHYNLDEIHFKNDNQGKNISDLSQALFKNHHVIRDAWDYDYDIVNAKAKSRQKPERYAEKRDKAFKKINSFSLSYLANILSQYDNQYANFVAQFKTRISVHIQNVQQMIADKTLDMRLDPLMLLKSISSDTKLVEDIKRVLDSLKDMQRMLTPLLGEGTEPNRDAMFYSDFEPLMNYVDTLTPLYNKVRNYITKKPYSTKKTSLYFGASNFGSGFDVTKLPVSHTIIMRDKGCYYLAVIDNNKLIDKLYDHNDNDGYEYMVYKQIPSPIKYFSLKNILPQDPPDDIRQLLEDRKNGAKWSHDDETRFIDYIVNEFLPTYPPIHDKNGNPYFSWKFKNPDEYESLNEFFDDVSKQAYQTSFRFVSRDFVDDAVENGDIFFFQIYNQDFSPASHGKPSPHTLWFRALFSDVNLETKDIRLKGNATAYFRPASIFYTDEKWRKGHHYEQLKNKFKFPIIKDKRYALDKFFFHITLEINCNATVEKYFNNRVNEEIRKADRYNILAINRGERNLLYAVVMDQDGTILEQKSFNIIKSELPNKTVKETDYWKKLHAREKERDTARKSWKSIECIKDLKKGYLSYVVKTITDMMFEYNAVLVMENLDIEMKRSRQKIEKNVYAQFQNAIIQKLSMYVNKDIDLHIARTAPGGTLNPYQLTYIPASRTKTPKQNGFVFFLNPWNITEIDPTTGFVDLFQTCFRTKNEYKDFFAKFKDIRYNEAQGWFEFDTDYTYFRDKEKAGKRTRWNICSYGTRLRRFRNPDKNYAEDAMTVYPTQMLKDLFDEYNIPYAPASAKSTSISIKDDIIQIDKLDFYKKLLYILKLIVQLRNTSPSSTEQEDDYIISPVINEDTNWFYDSRDYNEESLLPCNTDANGAYSLALKCNMVIDRIKNTIPGEPVDMYISNADWLDARQ

SEQ ID NO: 299 - protein, 1328 aa, synthetic construct
MNSKTSIFDFSNIFGRDITLRFKLTPVTINSKGEVKDANGADPYRPYLSADEELQEQYELLKTAIDAYHQMYIDKKLKHILCLPLTEKGKDGVEHDTAKSKFVKSCLAYIKDYGEKDKKRQTADLRTFISRVFADDNISSLPPYKVKSDFITKTLRQWLEQPDTKVEKKEAILDLIEKNGSKLYANCQGLLEARQRLYEKDGKSTSVPYRCIDRNLPRFSKDYHLFEKILGDCSDVFDFEQLDKDFSEELKGIARLSGIRVESVREVFQPLLYLAYLNQEGIQYLNTIIGTKKEKGTSALGLNEYINQYNQKQGIKKKKDGIPMLNKLNNQILFGDEVFIETLAEHKEAIPVIKKVVSSLGKLGAFDGECHENKLYQFLLSLSSYAGNIYVNTKVVAQISSSLWGDYSILYDAVKHDKNGRLIQKSVTLGELNEKIERLKLEDNRDAFEYFRRSQVKDVVHGSSNVGVFEQLKNCYNDFVEKKILKCSFFSEDQVLVIQRLFDSILSLQRIFKVFCPSLYEVDSDGLFVAKFSDYWNVLRGFDKDYDLLRNLFKRKPYSTDKIRVHFGLSNLMDGFVDSWTDKKDKGTQYNGYILRQAHSFVDENTSKELQEFQRYNYYLVISGNVRLFREKGNALVCEKKKEKLVASDEFSGFERFDYYQSSINNFNREFKRLTGRDRKSFTDEILQNEGKKELKSTYIENLIKVAKSMKRLTALQNLVSDEKVRKYSENLDYETLSAEIGQILATGRERKYVPVSTNEMKNLLKSSKNNKGEEVRTFMFRISNKDLSYAETMQKGERKSHGAENMHTMYFRALLDTLQNTFDIGTGTVYFRKASDKRKMKYDEKNPTHRKGDELAFKNPYNKGKKKSVFGYDLIKDRRYTKDSYLFHLSITQNYQKKGNAEDLNAMVRDYIRTQEDLRVIGIDRGERNLLYATMIDGEGHILAQKSFNVIGYQGTTASGESFQVETDYHQLLNEKAEKMRSLQREWKEMDKIQDMKDGYLSVVVHELAKMVVENNAIIVMEDLNMGFMESRQSQLANVYQKFEEKLRNKLQFYVDKRKRNDEPSGLYHALQLAGTETKDNQNGFIFYIPAWNTSKIDSVTGFVNLFNLKYTNIKDAKAFFSTFEKIEKNVETGHYDFTFSYSSMARKKMAKRMDGTRDSWTISTHGSRIVREQKGNYWEYREIESLTSEFDALFEKYSIDTRCRLKEAIDKCGEAEFFKELIRLMKWTLQLRNYDDRGNDYIVSPVCYRGNEYYCSLDYDNEEGMCISKIPCQMPKDADANGAFNIARKGLMLCERLKKGEKIGVIKGTEWLQYVQNMSERYVGMV

SEQ ID NO: 300 - protein, 1434 aa, synthetic construct
MINTMEQPKKSIWDEFTNLYSLQKTLRFELKPQGKTKELVRTLFINPEEHHHKLISDDLELSKNYKKVKKLIDCMHRNIINNVLSKHQFTGEELKKLDKNSNAEDNDTETDNADKKDPFAKIRERLTKALNEESKIMFDNKLLNPKKGKNKGECELKKWMDKAEDKYFELGNNEKIDKEAVKADMERLEGFFTYFGGFNKNRENVYSSKKIATAIPFRIIHDNFPIFKKNIENYKKITEKHPELAKLLNEKGANEIFQLEHFNKCLTQDGIDVYNNEKLGIIAKEQGKEQDKGINQLINEYAQKKNKEIKENAKGGEKPKKIKIAVFDKLKKQILSISKTKSFQFEVFEDTSDIINGINKRYTFLTEAKEGMSIVDEIKKIIGSVGDEKYSLDEIYLKEKFISTLSKKLFNYSRYIEVALEKWYDDRYDDKINKSGTDKRKFISAKQFSITSIQDAINYYLEKYEKDEELSKKYTGKNIIVDYFKNPTITIEHKQKEEVISEEKDLFKELEVRRNVIQHILNGDYKKDLKEEKQQDGDSEKVKAFLDALLEFNYILNPFIIKDKNLRKEQEKDEEFYNEIKKLQESIFEAEILDLYNQTRNYITKKPYKLDKFKLTFGSGYFLSGWSNDMEEREGSILIKYNEDRSKNYYLIIMAKPLTDDDKKQLFSDNGTHSKICIYEFQKMDMKNFPRMFINSKGSNPAPAIEKYNLPIKTIWADYQKYKNLNQKGKDKFLEENPDFRHNLIGYFKICAEKHESLAPFKHQFSSIWKPTKEYENLAQFYKDTLEACYNLKFENVNFDNISQLVSSGKLHLFKIHNKDFNPGSTGKKNLHTLYWEMLFDEKNLQDVIFKLSGGAELFYREASILKNKIIHKIGEKVLKKFFKLPDGKLEPVPAESIKNLSAYFRKELPEHELTEIDRKYIDNYSIIGKKDDKLGIMKDERFTVDKIQFHCPITINFKSKNKNFINDDVLEYLHKRDDVHIIGLDRGERHLIYLTMINKDGKIVDNMQFSLNELQRRYKINGNEEIQKINYQKLLDTREVSRTEARRNWQTIENIKNLKEGYLSLIVHQLAKLMIEKNAIVVMENLNYGFKDSRARVEKQIYQKFESILIKKLQYLVMDKNNLYDSGGVLSAYQLTNQEVPAYKYISKQNGFLFYVPPDYTSKIDPETGFINLLDTRYYSRKNAVALLNKFDKIYYDRDNKYFRFDFDYNSTDSNGNKNFDKLRVDISELTRTKWSVCSHPAKRSITVQINNKWVRQPINDVTDKLIKLFEDKQIGYESGKCLKDEILKVEDAKFFEDLLRYLSVLLALRHTYTENGVEYDLIISSVEKAPGSNEFFVSGKDNNLPANADANGAYNIARKGLWLLRKLDEIDNQELAIKKFNELKHAKEIKKNGEESKEDKGDRKRKKKWVSQWCPNKEWLAFAQSMQDVSEK

SEQ ID NO: 301 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 302 - protein, 1283 aa, synthetic construct
MSNLNTFISPEFTGKIKMTKSLKVSMIPIGETEHWIAKHKVFEKDRELFDKNLKARPILDEFIKYTVSRALPNLLFDFEAYYLVKKDRTKARAFEKELAKTVTDLILKEMDELKSASLIDSADFVKTTLKKFAGTHDIPGLSRIEAIESLEAASKLTALNGKFNTSRIAIINTLIPKRIIENFDIYLSNMEKIRNVYESGEFGFLFERYPDTLLFMEPANYRTVCSPEAIEDYNRFISGYGDSTESWIKGFNQELSEASNSSKSSNGGVRRYSLIKPLHKQHLFETKKFFTFASISSDDDVRELINSVKGSTEDACLNALAFFSSSDPKTLFVKGSYLHTLSAFLYGSANSYILPERIKEGEKARLTAEYDSVAKKTKAVTTRYNVAMNNISKKINEKIFSLADIDAYCCDISKRRSVREILLGIMQEMYAAVYGENGKWSNIEAEAVLDSKTKIWKAKNGAVAKAVNDYLTAILEIRKFIRPFALRMEELEELGLDTSSALDAGEITNTLFEAVRAQKLVHAYLTRNDADIALSTQVYFGGTQKAAASWWNYETGDIQNRQIALAKKDGMYYFIGTFDERGSYSIEPASPGEDYYEMLDVKKGQDANKQIKKVLFSNKAIREHFADSSNDYVITTKVNSPITVRREIFDKYQAGEFKLTSQKIRKGDLVGEKEMTYYREYMDLLFQMAKGYTEYSRFNMDTLLPIEEYDTENDLLDDVNTNTIDYRWVRISAACIDDGVRNGDIFVFRAQTSSMYGKRENKKGYTGLFLELVSDENLLVTRGMSLNSAMSIYYRAKVHDAITVHKKGDVLVNKFTNARERIPENSYKAICAFYNSGKSIEELTIEDRDWLAKATTRICSGEIIKDRRYTKNQYSISISYNINRSVNNRKRVDLATIVDDTASAGRIISVTRGTKDLVYYTVIDDGGSVIEARSLNVINGINYAKMLAQISEERHDSNANFDIPKRVETIKEAYCAFAVHEIISAALKHNALIVVELISDAIKDKYSLLDNQVFLKFENVLKNCLMSVKVKGARGMEPGSISNPLQLCNADDKSFRNGILYQIPSSYINICPVTGYADIIDYYNIVSAGDIRNFFVRFENIVYNKEKARFEFSFDLKNIPIKLEKCPDRTKWTVLGRGEITTYDPLTKSNHYVFDAAQMLAETVSKEGLDPCANIVEHIDELSAATLKKMFNTFRNIAKGIVSECDEVPVSYYKSPVIDEADIKNKSLDNKSISEIKCYNLDEKARYYLALAKSSSDGENKNRYVSSTAIEWLNYIQEKRTHE

SEQ ID NO: 303 - protein, 500 aa, unidentified; identical to SEQ ID NO: 10

SEQ ID NO: 304 - protein, 507 aa, unidentified
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 305 - protein, 529 aa, unidentified; identical to SEQ ID NO: 3

SEQ ID NO: 306 - protein, 726 aa, unidentified
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 307 - protein, 517 aa, unidentified
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 308 - protein, 481 aa, unidentified
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 309 - protein, 358 aa, unidentified
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 310 - protein, 507 aa, unidentified
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 311 - protein, 534 aa, unidentified
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 312 - protein, 537 aa, unidentified
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 313 - protein, 540 aa, unidentified
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 314 - protein, 542 aa, unidentified
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 315 - protein, 564 aa, unidentified
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 316 - protein, 610 aa, unidentified
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 317 - protein, 369 aa, unidentified
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 318 - protein, 552 aa, unidentified
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 319 - protein, 534 aa, unidentified
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 320 - protein, 577 aa, unidentified
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 321 - protein, 613 aa, unidentified
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 322 - protein, 615 aa, unidentified
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 323 - protein, 775 aa, unidentified
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 324 - protein, 777 aa, unidentified
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 325 - protein, 610 aa, unidentified
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 326 - protein, 632 aa, unidentified
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 327 - protein, 625 aa, unidentified
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 328 - protein, 517 aa, unidentified
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 329 - protein, 410 aa, unidentified
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 330 - protein, 486 aa, unidentified
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 331 - protein, 602 aa, unidentified
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 332 - protein, 494 aa, unidentified
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 333 - protein, 526 aa, unidentified
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 334 - protein, 649 aa, unidentified
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 335 - protein, 414 aa, unidentified
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 336 - protein, 413 aa, unidentified
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 337 - protein, 449 aa, unidentified
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 338 - protein, 711 aa, unidentified
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 339 - protein, 574 aa, unidentified
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 340 - protein, 400 aa, unidentified
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 341 - protein, 743 aa, unidentified
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 342 - protein, 769 aa, unidentified
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 343 - protein, 666 aa, unidentified
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 344 - protein, 564 aa, unidentified
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 345 - protein, 565 aa, unidentified
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 346 - protein, 499 aa, unidentified
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 347 - protein, 560 aa, unidentified
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 348 - protein, 404 aa, unidentified
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 349 - protein, 392 aa, unidentified
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 350 - protein, 500 aa, unidentified
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 351 - protein, 507 aa, unidentified
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 352 - protein, 529 aa, unidentified
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 353 - protein, 726 aa, unidentified
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 354 - protein, 517 aa, unidentified
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 355 - protein, 481 aa, unidentified
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 356 - protein, 534 aa, unidentified
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 357 - protein, 537 aa, unidentified
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 358 - protein, 541 aa, unidentified
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 359 - protein, 542 aa, unidentified
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 360 - protein, 564 aa, unidentified
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 361 - protein, 610 aa, unidentified
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 362 - protein, 552 aa, unidentified
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 363 - protein, 534 aa, unidentified
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 364 - protein, 577 aa, unidentified
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 365 - protein, 613 aa, unidentified
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 366 - protein, 615 aa, unidentified
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 367 - protein, 775 aa, unidentified
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 368 - protein, 777 aa, unidentified
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 369 - protein, 610 aa, unidentified
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 370 - protein, 632 aa, unidentified
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 371 - protein, 625 aa, unidentified
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 372 - protein, 517 aa, unidentified
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 373 - protein, 410 aa, unidentified
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 374 - protein, 602 aa, unidentified
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 375 - protein, 494 aa, unidentified
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 376 - protein, 649 aa, unidentified
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 377 - protein, 414 aa, unidentified
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 378 - protein, 413 aa, unidentified
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 379 - protein, 449 aa, unidentified
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 380 - protein, 711 aa, unidentified
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 381 - protein, 574 aa, unidentified
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 382 - protein, 743 aa, unidentified
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 383 - protein, 769 aa, unidentified
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 384 - protein, 564 aa, unidentified
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 385 - protein, 565 aa, unidentified
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 386 - protein, 499 aa, unidentified
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 387 - protein, 358 aa, unidentified
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 388 - protein, 369 aa, unidentified
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 389 - protein, 486 aa, unidentified
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 390 - protein, 400 aa, unidentified
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 391 - protein, 666 aa, unidentified
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 392 - protein, 560 aa, unidentified
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 393 - protein, 404 aa, unidentified
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 394 - protein, 392 aa, unidentified
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 395 - protein, 707 aa, unidentified
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 396 - protein, 757 aa, unidentified; identical to SEQ ID NO: 58

SEQ ID NO: 397 - protein, 765 aa, unidentified
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 398 - protein, 766 aa, unidentified; identical to SEQ ID NO: 54

SEQ ID NO: 399 - protein, 812 aa, unidentified
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 400 - protein, 812 aa, unidentified
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 401 - protein, 793 aa, unidentified
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 402 - protein, 441 aa, unidentified
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 403 - protein, 812 aa, unidentified
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 404 - protein, 812 aa, unidentified
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 405 - protein, 761 aa, unidentified; identical to SEQ ID NO: 59

SEQ ID NO: 406 - protein, 717 aa, unidentified; identical to SEQ ID NO: 55

SEQ ID NO: 407 - protein, 781 aa, unidentified
MRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 408 - protein, 793 aa, unidentified; identical to SEQ ID NO: 401

SEQ ID NO: 409 - protein, 717 aa, unidentified; identical to SEQ ID NO: 55

SEQ ID NO: 410 - protein, 761 aa, unidentified; identical to SEQ ID NO: 59

SEQ ID NO: 411 - protein, 765 aa, unidentified
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 412 - protein, 766 aa, unidentified; identical to SEQ ID NO: 56

SEQ ID NO: 413 - protein, 870 aa, unidentified
MLVRTSTLVQDNKNSRSASRAFLKKPKMPKNKHIKEPTELAKLIRELFPGQRFTRAINTQAGKILKHKGRDEVVEFLKNKGIDKEQFMDFRPPTKARIVATSGAIEEFSYLRVSMAIQECCFGKYKFPKEKVNGKLVLETVGLTKEELDDFLPKKYYENKKSRDRFFLKTGICDYGYTYAQGLNEIFRNTRAIYEGVFTKVNNRNEKRREKKDKYNEERRSKGLSEEPYDEDESATDESGHLINPPGVNLNIWTCEGFCKGPYVTKLSGTPGYEVILPKVFDGYNRDPNEIISCGITDRFAIPEGEPGHIPWHQRLEIPEGQPGYVPGHQRFADTGQNNSGKANPNKKGRMRKYYGHGTKYTQPGEYQEVFRKGHREGNKRRYWEEDFRSEAHDCILYVIHIGDDWVVCDLRGPLRDAYRRGLVPKEGITTQELCNLFSGDPVIDPKHGVVTFCYKNGLVRAQKTISAGKKSRELLGALTSQGPIALIGVDLGQTEPVGARAFIVNQARGSLSLPTLKGSFLLTAENSSSWNVFKGEIKAYREAIDDLAIRLKKEAVATLSVEQQTEIESYEAFSAEDAKQLACEKFGVDSSFILWEDMTPYHTGPATYYFAKQFLKKNGGNKSLIEYIPYQKKKSKKTPKAVLRSDYNIACCVRPKLLPETRKALNEAIRIVQKNSDEYQRLSKRKLEFCRRVVNYLVRKAKKLTGLERVIIAIEDLKSLEKFFTGSGKRDNGWSNFFRPKKENRWFIPAFHKAFSELAPNRGFYVIECNPARTSITDPDCGYCDGDNRDGIKFECKKCGAKHHTDLDVAPLNIAIVAVTGRPMPKTVSNKSKRERSGGEKSVGASRKRNHRKSKANQEMLDATSSAAE

SEQ ID NO: 414 - protein, 830 aa, unidentified
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 415 - protein, 790 aa, unidentified
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLNADLDVATTNLVRVALTGKVMPRSERSGDAQTPGPARKARTGKIKGSKPTSAPQGATQTDAKAHLSQTGV

SEQ ID NO: 416 - protein, 790 aa, unidentified
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLHADLDVATTNLVRVALTGKVMPRSERSGDAQTPGPARKARTGKIKGSKPTSAPQGATQTDAKAHLSQTGV

SEQ ID NO: 417 - protein, 782 aa, unidentified
MKTEKPKTALTLLREEVFPGKKYRLDVLKEAGKKLSTKGREATIEFLTGKDEERPQNFQPPAKTSIVAQSRPFDQWPIVQVSLAVQKYIYGLTQSEFEANKKALYGETGKAISTESRRAWFEATGVDNFGFTAAQGINPIFSQAVARYEGVIKKVENRNEKKLKKLTKKNLLRLESGEEIEDFEPEATFNEEGRLLQPPGANPNIYCYQQISPRIYDPSDPKGVILPQIYAGYDRKPEDIISAGVPNRLAIPEGQPGYIPEHQRAGLKTQGRIRCRASVEAKARAAILAVVHLGEDWVVLDLRGLLRNVYWRKLASPGTLTLKGLLDFFTGGPVLDARRGIATFSYTLKSAAAVHAENTYKGKGTREVLLKLTENNSVALVTVDLGQRNPLAAMIARVSRTSQGDLTYPESVEPLTRLFLPDPFLEEVRKYRSSYDALRLSIREAAIASLTPEQQAEIRYIEKFSAGDAKKNVAEVFGIDPTQLPWDAMTPRTTYISDLFLRMGGDRSRVFFEVPPKKAKKAPKKPPKKPAGPRIVKRTDGMIARLREIRPRLSAETNKAFQEARWEGERSNVAFQKLSVRRKQFARTVVNHLVQTAQKMSRCDTVVLGIEDLNVPFFHGRGKYQPGWEGFFRQKKENRWLINDMHKALSERGPHRGGYVLELTPFWTSLRCPKCGHTDSANRDGDDFVCVKCGAKLHSDLEVATANLALVAITGQSIPRPPREQSSGKKSTGTARMKKTSGETQGKGSKACVSEALNKIEQGTARDPVYNPLNSQVSCPAP

SEQ ID NO: 418 - protein, 774 aa, unidentified
VYNPDMKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 419 - protein, 769 aa, unidentified
MKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 420 - protein, 767 aa, unidentified
VIKTHFPAGRFRKDHQKTAGKKLKHEGEEACVEYLRNKVSDYPPNFKPPAKGTIVAQSRPFSEWPIVRASEAIQKYVYGLTVAELDVFSPGTSKPSHAEWFAKTGVENYGYRQVQGLNTIFQNTVNRFKGVLKKVENRNKKSLKRQEGANRRRVEEGLPEVPVTVESATDDEGRLLQPPGVNPSIYGYQGVAPRVCTDLQGFSGMSVDFAGYRRDPDAVLVESLPEGRLSIPKGERGYVPEWQRDPERNKFPLREGSRRQRKWYSNACHKPKPGRTSKYDPEALKKASAKDALLVSISIGEDWAIIDVRGLLRDARRRGFTPEEGLSLNSLLGLFTEYPVFDVQRGLITFTYKLGQVDVHSRKTVPTFRSRALLESLVAKEEIALVSVDLGQTNPASMKVSRVRAQEGALVAEPVHRMFLSDVLLGELSSYRKRMDAFEDAIRAQAFETMTPEQQAEITRVCDVSVEVARRRVCEKYSISPQDVPWGEMTGHSTFIVDAVLRKGGDESLVYFKNKEGETLKFRDLRISRMEGVRPRLTKDTRDALNKAVLDLKRAHPTFAKLAKQKLELARRCVNFIEREAKRYTQCERVVFVIEDLNVGFFHGKGKRDRGWDAFFTAKKENRWVIQALHKAFSDLGLHRGSYVIEVTPQRTSMTCPRCGHCDKGNRNGEKFVCLQCGATLHADLEVATDNIERVALTGKAMPKPPVRERSGDVQKAGTARKARKPLKPKQKTEPSVQEGSSDDGVDKSPGDASRNPVYNPSDTLSI

SEQ ID NO: 421 - protein, 763 aa, unidentified
MAKAKTLAALLRELLPGQHLAPHHRWVANKLLMTSGDAAAFVIGKSVSDPVRGSFRKDVITKAGRIFKKDGPDAAAAFLDGKWEDRPPNFQPPAKAAIVAISRSFDEWPIVKVSCAIQQYLYALPVQEFESSVPEARAQAHAAWFQDTGVDDCNFKSTQGLNAIFNHGKRTYEGVLKKAQNRNDKKNLRLERINAKRAEAGQAPLVAGPDESPTDDAGCLLHPPGINANIYCYQQVSPRPYEQSCGIQLPPEYAGYNRLSNVAIPPMPNRLDIPQGQPGYVPEHHRHGIKKFGRVRKRYGVVPGRNRDADGKRTRQVLTEAGAAAKARDSVLAVIRIGDDWTVVDLRGLLRNAQWRKLVPDGGITVQGLLDLFTGDPVIDPRRGVVTFIYKADSVGIHSEKVCRGKQSKNLLERLCAMPEKSSTRLDCARQAVALVSVDLGQRNPVAARFSRVSLAEGQLQAQLVSAQFLDDAMVAMIRSYREEYDRFESLVREQAKAALSPEQLSEIVRHEADSAESVKSCVCAKFGIDPAGLSWDKMTSGTWRIADHVQAAGGDVEWFFFKTCGKGKEIKTVRRSDFNVAKQFRLRLSPETRKDWNDAIWELKRGNPAYVSFSKRKSEFARRVVNDLVHRARRAVRCDEVVFAIEDLNISFFHGKGQRQMGWDAFFEVKQENRWFIQALHKAFVERATHKGGYVLEVAPARTSTTCPECRHCDPESRRGEQFCCIKCRHTCHADLEVATFNIEQVALTGVSLPKRLSSTLL

SEQ ID NO: 422 - protein, 761 aa, unidentified
MSKEKTPPSAYAILKAKHFPDLDFEKKHKMMAGRMFKNGASEQEVVQYLQGKGSESLMDVKPPAKSPILAQSRPFDEWEMVRTSRLIQETIFGIPKRGSIPKRDGLSETQFNELVASLEVGGKPMLNKQTRAIFYGLLGIKPPTFHAMAQNILIDLAINIRKGVLKKVDNLNEKNRKKVKRIRDAGEQDVMVPAEVTAHDDRGYLNHPPGVNPTIPGYQGVVIPFPEGFEGLPSGMTPVDWSHVLVDYLPHDRLSIPKGSPGYIPEWQRPLLNRHKGRRHRSWYANSLNKPRKSRTEEAKDRQNAGKRTALIEAERLKGVLPVLMRFKEDWLIIDARGLLRNARYRGVLPEGSTLGNLIDLFSDSPRVDTRRGICTFLYRKGRAYSTKPVKRKESKETLLKLTEKSTIALVSIDLGQTNPLTAKLSKVRQVDGCLVAEPVLRKLIDNASEDGKEIARYRVAHDLLRARILEDAIDLLGIYKDEVVRARSDTPDLCKERVCRFLGLDSQAIDWDRMTPYTDFIAQAFVAKGGDPKVVTIKPNGKPKMFRKDRSIKNMKGIRLDISKEASSAYREAQWAIQRESPDFQRLAVWQSQLTKRIVNQLVAWAKKCTQCDTVVLAFEDLNIGMMHGSGKWANGGWNALFLHKQENRWFMQAFHKALTELSAHKGIPTIEVLPHRTSITCTQCGHCHPGNRDGERFKCLKCEFLANTDLEIATDNIERVALTGLPMPKGERSSAKRKPGGTRKTKKSKHSGNSPLAAE

SEQ ID NO: 423 - protein, 746 aa, unidentified
MEKAGPTSPLSVLIHKNFEGCRFQIDHLKIAGRKLAREGEAAAIEYLLDKKCEGLPPNFQPPAKGNVIAQSRPFTEWAPYRASVAIQKYIYSLSVDERKVCDPGSSSDSHEKWFKQTGVQNYGYTHVQGLNLIFKHALARYDGVLKKVDNRNEKNRKKAERVNSFRREEGLPEEVFEEEKATDETGHLLQPPGVNHSIYCYQSVRPKPFNPRKPGGISLPEAYSGYSLKPQDELPIGSLDRLSIPPGQPGYVPEWQRSQLTTQKHRRKRSWYSAQKWKPRTGRTSTFDPDRLNCARAQGAILAVVRIHEDWVVFDVRGLLRNALWRELAGKGLTVRDLLDFFTGDPVVDTKRGVVTFTYKLGKVDVHSLRTVRGKRSKKVLEDLTLSSDVGLVTIDLGQTNVLAADYSKVTRSENGELLAVPLSKSFLPKHLLHEVTAYRTSYDQMEEGFRRKALLTLTEDQQVEVTLVRDFSVESSKTKLLQLGVDVTSLPWEKMSSNTTYISDQLLQQGADPASLFFDGERDGKPCRHKKKDRTWAYLVRPKVSPETRKALNEALWALKNTSPEFESLSKRKIQFSRRCMNYLLNEAKRISGCGQVVFVIEDLNVRVHHGRGKRAIGWDNFFKPKRENRWFMQALHKAASELAIHRGMHIIEACPARSSITCPKCGHCDPENRCSSDREKFLCVKCGAAFHADLEVATFNLRKVALTGTALPKSIDHSRDGLIPKGARNRKLKEPQANDEKACA

SEQ ID NO: 424 - protein, 735 aa, unidentified
MKEQSPLSSVLKSNFPGKKFLSADIRVAGRKLAQLGEAAAVEYLSPRQRDSVPNFRPPAFCTVVAKSRPFEEWPIYKASVLLQEQIYGMTGQEFEERCGSIPTSLSGLRQWASSVGLGAAMEGLHVQGMNLMVKNAINRYKGVLVKVENRNKKLVEANEAKNSSREERGLPPLRPPELGSAFGPDGRLVNPPGIDKSIRLYQGVSPVPVVKTTGRPTVHRLDIPAGEKGHVPLWQREAGLVKEGPRRRRMWYSNSNLKRSRKDRSAEASEARKADSVVVRVSVKEDWVDIDVRGLLRNVAWRGIERAGESTEDLLSLFSGDPVVDPSRDSVVFLYKEGVVDVLSKKVVGAGKSRKQLEKMVSEGPVALVSCDLGQTNYVAARVSVLDESLSPVRSFRVDPREFPSADGSQGVVGSLDRIRADSDRLEAKLLSEAEASLPEPVRAEIEFLRSERPSAVAGRLCLKLGIDPRSIPWEKMGSTTSFISEALSAKGSPLALHDGAPIKDSRFAHAARGRLSPESRKALNEALWERKSSSREYGVISRRKSEASRRMANAVLSESRRLTGLAVVAVNLEDLNMVSKFFHGRGKRAPGWAGFFTPKMENRWFIRSIHKAMCDLSKHRGITVIESRPERTSISCPECGHCDPENRSGERFSCKSCGVSLHADFEVATRNLERVALTGKPMPRRENLHSPEGATASRKTRKKPREATASTFLDLRSVLSSAENEGSGPAARAG

SEQ ID NO: 425 - protein, 725 aa, unidentified
MLPPSNKIGKSMSLKEFINKRNFKSSIIKQAGKILKKEGEEAVKKYLDDNYVEGYKKRDFPITAKCNIVASNRKIEDFDISKFSSFIQNYVFNLNKDNFEEFSKIKYNRKSFDELYKKIANEIGLEKPNYENIQGEIAVIRNAINIYNGVLKKVENRNKKIQEKNQSKDPPKLLSAFDDNGFLAERPGINETIYGYQSVRLRHLDVEKDKDIIVQLPDIYQKYNKKSTDKISVKKRLNKYNVDEYGKLISKRRKERINKDDAILCVSNFGDDWIIFDARGLLRQTYRYKLKKKGLCIKDLLNLFTGDPIINPTKTDLKEALSLSFKDGIINNRTLKVKNYKKCPELISELIRDKGKVAMISIDLGQTNPISYRLSKFTANNVAYIENGVISEDDIVKMKKWREKSDKLENLIKEEAIASLSDDEQREVRLYENDIADNTKKKILEKFNIREEDLDFSKMSNNTYFIRDCLKNKNIDESEFTFEKNGKKLDPTDACFAREYKNKLSELTRKKINEKIWEIKKNSKEYHKISIYKKETIRYIVNKLIKQSKEKSECDDIIVNIEKLQIGGNFFGGRGKRDPGWNNFFLPKEENRWFINACHKAFSELAPHKGIIVIESDPAYTSQTCPKCENCDKENRNGEKFKCKKCNYEANADIDVATENLEKIAKNGRRLIKNFDQLGERLPGAEMPGGARKRKPSKSLPKNGRGAGVGSEPELINQSPSQVIA

SEQ ID NO: 426 - protein, 718 aa, unidentified; identical to SEQ ID NO: 57

SEQ ID NO: 427 - protein, 708 aa, unidentified; identical to SEQ ID NO: 60

SEQ ID NO: 428 - protein, 491 aa, unidentified
VLLSDRIQYTDPSAPIPAMTVVDRRKIKKGEPGYVPPFMRKNLSTNKHRRMRLSRGQKEACALPVGLRLPDGKDGWDFIIFDGRALLRACRRLRLEVTSMDDVLDKFTGDPRIQLSPAGETIVTCMLKPQHTGVIQQKLITGKMKDRLVQLTAEAPIAMLTVDLGEHNLVACGAYTVGQRRGKLQSERLEAFLLPEKVLADFEGYRRDSDEHSETLRHEALKALSKRQQREVLDMLRTGADQARESLCYKYGLDLQALPWDKMSSNSTFIAQHLMSLGFGESATHVRYRPKRKASERTILKYDSRFAAEEKIKLTDETRRAWNEAIWECQRASQEFRCLSVRKLQLARAAVNWTLTQAKQRSRCPRVVVVVEDLNVRFMHGGGKRQEGWAGFFKARSEKRWFIQALHKAYTELPTNRGIHVMEVNPARTSITCTKCGYCDPENRYGEDFHCRNPKCKVRGGHVANADLDIATENLARVALSGPMPKAPKLK

SEQ ID NO: 429 - protein, 848 aa, unidentified
MTPSFGYQMIIVTPIHHASGAWATLRLLFLNPKTSGVMLGMTKTKSAFALMREEVFPGLLFKSADLKMAGRKFAKEGREAAIEYLRGKDEERPANFKPPAKGDIIAQSRPFDQWPIVQVSQAIQKYIFGLTKAEFDATKTLLYGEGNHPTTESRRRWFEATGVPDFGFTSAQGLNAIFSSALARYEGVIQKVENRNEKRLKKLSEKNQRLVEEGHAVEAYVPETAFHTLESLKALSEKSLVPLDDLMDKIDRLAQPPGINPCLYGYQQVAPYIYDPENPRGVVLPDLYLGYCRKPDDPITACPNRLDIPKGQPGYIPEHQRGQLKKHGRVRRFRYTNPQAKARAKAQTAILAVLRIDEDWVVMDLRGLLRNVYFREVAAPGELTARTLLDTFTGCPVLNLRSNVVTFCYDIESKGALHAEYVRKGWATRNKLLDLTKDGQSVALLSVDLGQRHPVAVMISRLKRDDKGDLSEKSIQVVSRTFADQYVDKLKRYRVQYDALRKEIYDAALVSLPPEQQAEIRAYEAFAPGDAKANVLSVMFQGEVSPDELPWDKMNTNTHYISDLYLRRGGDPSRVFFVPQPSTPKKNAKKPPAPRKPVKRTDENVSHMPEFRPHLSNETREAFQKAKWTMERGNVRYAQLSRFLNQIVREANNWLVSEAKKLTQCQTVVWAIEDLHVPFFHGKGKYHETWDGFFRQKKEDRWFVNVFHKAISERAPNKGEYVMEVAPYRTSQRCPVCGFVDADNRHGDHFKCLRCGVELHADLEVATWNIALVAVQGHGIAGPPREQSCGGETAGTARKGKNIKKNKGLADAVTVEAQDSEGGSKKDAGTARNPVYIPSESQVNCPAP

SEQ ID NO: 430 - protein, 781 aa, unidentified
MKPKTPKPPKTPVAALIDKHFPGKRFRASYLKSVGKKLKNQGEDVAVRFLTGKDEERPPNFQPPAKSNIVAQSRPIEEWPIHKVSVAVQEYVYGLTVAEKEACSDAGESSSSHAAWFAKTGVENFGYTSVQGLNKIFPPTFNRFDGVIKKVENRNEKKRQKATRINEAKRNKGQSEDPPEAEVKATDDAGYLLQPPGINHSVYGYQSITLCPYTAEKFPTIKLPEEYAGYHSNPDAPIPAGVPDRLAIPEGQPGHVPEEHRAGLSTKKHRRVRQWYAMANWKPKPKRTSKPDYDRLAKARAQGALLIVIRIDEDWVVVDARGLLRNVRWRSLGKREITPNELLDLFTGDPVLDLKRGVVTFTYAEGVVNVCSRSTTKGKQTKVLLDAMTAPRDGKKRQIGMVAVDLGQTNPIAAEYSRVGKNAAGTLEATPLSRSTLPDELLREIALYRKAHDRLEAQLREEAVLKLTAEQQAENARYVETSEEGAKLALANLGVDTSTLPWDAMTGWSTCISDHLINHGGDTSAVFFQTIRKGTKKLETIKRKDSSWADIVRPRLTKETREALNDFLWELKRSHEGYEKLSKRLEELARRAVNHVVQEVKWLTQCQDIVIVIEDLNVRNFHGGGKRGGGWSNFFTVKKENRWFMQALHKAFSDLAAHRGIPVLEVYPARTSITCLGCGHCDPENRDGEAFVCQQCGATFHADLEVATRNIARVALTGEAMPKAPAREQPGGAKKRGTSRRRKLTEVAVKSAEPTIHQAKNQQLNGTSRDPVYKGSELPAL

SEQ ID NO: 431 - protein, 767 aa, unidentified
MSEITDLLKANFKGKTFKSADMRMAGRILKKSGAQAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRGVQTTGTARNPGRSLKSNKNPSEPKRVLQSKTRKKITSTETQNEPLATDLKT

SEQ ID NO: 432 - protein, 760 aa, unidentified
MTPKTESPLSALCKKHFPGKRFRTNYLKDAGKILKKHGEDAVVAFLSDKQEDEPANFCPPAKVHILAQSRPFEDWPINLASKAIQTYVYGLTADERKTCEPGTSKESHDRWFKETGVDHHGFTSVQGLNLIFKHTLNRYDGVIKKVETRNEKRRSSVVRINEKKAAEGLPLIAAEAEETAFGEDGRLLQPPGVNHSIYCFQQVSPQPYSSKKHPQVVLPHAVQGVDPDAPIPVGRPNRLDIPKGQPGYVPEWQRPHLSMKCKRVRMWYARANWRRKPGRRSVLNEARLKEASAKGALPIVLVIGDDWLVMDARGLLRSVFWRRVAKPGLSLSELLNVTPTGLFSGDPVIDPKRGLVTFTSKLGVVAVHSRKPTRGKKSKDLLLKMTKPTDDGMPRHVGMVAIDLGQTNPVAAEYSRVVQSDAGTLKQEPVSRGVLPDDLLKDVARYRRAYDLTEESIRQEAIALLSEGHRAEVTKLDQTTANETKRLLVDRGVSESLPWEKMSSNTTYISDCLVALGKTDDVFFVPKAKKGKKETGIAVKRKDHGWSKLLRPRTSPEARKALNENQWAVKRASPEYERLSRRKLELGRRCVNHIIQETKRWTQCEDIVVVLEDLNVGFFHGSGKRPDGWDNFFVSKRENRWFIQVLHKAFGDLATHRGTHVIEVHPARTSITCIKCGHCDAGNRDGESFVCLASACGDRRHADLEVATRNVARVAITGERMPPSEQARDVQKAGGARKRKPSARNVKSSYPAVEPAPASP

SEQ ID NO: 433 - protein, 752 aa, unidentified
MSDNKMKKLSKEEKPLTPLQILIRKYIDKSQYPSGFKTTIIKQAGVRIKSVKSEQDEINLANWIISKYDPTYIKRDFNPSAKCQIIATSRSVADFDIVKMSNKVQEIFFASSHLDKNVFDIGKSKSDHDSWFERNNVDRGIYTYSNVQGMNLIFSNTKNTYLGVAVKAQNKFSSKMKRIQDINNFRITNHQSPLPIPDEIKIYDDAGFLLNPPGVNPNIFGYQSCLLKPLENKEIISKTSFPEYSRLPADMIEVNYKISNRLKFSNDQKGFIQFKDKLNLFKINSQELFSKRRRLSGQPILLVASFGDDWVVLDGRGLLRQVYYRGIAKPGSITISELLGFFTGDPIVDPIRGVVSLGFKPGVLSQETLKTTSARIFAEKLPNLVLNNNVGLMSIDLGQTNPVSYRLSEITSNMSVEHICSDFLSQDQISSIEKAKTSLDNLEEEIAIKAVDHLSDEDKINFANFSKLNLPEDTRQSLFEKYPELIGSKLDFGSMGSGTSYIADELIKFENKDAFYPSGKKKFDLSFSRDLRKKLSDETRKSYNDALFLEKRTNDKYLKNAKRRKQIVRTVANSLVSKIEELGLTPVINIENLAMSGGFFDGRGKREKGWDNFFKVKKENRWVMKDFHKAFSELSPHHGVIVIESPPYCTSVTCTKCNFCDKKNRNGHKFTCQRCGLDANADLDIATENLEKVAISGKRMPGSERSSDERKVAVARKAKSPKGKAIKGVKCTITDEPALLSANSQDCSQSTS

SEQ ID NO: 434 - protein, 747 aa, unidentified
MALSLAEVRERHFKGLRFRSSYLKRAGKILKKEGEAACVAYLTGKDEESPPNFKPPAKCDVVAQSRPFEEWPIVQASVAVQSYVYGLTKEAFEAFNPGTTKQSHEACLAATGIDTCGYSNVQGLNLIFRQAKNRYEGVITKVENRNKKAKKKLTRKNEWRQKNGHSELPEAPEELTFNDEGRLLQPPGINPSLYTYQQISPTPWSPKDSSILPPQYAGYERDPNAPIPFGVAKDRLTIASGCPGYIPEWMRTAGEKTNPRTQKKFMHPGLSTRKNKRMRLPRSVRSAPLGALLVTIHLGEDWLVLDVRGLLRNARWRGVAPKDISTQGLLNLFTGDPVIDTRRGVVTFTYKPETVGIHSRTWLYKGKQTKEVLEKLTQDQTVALVAIDLGQTNPVSAAASRVSRSGENLSIETVDRFFLPDELIKELRLYRMAHDRLEERIREESTLALTEAQQAEVRALEHVVRDDAKNKVCAAFNLDAASLPWDQMTSNTTYLSEAILAQGVSRDQVFFTPNPKKGSKEPVEVMRKDRAWVYAFKAKLSEETRKAKNEALWALKRASPDYARLSKRREELCRRSVNMVINRAKKRTQCQVVIPVLEDLNIGFFHGSGKRLPGWDNFFVAKKENRWLMNGLHKSFSDLAVHRGFYVFEVMPHRTSITCPACGHCDSENRDGEAFVCLSCKRTYHADLDVATHNLTQVAGTGLPMPEREHPGGTKKPGGSRKPESPQTHAPILHRTDYSESADRLGS

SEQ ID NO: 435 - protein, 733 aa, unidentified
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 436 - protein, 702 aa, unidentified
MIKEQSELSKLIEKYYPGKKFYSNDLKQAGKHLKKSEHLTAKESEELTVEFLKSCKEKLYDFRPPAKALIISTSRPFEEWPIYKASESIQKYIYSLTKEELEKYNISTDKTSQENFFKESLIDNYGFANVSGLNLIFQHTKAIYDGVLKKVNNRNNKILKKYKRKIEEGIEIDSPELEKAIDESGHFINPPGINKNIYCYQQVSPTIFNSFKETKIICPFNYKRNPNDIIQKGVIDRLAIPFGEPGYIPDHQRDKVNKHKKRIRKYYKNNENKNKDAILAKINIGEDWVLFDLRGLLRNAYWRKLIPKQGITPQQLLDMFSGDPVIDPIKNNITFIYKESIIPIHSESIIKTKKSKELLEKLTKDEQIALVSIDLGQTNPVAARFSRLSSDLKPEHVSSSFLPDELKNEICRYREKSDLLEIEIKNKAIKMLSQEQQDEIKLVNDISSEELKNSVCKKYNIDNSKIPWDKMNGFTTFIADEFINNGGDKSLVYFTAKDKKSKKEKLVKLSDKKIANSFKPKISKETREILNKITWDEKISSNEYKKLSKRKLEFARRATNYLINQAKKATRLNNVVLVVEDLNSKFFHGSGKREDGWDNFFIPKKENRWFIQALHKSLTDVSIHRGINVIEVRPERTSITCPKCGCCDKENRKGEDFKCIKCDSVYHADLEVATFNIEKVAITGESMPKPDCERLGGEESIG

SEQ ID NO: 437 - protein, 661 aa, unidentified; identical to SEQ ID NO: 61

SEQ ID NO: 438 - protein, 531 aa, unidentified
LEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGIITFSYKEGVVPVFSQKIVSRFKSRDTLEKLTSQGPVALLSVDLGQNEPVAARVCSLKNINDKIALDNSCRIPFLDDYKKQIKDYRDSLDELEIKIRLEAINSLDVNQQVEIRDLDVFSADRAKASTVDMFDIDPNLISWDSMSDARFSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKSFSELSSNRGLCVIEVNPAWTSATCPDCGFCSKENRDGINFTCRKCGVSYHADIDVATLNIARVAVLGKPMSGPADRERLGGTKKPRVARSRKDMKRKDISNGTVEVMVTA

SEQ ID NO: 439 - protein, 479 aa, unidentified
IPSFGYLDRLKIAKGQPGYIPEWQRETINPSKKVRRYWATNHEKIRNAIPLVVFIGDDWVIIDGRGLLRDARRRKLADKNTTIEQLLEMVSNDPVIDSTRGIATLSYVEGVVPVRSFIPIGEKKGREYLEKSTQKESVTLLSVDIGQINPVSCGVYKVSNGCSKIDFLDKFFLDKKHLDAIQKYRTLQDSLEASIVNEALDEIDPSFKKEYQNINSQTSNDVKKSLCTEYNIDPEAISWQDITAHSTLISDYLIDNNITNDVYRTVNKAKYKTNDFGWYKKFSAKLSKEAREALNEKIWELKIASSKYKKLSVRKKEIARTIANDCVKRAETYGDNVVVAMESLTKNNKVMSGRGKRDPGWHNLGQAKVENRWFIQAISSAFEDKATHHGTPVLKVNPAYTSQTCPSCGHCSKDNRSSKDRTIFVCKSCGEKFNADLDVATYNIAHVAFSGKKLSPPSEKSSATKKPRSARKSKKSRKS

SEQ ID NO: 440 - protein, 452 aa, unidentified
SPIEKLLNGLLVKITFGNDWIICDARGLLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 441 - protein, 425 aa, unidentified
LLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 442 - protein, 735 aa, unidentified
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 443 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 444 - protein, 1159 aa, Leptotrichia buccalis; identical to SEQ ID NO: 21

SEQ ID NO: 445 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 446 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 447 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 448 - protein, 1285 aa, Herbinix hemicellulosilytica; identical to SEQ ID NO: 71

SEQ ID NO: 449 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 450 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 451 - protein, 1224 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 452 - protein, 1126 aa, Prevotella intermedia
MEDDKKTTDSIRYELKDKHFWAAFLNLARHNVYITVNHINKILEEGEINRDGYETTLKNTWNEIKDINKKDRLSKLIIKHFPFLEAATYRLNPTDTTKQKEEKQAEAQSLESLRKSFFVFIYKLRDLRNHYSHYKHSKSLERPKFEEGLLEKMYNIFNASIRLVKEDYQYNKDINPDEDFKHLDRTEEEFNYYFTKDNEGNITESGLLFFVSLFLEKKDAIWMQQKLRGFKDNRENKKKMTNEVFCRSRMLLPKLRLQSTQTQDWILLDMLNELIRCPKSLYERLREEDREKFRVPIEIADEDYDAEQEPFKNTLVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDTFANGEIKSIDELEEYCKGKDIEIGHLPKQMIAILKDEHKVMATEAERKQEEMLVDVQKSLESLDNQINEEIENVERKNSSLKSGKIASWLVNDMMRFQPVQKDNEGKPLNNSKANSTEYQLLQRTLAFFGSEHERLAPYFKQTKLIESSNPHPFLKDTEWEKCNNILSFYRSYLEAKKNFLESLKPEDWEKNQYFLKLKEPKTKPKTLVQGWKNGFNLPRGIFTEPIRKWFMKHRENITVAELKRVGLVAKVIPLFFSEEYKDSVQPFYNYHFNVGNINKPDEKNFLNCEERRELLRKKKDEFKKMTDKEKEENPSYLEFKSWNKFERELRLVRNQDIVTWLLCMELFNKKKIKELNVEKIYLKNINTNTTKKEKNTEEKNGEEKNIKEKNNILNRIMPMRLPIKVYGRENFSKNKKKKIRRNTFFTVYIEEKGTKLLKQGNFKALERDRRLGGLFSFVKTPSKAESKSNTISKLRVEYELGEYQKARIEIIKDMLALEKTLIDKYNSLDTDNFNKMLTDWLELKGEPDKASFQNDVDLLIAVRNAFSHNQYPMRNRIAFANINPFSLSSANTSEEKGLGIANQLKDKTHKTIEKIIEIEKPIETKE

SEQ ID NO: 453 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 454 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFAVFFKPDDFVLAKNRKEQLISVADGKECLTVSGFAFFICLFLDREQASGMLSRIRGFKRTDENWARAVHETFCDLCIRHPHDRLESSNTKEALLLDMLNELNRCPRILYDMLPEEERAQFLPALDENSMNNLSENSLDEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELRLLDPSSGHPFLSATMETAHRYTEGFYKCYLEKKREWLAKIFYRPEQDENTKRRISVFFVPDGEARKLLPLLIRRRMKEQNDLQDWIRNKQAHPIDLPSHLFDSKVMELLKVKDGKKKWNEAFKDWWSTKYPDGMQPFYGLRRELNIHGKSVSYIPSDGKKFADCYTHLMEKTVRDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 455 - protein, 1115 aa, Bacteroides pyogenes
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETAECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTSGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPLPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 456 - protein, 668 aa, unidentified
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKIKKFL

SEQ ID NO: 457 - protein, 796 aa, unidentified
MEKDKKGEKIDISQEMIEEDLRKILILFSRLRHSMVHYDYEFYQALYSGKDFVISDKNNLENRMISQLLDLNIFKELSKVKLIKDKAISNYLDKNTTIHVLGQDIKAIRLLDIYRDICGSKNGFNKFINTMITISGEEDREYKEKVIEHFNKKMENLSTYLEKLEKQDNAKRNNKRVYNLLKQKLIEQQKLKEWFGGPYVYDIHSSKRYKELYIERKKLVDRHSKLFEEGLDEKNKKELTKINDELSKLNSEMKEMTKLNSKYRLQYKLQLAFGFILEEFDLNIDTFINNFDKDKDLIISNFMKKRDIYLNRVLDRGDNRLKNIIKEYKFRDTEDIFCNDRDNNLVKLYILMYILLPVEIRGDFLGFVKKNYYDMKHVDFIDKKDKEDKDTFFHDLRLFEKNIRKLEITDYSLSSGFLSKEHKVDIEKKINDFINRNGAMKLPEDITIEEFNKSLILPIMKNYQINFKLLNDIEISALFKIAKDRSITFKQAIDEIKNEDIKKNSKKNDKNNHKDKNINFTQLMKRALHEKIPYKAGMYQIRNNISHIDMEQLYIDPLNSYMNSNKNNITISEQIEKIIDVCVTGGVTGKELNNNIINDYYMKKEKLVFNLKLRKQNDIVSIESQEKNKREEFVFKKYGLDYKDGEINIIEVIQKVNSLQEELRNIKETSKEKLKNKETLFRDISLINGTIRKNINFKIKEMVLDIVRMDEIRHINIHIYYKGENYTRSNIIKFKYAIDGENKKYYLKQHEINDINLELKDKFVTLICNMDKHPNKNKQTINLESNYIQNVKFIIP

SEQ ID NO: 458 - protein, 897 aa, unidentified
MENKGNNKKIDFDENYNILVAQIKEYFTKEIENYNNRIDNIIDKKELLKYSEKKEESEKNKKLEELNKLKSQKLKILTDEEIKADVIKIIKIFSDLRHSLMHYEYKYFENLFENKKNEELAELLNLNLFKNLTLLRQMKIENKTNYLEGREEFNIIGKNIKAKEVLGHYNLLAEQKNGFNNFINSFFVQDGTENLEFKKLIDEHFVNAKKRLERNIKKSKKLEKELEKMEQHYQRLNCAYVWDIHTSTTYKKLYNKRKSLIEEYNKQINEIKDKEVITAINVELLRIKKEMEEITKSNSLFRLKYKMQIAYAFLEIEFGGNIAKFKDEFDCSKMEEVQKYLKKGVKYLKYYKDKEAQKNYEFPFEEIFENKDTHNEEWLENTSENNLFKFYILTYLLLPMEFKGDFLGVVKKHYYDIKNVDFTDESEKELSQVQLDKMIGDSFFHKIRLFEKNTKRYEIIKYSILTSDEIKRYFRLLELDVPYFEYEKGTDEIGIFNKNIILTIFKYYQIIFRLYNDLEIHGLFNISSDLDKILRDLKSYGNKNINFREFLYVIKQNNNSSTEEEYRKIWENLEAKYLRLHLLTPEKEEIKTKTKEELEKLNEISNLRNGICHLNYKEIIEEILKTEISEKNKEATLNEKIRKVINFIKENELDKVELGFNFINDFFMKKEQFMFGQIKQVKEGNSDSITTERERKEKNNKKLKETYELNCDNLSEFYETSNNLRERANSSSLLEDSAFLKKIGLYKVKNNKVNSKVKDEEKRIENIKRKLLKDSSDIMGMYKAEVVKKLKEKLILIFKHDEEKRIYVTVYDTSKAVPENISKEILVKRNNSKEEYFFEDNNKKYVTEYYTLEITETNELKVIPAKKLEGKEFKTEKNKENKLMLNNHYCFNVKIIY

SEQ ID NO: 459 - protein, 919 aa, unidentified
MEEIKHKKNKSSIIRVIVSNYDMTGIKEIKVLYQKQGGVDTFNLKTIINLESGNLEIISCKPKEREKYRYEFNCKTEINTISITKKDKVLKKEIRKYSLELYFKNEKKDTVVAKVTDLLKAPDKIEGERNHLRKLSSSTERKLLSKTLCKNYSEISKTPIEEIDSIKIYKIKRFLNYRSNFLIYFALINDFLCAGVKEDDINEVWLIQDKEHTAFLENRIEKITDYIFDKLSKDIENKKNQFEKRIKKYKTSLEELKTETLEKNKTFYIDSIKTKITNLENKITELSLYNSKESLKEDLIKIISIFTNLRHSLMHYDYKSFENLFENIENEELKNLLDLNLFKSIRMSDEFKTKNRTNYLDGTESFTIVKKHQNLKKLYTYYNNLCDKKNGFNTFINSFFVTDGIENTDFKNLIILHFEKEMEEYKKSIEYYKIKISNEKNKSKKEKLKEKIDLLQSELINMREHKNLLKQIYFFDIHNSIKYKELYSERKNLIEQYNLQINGVKDVTAINHINTKLLSLKNKMDKITKQNSLYRLKYKLKIAYSFLMIEFDGDVSKFKNNFDPTNLEKRVEYLDKKEEYLNYTAPKNKFNFAKLEEELQKIQSTSEMGADYLNVSPENNLFKFYILTYIMLPVEFKGDFLGFVKNHYYNIKNVDFMDESLLDENEVDSNKLNEKIENLKDSSFFNKIRLFEKNIKKYEIVKYSVSTQENMKEYFKQLNLDIPYLDYKSTDEIGIFNKNMILPIFKYYQNVFKLCNDIEIHALLALANKKQQNLEYAIYCCSKKNSLNYNELLKTFNRKTYQNLSFIRNKIAHLNYKELFSDLFNNELDLNTKVRCLIEFSQNNKFDQIDLGMNFINDYYMKKTRFIFNQRRLRDLNVPSKEKIIDGKRKQQNDSNNELLKKYGLSRTNIKDIFNKAWY

SEQ ID NO: 460 - protein, 1110 aa, unidentified
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETLMIEKNTLKISSNGKIISLIPHYSYSIDVKY

SEQ ID NO: 461-462 - skipped (2 entries)

SEQ ID NO: 463 - RNA, 10 nt, synthetic construct
  source 1..10: mol_type = other RNA
tttttttttt

SEQ ID NO: 464 - DNA, 10 bp, synthetic construct
  source 1..10: mol_type = other DNA
  misc_feature 1..4: note = DNA
  misc_feature 5..6: note = RNA
  misc_feature 7..10: note = DNA
tttttttttt

SEQ ID NO: 465-471 - skipped (7 entries)

SEQ ID NO: 472 - DNA, 10 bp, synthetic construct
  source 1..10: mol_type = other DNA
tttttttttt

SEQ ID NO: 473 - DNA, 12 bp, synthetic construct
  source 1..12: mol_type = other DNA
tttttttttttt

SEQ ID NO: 474 - DNA, 14 bp, synthetic construct
  source 1..14: mol_type = other DNA
tttttttttttttt

SEQ ID NO: 475-477 - skipped (3 entries)

SEQ ID NO: 478 - protein, 18 aa, unidentified
KRPAATKKAGQAKKKKEF

(19) United States
(12) Patent Application Publication
(10) Pub. No.: US 2025/0263803 A1
(43) Pub. Date: Aug. 21, 2025

(54) METHODS FOR QUANTITATION OF NUCLEIC ACID TARGETS

(71) Applicant: Mammoth Biosciences, Inc., Brisbane, CA (US)

(72) Inventors: Clare Louise FASCHING, Redwood City, CA (US); Carley Gelenter HENDRIKS, Burlingame, CA (US); Bridget Ann Paine MCKAY, Portola Valley, CA (US); Janice Sha CHEN, San Francisco, CA (US); James Paul BROUGHTON, South San Francisco, CA (US); Vladyslava RATUSHNA, Chula Vista, CA (US)

(73) Assignee: Mammoth Biosciences, Inc., Brisbane, CA (US)

(21) Appl. No.: 19/203,260

(22) Filed: May 9, 2025

Related U.S. Application Data
(63) Continuation of application No. PCT/US2023/080111, filed on Nov. 16, 2023.
(60) Provisional application No. 63/384,212, filed on Nov. 17, 2022.
(60) Provisional application No. 63/384,078, filed on Nov. 16, 2022.

(51) Int. Cl. C12Q 1/70; C12Q 1/34; C12Q 1/6806; C12Q 1/6823

(52) U.S. Cl. CPC C12Q 1/70; C12Q 1/34; C12Q 1/6806; C12Q 1/6823; G01N 2333/922

(57) ABSTRACT

Provided herein are compositions, systems, and methods comprising effector proteins (e.g., CRISPR-associated (Cas) proteins), and uses thereof. Various compositions, systems, and methods of the present disclosure may leverage the activities of these effector proteins for the detection and quantitation of nucleic acids.

### CROSS-REFERENCE TO RELATED APPLICATIONS

This application is a continuation of International PCT Application No. PCT/US2023/080111, filed Nov. 16, 2023, which claims priority to U.S. Provisional Patent Application No. 63/384,078, filed Nov. 16, 2022, and U.S. Provisional Patent Application No. 63/384,212, filed Nov. 17, 2022, each of which is incorporated by reference herein in its entirety for all purposes.

### REFERENCE TO AN ELECTRONIC SEQUENCE LISTING

The contents of the electronic sequence listing (MABI_027_02US_SeqList_ST26.xml; Size: 669,197 bytes; and Date of Creation: Apr. 28, 2025) are herein incorporated by reference in their entirety.

### BACKGROUND

The ability to quantitate the amount of target nucleic acid in a sample without a need to amplify the target nucleic acid prior to detection would be valuable in diagnostics, therapeutics, and epidemiology. For instance, quantitating the amount of viral nucleic acid in a patient sample over time can help with designing a treatment strategy, and quantitating the amount of viral nucleic acid in wastewater over time can be used to predict and prevent the spread of infectious diseases. Thus, there continues to be a need for assays for the quantitative detection of nucleic acids.

### SUMMARY

In a first aspect, the disclosure provides methods of quantitating a target nucleic acid in a test sample, the method comprising: (a) contacting the test sample with the following components (i) through (iii), resulting in at least two nanovolumes of reaction mixture: (i) a CRISPR/Cas effector protein, (ii) a guide RNA, comprising a region that is capable of binding to the CRISPR/Cas effector protein, and a guide sequence that is capable of hybridizing with the target nucleic acid, (iii) a reporter nucleic acid that is single stranded and does not hybridize with the guide sequence of the guide RNA; (b) measuring detectable signals detected from the at least two nanovolumes and generated by cleavage of the reporter nucleic acid by the CRISPR/Cas effector protein, and (c) quantitating the target nucleic acid in the test sample based on the measured signals from the at least two nanovolumes.

In some embodiments, the contacting step comprises sequentially adding each of the components (i) through (iii) to the test sample in the at least two nanovolumes. In some embodiments, the contacting step comprises: (a) adding each of the components (i) through (iii) to the test sample to generate a master reaction mixture, wherein the master reaction mixture has a volume of more than 1 nL, and (b) distributing the master reaction mixture into the at least two nanovolumes. In some embodiments, each of the at least two nanovolumes comprises no more than 1 molecule of the target nucleic acid. In some embodiments, the CRISPR/Cas effector protein and the guide RNA are incubated with each other prior to step (a). In some embodiments, the CRISPR/Cas effector protein and the guide RNA are not incubated with each other prior to step (a).

In some embodiments, the methods further comprise quantitating multiple target nucleic acids in the test sample, wherein the at least two nanovolumes of reaction mixture comprises one or more guide RNAs, wherein at least one of the one or more guide RNAs comprises a guide sequence that is capable of hybridizing with each of the multiple target nucleic acids. In some embodiments, each of the at least two nanovolumes of reaction mixture comprises at least one of the one or more guide RNAs comprising a guide sequence that is capable of hybridizing with each of the multiple target nucleic acids. In some embodiments, each of the at least two nanovolumes of reaction mixture comprises at least one of the one or more guide RNAs comprising a guide sequence that is capable of hybridizing with no more than one of the multiple target nucleic acids.

In some embodiments, the method does not comprise amplifying the target nucleic acid in the test sample. In some embodiments, the test sample comprises about 10,000 molecules to about 100,000 molecules of the target nucleic acid. In some embodiments, the test sample comprises about 50,000 molecules of the target nucleic acid. In some embodiments, the contacting step results in a number of nanovolumes in the range of about 5000 nanovolumes to about 100,000 nanovolumes. In some embodiments, step (b) comprises measuring a binary signal from each of the at least two nanovolumes. In some embodiments, the methods comprise contacting the test sample with a precursor guide RNA array, wherein the CRISPR/Cas effector protein cleaves the precursor guide RNA array to produce the guide RNA.

In some embodiments, the target nucleic acid is DNA or RNA. In some embodiments, the target nucleic acid is a viral nucleic acid or a bacterial nucleic acid. In some embodiments, the target nucleic acid is a viral nucleic acid. In some embodiments, the target nucleic acid is derived from a papovavirus, a human papillomavirus (HPV), a hepadnavirus, a Hepatitis B Virus (HBV), a herpesvirus, a varicella zoster virus (VZV), an Epstein Barr virus (EBY), a Kaposi's sarcoma-associated herpesvirus, an adenovirus, a poxvirus, a parvovirus, an influenza virus, a respiratory syncytial virus, or a coronavirus. In some embodiments, the target nucleic acid is derived from a severe acute respiratory syndrome coronavirus 2 (SARS-CoV-2).

In some embodiments, the target nucleic acid is derived from a human cell. In some embodiments, the target nucleic acid is a human fetal nucleic acid or a cancer cell nucleic acid. In some embodiments, the target nucleic acid is single stranded. In some embodiments, the target nucleic acid is double stranded. In some embodiments, the test sample comprises DNA from a cell lysate. In some embodiments, the test sample comprises cells. In some embodiments, the test sample is a blood, serum, plasma, urine, aspirate, fecal or biopsy sample.

In some embodiments, the methods further comprise quantitating a positive control target nucleic acid in a positive control sample, the method comprising: (a) contacting the positive control sample with the following components (i) through (iii) resulting in at least two nanovolumes of reaction mixture: (i) a CRISPR/Cas effector protein; (ii) a positive control guide RNA, comprising a region that is capable of binding to the CRISPR/Cas effector protein, and a guide sequence that is capable of hybridizing with the positive control target nucleic acid; and (iii) a reporter nucleic acid that is single stranded and does not hybridize with the guide sequence of the positive control guide RNA; (b) measuring detectable signals detected from the at least two nanovolumes and produced by cleavage of the reporter nucleic acid by the CRISPR/Cas effector protein, and (c) quantitating the amount of positive control target nucleic acid in the positive control sample based on the measured signals from the at least two nanovolumes.

In some embodiments, the methods further comprise quantitating the target nucleic acid in a positive control sample, the method comprising: (a) contacting the positive control sample with the following components (i) through (iii) resulting in at least two nanovolumes of reaction mixture: (i) a CRISPR/Cas effector protein; (ii) the guide RNA, comprising a region that is capable of binding to the CRISPR/Cas effector protein, and a guide sequence that is capable of hybridizing with the target nucleic acid; and (iii) a reporter nucleic acid that is single stranded and does not hybridize with the guide sequence of the guide RNA; (b) measuring detectable signals detected from the at least two nanovolumes produced by cleavage of the reporter nucleic acid by the CRISPR/Cas effector protein, and (c) quantitating the amount of target nucleic acid in the positive control sample based on the measured signals from the at least two nanovolumes.

In some embodiments, the method comprises generating a standard curve for the target nucleic acid in the positive control sample, and obtaining an absolute quantitation of the target nucleic acid in the test sample based on the standard curve. In some embodiments, the method comprises obtaining a relative quantitation of the target nucleic acid in the test sample based on the quantitation of the target nucleic acid in a positive control sample. In some embodiments, the detectable signal is detectable in less than 90 minutes. In some embodiments, the detectable signal is detectable in less than 30 minutes. In some embodiments, the CRISPR/Cas effector protein comprises an amino acid sequence with at least 90% identity to the amino acid sequence of any one of SEQ ID NOs: 1-72. In some embodiments, the CRISPR/Cas effector protein comprises the amino acid sequence of any one of SEQ ID NOS: 1-72.

In some embodiments, the target nucleic acid is an RNA and the CRISPR/Cas effector protein is an RNA-targeting CRISPR/Cas effector protein. In some embodiments, the RNA-targeting CRISPR/Cas effector protein comprises an amino acid sequence with at least 90% identity to the amino acid sequence of SEQ ID NO: 21, 62, 43, 41, or 42. In some embodiments, the RNA-targeting CRISPR/Cas effector protein comprises the amino acid sequence of SEQ ID NO: 21, 62, 43, 41, or 42.

In some embodiments, the target nucleic acid is a DNA and the CRISPR/Cas effector protein is a DNA-targeting CRISPR/Cas effector protein. In some embodiments, the DNA-targeting CRISPR/Cas effector protein comprises an amino acid sequence with at least 90% identity to the amino acid sequence of SEQ ID NO: 3, 34, 57, 36, 65, 67, 68, 89, 90, 91, or 17. In some embodiments, the DNA-targeting CRISPR/Cas effector protein comprises the amino acid sequence of SEQ ID NO: 3, 34, 57, 36, 65, 67, 68, 89, 90, 91, or 17.

In some embodiments, the reaction mixture comprises a buffer, wherein the buffer comprises tricine, MgOAc, BSA, TCEP, imidazole, KCl, MgCl₂, BSA, Igepal Ca-630, glycerol, HEPES, KOAc, Triton-X 100, Tris-HCl, (NH4)2SO4, Tween-20, TMAO, or any combination thereof. In some embodiments, the reporter nuclear acid is a RNA. In some embodiments, the reporter nuclear acid is a DNA. In some embodiments, the reporter nucleic acid comprises a modified nucleobase, a modified sugar moiety, and/or a modified nucleic acid linkage.

In another aspect, the present disclosure provides a method of assaying for a target nucleic acid in a sample, the method comprising a) amplifying the target nucleic acid using at least one amplification primer; b) contacting the sample to a reporter and a composition comprising a programmable nuclease and a guide nucleic acid that hybridizes to the target nucleic acid or an amplified product thereof, wherein the programmable nuclease cleaves the reporter upon hybridization of the guide nucleic acid to the target nucleic acid or the amplification product thereof; and c) assaying for a change in a signal, wherein the change in the signal is produced by cleavage of the reporter; wherein the target nucleic acid is a gene of a monkeypox virus or a segment thereof; and optionally wherein the at least one amplification primer comprises a nucleotide sequence at least 85%, at least 87%, at least 90%, at least 92%, at least 94%, at least 95%, at least 96%, at least 98%, or 100% identical to any one of SEQ ID NOs: 92-235.

In some embodiments, the gene of the monkeypox virus is selected from the group consisting of OPG123, OPG038, OPG094, OPG037, OPG151, OPG105, and OPG199.

In some embodiments, the at least one amplification primer comprises at least six amplification primers. The at least six amplification primers may comprise a FIP primer, a BIP primer, a B3 primer, a F3 primer, a LB primer, and a LF primer. In some embodiments, the FIP primer comprises a nucleotide sequence at least 85%, at least 87%, at least 90%, at least 92%, at least 94%, at least 95%, at least 96%, at least 98%, or 100% identical to any one of SEQ ID NOs: 94, 100, 106, 112, 118, 124, 130, 136, 142, 148, 154, 160, 166, 171, 178, 184, 190, 196, 202, 208, 214, 220, 226, or 232. In some embodiments, the BIP primer comprises a nucleotide sequence at least 85%, at least 87%, at least 90%, at least 92%, at least 94%, at least 95%, at least 96%, at least 98%, or 100% identical to any one of SEQ ID NOs: 95, 101, 107, 113, 119, 125, 131, 137, 143, 149, 155, 161, 167, 172, 179, 185, 191, 197, 203, 209, 215, 221, 227, or 233. In some embodiments, the B3 primer comprises a nucleotide sequence at least 85%, at least 87%, at least 90%, at least 92%, at least 94%, at least 95%, at least 96%, at least 98%, or 100% identical to any one of SEQ ID NOs: 93, 99, 105, 111, 117, 123, 129, 135, 141, 147, 153, 159, 165, 170, 177, 183, 189, 195, 201, 207, 213, 219, 225, or 231. In some embodiments, the F3 primer comprises a nucleotide sequence at least 85%, at least 87%, at least 90%, at least 92%, at least 94%, at least 95%, at least 96%, at least 98%, or 100% identical to any one of SEQ ID NOs: 92, 98, 104, 110, 116, 122, 128, 134, 140, 146, 152, 158, 164, 170, 176, 182, 188, 194, 200, 206, 212, 218, 224, or 230. In some embodiments, the LB primer comprises a nucleotide sequence at least 85%, at least 87%, at least 90%, at least 92%, at least 94%, at least 95%, at least 96%, at least 98%, or 100% identical to any one of SEQ ID NOs: 6, 12,18, 24, 30, 36, 42, 48, 54, 60, 66, 72, 78, 84, 90, 96, 102, 108, 114, 120, 126, 132, 138, or 144. In some embodiments, the LF primer comprises a nucleotide sequence at least 85%, at least 87%, at least 90%, at least 92%, at least 94%, at least 95%, at least 96%, at least 98%, or 100% identical to any one of SEQ ID NOs: 96, 102, 108, 114, 120, 126, 132, 138, 144, 150, 156, 162, 168, 173, 180, 186, 192, 198, 204, 210, 216, 222, 228, or 234.

In some embodiments, the at least six amplification primers comprise six nucleotide sequences at least 85%, at least 87%, at least 90%, at least 92%, at least 94%, at least 95%, at least 96%, at least 98%, or 100% identical to SEQ ID NOs: 92-97, SEQ ID NOs: 98-103, SEQ ID NOs: 104-109, SEQ ID NOs: 110⁻¹¹⁵, SEQ ID NOs: 116-121, SEQ ID NOs: 122-127, SEQ ID NOs: 128-133, SEQ ID NOs: 134-139, SEQ ID NOs: 140-145, SEQ ID NOs: 146-151, SEQ ID NOs: 152-157, SEQ ID NOs: 158-163, SEQ ID NOs: 164-169, SEQ ID NOs: 170-175, SEQ ID NOs: 176-181, SEQ ID NOs: 182-187, SEQ ID NOs: 188-193, SEQ ID NOs: 194-199, SEQ ID NOs: 200-205, SEQ ID NOs: 206-211, SEQ ID NOs: 212-217, SEQ ID NOs: 218-223, SEQ ID NOs: 224-229, or SEQ ID NOs: 230-235.

In some embodiments, the at least one amplification primer comprises at least three amplification primers. The at least three amplification primers may comprise a BIP primer, a B3 primer, and a LB primer. The at least three amplification primers comprise a FIP primer, a F3 primer, and a LF primer.

In some embodiments, the amplifying comprises isothermal amplification. The amplifying may comprise helicase dependent amplification (HDA), circular helicase dependent amplification (cHDA), strand displacement amplification (SDA), loop mediated amplification (LAMP), exponential amplification reaction (EXPAR), rolling circle amplification (RCA), ligase chain reaction (LCR), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The amplifying may comprise a thermal cycling amplification such as polymerase chain reaction (PCR).

In some embodiments, the amplifying comprises contacting the sample to reagents for amplification. In some embodiments, the contacting the sample to reagents for amplification occurs concurrent to the contacting the sample to the reporter and the composition comprising the programmable nuclease and the guide nucleic acid. In some embodiments, the reagents for amplification comprise a polymerase and dNTPs.

In some embodiments, the guide nucleic acid comprises a nucleobase sequence at least 85%, at least 87%, at least 90%, at least 92%, at least 94%, at least 95%, at least 96%, at least 98%, or 100% identical to any one of SEQ ID NOs: 236-247.

In some embodiments, the programmable nuclease is a type V CRISPR/Cas effector protein. In some embodiments, the type V CRISPR/Cas effector protein is a Cas14 protein. In some embodiments, the Cas 14 protein comprises a Cas 14a polypeptide, a Cas 14b polypeptide, a Cas14c polypeptide, a Cas14d polypeptide, a Cas14e polypeptide, a Cas 14f polypeptide, a Cas14g polypeptide, a Cas14h polypeptide, a Cas14i polypeptide, a Cas14j polypeptide, or a Cas14k polypeptide. In some embodiments, the Cas14 protein comprises an amino acid sequence at least 80%, 85%, at least 87%, at least 90%, at least 92%, at least 94%, at least 95%, at least 96%, at least 98%, or 100% identical to Cas14a.1.

In some embodiments, the method further comprising lysing the sample. In some embodiments, the lysing comprises contacting the sample to a lysis buffer.

In some embodiments, the method further comprising multiplexed detection of one or more additional target nucleic acid(s). In some embodiments, the one or more additional target nucleic acid(s) is: a) a different gene of the monkeypox virus or a segment thereof, b) a different segment of the same gene of the monkeypox virus, and/or c) a gene of another Orthopoxvirus or a segment thereof. In some embodiments, the other Orthopoxvirus is selected from the group consisting of abatino macacapox virus, akhmeta virus, alaskapox virus, camelpox virus, cowpox virus, ectromelia virus, raccoonpox virus, skunkpox virus, taterapox virus, vaccinia virus, variola virus, and volepox virus.

In some embodiments, sample lysis, amplification, detection, or any combination thereof is carried out in a single volume.

In some embodiments, sample lysis, amplification, detection, or any combination thereof is carried out in separate volumes.

In some embodiments, the sample is a saliva sample or a wound swab sample.

In some embodiments, the method further comprising repeating one or more method steps to assay for a negative control nucleic acid not comprising a gene of the monkeypox virus or a segment thereof.

Another aspect of the present disclosure provides a composition comprising a non-naturally occurring guide nucleic acid comprising a nucleotide sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 236-247.

Yet another aspect of the present disclosure provides a composition comprising an amplification primer comprising a nucleotide sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 92-235.

In another aspect, the present disclosure provides a method of assaying for a target nucleic acid in a sample, the method comprising a) contacting the sample to a reporter and a composition comprising a programmable nuclease and a guide nucleic acid that hybridizes to the target nucleic acid or an amplified product thereof, wherein the programmable nuclease cleaves the reporter upon hybridization of the guide nucleic acid to the target nucleic acid or the amplification product thereof; and b) assaying for a change in a signal, wherein the change in the signal is produced by cleavage of the reporter; wherein the target nucleic acid is a gene of a monkeypox virus or a segment thereof; and optionally wherein the guide nucleic acid comprises a nucleobase sequence at least 85%, at least 87%, at least 90%, at least 92%, at least 94%, at least 95%, at least 96%, at least 98%, or 100% identical to any one of SEQ ID NOS: 236-247.

These and other embodiments are addressed in more detail in the detailed description set forth below.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows images of sections of partitioning chambers loaded with the reaction mixtures described in Example 1 to compare the use of different RNA-targeting Cas proteins, comprising the amino acid sequence of SEQ ID NO: 21, 62 or 43. Varying amounts of the target nucleic acid (columns I through VIII) and a SARS-CoV-2 N-gene targeting guide RNA (columns I through VI and VIII) or an off-target guide (OTG, column VII) were used in each of the chambers, as depicted. Each black square is an image of a section of one partitioning chamber, while the mini dots within the chamber images are individual nanovolumes. The presence of the target nucleic acid in a particular nanovolume in the chamber is detected by the presence of a positive signal in that nanovolume.

FIG. 2 shows images of sections of partitioning chambers loaded with the reaction mixtures described in Example 2, having varying concentrations (0 fM to 10 nM) of the Cas protein comprising the amino acid sequence of SEQ ID NO: 21, and a fixed amount (600 copies/chamber) of the Twist Synthetic SARS-CoV-2 Synthetic RNA Control target nucleic acid or a no-target control (NTC, 0 copies/chamber). Each mixture also comprised a SARS-CoV-2 N-gene targeting guide RNA or a control using an off-target guide RNA (OTG).

FIG. 3 shows images of sections of partitioning chambers loaded with the reaction mixtures described in Example 2, having varying concentrations (0 fM to 10 nM) of the Cas protein comprising the amino acid sequence of SEQ ID NO: 21, and a fixed amount (600 copies/chamber) of the ATCC® synthetic SARS-CoV-2 RNA target nucleic acid, a no-target control (NTC, 0 copies/chamber), or an off-target control nucleic acid (OTC, 600 copies/chamber). Each mixture also comprised a SARS-CoV-2 N-gene targeting guide RNA or a control using an off-target guide RNA (OTG).

FIG. 4 shows images of sections of partitioning chambers loaded with the reaction mixtures described in Example 3 but having different proportional volumes of a low pH crude lysis buffer (0%, 5%, 10% or 20%), 5000 copies/chamber of Twist Synthetic SARS-CoV-2 Synthetic RNA Control target nucleic acid, and the Cas protein comprising the amino acid sequence of SEQ ID NO: 21. Each mixture also comprised a SARS-CoV-2 N-gene targeting guide RNA (R4684) or an off-target guide (OTG, R5882) as a negative control.

FIG. 5 shows images of sections of partitioning chambers loaded with the reaction mixtures described in Example 3, but in the presence of different carrier molecules (yeast RNA, glycogen, linear acrylamide or PVP at different concentrations), Twist Synthetic SARS-CoV-2 Synthetic RNA Control target nucleic acid, and the Cas protein comprising the amino acid sequence of SEQ ID NO: 21. A no-target control (NTC) was used as a negative control.

FIG. 6 shows images of sections of partitioning chambers loaded with the reaction mixtures described in Example 4 and Table A, comprising three types of RNAs in a mixture (the target SARS-CoV-2 RNA and the off-target Influenza A and B RNAs). Varying amounts of the target nucleic acid and/or off-target nucleic acids were used in each of the chambers, as depicted (4000 copies/chamber, 400 copies/chamber, 40 copies/chamber and 0 copies/chamber). Either decreasing the concentration of all the three RNAs (top row) or decreasing the concentration of just the SARS CoV-2 target nucleic acid (middle and bottom rows) resulted in a similar decrease in the number of positive nanovolumes.

FIG. 7 shows images of partitioning chambers loaded with the reaction mixtures described in Example 5 to test the function of a DNA-targeting Cas protein comprising the amino acid sequence of SEQ ID NO: 34. Varying amounts (6,250 copies/reaction, 12,500 copies, reaction, 18,750 copies/reaction or 25,000 copies/reaction) of the synthetic dsDNA target nucleic acid, a no-target control (NTC, 0 copies/reaction) or an off-target control (OTC) nucleic acid were used in each of the chambers, as depicted.

FIG. 8 shows images of partitioning chambers loaded with the reaction mixtures described in Example 5 to test the function of a DNA-targeting Cas protein comprising the amino acid sequence of SEQ ID NO: 3. The synthetic SARS-CoV-2 dsDNA target nucleic acid or a no-target control (NTC) was used, as indicated.

FIG. 9A is a bar graph depicting the number of copies/reaction of SARS-CoV-2 target nucleic acid or PMMoV target nucleic acid (off-target control, OTC) detected using the quantitative assays described herein employing a SARS-CoV-2 crRNA for various concentrations of synthetic SARS-CoV-2 target as an input (from dilution D1 down to dilution D6). FIG. 9B is a bar graph depicting the number of copies/reaction of PMMoV target nucleic acid or SARS-CoV-2 target nucleic acid (off-target control, OTC) detected using the quantitative assays described herein employing a PMMoV crRNA for various concentrations of synthetic PMMoV target as an input (from dilution D1 down to dilution D6). See Example 6.

FIG. 10A is a standard curve obtained using the quantitative assays described herein for the SARS-CoV-2 target nucleic acid, showing the relatively linear relationship between the number of target copies per chamber (x-axis) and the number of positive nanovolumes observed (y-axis). FIG. 10B is a standard curve obtained using the quantitative assays described herein for the PMMOV target nucleic acid, showing the relatively linear relationship between the number of target copies per chamber (x-axis) and the number of positive nanovolumes observed (y-axis).

FIG. 11 shows images of sections of partitioning chambers loaded with the reaction mixtures described in Example 7 to compare the use of an RNA-targeting Cas protein, comprising the amino acid sequence of SEQ ID NO: 21, over a range of target nucleic acid concentrations (3,333 copies/μL to 0 copies/μL) or an off-target guide (OTG), as depicted. Each black square is an image of a section of one partitioning chamber, while the mini dots within the chamber images are individual nanovolumes. The presence of the target nucleic acid in a particular nanovolume in the chamber is detected by the presence of a positive signal in that nanovolume.

FIG. 12 shows images of sections of partitioning chambers loaded with the reaction mixtures described in Example 8 to compare the use of an RNA-targeting Cas protein, comprising the amino acid sequence of SEQ ID NO: 21 in a digital DETECTR reaction with the gold standard ddPCR assay on viral nucleic acid samples extracted from SARS-CoV-2-positive clinical samples. Each black square is an image of a section of one partitioning chamber, while the mini dots within the chamber images are individual nanovolumes. The presence of the target nucleic acid in a particular nanovolume in the chamber is detected by the presence of a positive signal in that nanovolume.

FIG. 13 shows a head-to-head comparison of data generated from ddPCR and digital DETECTR run on SARS-CoV-2-positive clinical samples as described in Example 8. Data collected showed high linearity across the range of extracted sample dilutions tested.

FIG. 14 shows a comparison of the number of copies per microliter (cp/μL) determined for three wastewater samples using digital DETECTR and ddPCR. These data show that digital DETECTR has a roughly equivalent dynamic range to digital PCR for viral quantification under the conditions tested for both SARS-CoV-2 and PMMOV RNA in wastewater samples.

FIG. 15A shows a head-to-head comparison of digital DETECTR and dPCR signals obtained from wastewater samples for SARS-CoV-2 quantitation, graphed with the same scaling along the Y-axis. FIG. 15B shows the digital DETECTR and dPCR signals graphed with different Y-axis scaling, in order to highlight the stark differences in signals obtained by each method.

FIG. 16 shows bar graphs depicting the number of copies/reaction of SARS-CoV-2 target dsDNA nucleic acid detected using the quantitative assays described herein employing a different DNA-targeting Cas proteins (comprising the amino acid sequence of SEQ ID NO: 65, 67, 68, 34, 17, 89, 90, or 91) at 37C or 50C reaction temperatures.

FIG. 17 shows a schematic of an exemplary warm-start strategy which may be employed to delay activation of the CRISPR/Cas complex until the complex has reached a predetermined reaction temperature.

### DETAILED DESCRIPTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any and all combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers +/−10% thereof, or 10% below the lower listed limit and 10% above the higher listed limit for the values listed for a range.

The terms, “% identical,” “% identity,” “percent identity,” and grammatical equivalents thereof, as used herein, in the context of an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4 (1): 11-7), FASTA (Pearson and Lipman, Proc Natl Acad Sci USA. 1988 Apr; 85 (8): 2444-8; Pearson, Methods Enzymol. 1990; 183: 63-98) and gapped BLAST (Altschul et al., Nucleic Acids Res. 1997 Sep. 1; 25 (17): 3389-40), BLASTP, BLASTN, or GCG (Devereux et al., Nucleic Acids Res. 1984 Jan. 11; 12 (1 Pt 1): 387-95).

The terms, “% complementary”, “% complementarity”, “percent complementary”, “percent complementarity” and grammatical equivalents thereof, as used interchangeably herein, in the context of two or more nucleic acid molecules, refer to the percent of nucleotides in two nucleotide sequences in said nucleic acid molecules of equal length that can undergo cumulative base pairing at two or more individual corresponding positions in an antiparallel orientation. Accordingly, the terms include nucleic acid sequences that are not completely complementary over their entire length, which indicates that the two or more nucleic acid molecules include one or more mismatches. A “mismatch” is present at any position in the two opposed nucleotides that are not complementary. The % complementary is calculated by dividing the total number of the complementary residues by the total number of the nucleotides in one of the equal length sequences, and multiplying by 100. Complete or total complementarity describes nucleotide sequences in 100% of the residues of a nucleotide sequence are complementary to residues in a reference nucleotide sequence. “Partially complementarity” describes nucleotide sequences in which at least 20%, but less than 100%, of the residues of a nucleotide sequence are complementary to residues in a reference nucleotide sequence. In some instances, at least 50%, but less than 100%, of the residues of a nucleotide sequence are complementary to residues in a reference nucleotide sequence. In some instances, at least 70%, 80%, 90% or 95%, but less than 100%, of the residues of a nucleotide sequence are complementary to residues in a reference nucleotide sequence. “Noncomplementary” describes nucleotide sequences in which less than 20% of the residues of a nucleotide sequence are complementary to residues in a reference nucleotide sequence.

The terms, “amplification,” “amplifying,” and grammatical equivalents thereof, as used herein, refer to a process by which a nucleic acid molecule is enzymatically copied to generate a plurality of nucleic acid molecules containing the same sequence as the original nucleic acid molecule or a distinguishable portion thereof.

The terms, “bind,” “binding,” “interact” and “interacting,” as used herein, refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

The term, “cis cleavage,” as used herein refers to cleavage (hydrolysis of a phosphodiester bond) of a target nucleic acid by an effector protein complexed with a guide nucleic acid refers to cleavage of a target nucleic acid that is hybridized to a guide nucleic acid, wherein cleavage occurs within or directly adjacent to the region of the target nucleic acid that is hybridized to the guide nucleic acid.

The term, “codon optimized,” as used herein, refers to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon.

The terms, “complementary” and “complementarity,” as used herein, in the context of a nucleic acid molecule or nucleotide sequence, refer to the characteristic of a polynucleotide having nucleotides that can undergo cumulative base pairing with their Watson-Crick counterparts (C with G; or A with T) in a reference nucleic acid in antiparallel orientation. For example, when every nucleotide in a polynucleotide or a specified portion thereof forms a base pair with every nucleotide in an equal length sequence of a reference nucleic acid, that polynucleotide is said to be 100% complementary to the sequence of the reference nucleic acid. In a double stranded DNA or RNA sequence, the upper (sense) strand sequence is, in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′-to its 5′-end, while the “reverse complement” sequence or the “reverse complementary” sequence is understood as the sequence of the lower strand in the direction of its 5′-to its 3′-end. Each nucleotide in a double stranded DNA or RNA molecule that is paired with its Watson-Crick counterpart can be referred to as its complementary nucleotide. The complementarity of modified or artificial base pairs can be based on other types of hydrogen bonding and/or hydrophobicity of bases and/or shape complementarity between bases.

The term, “cleavage assay,” as used herein, refers to an assay designed to visualize, quantitate or identify cleavage of a nucleic acid. In some instances, the cleavage activity may be cis-cleavage activity. In some instances, the cleavage activity may be trans-cleavage activity.

The terms, “cleave,” “cleaving” and “cleavage,” as used herein, in the context of a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from another organism.

The term, “conservative substitution,” as used herein, refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

The terms, “CRISPR RNA” and “crRNA,” as used herein, refer to a type of guide nucleic acid that is RNA comprising a first sequence that is capable of hybridizing to a target sequence of a target nucleic acid and a second sequence that is capable of interacting with an effector protein either directly (by being bound by an effector protein) or indirectly (e.g., by hybridization with a second nucleic acid molecule that can be bound by an effector). The first sequence and the second sequence are directly connected to each other or by a linker.

The term, “detectable signal,” as used herein, refers to a signal that can be detected using optical, fluorescent, chemiluminescent, electrochemical or other detection methods known in the art.

The term, “detecting a nucleic acid” and its grammatical equivalents, as used herein refers to detecting the presence or absence of the target nucleic acid in a sample that potentially contains the nucleic acid being detected.

The term, “detection moiety,” as used herein refers to a molecule that can release a signal that can be detected using optical, fluorescent, chemiluminescent, electrochemical, calorimetric and other detection methods known in the art.

The term, “effector protein,” as used herein, refers to a protein, polypeptide, or peptide that is capable of interacting with a nucleic acid, such as a guide nucleic acid, to form a complex (e.g., a RNP complex), wherein the complex interacts with a target nucleic acid.

The terms, “effector partner” and “partner polypeptide” as used herein, refer to a polypeptide that does not have 100% sequence identity with an effector protein described herein. In some instances, an effector partner described herein may be found in a homologous genome as an effector protein described herein.

The term, “engineered modification,” as used herein, refers to a structural change of one or more nucleic acid residues of a nucleotide sequence or one or more amino acid residue of an amino acid sequence, such as chemical modification of one or more nucleobases; or a chemical change to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “guide nucleic acid,” as used herein, refers to a nucleic acid that, when in a complex with one or more polypeptides described herein (e.g., an RNP complex) can impart sequence selectivity to the complex when the complex interacts with a target nucleic acid. A guide nucleic acid may be referred to interchangeably as a guide RNA, however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof. In some embodiments, the guide nucleic acid comprises: a first nucleotide sequence that hybridizes to a target nucleic acid; and a second nucleotide sequence that is capable of being non-covalently bound by an effector protein, such as, Cas. The first sequence may be referred to herein as a spacer sequence. The second sequence may be referred to herein as a repeat sequence. In some embodiments, the first sequence is located 5′ of the second nucleotide sequence. In some embodiments, the first sequence is located 3′ of the second nucleotide sequence.

The term, “heterologous,” as used herein, refers to at least two different polypeptide sequences that are not found similarly connected to one another in a native nucleic acid or protein. A protein that is heterologous to the effector protein is a protein that is not covalently linked by an amide bond to the effector protein in nature. In some instances, a heterologous protein is not encoded by a species that encodes the effector protein. A guide nucleic acid may comprise “heterologous” sequences, which means that it includes a first sequence and a second sequence, wherein the first sequence and the second sequence are not found covalently linked by a phosphodiester bond in nature. Thus, the first sequence is considered to be heterologous with the second sequence, and the guide nucleic acid may be referred to as a heterologous guide nucleic acid.

The terms, “hybridize,” “hybridizable” and grammatical equivalents thereof, refer to a nucleotide sequence that is able to noncovalently interact, i.e., form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g., complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001).

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary RNA sequence, that is capable of being non-covalently bound by an effector protein. In some instances, the handle sequence further comprises a repeat sequence. In such instances, the intermediary RNA sequence or a combination of the intermediary RNA and the repeat sequence is capable of being non-covalently bound by an effector protein.

The terms, “intermediary RNA” and “intermediary RNA sequence,” as used herein, in a context of a single nucleic acid system, refers to a nucleotide sequence in a handle sequence, wherein the intermediary RNA sequence is capable of, at least partially, being non-covalently bound to an effector protein to form a complex (e.g., an RNP complex). An intermediary RNA sequence is not a transactivating nucleic acid in systems, methods, and compositions described herein.

The term, “in vitro,” as used herein, refers to describing something outside an organism. An in vitro system, composition or method may take place in a container for holding laboratory reagents such that it is separated from the biological source from which a material in the container is obtained. In vitro assays can encompass cell-based assays in which living or dead cells are employed. In vitro assays can also encompass a cell-free assay in which no intact cells are employed. The term “in vivo” is used to describe an event that takes place within an organism. The term “ex vivo” is used to describe an event that takes place in a cell that has been obtained from an organism. An ex vivo assay is not performed on a subject. Rather, it is performed upon a sample separate from a subject.

The terms, “length” and “linked nucleosides,” as used herein, refer to a nucleic acid (polynucleotide) or polypeptide, may be expressed as “kilobases” (kb) or “base pairs (bp),”. Thus, a length of 1 kb refers to a length of 1000 linked nucleosides, and a length of 500 bp refers to a length of 500 linked nucleosides. Similarly, a protein having a length of 500 linked amino acids may also be simply described as having a length of 500 amino acids.

The term, “linker,” as used herein, refers to a covalent bond or molecule that links a first polypeptide to a second polypeptide (e.g., by an amide bond) or a first nucleic acid to a second nucleic acid (e.g., by a phosphodiester bond).

The term, “mutation,” as used herein, refers to an alteration that changes an amino acid residue or a nucleotide as described herein. Such an alteration can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation. Methods of mutating an amino acid residue or a nucleotide are well known.

The terms, “non-naturally occurring” and “engineered,” as used herein, refer to indicate involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The terms, “nuclease” and “endonuclease” as used herein, refer to an enzyme which possesses catalytic activity for nucleic acid cleavage.

The term, “nuclease activity,” as used herein, refers to catalytic activity that results in nucleic acid cleavage (e.g., ribonuclease activity (ribonucleic acid cleavage), or deoxyribonuclease activity (deoxyribonucleic acid cleavage), etc.).

The term, “nucleic acid,” as used herein, refers to a polymer of nucleotides. A nucleic acid may comprise ribonucleotides, deoxyribonucleotides, combinations thereof, and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more engineered modifications, or both.

The term, “nucleic acid expression vector,” as used herein, refers to a plasmid that can be used to express a nucleic acid of interest.

The terms, “nucleotide(s)” and “nucleoside(s)”, as used herein, in the context of a nucleic acid molecule having multiple residues, refer to describing the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

The term, “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

The terms, “polypeptide” and “protein,” as used herein, refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some embodiments, when a heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The terms, “protospacer adjacent motif” and “PAM,” as used herein, refer to a nucleotide sequence found in a target nucleic acid that directs an effector protein to edit the target nucleic acid at a specific location. In some instances, a PAM is required for a complex of an effector protein and a guide nucleic acid (e.g., an RNP complex) to hybridize to and edit the target nucleic acid. In some instances, the complex does not require a PAM to edit the target nucleic acid.

The term, “recombinant,” as used herein, in the context of proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The terms, “reporter” and “reporter nucleic acid,” as used herein, refer to a non-target nucleic acid molecule that can provide a detectable signal upon cleavage by an effector protein. Examples of detectable signals and detectable moieties that generate detectable signals are provided herein. In some embodiments, the reporter conveys the presence of a target nucleic acid.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “sample,” as used herein generally refers to something comprising a target nucleic acid. In some embodiments, the sample is a biological sample, such as a saliva sample or a wound swab sample.

The terms, “single guide nucleic acid”, “single guide RNA” and “sgRNA,” as used herein, in the context of a single nucleic acid system, refers to a guide nucleic acid, wherein the guide nucleic acid is a single polynucleotide chain having all the required sequence for a functional complex with an effector protein (e.g., being bound by an effector protein, including in some instances activating the effector protein, and hybridizing to a target nucleic acid, without the need for a second nucleic acid molecule). For example, an sgRNA can have two or more linked guide nucleic acid components (e.g., an intermediary RNA sequence, a repeat sequence, a spacer sequence and optionally a linker, or a handle sequence and a spacer sequence). A handle sequence may comprise at least a portion of a tracrRNA sequence, at least a portion of a repeat sequence, or a combination thereof. A sgRNA does not comprise a tracrRNA.

The term, “spacer sequence,” as used herein, refers to a nucleotide sequence in a guide nucleic acid that is capable of, at least partially, hybridizing to an equal length portion of a sequence (e.g., a target sequence) of a target nucleic acid.

The term, “subject,” as used herein, refers to an animal. The subject may be a mammal. The subject may be a human. The subject may be diagnosed or at risk for a disease.

The term, “sufficiently complementary,” as used herein, refers to a first nucleotide sequence that is partially complementarity to a second nucleotide sequence while still allowing the first nucleotide sequence to hybridize to the second nucleotide sequence with enough affinity to permit a biological activity to occur. Depending on the context, a biological activity may be the formation of a complex between two or more components described herein, such as an effector protein and a guide nucleic acid. A biological activity may also be bringing one or more components described herein into proximity of another component, such as bringing an effector protein-guide nucleic acid complex into proximity of a target nucleic acid. A biological activity may additionally be permitting a component described herein to act on another component described herein, such as permitting an effector protein to cleave a target nucleic acid. In some instances, sequences are said to be sufficiently complementary when at least 85% of the residues of a nucleotide sequence are complementary to residues in a reference nucleotide sequence.

The term, “target nucleic acid,” as used herein, refers to a nucleic acid that is selected as the nucleic acid for editing, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, in the context of a target nucleic acid, refers to a nucleotide sequence found within a target nucleic acid. Such a nucleotide sequence can, for example, hybridize to a respective length portion of a guide nucleic acid.

The terms, “trans-activating RNA”, “transactivating RNA” and “tracrRNA,” refer to a transactivating or transactivated nucleic acid in a dual nucleic acid system that is capable of hybridizing, at least partially, to a crRNA to form a tracrRNA-crRNA duplex, and of interacting with an effector protein to form a complex (e.g., an RNP complex).

The terms, “transactivating”, “trans-activating”, “trans-activated”, “transactivated” and grammatical equivalents thereof, as used herein, in the context of a dual nucleic acid system refers to an outcome of the system, wherein a polypeptide is enabled to have a binding and/or nuclease activity on a target nucleic acid, by a tracrRNA or a tracrRNA-crRNA duplex.

The term, “trans cleavage,” as used herein, in the context of cleavage (e.g., hydrolysis of a phosphodiester bond) of one or more target nucleic acids or non-target nucleic acids, or both, by an effector protein that is complexed with a guide nucleic acid and the target nucleic acid. Trans cleavage activity may be triggered by the hybridization of a guide nucleic acid to a target nucleic acid. The effector may cleave a target strand as well as non-target strand, wherein the target nucleic is a double stranded nucleic acid. Trans cleavage of the target nucleic acid may occur away from (e.g., not within or directly adjacent to) the portion of the target nucleic acid that is hybridized to the portion of the guide nucleic acid.

The terms, “treatment” and “treating,” as used herein, refer to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “variant,” as used herein, refers to a form or version of a protein that differs from the wild-type protein. A variant may have a different function or activity relative to the wild-type protein.

As used herein, a “reaction mixture” refers to a composition comprising an effector protein (e.g., a CRISPR/Cas effector protein), a guide nucleic acid (e.g., a guide RNA) and a reporter nucleic acid. In some embodiments, the reaction mixture comprises a target nucleic acid capable of binding to the guide nucleic acid.

As used herein, a “no target control” or “NTC” refers to a reaction mixture that may be used as a negative control in the methods described herein, which does not contain a target nucleic acid that is capable of binding to the guide nucleic acid.

As used herein, an “off-target control” or “OTC” refers to a nucleic acid that may be used as a negative control in the methods described herein having a sequence that is not capable of binding to the guide nucleic acid, or to a reaction mixture that may be used as a negative control in the methods described herein containing a negative control nucleic acid having a sequence that is not capable of binding to the guide nucleic acid.

As used herein, an “off-target guide” or “OTG” refers to a guide nucleic acid used as a negative control in the methods described herein, which is not capable of binding to the target nucleic acid molecule, or to a reaction mixture used as a negative control in the methods described herein which contains a guide nucleic acid that is not capable of binding to the target nucleic acid molecule.

As used herein, a “nanovolume” refers to an individual volume of less than or equal of 100 nL, preferably less than or equal to 1 nL. The form factor that can partition the nanovolumes from a larger input volume is not limited, and may be, for instance, a droplet, a well, a microwell, a nanowell, and the like.

As used herein, a “test sample” (or “sample”) refers to a composition, comprising a target nucleic acid. In some instances, the test sample is a biological sample, such as a biological fluid or tissue sample. In some instances, the test sample is an environmental sample. The test sample may be a biological sample or environmental sample that is modified or manipulated. For instance, in some embodiments, the test samples may be modified or manipulated with purification techniques, heat, salts, buffers, or any combination thereof.

As used herein, a “positive control sample” refers to a sample in which the amount or concentration of a nucleic acid (e.g., target nucleic acid, or positive control target nucleic acid) is known prior to performing the methods for quantitation disclosed herein.

As used herein, a “positive control target nucleic acid” is a nucleic acid that is used as a control for the reaction conditions of the methods disclosed herein, such as, the performance of the effector protein, the functionality of the reporter nucleic acid and/or the detection of the detectable signals.

### Methods For Quantitation of Nucleic Acid Targets

Provided herein are compositions, systems, and methods of quantitating a target nucleic acid.

For instance, the disclosure provides methods for quantitating a target nucleic acid in a test sample, comprising (a) contacting the test sample with the following components (i) through (iii) resulting in at least two nanovolumes of a reaction mixture: (i) any one or more of the effector proteins described herein, (ii) a guide nucleic acid, comprising a region that is capable of binding to the effector protein, and (iii) a guide sequence that is capable of hybridizing with the target nucleic acid, and (iv) a reporter that is single stranded and does not hybridize with the guide sequence of the guide nucleic acid, (b) measuring signals detected from the at least two nanovolumes and generated by cleavage of the reporter by the effector protein, and (c) quantitating the target nucleic acid in the test sample based on the measured signals. The disclosure provides methods for quantitating a target nucleic acid that do not comprise amplifying the target nucleic acid in the test sample.

In some embodiments, the contacting step comprises sequentially adding each of the components (i) through (iii) to the test sample in at least two nanovolumes. The order of adding the components is not limited. In some embodiments, two or more of the components may be added together at the same time. In some embodiments, the contacting step comprises: (a) adding each of the components (i) through (iii) to the test sample to generate a master reaction mixture, wherein the master reaction mixture has a volume of more than 1 nL, and (b) distributing the master reaction mixture into at least two nanovolumes.

In some embodiments, the methods disclosed herein utilize an instrument that is capable of partitioning the reaction mixture into two or more nanovolumes (within two or more compartments or partitions), wherein each of the compartments comprises a nanovolume to be used in the methods described herein. Non-limiting examples are such instruments include a fluidic device (e.g., a Beckman Coulter Echo 525 Liquid Handler), or a digital PCR machine (e.g., QIAcuity Digital PCR System). In some embodiments, the methods disclosed herein utilize chambers comprising two or more compartments or partitions, wherein the two or more compartments comprise the two or more nanovolumes, as described herein.

In some embodiments, the contacting step comprises contacting the test sample with components (i) through (iii) at a first pre-determined temperature. The method further comprises changing the temperature from the first pre-determined temperature to a second pre-determined temperature after the reaction mixture is partitioned into the at least two nanovolumes. The first pre-determined temperature may be selected to reduce or prevent activity of and/or interaction between one or more of the components of the reaction mixture. The second pre-determined temperature may be selected to increase or enable activity of and/or interaction between one or more of the components of the reaction mixture.

In some embodiments, the first pre-determined temperature may be a temperature at which the effector protein has reduced or no catalytic activity, such that the effector protein remains inactive during partitioning in order to delay the start of the detection reaction (and generation of detectable signals by cleavage of the reporter by the effector protein) until the at least two nanovolumes have been formed. The second pre-determined temperature may be a temperature at which the effector protein has improved catalytic activity relative to the first pre-determined temperature, such that changing the temperature from the first pre-determined temperature to a second pre-determined temperature activates the effector protein and begins the detection reaction. For example, an effector protein may be selected which has little to no catalytic activity at a partitioning temperature of 4C, room temperature, or 37C, but is highly active at a reaction temperature of 50C, 55C, or 60C. In another example, an effector protein may be selected which has some catalytic activity at a partitioning temperature of 4C, room temperature, or 37C, but is more active at a reaction temperature of 50C, 55C, or 60C, thus enabling the majority of reporter cleavage to occur after the reaction mixture has been partitioned into nanovolumes. In some embodiments, the effector protein may be engineered for specific levels (or not) of catalytic activity at specific temperatures as described herein. Such temperature shifting warm start strategies may be particularly useful when the chosen effector protein is highly active at its preferred operating temperature, which could result in high background within nanovolumes if the effector protein were active from the moment of contacting and throughout the partitioning process.

In some embodiments, the effector protein may be associated with one or more blockers (e.g., aptamers, antibodies, etc.) which prevent catalytic activity until disassociated therefrom. Blockers may be selected for their temperature sensitivity such that they remain associated with the effector protein at the first pre-determined temperature and disassociate from the effector protein (e.g., by denaturing) at the second pre-determined temperature. Upon disassociation, the effector protein is then active and free to begin the detection reaction within the nanovolumes. Such warm start strategies may be particularly useful when the chosen effector protein is highly active across a range of temperatures (e.g., at room temperature and at temperatures higher than the disassociation temperature of the blocker), which could result in high background within nanovolumes if the effector protein were active from the moment of contacting and throughout the partitioning process.

In some embodiments, the contacting step comprises contacting the test sample with one or more signal amplification reagents. For example, the one or more signal amplification reagents may comprise (i) a second effector protein and (ii) a second reporter that is single stranded and does not hybridize with the guide sequence of the guide nucleic acid. The second reporter may comprise a first nucleic acid section and a second nucleic acid section. The first nucleic acid section may act as an activator for the second effector protein when separated from the second nucleic acid section. The second nucleic acid section may act as a blocker nucleic acid and prevent the first nucleic acid from binding to and/or activating the second effector protein. In some embodiments, the second effector protein may comprise a Type III Cas protein such as NucC, Csm6, etc. and the second reporter is a capped activator thereof. Presence of the target nucleic acid may activate the first effector protein, which may cleave the first nucleic acid section from the second nucleic acid section, thereby freeing the first nucleic acid section to bind to and activate the second effector protein. The activated second effector protein then cleaves the first reporter to generate the signal detected in the measuring step. In some embodiments, the first effector protein may cleave both the first reporter and the second reporter.

In some embodiments, each of the at least two nanovolumes comprises no more than 1 molecule of the target nucleic acid. In some embodiments, the contacting step results in a number of nanovolumes in the range of about 5000 nanovolumes to about 500,000 nanovolumes, for example, about 6000, about 7000, about 8000, about 9000, about 10,000, about 20,000, about 30,000, about 40,000, about 50,000, about 60,000, about 70,000, about 80,000, about 90,000, about 100,000 nanovolumes, about 200,000 nanovolumes, about 300,000 nanovolumes, about 400,000 nanovolumes, or about 500,000 nanovolumes. In some embodiments, step (b) comprises measuring a binary signal (e.g., positive or negative) from each of the at least two nanovolumes. In some embodiments, step (c) comprises counting the number of nanovolumes providing a signal indicative of the presence of the target nucleic acid therein.

In some embodiments, the methods disclosed herein comprise contacting the test sample with a precursor guide RNA array, wherein the effector protein cleaves the precursor guide RNA array to produce the guide RNA. In some embodiments, the effector protein and the guide RNA are incubated with each other prior to step (a). In some embodiments, the effector protein and the guide RNA are not incubated with each other prior to step (a).

In some embodiments, the methods further comprise quantitating multiple different target nucleic acids in the test sample, wherein the at least two nanovolumes of reaction mixture comprises one or more guide RNAs, wherein at least one guide RNA is capable of hybridizing with each of the multiple target nucleic acids. In some embodiments, each of the at least two nanovolumes of reaction mixture comprises at least one guide RNA that is capable of hybridizing with each of the multiple target nucleic acids. In some embodiments, no two of the one or more guide RNAs are capable of binding to the same target nucleic acid. In other words, in some embodiments, each of the one or more guide RNAs is capable of binding a different target nucleic acid among the multiple target nucleic acids in the test sample.

In some embodiments, methods of quantitating multiple target nucleic acids may comprise using one or more guide RNAs comprising a guide sequence that is capable of hybridizing with target nucleic acids (e.g., bacterial or viral target nucleic acids) in a patient sample to inform treatment strategy. For instance, detection of a bacterial target nucleic acid in the sample can guide the administration of antibiotics. In some embodiments, the methods disclosed herein may be used for pan-disease determination (e.g., pan-influenza testing with guide nucleic acids targeting multiple H/N subtypes), which may be especially useful when exact knowledge of the disease-causing agent or subtype is unnecessary to properly inform treatment.

In some embodiments, each of the at least two nanovolumes of reaction mixture comprises at least one of the one or more guide RNAs comprising a guide sequence that is capable of hybridizing with no more than one of the multiple target nucleic acids. In some embodiments, each of the at least two nanovolumes of reaction mixture comprise different guide RNAs so as to detect different target nucleic acids.

In some embodiments, the methods further comprise quantitating a positive control target nucleic acid in a positive control sample, the method comprising: (a) contacting the positive control sample with the following components (i) through (iii) resulting in at least two nanovolumes of reaction mixture: (i) a effector protein; (ii) a positive control guide RNA, comprising a region that is capable of binding to the effector protein, and a guide sequence that is capable of hybridizing with the positive control target nucleic acid; and (iii) a reporter nucleic acid that is single stranded and does not hybridize with the guide sequence of the positive control guide RNA; (b) measuring signals detected from the at least two nanovolumes and produced by cleavage of the reporter nucleic acid by the effector protein, and (c) quantitating the amount of positive control target nucleic acid in the positive control sample based on the measured signals. In some embodiments, the at least two nanovolumes comprises more than one type of CRISPR/Cas effector protein, and one or more positive control guide RNAs capable of binding to each of the more than one type of CRISPR/Cas effector protein. In some embodiments, the at least two nanovolumes comprises a DNA-targeting effector protein (e.g., a Cas12 protein) and an RNA-targeting effector protein (e.g., a Cas13 protein), a positive control guide RNA comprising a region that is capable of binding to the DNA-targeting effector protein and a positive control guide RNA comprising a region that is capable of binding to the RNA-targeting effector protein.

In some embodiments, the methods further comprise quantitating the target nucleic acid in a positive control sample, the method comprising: (a) contacting the positive control sample with the following components (i) through (iii) resulting in at least two nanovolumes of reaction mixture: (i) a CRISPR/Cas effector protein; (ii) the guide RNA, comprising a region that is capable of binding to the CRISPR/Cas effector protein, and a guide sequence that is capable of hybridizing with the target nucleic acid; and (iii) a reporter nucleic acid that is single stranded and does not hybridize with the guide sequence of the guide RNA; (b) measuring signals detected from the at least two nanovolumes produced by cleavage of the reporter nucleic acid by the CRISPR/Cas effector protein, and (c) quantitating the amount of target nucleic acid in the positive control sample based on the measured signals.

In some embodiments, the methods comprise generating a standard curve for the target nucleic acid in the positive control sample, and obtaining an absolute quantitation of the target nucleic acid in the test sample based on the standard curve. In some embodiments, the method comprises obtaining a relative quantitation of the target nucleic acid in the test sample based on the quantitation of the target nucleic acid in a positive control sample.

The methods disclosed herein may be used for a wide range of applications, including, disease surveillance (e.g., wastewater-based epidemiology), cell and gene therapy, therapeutic selection, therapeutic monitoring, minimum residual disease (e.g., for oncology applications, etc.), carrier screening, oncology, cDNA conversion rates, and mRNA quality control (mRNA therapeutics, mRNA vaccines, mRNA reagents). For example, the methods disclosed herein may be used for disease surveillance as described in Examples 6 and 9. In another example, the methods disclosed herein may be used for various cell and gene therapy applications, including quantifying mRNA and/or gRNA payloads of lipid nanoparticles (LNPs) or other nucleic acid delivery vehicles such as adeno-associated viruses (AAVs), quantifying gene silencing efficiency in treated tissue samples, quantifying nucleic acid delivery to tissues (e.g., pharmacokinetics), etc. Current methods for RNA quantitation are typically unable to discriminate between mRNA and gRNA, which is less than ideal for determining therapeutic dosages, manufacturing purity, etc. Similar challenges exist for distinguishing between gRNA and mRNA in tissues when quantifying gene silencing efficiencies. The methods disclosed herein may be used to quantify and distinguish between different RNA specifies within a single delivery vehicle or tissue sample, e.g., using a multiplexing and/or guide pooling strategy as described herein. In another example, the methods disclosed herein may be used to monitor nucleic acid targets within a cell or tissue which are associated with a particular disease state or severity, e.g., to monitor the efficacy of a therapeutic agent within a tissue by determining the amount of relevant biomarker present in the tissue. In another example, the methods disclosed herein may be used for mRNA quality control. The methods disclosed herein may be used to identify and/or quantify mRNA capping impurities, e.g., in in vitro transcribed mRNA products which may be incorporated into delivery vehicles, such as for mRNA-based vaccines or gene therapies. Alternatively, or in combination, the methods disclosed herein may be used to identify and/or quantify unwanted nucleic acid impurities within a given samples, e.g., dsRNA contaminating in vitro transcribed mRNA products. A person of ordinary skill in the art will understand based on the disclosure herein that these and other applications are within the scope of the claims and embodiments described herein.

### Effector Proteins

An effector protein provided herein interacts with a guide nucleic acid to form a complex. In some embodiments, the complex interacts with a target nucleic acid. In some embodiments, an interaction between the complex and a target nucleic acid comprises one or more of: recognition of a protospacer adjacent motif (PAM) sequence within the target nucleic acid by the effector protein, hybridization of the guide nucleic acid to the target nucleic acid, modification of the target nucleic acid by the effector protein, or combinations thereof. In some embodiments, recognition of a PAM sequence within a target nucleic acid may direct the modification activity of an effector protein.

In some embodiments, the effector protein is a CRISPR-associated (“Cas”) protein. An effector protein may function as a single protein, including a single protein that is capable of binding to a guide nucleic acid and editing a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., editing a target nucleic acid). In some embodiments, an effector protein, when functioning in a multiprotein complex, may have differing and/or complementary functional activity to other effector proteins in the multiprotein complex. An effector protein may be a modified effector protein having increased modification activity and/or increased substrate binding activity (e.g., substrate selectivity, specificity, and/or affinity).

An effector protein may be small, which may be beneficial for nucleic acid detection (for example, the effector protein may be less likely to adsorb to a surface due to its small size). The smaller nature of these effector proteins may allow for them to be more readily incorporated as a reagent in an assay. In some embodiments, the length of the effector protein is at least about 100, about 200, about 300, about 400, about 500, about 600, about 700, about 800, about 900, about 1000, about 1100, about 1200, about 1300, about 1400, about 1500, or more linked amino acid residues.

TABLE 1 provides illustrative amino acid sequences of effector proteins that are useful in the compositions, systems and methods described herein.

In some embodiments, compositions, systems and methods described herein comprise an effector protein, or a nucleic acid encoding the effector protein, wherein the amino acid sequence of the effector protein comprises at least about 200 contiguous amino acids or more of any one of the sequences recited in Error! Reference source not found. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, at least about 620 contiguous amino acids, at least about 640 contiguous amino acids, at least about 660 contiguous amino acids, at least about 680 contiguous amino acids, at least about 700, at least about 800, at least about 900, at least about 1000, at least about 1100, at least about 1200, at least about 1300, at least about 1400, at least about 1500, contiguous amino acids, or more of any one of the sequences of Error! Reference source not found.

In some embodiments, compositions, systems and methods described herein comprise an effector protein or a nucleic acid encoding the effector protein, wherein the effector protein comprises a portion of any one of the sequences recited in TABLE 1. In some embodiments, the effector protein comprises a portion of any one of the sequences recited in TABLE 1, wherein the portion does not comprise at least the first 10 amino acids, first 20 amino acids, 40 amino acids, 60 amino acids, 80 amino acids, 100 amino acids, 120 amino acids, 140 amino acids, 160 amino acids, 180 amino acids, or 200 amino acids of any one of the sequences recited in TABLE 1. In some embodiments, the effector protein comprises a portion of any one of the sequences recited in TABLE 1, wherein the portion does not comprise the last 10 amino acids, the last 20 amino acids, 40 amino acids, 60 amino acids, 80 amino acids, 100 amino acids, 120 amino acids, 140 amino acids, 160 amino acids, 180 amino acids, or 200 amino acids of any one of the sequences recited in TABLE 1.

In some embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to any one of the sequences as set forth in TABLE 1. In some embodiments, an effector protein provided herein comprises an amino acid sequence that is at least 65% identical to any one of the sequences as set forth in TABLE 1. In some embodiments, an effector protein provided herein comprises an amino acid sequence that is at least 70% identical to any one of the sequences as set forth in TABLE 1. In some embodiments, an effector protein provided herein comprises an amino acid sequence that is at least 75% identical to any one of the sequences as set forth in TABLE 1. In some embodiments, an effector protein provided herein comprises an amino acid sequence that is at least 80% identical to any one of the sequences as set forth in TABLE 1. In some embodiments, an effector protein provided herein comprises an amino acid sequence that is at least 85% identical to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% identical to any one of the sequences as set forth in TABLE 1. In some embodiments, an effector protein provided herein comprises an amino acid sequence that is at least 97% identical to any one of the sequences as set forth in TABLE 1. In some embodiments, an effector protein provided herein comprises an amino acid sequence that is at least 98% identical to any one of the sequences as set forth in TABLE 1. In some embodiments, an effector protein provided herein comprises an amino acid sequence that is at least 99% identical to any one of the sequences as set forth in TABLE 1. In some embodiments, an effector protein provided herein comprises an amino acid sequence that is identical to any one of the sequences as set forth in TABLE 1.

In some embodiments, compositions, systems, and methods described herein comprise an effector protein, or a nucleic acid encoding the effector protein, wherein the effector protein comprises one or more amino acid alterations relative to any one of the sequences recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, conservative deletions, non-conservative deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences recited in TABLE 1.

In some embodiments, the one or more amino acid alterations may result in a change in activity of the effector protein relative to a naturally-occurring counterpart. For example, and as described in further detail below, the one or more amino acid alteration increases or decreases catalytic activity of the effector protein relative to a naturally-occurring counterpart. In some embodiments, the one or more amino acid alterations results in an effector protein variant that has lower catalytic activity at low temperatures (e.g., room temperature) compared to higher temperatures (e.g., 50C), relative to a naturally-occurring counterpart, e.g., in order to facilitate a warm-start detection method. In some embodiments, the one or more amino acid alteration increases catalytic activity of the effector protein at elevated temperatures relative to a naturally-occurring counterpart. In some embodiments, the one or more amino acid alteration improves stability and/or manufacturability (e.g., expressibility, solubility, purification, etc.) of the effector protein relative to a naturally-occurring counterpart. In some embodiments, the one or more amino acid alteration improves efficiency of guide RNA complexing relative to a naturally-occurring counterpart.

TABLE 1
Amino Acid Sequences of Exemplary CRISPR/Cas effector proteins

| SEQ ID | |
|---|---|
| NO: | Programmable Nuclease Amino Acid Sequence |
| SEQ ID: 1 | MADLSQFTHKYQVPKTLRFELIPQGKTLENLSAYGMVADDKQRSENYKKLKPVIDRIYK |
| | YFIEESLKNTNLDWNPLYEAIREYRKEKTTATITNLKEQQDICRRAIASRFEGKVPDKGD |
| | KSVKDFNKKQSKLFKELFGKELFTDSVLEQLPGVSLSDEDKALLKSFDKFTTYFVGFYD |
| | NRKNVFSSDDISTGIPHRLVQENFPKFIDNCDDYKRLVLVAPELKEKLEKAAEATKIFED |
| | VSLDEIFSIKFYNRLLQQNQIDQFNQLLGGIAGAPGTPKIQGLNETLNLSMQQDKTLEQK |
| | LKSVPHRFSPLYKQILSDRSSLSFIPESFSCDAEVLLAVQEYLDNLKTEHVIEDLKEVENR |
| | LTTLDLKHIYVNSTKVTAFSQALFGDWNLCREQLRVYKMSNGNEKITKKALGELESWL |
| | KNSDIAFTELQEALADEALPAKVNLKVQEAISGLNEQMAKSLPKELKIPEEKEELKALLD |
| | AIQEVYHTLEWFIVSDDVETDTDFYVPLKETLQIIQPIIPLYNKVRNFATQKPYSVEKFKL |
| | NFANPTLADGWDENKEQQNCAVLFQKGNNYYLGILNPKNKPDFDNVDTEKQGNCYQK |
| | MVYKQFPDFSKMMPKCTTQLKEVKQHFEGKDSDYILNNKNFIKPLTITREVYDLNNVL |
| | YDGKKKFQIDYLRKTKDEDGYYTALHTWIDFAKKFVASYKSTSIYDTSTILPPEKYEKL |
| | NEFYGALDNLFYQIKFENIPEEIIDTYVEDGKLFLFQIYNKDFAAGATGAPNLHTIYWKA |
| | VFDPENVKDVVVKLNGQAELFYRPKSNMDVIRHKVGEKLVNRTLKDGSILTDELHKEL |
| | YLYANGSLKKGLSEDAKIILDKNLAVIYDVHHEIVKDRRFTTDKFFFHVPLTLNYKCDK |
| | NPVKFNAEVQEYLKENPDTYVIGIDRGERNLIYAVVIDPKGRIVEQKSFNVINGFDYHGK |
| | LDQREKERVKARQAWTAVGKIKELKQGYLSLVVHEISKMMVRYQAVVVLENLNVGFK |
| | RVRSGIAEKAVYQQFEKMLINKLNYLMFKDAGGTEPGSVLNAYQLTDRFESFAKMGLQ |
| | TGFLFYIPAAFTSKIDPATGFVDPFRWGAIKTLADKREFLSGFESLKFDSTTGNFILHFDVS |
| | KNKNFQKKLEGFVPDWDIIIEANKMKTGKGATYIAGKRIEFVRDNNSQGHYEDYLPCN |
| | ALAETLRQCDIPYEEGKDILPLILEKNDSKLLHSVFKVVRLTLQMRNSNAETGEDYISSPV |
| | EDVSGSCFDSRMENEKLPKDADANGAYHIALKGMLALERLRKDEKMAISNNDWLNYI |
| | QEKRA* |
| SEQ ID: 2 | MAGKKKDKDVINKTLSVRIIRPRYSDDIEKEISDEKAKRKQDGKTGELDRAFFSELKSRN |
| | PDIITNDELFPLFTEIQKNLTEIYNKSISLLYMKLIVEEEGGSTASALSAGPYKECKARENS |
| | YISLGLRQKIQSNFRRKELKGFQVSLPTAKSDRFPIPFCHQVENGKGGFKVYETGDDFIFE |
| | VPLIKYTATNKKSTSGKNYTKVQLNNPPVPMNVPLLLSTMRRRQTKKGMQWNKDEGT |
| | NAELRRVMSGEYKVSYAEIIRRTRFGKHDDWFVNFSIKFKNKTDELNQNVRGGIDIGVS |
| | NPLVCAVTNGLDRYIVANNDIMAFNERAMARRRTLLRKNRFKRSGHGAKNKLEPITVL |
| | TEKNERFRKSILQRWAREVAEFFKRTSASVVNMEDLSGITEREDFFSTKLRTTWNYRLM |
| | QTTIENKLKEYGIAVNYISPKYTSQTCHSCGKRNDYFTFSYRSENNYPPFECKECNKVKC |
| | NADFNAAKNIALKVVL |
| SEQ ID: 3 | MAKNTITKTLKLRIVRPYNSAEVEKIVADEKNNREKIALEKNKDKVKEACSKHLKVAA |
| | YCTTQVERNACLFCKARKLDDKFYQKLRGQFPDAVFWQEISEIFRQLQKQAAEIYNQSL |
| | IELYYEIFIKGKGIANASSVEHYLSDVCYTRAAELFKNAAIASGLRSKIKSNFRLKELKNM |
| | KSGLPTTKSDNFPIPLVKQKGGQYTGFEISNHNSDFIIKIPFGRWQVKKEIDKYRPWEKFD |
| | FEQVQKSPKPISLLLSTQRRKRNKGWSKDEGTEAEIKKVMNGDYQTSYIEVKRGSKIGE |
| | KSAWMLNLSIDVPKIDKGVDPSIIGGIDVGVKSPLVCAINNAFSRYSISDNDLFHFNKKM |
| | FARRRILLKKNRHKRAGHGAKNKLKPITILTEKSERFRKKLIERWACEIADFFIKNKVGT |
| | VQMENLESMKRKEDSYFNIRLRGFWPYAEMQNKIEFKLKQYGIEIRKVAPNNTSKTCSK |
| | CGHLNNYFNFEYRKKNKFPHFKCEKCNFKENADYNAALNISNPKLKSTKEEP |
| SEQ ID: 4 | MATLVSFTKQYQVQKTLRFELIPQGKTQANIDAKGFINDDLKRDENYMKVKGVIDELH |
| | KNFIEQTLVNVDYDWRSLATAIKNYRKDRSDTNKKNLEKTQEAARKEIIAWFEGKRGN |
| | SAFKNNQKSFYGKLFKKELFSEILRSDDLEYDEETQDAIACFDKFTTYFVGFHENRKNM |
| | YSTEAKSTSVAYRVVNENFSKFLSNCEAFSVLEAVCPNVLVEAEQELHLHKAFSDLKLS |
| | DVFKVEAYNKYLSQTGIDYYNQIIGGISSAEGVRKIRGVNEVVNNAIQQNDELKVALRN |
| | KQFTMVQLFKQILSDRSTLSFVSEQFTSDQEVITVVKQFNDDIVNNKVLAVVKTLFENFN |
| | SYDLEKIYINSKELASVSNALLKDWSKIRNAVLENKIIELGANPPKTKISAVEKEVKNKDF |
| | SIAELASYNDKYLDKEGNDKEICSIANVVLEAVGALEIMLAESLPADLKTLENKNKVKGI |
| | LDAYENLLHLLNYFKVSAVNDVDLAFYGAFEKVYVDISGVMPLYNKVRNYATKKPYS |
| | VEKFKLNFAMPTLADGWDKNKERDNGSIILLKDGQYYLGVMNPQNKPVIDNAVCNDA |
| | KGYQKMVYKMFPEISKMVTKCSTQLNAVKAHFEDNTNDFVLDDTDKFISDLTITKEIYD |
| | LNNVLYDGKKKFQIDYLRNTGDFAGYHKALETWIDFVKEFLSKYRSTAIYDLTTLLPTN |
| | YYEKLDVFYSDVNNLCYKIDYENISVEQVNEWVEEGNLYLFKIYNKDFATGSTGKPNL |
| | HTMYWNAVFAEENLHDVVVKLNGGAELFYRPKSNMPKVEHRVGEKLVNRKNVNGEPI |
| | ADSVHKEIYAYANGKISKSELSENAQEELPLAIIKDVKHNITKDKRYLSDKYFFHVPITLN |
| | YKANGNPSAFNTKVQAFLKNNPDVNIIGIDRGERNLLYVVVIDQQGNIIDKKQVSYNKV |
| | NGYDYYEKLNQREKERIEARQSWGAVGKIKELKEGYLSLVVREIADMMVKYNAIVVM |
| | ENLNAGFKRVRGGIAEKAVYQKFEKMLIDKLNYLVFKDVEAKEAGGVLNAYQLTDKF |
| | DSFEKMGNQSGFLFYVPAAYTSKIDPVTGFANVFSTKHITNTEAKKEFICSFNSLRYDEA |
| | KDKFVLECDLNKFKIVANSHIKNWKFIIGGKRIVYNSKNKTYMEKYPCEDLKATLNASG |
| | IDFSSSEIINLLKNVPANREYGKLFDETYWAIMNTLQMRNSNALTGEDYIISAVADDNEK |
| | VFDSRTCGAELPKDADANGAYHIALKGLYLLQRIDISEEGEKVDLSIKNEEWFKFVQQK |
| | EYAR* |
| SEQ ID: 5 | MCMKITKIDGISHKKYKEKGKLIKNNDTAKDIIEERFNDIEKKTKELFQKTLDFYVKNYE |
| | KCKEQNKERREKAKNYFSKVKILVDNKKITICNENTEKMEIEDFNEYDVRSGKYFNVLN |
| | KILNGENYTEEDLEVFENDLQKRTGRIKSIKNSLEENKAHFKKESINNNIIYDRVKGNNK |
| | KSLFYEYYRISSKHQEYVNNIFEAFDKLYSNSHEAMNNLFSEITKDSKDRNIRKIREAYH |
| | EILNKNKTEFGEELYKKIQDNRNNFDKLLEIEPEIKELTKSQIFYKYYIDKVNLDETSIKHC |
| | FCHLVEIEVNQLLKNYVYSKRNINKEKLENIFEYCKLKNLIKNKLVNKLNNYIRNCGKY |
| | NAYISNNDVVVNSEKISEIRTKEAFLRSIIGVSSSAYFSLRNILNTDNTQDITNKVDKEVD |
| | KLYQENKKIELEERLKLFFGNYFDINNQQEIKVFLMNIDKIISSIRHEIIHFKMETNAQNIF |
| | DENNVNLGNTAKNIFSNEINEEKIKFKIFKQLNSANVFDYLSNKDITEYMDKVVFSFTNR |
| | NVSFVPSFTKIYNRVQDLANSLEIKKWKIPDKSEGKDAQIYLLKNIYYGKFLDEFLNEEN |
| | GIFISIKDKIIELNRNQNKRTGFYKLEKFEKIEETNPKKYLEIIQSLYMINIEEIDSEGKNIFL |
| | DFIQKIFLKGFFEFIKNNYNYLLELKKIQDKKNIFDSEMSEYIAGEKTLEDIGEINEIIQDIKI |
| | TEIDKILNQTDKINCFYLLLKLLNYKEITELKGNLEKYQILSKTNVYEKELMLLNIVNLDN |
| | NKVKIENFKILAEEIGKFIEKINIEEINKNKKIKTFEELRNFEKGENTGEYYNIYSDDKNIK |
| | NIRNLYNIKKYGMLDLLEKISEKTNYCIKKKDLEEYSELRKQLEDEKTNFYKIQEYLHSK |
| | YQQKPKKILLKNNKNDYEKYKKSIENIEKYVHLKNKIEFNELNLLQSLLLKILHRLVGFT |
| | SIWERDLRFRLIGEFPDELDVEDIFDHRKRYKGTGKGICKKYDRFINTHTEYKNNNKME |
| | NVKFADNNPVRNYIAHFNYLPNPKYSILKMMEKLRKLLDYDRKLKNAVMKSIKDILEE |
| | YGFKAEFIINSDKEIILNLVKSVEIIHLGKEDLKSRRNSEDLCKLVKAMLEYSK* |
| SEQ ID: 6 | MEDKQFLERYKEFIGLNSLSKTLRNSLIPVGSTLKHIQEYGILEEDSLRAQKREELKGIMD |
| | DYYRNYIEMHLRDVHDIDWNELFEALTEVKKNQTDDAKKRLEKIQEKKRKEIYQYLSD |
| | DAVESEMFKEKMISGILPDFIRCNEGYSEEEKEEKLKTVALFHRFTSSFNDFFLNRKNVFT |
| | KEAIVTAIGYRVVHENAEIFLENMVAFQNIQKSAESQISIIERKNEHYFMEWKLSHIFTAD |
| | YYMMLMTQKAIEHYNEMCGVVNQQMREYCQKEKKNWNLYRMKRLHKQILSNASTSF |
| | KIPEKYENDAEVYESVNSFLQNVMEKTVMERIAVLKNSTDNFDLSKIYITAPYYEKISNY |
| | LCGSWNTITDCLTHYYEQQIAGKGARKDQKVKAAVKADKWKSLSEIEQLLKEYARAEE |
| | VKRKPEEYIAEIENIVSLKEAHLLEYHPEVNLIENEKYATEIKDVLDNYMELFHWMKWF |
| | YIEEAVEKEVNFYGELDDLYEEIKDIVPLYNKVRNYVTQKPYSDTKIKLNFGTPTLANG |
| | WSKSKEYDYNAILLQKDGKYYMGIFNPIQKPEKEIIEGHSQPLEGNEYKKMVYYYLPSA |
| | NKMLPKVLLSKKGMEIYQPSEYIINGYKERRHIKSEEKFDLQFCHDLIDYFKSGIERNSD |
| | WKVFGFDFSDTDTYQDISGFYREVEDQGYKIDWTYIKEADIDRLNEEGKLYLFQIYNKD |
| | FSEKSTGRENLHTMYLKNLFSEENVREQVLKLNGEAEIFFRKSSVKKPIIHKKGTMLVNR |
| | TYMEEVNGNSVRRNIPEKEYQEIYNYKNHRLKGELSTEAKKYLEKAVCHETKKDIVKD |
| | YRYSVDKFFIHLPITINYRASGKETLNSVAQRYIAHQNDMHVIGIDRGERNLIYVSVINM |
| | QGEIKEQKSFNIINEFNYKEKLKEREQSRGAARRNWKEIGQIKDLKEGYLSGVIHEIAKM |
| | MIKYHAIIAMEDLNYGFKRGRFKVERQVYQKFENMLIQKLNYLVFKDRPADEDGGVLR |
| | GYQLAYIPDSVKKMGRQCGMIFYVPAAFTSKIDPTTGFVDIFKHKVYTTEQAKREFILSF |
| | DEICYDVERQLFRFTFDYANFVTQNVTLARNNWTIYTNGTRAQKEFGNGRMRDKEDYN |
| | PKDKMVELLESEGIEFKSGKNLLPALKKVSNAKVFEELQKIVRFTVQLRNSKSEENDVD |
| | YDHVISPVLNEEGNFFDSSKYKNKEEKKESLLPVDADANGAYCIALKGLYIMQAIQKNW |
| | SEEKALSPDVLRLNNNDWFDYIQNKRYR* |
| SEQ ID: 7 | MEEKKMSKIEKFIGKYKISKTLRFRAVPVGKTQDNIEKKGILEKDKKRSEDYEKVKAYL |
| | DSLHRDFIENTLKKVKLNELNEYACLFFSGTKDDGDKKKMEKLEEKMRKTISNEFCNDE |
| | MYKKIFSEKILSENNEEDVSDIVSSYKGFFTSLNGYVNNRKNLYVSDAKPTSIAYRCINE |
| | NLPKFLRNVECYKKVVQVIPKEQIEYMSNNLNLSPYRIEDCFNIDFFEFCLSQGGIDLYNT |
| | FIGGYSKKDGTKVQGINEIVNLYNQKNKKDKEKYKLPQFTPLFKQILSDRDTKSFSIEKL |
| | ENIYEVVELVKKSYSDEMFDDIETVFSNLNYYDASGIYVKNGPAITHISMNLTKDWATIR |
| | NNWNYEYDEKHSTKKNKNIEKYEDTRNTMYKKIDSFTLEYISRLVGKDIDELVKYFENE |
| | VANFVMDIKKTYSKLTPLFDRCQKENFDISEDEVNDIKGYLDNVKLLESFMKSFTINGKE |
| | NNIDYVFYGKFTDDYDKLHEFDHIYNKVRNYITTSRKPYKLDKYKLYFDNPQLLGGWD |
| | INKEKDYRTVMLTKDGKYYFAIIDKGEHPFDNIPKDYFDNNGYYKKIIYRQIPNAAKYLS |
| | SKQIVPQNPPEEVKRILDKKKADSKSLTEEEKNIFIDYIKSDFLKNYKLLFDKNNNPYFNF |
| | AFRESSTYESLNEFFEDVERQAYSVRYENLPADYIDNLVNEGKIYLFEIYSKDFSEYSKGT |
| | NNLHTMYFKALFDNDNLKNTVFKLSGNAELFIRPASIKKDELVIHPKNQLLQNKNPLNP |
| | KKQSIFDYDLVKDKRFFENQYMLHISIEINKNERDAKKIKNINEMVRKELKDSDDNYIIGI |
| | DRGERNLLYVCVINSAGKIVEQMSLNEIINEYNGIKHTVDYQGLLDKCEKERNAQRQSW |
| | KSIENIKELKDGYISQVVHKLCQLVEKYDAIIAMENLNGGFKRGRTKFEKQVYQKFENK |
| | LINKMEYMADKKRKTTENGGILRGYQLTNGCINNSYQNGFIFYVPAWLTSKIDPTTGFV |
| | DLLKPKYTNVEEAHLWINKFNSITYDKKLDMFAFNINYSQFPRADIDYRKIWTFYTNGY |
| | RIETFRNSEKNNEFDWKEVHLTSVIKKLLEEYQINYISGKNIIDDLIQIKDKPFWNSFIKYI |
| | RLTLQMRNSITGRTDVDYIISPVINNEGTFYDSRKDLDEITLPQDADANGAYNIARKALW |
| | IIEKLKESPDEELNKVKLAITQREWLEYAQINI* |
| SEQ ID: 8 | MEKIKKPSNRNSIPSIIISDYDANKIKEIKVKYLKLARLDKITIQDMEIVDNIVEFKKILLNG |
| | VEHTIIDNQKIEFDNYEITGCIKPSNKRRDGRISQAKYVVTITDKYLRENEKEKRFKSTER |
| | ELPNNTLLSRYKQISGFDTLTSKDIYKIKRYIDFKNEMLFYFQFIEEFFNPLLPKGKNFYDL |
| | NIEQNKDKVAKFIVYRLNDDFKNKSLNSYITDTCMIINDFKKIQKILSDFRHALAHFDFDF |
| | IQKFFDDQLDKNKFDINTISLIETLLDQKEEKNYQEKNNYIDDNDILTIFDEKGSKFSKLH |
| | NFYTKISQKKPAFNKLINSFLSQDGVPNEEFKSYLVTKKLDFFEDIHSNKEYKKIYIQHKN |
| | LVIKKQKEESQEKPDGQKLKNYNDELQKLKDEMNTITKQNSLNRLEVKLRLAFGFIANE |
| | YNYNFKNFNDEFTNDVKNEQKIKAFKNSSNEKLKEYFESTFIEKRFFHFSVNFFNKKTKK |
| | EETKQKNIFNSIENETLEELVKESPLLQIITLLYLFIPRELQGEFVGFILKIYHHTKNITSDTK |
| | EDEISIEDAQNSFSLKFKILAKNLRGLQLFHYSLSHNTLYNNKQCFFYEKGNRWQSVYKS |
| | FQISHNQDEFDIHLVIPVIKYYINLNKLMGDFEIYALLKYADKNSITVKLSDITSRDDLKY |
| | NGHYNFATLLFKTFGIDTNYKQNKVSIQNIKKTRNNLAHQNIENMLKAFENSEIFAQREE |
| | IVNYLQTEHRMQEVLHYNPINDFTMKTVQYLKSLSVHSQKEGKIADIHKKESLVPNDYY |
| | LIYKLKAIELLKQKVIEVIGESEDEKKIKNAIAKEEQIKKGNN |
| SEQ ID: | MEKSLNDFIGLYSVSKTLRFELKPVSETLENIKKFHFLEEDKKKANDYKDVKKIIDNYHK |
| 35 | YFIDDVLKNASFNWKKLEEAIREYNKNKSDDSALVAEQKKLGDAILKLFTSDKRYKALT |
| | AATPKELFESILPDWFGEQCNQDLNKAALKTFQKFTSYFTGFQENRKNVYSAEAIPTAVP |
| | YRIVNDNFPKFLQNVLIFKTIQEKCPQIIDEVEKELSSYLGKEKLAGIFTLESFNKYLGQG |
| | GKENQRGIDFYNQIIGGVVEKEGGINLRGVNQFLNLYWQQHPDFTKEDRRIKMVPLYK |
| | QILSDRSSLSFKIESIENDEELKNALLECADKLELKNDEKKSIFEEVCDLFSSVKNLDLSGI |
| | YINRKDINSVSRILTGDWSWLQSRMNVYAEEKFTTKAEKARWQKSLDDEGENKSKGFY |
| | SLTDLNEVLEYSSENVAETDIRITDYFEHRCRYYVDKETEMFVQGSELVALSLQEMCDDI |
| | LKKRKAMNTVLENLSSENKLREKTDDVAVIKEYLDAVQELLHRIKPLKVNGVGDSTFY |
| | SVYDSIYSALSEVISVYNKTRNYITKKAASPEKYKLNFDNPTLADGWDLNKEQANTSVIL |
| | RKDGMFYLGIMNPKNKPKFAEKYDCGNESCYEKMIYKQFDATKQIPKCSTQKKEVQKY |
| | FLSGATEPYILNDKKSFKSELIITKDIWFMNNHVWDGEKFVPKRDNETRPKKFQIGYFKQ |
| | TGDFDGYKNALSNWISFCKNFLQSYLSATVYDYNFKNSEEYEGLDEFYNYLNATCYKL |
| | NFINIPETEINKMVSEGKLYLFQIYNKDFASGSTGMPNMHTLYWKNLFSDENLKNVCLK |
| | LNGEAELFYRPAGIKEPVIHKEGSYLVNRTTEDGESIPEKIYFEIYKNANGKLEKLSDEAQ |
| | NYISNHEVVIKKAGHEIIKDRHYTEPKFLFHVPLTINFKASGNSYSINENVRKFLKNNPDV |
| | NIIGLDRGERHLIYLSLINQKGEIIKQFTFNEVERNKNGRTIKVNYHEKLDQREKERDAAR |
| | KSWQAIGKIAELKEGYLSAVIHQLTKLMVEYNAVVVMEDLNFGFKRGRFHVEKQVYQ |
| | KFEHILIDKSNYLVFKDRGLNEPGGVLNGYQIAGQFESFQKLGKQSGMLFYVPAGYTSKI |
| | DPKTGFVSMMNFKDLTNVHKKRDFFSKFDNIHYDEANGSFVFTFDYKKFDGKAKEEM |
| | KLTKWSVYSRDKRIVYFAKTKSYEDVLPTEKLQKIFESNGIDYKSGNNIQDSVMAIGAD |
| | LKEGAKPSKEISDFWDGLLSNFKLILQMRNSNARTGEDYIISPVMADDGTFFDSREEFKK |
| | GEDAKLPLDADANGAYHIALKGLSLINKINLSKDEELKKFDMKISNADWFKFAQEKNY |
| | AK* |
| SEQ ID: 9 | MENYGGFTGLYPLQKTLKFELRPQGRTMEHLVSSNFFEEDRDRAEKYKIVKKVIDNYH |
| | KDFINECLSKRSFDWTPLMKTSEKYYASKEKNGKKKQDLDQKIIPTIENLSEKDRKELEL |
| | EQKRMRKEIVSVFKEDKRFKYLFSEKLFSILLKDEDYSKEKLTEKEILALKSFNKFSGYFI |
| | GLHKNRANFYSEGDESTAIAYRIVNENFPKFLSNLKKYREVCEKYPEIIQDAEQSLAGLNI |
| | KMDDIFPMENFNKVMTQDGIDLYNLAIGGKAQALGEKQKGLNEFLNEVNQSYKKGND |
| | RIRMTPLFKQILSERTSYSYILDAFDDNSQLITSINGFFTEVEKDKEGNTFDRAVGLIASY |
| | MKYDLSRVYIRKADLNKVSMEIFGSWERLGGLLRIFKSELYGDVNAEKTSKKVDKWLN |
| | SGEFSLSDVINAIAGSKSAETFDEYILKMRVARGEIDNALEKIKCINGNFSEDENSKMIIKA |
| | ILDSVQRLFHLFSSFQVRADFSQDGDFYAEYNEIYEKLFAIVPLYNRVRNYLTKNNLSMK |
| | KIKLNFKNPALANGWDLNKEYDNTAVIFLREGKYYLGIMNPSKKKNIKFEEGSGTGPFY |
| | KKMAYKLLPDPNKMLPKVFFAKKNINYYNPSDEIVKGYKAGKYKKGENFDIDFCHKLI |
| | DFFKESIQKNEDWRAFNYLFSATESYKDISDFYSEVEDQGYRMYFLNVPVANIDEYVEK |
| | GDLFLFQIYNKDFASGAKGNKDMHTIYWNAAFSDENLRNVVVKLNGEAELFYRDKSIIE |
| | PICHKKGEMLVNRTCFDKTPVPDKIHKELFDYHNGRAKTLSIEAKGYLDRVGVFQASYE |
| | IIKDRRYSENKMYFHVPLKLNFKADGKKNLNKMVIEKFLSDKDVHIIGIDRGERNLLYYS |
| | VIDRRGNIIDQDSLNIIDGFDYQKKLGQREIERREARQSWNSIGKIKDLKEGYLSKAVHK |
| | VSKMVLEYNAIVVLEDLNFGFKRGRFKVEKQVYQKFEKMLIDKLNYLVFKEVLDSRDA |
| | GGVLNAYQLTTQLESFNKLGKQSGILFYVPAAYTSKIDPTTGFVSLENTSRIESDSEKKDF |
| | LSGFDSIVYSAKDGGIFAFKFDYRNRNFQREKTDHKNIWTVYTNGDRIKYKGRMKGYEI |
| | TSPTKRIKDVLSSSGIRYDDGQELRDSIIQSGNKVLINEVYNSFIDTLQMRNSDGEQDYIIS |
| | PVKNRNGEFFRTDPDRRELPVDADANGAYHIALRGELLMQKIAEDFDPKSDKFTMPKM |
| | EHKDWFEFMQTRGD* |
| SEQ ID: | MEVQKTVMKTLSLRILRPLYSQEIEKEIKEEKERRKQAGGTGELDGGFYKKLEKKHSEM |
| 10 | FSFDRLNLLLNQLQREIAKVYNHAISELYIATIAQGNKSNKHYISSIVYNRAYGYFYNAYI |
| | ALGICSKVEANFRSNELLTQQSALPTAKSDNFPIVLHKQKGAEGEDGGFRISTEGSDLIFE |
| | IPIPFYEYNGENRKEPYKWVKKGGQKPVLKLILSTFRRQRNKGWAKDEGTDAEIRKVTE |
| | GKYQVSQIEINRGKKLGEHQKWFANFSIEQPIYERKPNRSIVGGLDVGIRSPLVCAINNSF |
| | SRYSVDSNDVFKFSKQVFAFRRRLLSKNSLKRKGHGAAHKLEPITEMTEKNDKFRKKIIE |
| | RWAKEVTNFFVKNQVGIVQIEDLSTMKDREDHFFNQYLRGFWPYYQMQTLIENKLKEY |
| | GIEVKRVQAKYTSQLCSNPNCRYWNNYFNFEYRKVNKFPKFKCEKCNLEISADYNAAR |
| | NLSTPDIEKFVAKATKGINLPEK* |
| SEQ ID: | MIIHNCYIGGSFMKKIDSFTNCYSLSKTLRFKLIPIGATQSNFDLNKMLDEDKKRAENYS |
| 11 | KAKSIIDKYHRFFIDKVLSSVTENKAFDSFLEDVRAYAELYYRSNKDDSDKASMKTLES |
| | KMRKFIALALQSDEGFKDLFGQNLIKKTLPEFLESDTDKEIIAEFDGFSTYFTGFFNNRKN |
| | MYSADDQPTAISYRCINDNLPKFLDNVRTFKNSDVASILNDNLKILNEDFDGIYGTSAED |
| | VFNVDYFPFVLSQKGIEAYNSILGGYTNSDGSKIKGLNEYINLYNQKNENIHRIPKMKQL |
| | FKQILSERESVSFIPEKFDSDDDVLSSINDYYLERDGGKVLSIEKTVEKIEKLFSAVTDYST |
| | DGIFVKNAAELTAVCSGAFGYWGTVQNAWNNEYDALNGYKETEKYIDKRKKAYKSIE |
| | SFSLADIQKYADVSESSETNAEVTEWLRNEIKEKCNLAVQGYESSKDLISKPYTESKKLF |
| | NNDNAVELIKNALDSVKELENVLRLLLGTGKEESKDENFYGEFLPCYERICEVDSLYDK |
| | VRNYMTQKLYKTDKIKLNFQNPQFLGGWDRNKEADYSAVLLRRNSLYYIAIMPSGYKR |
| | VFEKIPAPKADETVYEKVIYKLLPGPNKMLPKVFFSKKGIETFNPPKEILEKYELGTHKTG |
| | DGFNLDDCHALIDYFKSALDVHSDWSNFGFRFSDTSTYKNIADFYNEVKNQGYKITFCD |
| | VPQSYINELVDEGKLYLFQLYNKDFSEHSKGTPNLHTLYFKMLFDERNLENVVFKLNGE |
| | AEMFYREASISKDDMIVHPKNQPIKNKNEQNSRKQSTFEYDIVKDRRYTVDQFMLHIPIT |
| | LNFTANGGTNINNEVRKALKDCDKNYVIGIDRGERNLLYICVVDSEGRIIEQYSLNEIINE |
| | YNGNTYSTDYHALLDKKEKERLESRKAWKTVENIKELKEGYISQVVHKICELVEKYDA |
| | VIVMEDLNLGFKQGRSGKFEKSVYQKFEKMLIDKLNYFADKKKSPEEIGSVLNAYQLTN |
| | AFESFEKMGKQNGFIFYVPAYLTSKIDPTTGFADLLHPSSKQSKESMRDFVGRFDSITFN |
| | KTENYFEFELDYNKFPRCNTDYRKKWTVCTYGSRIKTFRNPEKNSEWDNKTVELTPAF |
| | MALFEKYSIDVNGDIKAQIMSVDKKDFFVELIGLLRLTLQMRNSETGKVDRDYLISPVK |
| | NSEGVFYNSDDYKGIENASLPKDADANGAYNIARKGLWIIEQIKACENDAELNKIRLAIS |
| | NAEWLEYAQKK* |
| SEQ ID: | MKDYIRKTLSLRILRPYYGEEIEKEIAAAKKKSQAEGGDGALDNKFWDRLKAEHPEIISS |
| 12 | REFYDLLDAIQRETTLYYNRAISKLYHSLIVEREQVSTAKALSAGPYHEFREKFNAYISLG |
| | LREKIQSNFRRKELARYQVALPTAKSDTFPIPIYKGFDKNGKGGFKVREIENGDFVIDLPL |
| | MAYHRVGGKAGREYIELDRPPAVLNVPVILSTSRRRANKTWFRDEGTDAEIRRVMAGE |
| | YKVSWVEILQRKRFGKPYGGWYVNFTIKYQPRDYGLDPKVKGGIDIGLSSPLVCAVTNS |
| | LARLTIRDNDLVAFNRKAMARRRTLLRQNRYKRSGHGSANKLKPIEALTEKNELYRKAI |
| | MRRWAREAADFFRQHRAATVNMEDLTGIKDREDYFSQMLRCYWNYSQLQTMLENKL |
| | KEYGIAVKYIEPKDTSKTCHSCGHVNEYFDFNYRSAHKFPMFKCEKCGVECGADYNAA |
| | RNIAQA |
| SEQ ID: | MKEQFINRYPLSKTLRFSLIPVGETENNFNKNLLLKKDKQRAENYEKVKCYIDRFHKEYI |
| 13 | ESVLSKARIEKVNEYANLYWKSNKDDSDIKAMESLENDMRKQISKQLTSTEIYKKRLFG |
| | KELICEDLPSFLTDKDERETVECFRSFTTYFKGFNTNRENMYSSDGKSTAIAYRCINDNLP |
| | RFLDNVKSFQKVFDNLSDETITKLNTDLYNIFGRNIEDIFSVDYFEFVLTQSGIEIYNSMIG |
| | GYTCSDKTKIQGLNECINLYNQQVAKNEKSKKLPLMKPLYKQILSEKDSVSFIPEKFNSD |
| | NEVLHAIDDYYTGHIGDFDLLTELLQSLNTYNANGIFVKSGVAITDISNGAFNSWNVLRS |
| | AWNEKYEALHPVTSKTKIDKYIEKQDKIYKAIKSFSLFELQSLGNENGNEITDWYISSINE |
| | SNSKIKEAYLQAQKLLNSDYEKSYNKRLYKNEKATELVKNLLDAIKEFQKLIKPLNGTG |
| | KEENKDELFYGKFTSYYDSIADIDRLYDKVRNYITQKPYSKDKIKLNFDNPQLLGGWDK |
| | NKESDYRTVLLHKDGLYYLAVMDKSHSKAFVDAPEITSDDKDYYEKMEYKLLPGPNK |
| | MLPKVFFASKNIDTFQPSDRILDIRKRESFKKGATFNKAECHEFIDYFKDSIKKHDDWSQ |
| | FGFKFSPTESYNDISEFYREISDQGYSVRFNKISKNYIDGLVNNGYIYLFQIYNKDFSKYS |
| | KGTPNLHTLYFKMLFDERNLSNVVYKLNGEAEMFYREASIGDKEKITHYANQPIKNKNP |
| | DNEKKESVFEYDIVKDKRFTKRQFSLHLPITINFKAHGQEFLNYDVRKAVKYKDDNYVI |
| | GIDRGERNLIYISVINSNGEIVEQMSLNEIISDNGHKVDYQKLLDTKEKERDKARKNWTS |
| | VENIKELKEGYISQVVHKICELVIKYDAVIAMEDLNFGFKRGRFPVEKQVYQKFENMLIS |
| | KLNLLIDKKAEPTEDGGLLRAYQLTNKFDGVNKAKQNGIIFYVPAWDTSKIDPATGFVN |
| | LLKPKCNTSVPEAKKLFETIDDIKYNANTDMFEFYIDYSKFPRCNSDFKKSWTVCTNSSR |
| | ILTFRNKEKNNKWDNKQIVLTDEFKSLFNEFGIDYKGNLKDSILSISNADFYRRLIKLLSL |
| | TLQMRNSITGSTLPEDDYLISPVANKSGEFYDSRNYKGTNAALPCDADANGAYNIARKA |
| | LWAINVLKDTPDDMLNKAKLSITNAEWLEYTQK* |
| SEQ ID: | MKEQFVNQYPISKTLRFSLIPIGKTEENFNKNLLLKEDEKKAEEYQKVKGYIDRYHKFFI |
| 14 | ETALCNINFEGFEEYSLLYYKCSKDDNDLKTMEDIEIKLRKQISKTMTSHKLYKDLFGEN |
| | MIKTILPNFLDSDEEKNSLEMFRGFYTYFSGFNTNRKNMYTEEAKSTSIAYRCINDNLPK |
| | FLDNSKSFEKIKCALNKEELKAKNEEFYEIFQIYATDIFNIDFFNFVLTQPGIDKYNGIIGG |
| | YTCSDGTKVQGLNEIINLYNQQIAKDDKSKRLPLLKMLYKQILSDRETVSFIPEKFSSDNE |
| | VLESINNYFSKNVSNAIKSLKELFQGFEAYNMNGIFISSGVAITDLSNAVFGDWNAISTA |
| | WEKAYFETNPPKKNKSQEKYEEELKANYKKIKSFSLDEIQRLGSIAKSPDSIGSVAEYYKI |
| | TVTEKIDNITELYDGSKELLNCNYSESYDKKLIKNDTVIEKVKTLLDAVKSLEKLIKPLV |
| | GTGKEDKDELFYGTFLPLYTSLSAVDRLYDKVRNYATQKPYSKDKIKLNFNCSSFLSGW |
| | ATDYSSNGGLIFEKDGLYYLGIVNKKFTTEEIDYLQQNADENPAQRIVYDFQKPDNKNT |
| | PRLFIRSKGTNYSPSVKEYNLPVEEIVELYDKRYFTTEYRNKNPELYKASLVKLIDYFKL |
| | GFTRHESYRHYDFKWKKSEEYNDISEFYKDVEISCYSLKQEKINYNTLLNFVAENRIYLF |
| | QIYNKDFSKYSKGTPNLHTRYFKALFDENNLSDVVFKLNGGSEMFFRKASIKDNEKVVH |
| | PANQPIDNKNPDNSKKQSTFDYELIKDKRFTKHQFSIHIPITMNFKARGRDFINNDIRKAI |
| | KSEYKPYVIGIDRGERNLIYISVINNNGEIVEQMSLNDIISDNGYKVDYQRLLDRKEKERD |
| | NARKSWGTIENIKELKEGYISQVIHKICELVIKYDAVIAMEDLNFGFKRGRFNVEKQVYQ |
| | KFENMLISKLNYLCDKKSEANSEGGLLKAYQLTNKFDGVNKGKQNGIIFYVPAWLTSKI |
| | DPVTGFVDLLHPKYISVEETHSLFEKLDDIRYNFEKDMFEFDIDYSKLPKCNADFKQKW |
| | TVCTNADRIMTFRNSEKNNEWDNKRILLSDEFKRLFEEFGIDYCHNLKNKILSISNKDFC |
| | YRFIKLFALTMQMRNSITGSTNPEDDYLISPVRDENGVFYDSRNFIGSKAGLPIDADANG |
| | AYNIARKGLWAINAIKSTADDMLDKVDLSISNAKWLEYVQK* |
| SEQ ID: | MKITKIDGILHKKYIKEGKLVKSTSEENKTDERLSELLTIRLDTYIKNPDNASEEENRIRRE |
| 15 | TLKEFFSNKVLYLKDSILYLKDRREKNQLQNKNYSEEDISEYDLKNKNSFLVLKKILLNE |
| | DINSEELEIFRNDFEKKLDKINSLKYSLEENKANYQKINENNIKKVEGKSKRNIFYNYYK |
| | DSAKRNDYINNIQEAFDKLYKKEDIENLFFLIENSKKHEKYKIRECYHKIIGRKNDKENF |
| | ATIIYEEIQNVNNMKELIEKVPNVSELKKSQVFYKYYLNKEKLNDENIKYVFCHFVEIEM |
| | SKLLKNYVYKKPSNISNDKVKRIFEYQSLKKLIENKLLNKLDTYVRNCGKYSFYLQDGEI |
| | ATSDFIVGNRQNEAFLRNIIGVSSTAYFSLRNILETENENDITGRIKGKTVKNKKGEEKYIS |
| | GEIDKLYDNNKQNEVKKNLKMFYSYDFNMNRKKEIEDFFSNIDEAISSIRHGIVHFNLEL |
| | EGKDIFTFKNIVPSQISKKMFQNEINEKKLKLKIFRQLNSANVFRYLEKYKILNYLNRTRF |
| | EFVNKNIPFVPSFTKLYSRIDDLKNSLCIYWKIPKANDNNKTKEITDAQIYLLKNIYYGEF |
| | LNYFMSNNGNFFEIIKEIIELNKNDKRNLKTGFYKLQKFENLQEKTPKEYLANIQSFYMID |
| | AGNKDEEEKDAYIDFIQKIFLKGFMTYLANNGRLSLMYIGNDEQINTSLAGKKQEFDKF |
| | LKKYEQNNNIEIPHEINEFVREIKLGKILKYTESLNMFYLILKLLNHKELTNLKGSLEKYQ |
| | SANKEEAFSDQLELINLLNLDNNRVTEDFELEADEIGKFLDFNGNKVKDNKELKKFDTN |
| | KIYFDGENIIKHRAFYNIKKYGILNLLEKISDEAKYKISIEELKNYSNKKIEIEKNHTTQEN |
| | LHRKYARPRKDEKFNDEDYKKYEKTIRNIQQYTHLKNKVEFNELNLLQSLLLRILHRLV |
| | GYTSIWERDLRFRLKGEFPENQYIEEIFNFDNSKNVKYKNGQIVEKYISFYKELYKDDME |
| | KISIYSDKKVKELKKEKKDLYIRNYIAHFNYIPNAEVSLLEVLENLRKLLSYDRKLKNAI |
| | MKSIVDILKEYGFVVTFKIEKDKKIRIESLKSEEVVHLKKLKLKDNDKKKEPIKTYRNSK |
| | ELCKLVKVMFEYKMKEKKSEN* |
| SEQ ID: | MKITKIDGISHKKYIKEGKLVKSTSEENKTDERLSELLTIRLDTYIKNPDNASEEENRIRRE |
| 16 | NLKEFFSNKVLYLKDGILYLKDRREKNQLQNKNYSEEDISEYDLKNKNSFLVLKKILLN |
| | EDINSEELEIFRKDVEAKLNKINSLKYSFEENKANYQKINENNVEKVGGKSKRNIIYDYY |
| | RESAKRNDYINNVQEAFDKLYKKEDIEKLFFLIENSKKHEKYKIRECYHKIIGRKNDKEN |
| | FAKIIYEEIQNVNNIKELIEKVPDMSELKKSQVFYKYYLDKEELNDKNIKYAFCHFVEIE |
| | MSQLLKNYVYKRLSNISNDKIKRIFEYQNLKKLIENKLLNKLDTYVRNCGKYNYYLQD |
| | GEIATSDFIAGNRQNEAFLRNIIGVSSVAYFSLRNILETENKDDITGKMRGKTRIDSKTGE |
| | EKYIPGEVDQIYYENKQNEVKNKLKMFYGYDFDMDNKKEIEDFFANIDEAISSIRHGIVH |
| | FNLDLDGKDIFAFKNIVPSEISKKMFQNEINEKKLKLKIFRQLNSANVFRYLEKYKILNYL |
| | KRTRFEFVNKNIPFVPSFTKLYSRIDDLKNSLGIYWKTPKTNDDNKTKEIIDAQIYLLKNI |
| | YYGEFLNYFMSNNGNFFEISREIIELNKNDKRNLKTGFYKLQKFEDIQEKTPKKYLANIQ |
| | SLYMINAGNQDEEEKDTYIDFIQKIFLKGFMTYLANNGRLSLMYIGNDEQINTSLAGKK |
| | QEFDKFLKKYEQNNNIEIPHEINEFLREIKLGKILKYTESLNMFYLILKLLNHKELTNLKG |
| | SLEKYQSANKEETFSDELELINLLNLDNNRVTEDFELEANEIGKFLDFNGNKIKDRKELK |
| | KFDTKKIYFDGENIIKHRAFYNIKKYGMLNLLEKIADKAKYKISLKELKEYSNKKNEIEK |
| | NYTMQQNLHRKYARPKKDEKFNDEDYKEYEKAIGNIQKYTHLKNKVEFNELNLLQGL |
| | LLKILHRLVGYTSIWERDLRFRLKGEFPENQYIEEIFNFDNSKNVKYKSGQIVEKYINFYK |
| | ELYKDNVEKRSIYSDKKVKKLKQEKKDLYIRNYIAHFNYIPHAEISLLEVLENLRKLLSY |
| | DRKLKNAIMKSVVDILKEYGFVATFKIGADKKIGIQTLESEKIVHLKNLKKKKLMTDRN |
| | SEELCKLVKVMFEYKMEEKNLKTKKCKVI* |
| SEQ ID: | MKKIDNFVGCYPVSKTLRFKAIPIGKTQENIEKKRLVEEDEVRAKDYKAVKKLIDRYHR |
| 17 | EFIEGVLDNVKLDGLEEYYMLFNKSDREESDNKKIEIMEERFRRVISKSFKNNEEYKKIFS |
| | KKIIEEILPNYIKDEEEKELVKGFKGFYTAFVGYAQNRENMYSDEKKSTAISYRIVNENM |
| | PRFITNIKVFEKAKSILDVDKINEINEYILNNDYYVDDFFNIDFFNYVLNQKGIDIYNAIIG |
| | GIVTGDGRKIQGLNECINLYNQENKKIRLPQFKPLYKQILSESESMSFYIDEIESDDMLID |
| | MLKESLQIDSTINNAIDDLKVLFNNIFDYDLSGIFINNGLPITTISNDVYGQWSTISDGWNE |
| | RYDVLSNAKDKESEKYFEKRRKEYKKVKSFSISDLQELGGKDLSICKKINEIISEMIDDYK |
| | SKIEEIQYLFDIKELEKPLVTDLNKIELIKNSLDGLKRIERYVIPFLGTGKEQNRDEVFYGY |
| | FIKCIDAIKEIDGVYNKTRNYLTKKPYSKDKFKLYFENPQLMGGWDRNKESDYRSTLLR |
| | KNGKYYVAIIDKSSSNCMMNIEEDENDNYEKINYKLLPGPNKMLPKVFFSKKNREYFAP |
| | SKEIERIYSTGTFKKDTNFVKKDCENLITFYKDSLDRHEDWSKSFDFSFKESSAYRDISEF |
| | YRDVEKQGYRVSFDLLSSNAVNTLVEEGKLYLFQLYNKDFSEKSHGIPNLHTMYFRSLF |
| | DDNNKGNIRLNGGAEMFMRRASLNKQDVTVHKANQPIKNKNLLNPKKTTTLPYDVYK |
| | DKRFTEDQYEVHIPITMNKVPNNPYKINHMVREQLVKDDNPYVIGIDRGERNLIYVVVV |
| | DGQGHIVEQLSLNEIINENNGISIRTDYHTLLDAKERERDESRKQWKQIENIKELKEGYIS |
| | QVVHKICELVEKYDAVIALEDLNSGFKNSRVKVEKQVYQKFEKMLITKLNYMVDKKK |
| | DYNKPGGVLNGYQLTTQFESFSKMGTQNGIMFYIPAWLTSKMDPTTGFVDLLKPKYKN |
| | KADAQKFFSQFDSIRYDNQEDAFVFKVNYTKFPRTDADYNKEWEIYTNGERIRVFRNPK |
| | KNNEYDYETVNVSERMKELFDSYDLLYDKGELKETICEMEESKFFEELIKLFRLTLQMR |
| | NSISGRTDVDYLISPVKNSNGYFYNSNDYKKEGAKYPKDADANGAYNIARKVLWAIEQ |
| | FKMADEDKLDKTKISIKNQEWLEYAQTHCE |
| SEQ ID: | MKKIDSFVNYYPLSKTLRFSLIPVGKTEDNFNAKLLLEEDEKRAIEYEKVKRYIDRYHKH |
| 18 | FIETVLANFHLDDVNEYAELYYKAGKDDKDLKYMEKLEGKMRKSISAAFTKDKKYKEI |
| | FGQEIIKNILPEFLENEDEKESVKMFQGFFTYFTGFNDNRKNMYTHEAQTTAISYRCINEN |
| | LPKFLDNVQSFAKIKESISSDIMNKLDEVCMDLYGVYAQDMFCTDYFSFVLSQSGIDRY |
| | NNIIGGYVDDKGVKIQGINEYINLYNQQVDEKNKRLPLMKKLYKQILIEKESISFIPEKFE |
| | SDNIVINAISDYYHNNVENLFDDFNKLFNEFSEYDDNGIFVTSGLAVTDISNAVFGSWNII |
| | SDSWNEEYKDSHPMKKTTNAEKYYEDMKKEYKKNLSFTIAELQRLGEAGCNDECKGDI |
| | KEYYKTTVAEKIENIKNAYEISKDLLASDYEKSNDKKLCKNDSAISLLKNLLDSIKDLEK |
| | TIKPLLGTGKEENKDDVFYGKFTNLYEMISEIDRLYDKVRNYVTQKPYSKDKIKLNFENP |
| | QHLGGWDKNKERDYRSVLLKKEDKYYLAIMDKSNNKAFIDFPDDGECYEKIEYKLLPG |
| | PNKMLPKVFFASSNIEYFAPSKKILEIRSRESFKKGDMFNLKDCHEFIDFFKESIKKHEDW |
| | SQFGFEFSPTEKYNDISEFYNEVKIQGYSLKYKNVSKKYIDELIECGQLYLFQIYNKDFSV |
| | YAKGNPNLHTMYFKMLFDERNLANVVYQLNGGAEMFYRKASIKDSEKIVHHANQPIK |
| | NKNADNVKKESVFEYDIIKDKRFTKRQFSIHIPITLNFKAKGQNFINNDVRMALKKADEN |
| | YVIGIDRGERNLLYICVINSKGEIVEQKSLNEIIGDNGYRVDYHKLLDKKEAERDEARKS |
| | WGTIENIKELKEGYLSQIVHEISKLVIKYDAVIAIEDLNSGFKKGRFKVEKQVYQKFENM |
| | LCTKLNYLVDKNADANECGGLLKAYQLTNKEDGANRGRQNGIIFSVPAWLTSKIDPVT |
| | GFADLLRPKYKSVSESVEFISKIDNIRYNSKEDYFEFDIDYSKFPNSTASYKKKWTVCTY |
| | GERIINVRNKEKNNMWDNKTIVLTDEFKKLFADFGVDVSKNIKESVLAIDSKDFYYRFIN |
| | LLANTLQLRNSEVGNVDVDYLISPVKGVDGSFYDSRLVKEKTLPENADANGAYNIARK |
| | ALWAIDVLKQTKDEELKNANLSIKNAEWLEYVQK* |
| SEQ ID: | MKNQNTLPSNPTDILKDKPFWAAFFNLARHNVYLTVNHINKLLDLEKLYNKDKHKEIFE |
| 19 | HEDIFNISDDVMNDVNSNGKKRKLDIKKIWANLDTDLTRKYQLRELILKHFPFIQPAIIGA |
| | QTKERTTIDKDKRSTSTSNDSLKPTGEGDINDPLSLSNVKSIFFRLLQMLEQLRNYYSHV |
| | KHSKSATMPNFDEGLLKSMYNIFIDSVNKVKEDYSSNSVIDPNTSFSHLISKDEQGEIKPC |
| | RYSFTSKDGSINASGLLFFVSLFLEKQDSIWMQKKIPGFKKTSENYMKMTNEVFCRNHIL |
| | LPKMRLETVYDKDWMLLDMLNEVVRCPLSLYKRLAPADQNKFKVPEKSSDNANRQED |
| | DNPFSRILVRHQNRFPYFALRFFDLNEVFTTLRFQINLGCYHFAICKKQIGDKKEVHHLT |
| | RTLYGFSRLQNFTQNTRPEEWNTLVKTTEPSSGNDGKTVQGVPLPYISYTIPHYQIENEKI |
| | GIKIFDGDTAVDTDIWPSVSTEKQLNKPDKYTLTPGFKADVFLSVHELLPMMFYYQLLL |
| | CEGMLKTDAGNAVEKVLIDTRNAIFNLYDAFVQEKINTITDLENYLQDKPILIGHLPKQM |
| | IDLLKGHQRDMLKAVEQKKAMLIKDTERRLERLNKQPEQKPNVAAKNTGTLLRNGQIA |
| | DWLVKDMMRFQPVKRDKEGNPINCSKANSTEYQMLQRAFAFYTTDSYRLPRYFEQLH |
| | LINCDNSHLFLSRFEYDKQPNLIAFYAAYLEAKLEFLNELQPQNWASDNYFLLLRAPKN |
| | DRQKLAEGWKNGFNLPRGLFTEKIKTWFNEHKTIVDISDCDIFKNRVGQVARLIPVFFDK |
| | KFKDHSQPFYTYNFNVGNVSKITEANYLSKEKRENLFKSYQNKFKNNIPAEKTKEYREY |
| | KNFSSWKKFERELRLIKNQDILTWLMCKNLFDEKIKPKKDILEPRIAVSYIKLDSLQTNTS |
| | TAGSLNALAKVVPMTLAIHIDSPKPKGKAGNNEKENKEFTVYIKEEGTKLLKWGNFKTL |
| | LADRRIKGLFSYIEHDDINLEKYPLTKYQVDSELDLYQKYRIDIFKQTLDLEAQLLDKYS |
| | DLNTDNFNQMLSGWSEKEGIPRNIKQDVAFLIGVRNGFSHNQYPDSKRIAFSRIKKENPK |
| | TSSLQESEGLNIAKQMYEEAQQVVNKIKNIESFD* |
| SEQ ID: | MKVTKIDGISHKKFEDEGKLVKFTGHFNIKNEMKERLEKLKELKLSNYIKNPENVKNKD |
| 20 | KNKEKETKSRRENLKKYFSEIILRKKEEKYLLKKTRKFKNITEEINYDDIKKRENQQKIFD |
| | VLKELLEQRINENDKEEILNFDSVKLKEAFEEDFIKKELKIKAIEESLEKNRADYRKDYVE |
| | LENEKYEDVKGQNKRSLVFEYYKNPENREKFKENIKYAFENLYTEENIKNLYSEIKEIFE |
| | KVHLKSKVRYFYQNEIIGESEFSEKDEEGISILYKQIINSVEKKEKFIEFLQKVKIKDLTRS |
| | QIFYKYFLENEELNDENIKYVFSYFVEIEVNKLLKENVYKTKKFNEGNKYRVKNIFNYD |
| | KLKNLVVYKLENKLNNYVRNCGKYNYHMENGDIATSDINMKNRQTEAFLRSILGVSSF |
| | GYFSLRNILGVNDDDFYKIEKDERKNENFILKKAKEDFTSKNIFEKVVDKSFEKKGIYQI |
| | KENLKMFYGNSFDKVDKDELKKFFVNMLEAITSVRHRIVHYNINTNSENIFDFSNIEVSK |
| | LLKNIFEKEIDTRELKLKIFRQLNSAGVFDYWESWVIKKYLENVKFEFVNKNVPFVPSFK |
| | KLYDRIDNLKGWNALKLGNNINIPKRKEAKDSQIYLLKNIYYGEFVEKFVNDNKNFEKI |
| | VKEIIEINRGAGTNKKTGFYKLEKFETLKANTPTKYLEKLQSLHKISYDKEKIEEDKDVY |
| | VDFVQKIFLKGFVNYLKKLDSLKSLNLLNLRKDETITDKKSVHDEKLKLWENSGSNLSK |
| | MPEEIYEYVKKIKISNINYNDRMSIFYLLLKLIDYRELTNLRGNLEKYESMNKNKIYSEEL |
| | TIINLVNLDNNKVRTNFSLEAEDIGKFLKSSITIKNIAQLNNFSKIFADGENVIKHRSFYNI |
| | KKYGILDLLEKIVAKADLKITKEEIKKYENLQNELKRNDFYKIQEQIHRNYNQKPFSIKKI |
| | ENKKDFEKYKKVIEKIQDYTQLKNKIEFNDLNLLQSLIFRILHRLAGYTSLWERDLQFKL |
| | KGEFPEDKYIDEIFNSDGNNNQKYKHGGIADKYANFLIEKKEEKSGEILNKKQRKKKIKE |
| | DLEIRNYIAHFNYLPNAEKSILEILEELRELLKHDRKLKNAVMKSIKDIFREYGFIVEFTIS |
| | HTKNGKKIKVCSVKSEKIKHLKNNELITTRNSEDLCELVKIMLEHKELQK* |
| SEQ ID: | MKVTKVGGISHKKYTSEGRLVKSESEENRTDERLSALLNMRLDMYIKNPSSTETKENQK |
| 21 | RIGKLKKFFSNKMVYLKDNTLSLKNGKKENIDREYSETDILESDVRDKKNFAVLKKIYL |
| | NENVNSEELEVFRNDIKKKLNKINSLKYSFEKNKANYQKINENNIEKVEGKSKRNIIYDY |
| | YRESAKRDAYVSNVKEAFDKLYKEEDIAKLVLEIENLTKLEKYKIREFYHEIIGRKNDKE |
| | NFAKIIYEEIQNVNNMKELIEKVPDMSELKKSQVFYKYYLDKEELNDKNIKYAFCHFVEI |
| | EMSQLLKNYVYKRLSNISNDKIKRIFEYQNLKKLIENKLLNKLDTYVRNCGKYNYYLQD |
| | GEIATSDFIARNRQNEAFLRNIIGVSSVAYFSLRNILETENENDITGRMRGKTVKNNKGEE |
| | KYVSGEVDKIYNENKKNEVKENLKMFYSYDFNMDNKNEIEDFFANIDEAISSIRHGIVHF |
| | NLELEGKDIFAFKNIAPSEISKKMFQNEINEKKLKLKIFRQLNSANVFRYLEKYKILNYLK |
| | RTRFEFVNKNIPFVPSFTKLYSRIDDLKNSLGIYWKTPKTNDDNKTKEIIDAQIYLLKNIY |
| | YGEFLNYFMSNNGNFFEISKEIIELNKNDKRNLKTGFYKLQKFEDIQEKIPKEYLANIQSL |
| | YMINAGNQDEEEKDTYIDFIQKIFLKGFMTYLANNGRLSLIYIGSDEETNTSLAEKKQEF |
| | DKFLKKYEQNNNIKIPYEINEFLREIKLGNILKYTERLNMFYLILKLLNHKELTNLKGSLE |
| | KYQSANKEEAFSDQLELINLLNLDNNRVTEDFELEADEIGKFLDFNGNKVKDNKELKKF |
| | DTNKIYFDGENIIKHRAFYNIKKYGMLNLLEKIADKAGYKISIEELKKYSNKKNEIEKNH |
| | KMQENLHRKYARPRKDEKFTDEDYESYKQAIENIEEYTHLKNKVEFNELNLLQGLLLRI |
| | LHRLVGYTSIWERDLRFRLKGEFPENQYIEEIFNFENKKNVKYKGGQIVEKYIKFYKELH |
| | QNDEVKINKYSSANIKVLKQEKKDLYIRNYIAHFNYIPHAEISLLEVLENLRKLLSYDRK |
| | LKNAVMKSVVDILKEYGFVATFKIGADKKIGIQTLESEKIVHLKNLKKKKLMTDRNSEE |
| | LCKLVKIMFEYKMEEKKSEN |
| SEQ ID: | MNELVKNRCKQTKTICQKLIPIGKTRETIEKYNLMEIDRKIAANKELMNKLFSLIAGKHI |
| 22 | NDTLSKCTDLDFEPLLTSLSSLNNAKENDRDNLREYYDSVFEEKKTLAEEISSRLTAVKF |
| | AGKDFFTKNIPDFLETYEGDDKNEMSELVSLVIENTVTAGYVKKLEKIDRSMEYRLVSG |
| | TVVKRVLTDNADIYEKNIEKAKDFDYGVLNIDEASQFTTLVAKDYANYLTADGIAIYNV |
| | GIGKINLALNEYCQKNKEYSYNKLALLPLQKMLYGEKLSLFEKLEDFTSDEELINSYNKF |
| | AKTVNESGLAEIIKKAVPSYDEIVIKPNKISNYSNSITGHWSLVNRIMKDYLENNGIKNAD |
| | KYMEKGLTLSEIGDALENKNIKHSDFISNLINDLGHTYTEIKENKESLKKDESVNALIIKK |
| | ELDMLLSILQNLKVFDIDNEMFDTGFGIEVSKAIEILGYGVPLYNKIRNYITKKPDPKKKF |
| | MTKFGSATIGTGITTSVEGSKKATFLKDGDAVFLLLYNTAGCKANNVSVSNLADLINSSL |
| | EIENSGKCYQKMIYQTPGDIKKQIPRVFVYKSEDDDLIKDFKAGLHKTDLSFLNGRLIPY |
| | LKEAFATHETYKNYTFSYRNSYESYDEFCEHMSEQAYILEWKWIDKKLIDDLVEDGSLL |
| | MFRVWNRFMKKKEGKISKHAKIVNELFSDENASNAAIKLLSVFDIFYRDKQIDNPIVHK |
| | AGTTLYNKRTKDGEVIVDYTTMVKNKEKRPNVYTTTKKYDIIKDRRYTEEQFEIHLHVN |
| | IGKEENKEKLETSKVINEKKNTLVVTRSNEHLLYVVIFDENDNILLKKSLNTVKGMNFKS |
| | KLEVVEIQKKENMQSWKTVGSNQALMEGYLSFAIKEIADLVKEYDAILVLEQNSVGKNI |
| | LNERVYTRFKEMLITNLSLDVDYENKDFYSYTELGGKVASWRDCVTNGICIQVPSAYKY |
| | KDPTTSFSTISMYAKTTAEKSKKLKQIKSFKYNRERGLFELVIAKGVGLENNIVCDSFGS |
| | RSIIENDISKEVSCTLKIEKYLIDAGIEYNDEKEVLKDLDTAAKTDAVHKAVTLLLKCFNE |
| | SPDGRYYISPCGEHFTLCDAPEVLSAINYYIRSRYIREQIVEGVKKMEYKKTILLAK* |
| SEQ ID: | MNGNRIIVYREFVGVTPVAKTLRNELRPIGHTQEHIIHNGLIQEDELRQEKSTELKNIMDD |
| 23 | YYREYIDKSLSGVTDLDFTLLFELMNLVQSSPSKDNKKALEKEQSKMREQICTHMQSDS |
| | NYKNIFNAKFLKEILPDFIKNYNQYDAKDKAGKLETLALFNGFSTYFTDFFEKRKNVFTK |
| | EAVSTSIAYRIVHENSLTFLANMTSYKKISEKALDEIEVIEKNNQDKMGDWELNQIFNPD |
| | FYNMVLIQSGIDFYNEICGVVNAHMNLYCQQTKNNYNLFKMRKLHKQILAYTSTSFEVP |
| | KMFEDDMSVYNAVNAFIDETEKGNIIGKLKDIVNKYDELDEKRIYISKDFYETLSCFMSG |
| | NWNLITGCVENFYDENIHAKGKSKEEKVKKAVKEDKYKSINDVNDLVEKYIDEKERNE |
| | FKNSNAKQYIREISNIITDTETAHLEYDEHISLIESEEKADEMKKRLDMYMNMYHWAKA |
| | FIVDEVLDRDEMFYSDIDDIYNILENIVPLYNRVRNYVTQKPYNSKKIKLNFQSPTLANG |
| | WSQSKEFDNNAIILIRDNKYYLAIFNAKNKPDKKIIQGNSDKKNDNDYKKMVYNLLPGA |
| | NKMLPKVFLSKKGIETFKPSDYIISGYNAHKHIKTSENFDISFCRDLIDYFKNSIEKHAEW |
| | RKYEFKFSATDSYNDISEFYREVEMQGYRIDWTYISEADINKLDEEGKIYLFQIYNKDFA |
| | ENSTGKENLHTMYFKNIFSEENLKDIIIKLNGQAELFYRRASVKNPVKHKKDSVLVNKT |
| | YKNQLDNGDVVRIPIPDDIYNEIYKMYNGYIKENDLSEAAKEYLDKVEVRTAQKDIVKD |
| | YRYTVDKYFIHTPITINYKVTARNNVNDMAVKYIAQNDDIHVIGIDRGERNLIYISVIDSH |
| | GNIVKQKSYNILNNYDYKKKLVEKEKTREYARKNWKSIGNIKELKEGYISGVVHEIAML |
| | MVEYNAIIAMEDLNYGFKRGRFKVERQVYQKFESMLINKLNYFASKGKSVDEPGGLLK |
| | GYQLTYVPDNIKNLGKQCGVIFYVPAAFTSKIDPSTGFISAFNFKSISTNASRKQFFMQFD |
| | EIRYCAEKDMFSFGFDYNNFDTYNITMSKTQWTVYTNGERLQSEFNNARRTGKTKSINL |
| | TETIKLLLEDNEINYADGHDVRIDMEKMDEDKNSEFFAQLLSLYKLTVQMRNSYTEAEE |
| | QEKGISYDKIISPVINDEGEFFDSDNYKESDDKECKMPKDADANGAYCIALKGLYEVLKI |
| | KSEWTEDGFDRNCLKLPHAEWLDFIQNKRYE* |
| SEQ ID: | MNKDIRKNFTDFVGISEIQKTLRFILIPIGKTAQNIDKYNMFEDDEIRHEYYPILKEACDDF |
| 24 | YRNHIDQQFENLELDWSKLDEALASEDRDLINETRATYRQVLFNRLKNSVDIKGDSKKN |
| | KTLSLESSDKNLGKKKTKNTFQYNFNDLFKAKLIKAILPLYIEYIYEGEKLENAKKALKM |
| | YNRFTSRLSNFWQARANIFTDDEISTGSPYRLVNDNFTIFRINNSIYTKNKPFIEEDILEFEK |
| | KLKSKKIIKDFESVDDYFTVNAFNKLCTQNGIDKYNSILGGFTTKEREKVKGLNELFNLA |
| | QQSINKGKKGEYRKNIRLGKLTKLKKQILAISDSTSFLIEQIEDDQDLYNKIKDFFELLLK |
| | EEIENENIFTQYANLQKLIEQADLSKIYINAKHLNKISHQVTGKWDSLNKGIALLLENINI |
| | NEESKEKSEVISNGQTKDISSEAYKRYLQIQSEEKDIERLRTQIYFSLEDLEKALDLVLIDE |
| | NMDRSDKSILSYVQSPDLNVNFERDLTDLYSRIMKLEENNEKLLANHSAIDLIKEFLDLI |
| | MLRYSRWQILFCDSNYELDQTFYPIYDAVMEILSNIIRLYNLARNYLSRKPDRMKKKKIN |
| | FNNPTLADGWSESKIPDNSSMLFIKDGMYYLGIIKNRAAYSELLEAESLQSSEKKKSENS |
| | SYERMNYHFLPDAFRSIPKSSIAMKAVKEHFEINQKTADLLLDTDKFSKPLRITKEIFDMQ |
| | YVDLHKNKKKYQVDYLRDTGDKKGYRKALNTWLNFCKDFISKYKGRNLFDYSKIKDA |
| | DHYETVNEFYNDVDKYSYHIFFTSVAETTVEKFISEGKLYLFQLYNKDFSPHSTGKPNLH |
| | TIYWRALFSEENLTSKNIKLNGQAEIFFRPKQIETPFTHKKGSILVNRFDVNGNPIPINVYQ |
| | EIKGFKNNVIKWDDLNKTTQEGLENDQYLYFESEFEIIKDRRYTEDQLFFHVPISENWDI |
| | GSNPKINDLATQYIVNSNDIHIIGIDRGENHLIYYSVIDLQGAIVEQGSLNTITEYTENKFL |
| | NNKTNNLRKIPYKDILQQREDERADARIKWHAIDKIKDLKDGYLGQIVHFLAKLIIKYNA |
| | IVILEDLNYGFKRGRFKVERQVYQKFEMALMKKLNVLVFKDYDIDEIGGPLKPWQLTRP |
| | IDSYERMGRQNGILFYVPAAYTSAVDPVTGFANLFYLNNVKNSEKFHFFSKFESIKYHSD |
| | QDMFSFAFDYNNFGTTTRINDLSKSKWQVFTNHERSVWNNKEKNYVTQNLTDLIKKLL |
| | RTYNIEFKNNQNVLDSILKIENNTDKENFARELFRLFRLTIQLRNTTVNENNTEITENELD |
| | YIISPVKDKNGNFFDSRDELKNLPDNGDANGAYNIARKGLLYIEQLQESIKTGKLPTLSIS |
| | TLDWFNYIMK* |
| SEQ ID: | MNKGGYVIMEKMTEKNRWENQFRITKTIKEEIIPTGYTKVNLQRVNMLKREMERNEDL |
| 25 | KKMKEICDEYYRNMIDVSLRLEQVRTLGWESLIHKYRMLNKDEKEIKALEKEQEDLRK |
| | KISKGFGEKKAWTGEQFIKKILPQYLMDHYTGEELEEKLRIVKKFKGCTMFLSTFFKNRE |
| | NIFTDKPIHTAVGHRITSENAMLFAANINTYEKMESNVTLEIERLQREFWRRGINISEIFTD |
| | AYYVNVLTQKQIEAYNKICGDINQHMNEYCQKQKLKFSEFRMRELKKQILAVVGEHFEI |
| | PEKIESTKEVYRELNEYYESLKELHGQFEELKSVQLKYSQIYVQKKGYDRISRYIGGQW |
| | DLIQECMKKDCASGMKGTKKNHDAKIEEEVAKVKYQSIEHIQKLVCTYEEDRGHKVTD |
| | YVDEFIVSVCDLLGADHIITRDGERIELPLQYEPGTDLLKNDTINQRRLSDIKTILDWHMD |
| | MLEWLKTFLVNDLVIKDEEFYMAIEELNERMQCVISVYNRIRNYVTQKGYEPEKIRICFD |
| | KGTILTGWTTGDNYQYSGFLLMRNDKYYLGIINTNEKSVRKILDGNEECKDENDYIRVG |
| | YHLINDASKQLPRIFVMPKAGKKSEILMKDEQCDYIWDGYCHNKHNESKEFMRELIDY |
| | YKRSIMNYDKWEGYCFKFSSTESYDNMQDFYKEVREQSYNISFSYINENVLEQLDKDG |
| | KIYLFQVYNKDFAAGSTGTPNLHTMYLQNLFSSQNLELKRLRLGGNAELFYRPGTEKDV |
| | THRKGSILVDRTYVREEKDGIEVRDTVPEKEYLEIYRYLNGKQKGDLSESAKQWLDKV |
| | HYREAPCDIIKDKRYAQEKYFLHFSVEINPNAEGQTALNDNVRRWLSEEEDIHVIGIDRG |
| | ERNLIYVSLMDGKGRIKDQKSYNIVNSGNKEPVDYLAKLKVREKERDEARRNWKAIGK |
| | IKDIKTGYLSYVVHEIVEMAVREKAIIVMEDLNYGFKRGRFKVERQVYQKFEEMLINKL |
| | NYVVDKQLSVDEPGGLLRGYQLAFIPKDKKSSMRQNGIVFYVPAGYTSKIDPTTGFVNIF |
| | KFPQFGKGDDDGNGKDYDKIRAFFGKFDEIRYECDEKVTADNTREVKERYRFDFDYSK |
| | FETHLVHMKKTKWTVYAEGERIKRKKVGNYWTSEVISDIALRMSNTLNIAGIEYKDGH |
| | NLVNEICALRGKQAGIILNELLEIVRLTVQLRNSTTEGDVDERDEIISPVLNEKYGCFYHS |
| | TEYKQQNGDVLPKDADANGAYCIGLKGIYEIRQIKNKWKEDMTKGEGKALNEGMRISH |
| | DQWFEFIQNMNKGE* |
| SEQ ID: | MNNPRGAFGGFTNLYSLSKTLRFELKPYLEIPEGEKGKLFGDDKEYYKNCKTYTEYYLK |
| 26 | KANKEYYDNEKVKNTDLQLVNFLHDERIEDAYQVLKPVFDTLHEEFITDSLESAEAKKI |
| | DFGNYYGLYEKQKSEQNKDEKKKIDKPLETERGKLRKAFTPIYEAEGKNLKNKAGKEK |
| | KDKDILKESGFKVLIEAGILKYIKNNIDEFADKKLKNNEGKEITKKDIETALGAENIEGIFD |
| | GFFTYFSGFNQNRENYYSTEEKATAVASRIVDENLSKFCDNILLYRKNENDYLKIFNFLK |
| | NKGKDLKLKNSKFGKENEPEFIPAYDMKNDEKSFSVADFVNCLSQGEIEKYNAKIANAN |
| | YLINLYNQNKDGNSSKLSMFKILYKQIGCGEKKDFIKTIKDNAELKQILEKACEAGKKYF |
| | IRGKSEDGGVSNIFDFTDYIQSHENYKGVYWSDKAINTISGKYFANWDTLKNKLGDAKV |
| | FNKNTGEDKADVKYKVPQAVMLSELFAVLDDNAGEDWREKGIFFKASLFEGDQNKSEI |
| | IKNANRPSQALLKMICDDMESLAKNFIDSGDKILKISDRDYQKDENKQKIKNWLDNAL |
| | WINQILKYFKVKANKIKGDSIDARIDSGLDMLVFSSDNPAEDYDMIRNYLTQKPQDEINK |
| | LKLNFENSSLAGGWDENKEKDNSCIILKDEQDKQYLAVMKYENTKVFEQKNSQLYIAD |
| | NAAWKKMIYKLVPGASKTLPKVFFSKKWTANRPTPSDIVEIYQKGSFKKENVDFNDKK |
| | EKDESRKEKNREKIIAELQKTCWMDIRYNIDGKIESAKYVNKEKLAKLIDFYKENLKKY |
| | PSEEESWDRLFAFGFSDTKSYKSIDQFYIEVDKQGYKLEFVTINKARLDEYVRDGKIYLF |
| | EIRSRDNNLVNGEEKTSAKNLQTIYWNAAFGGDDNKPKLNGEAEIFYRPAIAENKLNKK |
| | KDKNGKEIIDGYRFSKEKFIFHCPITLNFCLKETKINDKLNAALAKPENGQGVYFLGIDRG |
| | EKHLAYYSLVNQKGEILEQGTLNLPFLDKNGKSRSIKVEKKSFEKDSNGGIIKDKDGND |
| | KIKIEFVECWNYNDLLDARAGDRDYARKNWTTIGTIKELKDGYISQVVRKIVDLSIYKN |
| | TETKEFREMPAFIVLEDLNIGFKRGRQKIEKQVYQKLELALAKKLNFLVDKKADIGEIGS |
| | VTKAIQLTPPVNNFGDMENRKQFGNMLYIRADYTSQTDPATGWRKSIYLKSGSESNVKE |
| | QIEKSFFDIRYESGDYCFEYRDRHGKMWQLYSSKNGVSLDRFHGERNNSKNVWESEKQ |
| | PLNEMLDILFDEKRFDKSKSLYEQMFKGGVALTRLPKEINKKDKPAWESLRFVIILIQQIR |
| | NTGKNGDDRNGDFIQSPVRDEKTGEHFDSRIYLDKEQKGEKADLPTSGDANGAYNIAR |
| | KGIVVAEHIKRGFDKLYISDEEWDTWLAGDEIWDKWLKENRESLTKTRK* |
| SEQ ID: | MNYKTGLEDFIGKESLSKTLRNALIPTESTKIHMEEMGVIRDDELRAEKQQELKEIMDDY |
| 27 | YRTFIEEKLGQIQGIQWNSLFQKMEETMEDISVRKDLDKIQNEKRKEICCYFTSDKRFKD |
| | LFNAKLITDILPNFIKDNKEYTEEEKAEKEQTRVLFQRFATAFTNYFNQRRNNFSEDNIST |
| | AISFRIVNENSEIHLQNMRAFQRIEQQYPEEVCGMEEEYKDMLQEWQMKHIYSVDFYDR |
| | ELTQPGIEYYNGICGKINEHMNQFCQKNRINKNDFRMKKLHKQILCKKSSYYEIPFRFES |
| | DQEVYDALNEFIKTMKKKEIIRRCVHLGQECDDYDLGKIYISSNKYEQISNALYGSWDTI |
| | RKCIKEEYMDALPGKGEKKEEKAEAAAKKEEYRSIADIDKIISLYGSEMDRTISAKKCIT |
| | EICDMAGQISIDPLVCNSDIKLLQNKEKTTEIKTILDSFLHVYQWGQTFIVSDIIEKDSYFY |
| | SELEDVLEDFEGITTLYNHVRSYVTQKPYSTVKFKLHFGSPTLANGWSQSKEYDNNAIL |
| | LMRDQKFYLGIFNVRNKPDKQIIKGHEKEEKGDYKKMIYNLLPGPSKMLPKVFITSRSG |
| | QETYKPSKHILDGYNEKRHIKSSPKFDLGYCWDLIDYYKECIHKHPDWKNYDFHFSDTK |
| | DYEDISGFYREVEMQGYQIKWTYISADEIQKLDEKGQIFLFQIYNKDFSVHSTGKDNLHT |
| | MYLKNLFSEENLKDIVLKLNGEAELFFRKASIKTPIVHKKGSVLVNRSYTQTVGNKEIRV |
| | SIPEEYYTEIYNYLNHIGKGKLSSEAQRYLDEGKIKSFTATKDIVKNYRYCCDHYFLHLPI |
| | TINFKAKSDVAVNERTLAYIAKKEDIHIIGIDRGERNLLYISVVDVHGNIREQRSFNIVNG |
| | YDYQQKLKDREKSRDAARKNWEEIEKIKELKEGYLSMVIHYIAQLVVKYNAVVAMED |
| | LNYGFKTGRFKVERQVYQKFETMLIEKLHYLVFKDREVCEEGGVLRGYQLTYIPESLKK |
| | VGKQCGFIFYVPAGYTSKIDPTTGFVNLFSFKNLTNRESRQDFVGKFDEIRYDRDKKMFE |
| | FSFDYNNYIKKGTILASTKWKVYTNGTRLKKIVVNGKYTSQSMEVELTDAMEKMLQRA |
| | GIEYHDGKDLKGQIVEKGIEAEIIDIFRLTVQMRNSRSESEDREYDRLISPVLNDKGEFFD |
| | TATADKTLPQDADANGAYCIALKGLYEVKQIKENWKENEQFPRNKLVQDNKTWFDFM |
| | QKKRYL* |
| SEQ ID: | MRISKTLSLRIVRPFYTPEVEAGIKAEKDKREAQGQTRSLDAKFFNELKKKHSEIILSSEF |
| 28 | YSLLSEVQRQLTSIYNHAMSNLYHKIIVEGEKTSTSKALSNIGYDECKAIFPSYMALGLR |
| | QKIQSNFRRRDLKNFRMAVPTAKSDKFPIPIYRQVDGSKGGFKISENDGKDFIVELPLVD |
| | YVAEEVKTAKGRFTKINISKPPKIKNIPVILSTLRRRQSGQWFSDDGTNAEIRRVISGEYK |
| | VSWIEIVRRTRFGKHDDWFVNMVIKYDKPEEGLDSKVVGGIDVGVSSPLVCALNNSLD |
| | RYFVKSSDIIAFNKRAMARRRTLLRQNKYKRSGHGSKNKLEPITVLTEKNERFKKSIMQ |
| | RWAKEVAEFFRGKGASVVRMEELSGLKEKDNFFSSYLRMYWNYGQLQQIIENKLKEY |
| | GIKVNYVSPKDTSKKCHSCTHINEFFTFEYRQKNNFPLFKCEKCGVECSADYNAAKNMA |
| | IA |
| SEQ ID: | MRTMVTFEDFTKQYQVSKTLRFELIPQGKTLENMKRDGIISVDRQRNEDYQKAKGILDK |
| 29 | LYKYILDFTMETVVIDWEALATATEEFRKSKDKKTYEKVQSKIRTALLEHVKKQKVGTE |
| | DLFKGMFSSKIITGEVLAAFPEIRLSDEENLILEKFKDFTTYFTGFFENRKNVFTDEALSTS |
| | FTYRLVNDNFIKFFDNCIVFKNVVNISPHMAKSLETCASDLGIFPGVSLEEVFSISFYNRLL |
| | TQTGIDQFNQLLGGISGKEGEHKQQGLNEIINLAMQQNLEVKEVLKNKAHRFTPLFKQIL |
| | SDRSTMSFIPDAFADDDEVLSAVDAYRKYLSEKNIGDRAFQLISDMEAYSPELMRIGGK |
| | YVSVLSQLLFYSWSEIRDGVKAYKESLITGKKTKKELENIDKEIKYGVTLQEIKEALPKK |
| | DIYEEVKKYAMSVVKDYHAGLAEPLPEKIETDDERASIKHIMDSMLGLYRFLEYFSHDSI |
| | EDTDPVFGECLDTILDDMNETVPLYNKVRNFSTRKVYSTEKFKLNFNNSSLANGWDKN |
| | KEQANGAILLRKEGEYFLGIFNSKNKPKLVSDGGAGIGYEKMIYKQFPDFKKMLPKCTIS |
| | LKDTKAHFQKSDEDFTLQTDKFEKSIVITKQIYDLGTQTVNGKKKFQVDYPRLTGDMEG |
| | YRAALKEWIDFGKEFIQAYTSTAIYDTSLFRDSSDYPDLPSFYKDVDNICYKLTFEWIPD |
| | AVIDDCIDDGSLYLFKLHNKDFSSGSIGKPNLHTLYWKALFEEENLSDVVVKLNGQAEL |
| | FYRPKSLTRPVVHEEGEVIINKTTSTGLPVPDDVYVELSKFVRNGKKGNLTDKAKNWLD |
| | KVTVRKMPHAITKDRRFTVDKFFFHVPITLNYKADSSPYRENDFVRQYIKDCSDVKIIGI |
| | DRGERNLIYAVVIDGKGNIIEQRSFNTVGTYNYQEKLEQKEKERQTARQDWATVTKIKD |
| | LKKGYLSAVVHELSKMIVKYKAIVALENLNVGFKRMRGGIAERSVYQQFEKALIDKLN |
| | YLVFKDEEQSGYGGVLNAYQLTDKFESFSKMGQQTGFLFYVPAAYTSKIDPLTGFINPFS |
| | WKHVKNREDRRNFLNLFSKLYYDVNTHDFVLAYHHSNKDSKYTIKGNWEIADWDILIQ |
| | ENKEVFGKTGTPYCVGKRIVYMDDSTTGHNRMCAYYPHTELKKLLSEYGIEYTSGQDL |
| | LKIIQEFDDDKLVKGLFYIIKAALQMRNSNSETGEDYISSPIEGRPGICFDSRAEADTLPYD |
| | ADANGAFHIAMKGLLLTERIRNDDKLAISNEEWLNYIQEMRG* |
| SEQ ID: | MSKLEKFTNCYSLSKTLRFKAIPVGKTQENIDNKRLLVEDEKRAEDYKGVKKLLDRYYL |
| 30 | SFINDVLHSIKLKNLNNYISLFRKKTRTEKENKELENLEINLRKEIAKAFKGNEGYKSLFK |
| | KDIIETILPEFLDDKDEIALVNSFNGFTTAFTGFFDNRENMFSEEAKSTSIAFRCINENLTR |
| | YISNMDIFEKVDAIFDKHEVQEIKEKILNSDYDVEDFFEGEFFNFVLTQEGIDVYNAIIGGF |
| | VTESGEKIKGLNEYINLYNQKTKQKLPKFKPLYKQVLSDRESLSFYGEGYTSDEEVLEVF |
| | RNTLNKNSEIFSSIKKLEKLFKNFDEYSSAGIFVKNGPAISTISKDIFGEWNVIRDKWNAE |
| | YDDIHLKKKAVVTEKYEDDRRKSFKKIGSFSLEQLQEYADADLSVVEKLKEIIIQKVDEI |
| | YKVYGSSEKLFDADFVLEKSLKKNDAVVAIMKDLLDSVKSFENYIKAFFGEGKETNRDE |
| | SFYGDFVLAYDILLKVDHIYDAIRNYVTQKPYSKDKFKLYFQNPQFMGGWDKDKETDY |
| | RATILRYGSKYYLAIMDKKYAKCLQKIDKDDVNGNYEKINYKLLPGPNKMLPKVFFSK |
| | KWMAYYNPSEDIQKIYKNGTFKKGDMFNLNDCHKLIDFFKDSISRYPKWSNAYDFNFS |
| | ETEKYKDIAGFYREVEEQGYKVSFESASKKEVDKLVEEGKLYMFQIYNKDFSDKSHGTP |
| | NLHTMYFKLLFDENNHGQIRLSGGAELFMRRASLKKEELVVHPANSPIANKNPDNPKKT |
| | TTLSYDVYKDKRFSEDQYELHIPIAINKCPKNIFKINTEVRVLLKHDDNPYVIGIDRGERN |
| | LLYIVVVDGKGNIVEQYSLNEIINNFNGIRIKTDYHSLLDKKEKERFEARQNWTSIENIKE |
| | LKAGYISQVVHKICELVEKYDAVIALEDLNSGFKNSRVKVEKQVYQKFEKMLIDKLNY |
| | MVDKKSNPCATGGALKGYQITNKFESFKSMSTQNGFIFYIPAWLTSKIDPSTGFVNLLKT |
| | KYTSIADSKKFISSFDRIMYVPEEDLFEFALDYKNFSRTDADYIKKWKLYSYGNRIRIFRN |
| | PKKNNVFDWEEVCLTSAYKELFNKYGINYQQGDIRALLCEQSDKAFYSSFMALMSLML |
| | QMRNSITGRTDVDFLISPVKNSDGIFYDSRNYEAQENAILPKNADANGAYNIARKVLWA |
| | IGQFKKAEDEKLDKVKIAISNKEWLEYAQTSVKH |
| SEQ ID: | MTNFDNFTKKYVNSKTIRLEAIPVGKTLKNIEKMGFIAADRQRDEDYQKAKSVIDHIYK |
| 31 | AFMDDCLKDLFLDWDPLYEAVVACWRERSPEGRQALQIMQADYRKKIADRFRNHELY |
| | GSLFTKKIFDGSVAQRLPDLEQSAEEKSLLSNFNKFTSYFRDFFDKRKRLFSDDEKHSAIA |
| | YRLINENFLKFVANCEAFRRMTERVPELREKLQNTGSLQVYNGLALDEVFSADFYNQLI |
| | VQKQIDLYNQLIGGIAGEPGTPNIQGLNATINLALQGDSSLHEKLAGIPHRFNPLYKQILS |
| | DVSTLSFVPSAFQSDGEMLAAVRGFKVQLESGRVLQNVRRLFNGLETEADLSRVYVNN |
| | SKLAAFSSMFFGRWNLCSDALFAWKKGKQKKITNKKLTEIKKWLKNSDIAIAEIQEAFG |
| | EDFPRGKINEKIQAQADALHSQLALPIPENLKALCAKDGLKSMLDTVLGLYRMLQWFIV |
| | GDDNEKDSDFYFGLGKILGSLDPVLVLYNRVRNYITKKPYSLTKFRLNFDNSQLLNGWD |
| | ENNLDTNCASIFIKDGKYYLGISNKNNRPQFDTVATSGKSGYQRMVYKQFANWGRDLP |
| | HSTTQMKKVKKHFSASDADYVLDGDKFIRPLIITKEIFDLNNVKFNGKKKLQVDYLRNT |
| | GDREGYTHALHTWINFAKDFCACYKSTSIYDISCLRPTDQYDNLMDFYADLGNLSHRIV |
| | WQTIPEEAIDNYVEQGQLFLFQLYNKDFAPGADGKPNLHTLYWKAVFNPENLEDVVVK |
| | LNGKAELFYRPRSNMDVVRHKVGEKLVNRKLKNGLTLPSRLHEEIYRYVNGTLNKDLS |
| | ADARSVLPLAVVRDVQHEIIKDRRFTADKFFFHASLTFNFKSSDKPVGFNEDVREYLREH |
| | PDTYVVGVDRGERNLIYIVVIDPQGNIVEQRSFNMINGIDYWSLLDQKEKERVEAKQAW |
| | ETVGKIKDLKCGYLSFLIHEITKIIIKYHAVVILENLSLGFKRVRTGIAEKAVYQQFERML |
| | VTKLGYVVFKDRAGKAPGGVLNAYQLTDNTRTAENTGIQNGFLFYVPAAFTSRVDPAT |
| | GFFDFYDWGKIKTATDKKNFIAGFNSVRYERSTGDFIVHVGAKNLAVRRVAEDVRTEW |
| | DIVIEANVRKMGIDGNSYISGKRIRYRSGEQGHGQYENHLPCQELIRALQQYGIQYETGK |
| | DILPAILQQDDAKLTDTVFDVFRLALQMRNTSAETGEDYFNSVVRDRSGRCFDTRRAEA |
| | AMPKEADANDAYHIALKGLFVLEKLRKGESIGIKNTEWLRYVQQRHS* |
| SEQ ID: | MTPIFCNFVVYQIMLFNNNININVKTMNKKHLSDFTNLFPVSKTLRFRLEPQGKTMENIV |
| 32 | KAQTIETDEERSHDYEKTKEYIDDYHRQFIDDTLDKFAFKVESTGNNDSLQDYLDAYLS |
| | ANDNRTKQTEEIQTNLRKAIVSAFKMQPQFNLLFKKEMVKHLLPQFVDTDDKKRIVAKF |
| | NDFTTYFTGFFTNRENMYSDEAKSTSIAYRIVNQNLIKFVENMLTFKSHILPILPQEQLAT |
| | LYDDFKEYLNVASIAEMFELDHFSIVLTQRQIEVYNSVIGGRKDENNKQIKPGLNQYINQ |
| | HNQAVKDKSARLPLLKPLFNQILSEKAGVSFLPKQFKSASEVVKSLNEAYAELSPVLAAI |
| | QDVVTNITDYDCNGIFIKNDLGLTDIAQRFYGNYDAVKRGLRNQYELETPMHNGQKAE |
| | KYEEQVAKHLKSIESVSLAQINQVVTDGGDICDYFKAFGATDDGDIQRENLLASINNAH |
| | TAISPVLNKENANDNELRKNTMLIKDLLDAIKRLQWFAKPLLGAGDETNKDQVFYGKF |
| | EPLYNQLDETISPLYDKVRSYLTKKPYSLDKFKINFEKSNLLGGWDPGADRKYQYNAVI |
| | LRKDNDFYLGIMRDEATSKRKCIQVLDCNDEGLDENFEKVEYKQIKPSQNMPRCAFAK |
| | KECEENADIMELKRKKNAKSYNTNKDDKNALIRHYQRYLDRTYPEFGFVYKDADEYD |
| | TVKAFTDSMDSQDYKLSFLQVSETGLNKLVDEGDLYLFKITNKDFSSYAKGRPNLHTIY |
| | WRMLFDPKNLANVVYKLEGKAEVFFRRKSLASTTTHKAKQAIKNKSRYNEAVKPQSTF |
| | DYDIIKDRRFTADKFEFHVPIKMNFKAAGWNSTRLTNEVREFIKSQGVRHIIGIDRGERH |
| | LLYLTMIDMDGNIVKQCSLNAPAQDNARASEVDYHQLLDSKEADRLAARRNWGTIENI |
| | KELKQGYLSQVVHLLATMMVDNDAILVLENLNAGFMRGRQKVEKSVYQKFEKMLIDK |
| | LNYIVDKGQSPDKPTGALHAVQLTGLYSDFNKSNMKRANVRQCGFVFYIPAWNTSKID |
| | PVTGFVNLFDTHLSSMGEIKAFFSKFDSIRYNQDKGWFEFKFDYSRFTTRAEGCRTQWT |
| | VCTYGERIWTHRSKNQNNQFVNDTVNVTQQMLQLLQDCGIDPNGNLKEAIANIDSKKS |
| | LETLLHLFKLTVQMRNSVTGSEVDYMISPVADERGHFFDSRESDEHLPANADANGAFNI |
| | ARKGLMVVRQIMATDDVSKIKFAVSNKDWLRFAQHIDD* |
| SEQ ID: | VKISKTLSLRIIRPYYTPEVESAIKAEKDKREAQGQTRNLDAKFFNELKKKHPQIILSGEF |
| 33 | YSLLFEMQRQLTSIYNRAMSSLYHKIIVEGEKTSTSKALSDIGYDECKSVFPSYIALGLRQ |
| | KIQSNFRRKELKGFRMAVPTAKSDKFPIPIYKQVDDGKGGFKISENKEGDFIVELPLVEY |
| | TAEDVKTAKGKFTKINISKPPKIKNIPVILSTLRRKQSGQWFSDEGTNAEIRRVISGEYKV |
| | SWIEVVRRTRFGKHDDWFLNIVIKYDKTEDGLDPEVVGGIDVGVSTPLVCAVNNSLDRY |
| | FVKSSDIIAFKKRAMARRRTLLRQNRFKRSGHGSKSKLEPITILTEKNERFKKSIMQRWA |
| | KEVAEFFKGERASVVQMEELSGLKEKDNFFGSYLRMYWNYGQLQQIIENKLKEYGIKV |
| | NYVSPKDTSKKCHSCGYINEFFTFEFRQKNNFPLFKCKKCGVECNADYNAAKNIAIA |
| SEQ ID: | MLKSYDYFTKLYSLQKTLRFELKPIGKTLEHIKNSGIIESDETLEEQYAIVKNIIDKLHRKH |
| 34 | IDEALSLVDFTKHLDTLKTFQELYLKRGKTDKEKEELEKLSADLRKLIVSYLKGNVKEK |
| | TQHNLNPIKERFEILFGKELFTNEEFFLLAENEKEKKAIQAFKGFTTYFKGFQENRKNMY |
| | SEEGNSTSIAYRIINENLPLFIENIARFQKVMSTIEKTTIKKLEQNLKTELKKHNLPGIFTIE |
| | YFNNVLTQEGISRYNTIIGGKTTHEGVKIQGLNEIINLYNQQSKDVKLPILKPLHKQILSEE |
| | YSTSFKIKAFENDNEVLKAIDTFWNEHIEKSIHPVTGNKFNILSKIENLCDQLQKYKDKDL |
| | EKLFIERKNLSTVSHQVYGQWNIIRDALRMHLEMNNKNIKEKDIDKYLDNDAFSWKEIK |
| | DSIKIYKEHVEDAKELNENGIIKYFSAMSINEEDDEKEYSISLIKNINEKYNNVKSILQEDR |
| | TGKSDLHQDKEKVGIIKEFLDSLKQLQWFLRLLYVTVPLDEKDYEFYNELEVYYEALLP |
| | LNSLYNKVRNYMTRKPYSVEKFKLNFNSPTLLDGWDKNKETANLSIILRKNGKYYLGI |
| | MNKENNTIFEYYPGTKSNDYYEKMIYKLLPGPNKMLPKVFFSKKGLEYYNPPKEILNIY |
| | EKGEFKKDKSGNFKKESLHTLIDFYKEAIAKNEDWEVFNFKFKNTKEYEDISQFYRDVE |
| | EQGYLITFEKVDANYVDKLVKEGKLYLFQIYNKDFSENKKSKGNPNLHTIYWKGLYDS |
| | ENLKNVVYKLNGEAEVFYRKKSIDYPEEIYNHGHHKEELLGKFNYPIIKDRRYTQDKFL |
| | FHVPITMNFISKEEKRVNQLACEYLSATKEDVHIIGIDRGERHLLYLSLIDKEGNIKKQLS |
| | LNTIKNENYDKEIDYRVKLDEKEKKRDEARKNWDVIENIKELKEGYMSQVIHIIAKMMV |
| | EEKAILIMEDLNIGFKRGRFKVEKQVYQKFEKMLIDKLNYLVFKNKNPLEPGGSLNAYQ |
| | LTSKFDSFKKLGKQSGFIFYVPSAYTSKIDPTTGFYNFIQVDVPNLEKGKEFFSKFEKIIYN |
| | TKEDYFEFHCKYGKFVSEPKNKDNDRKTKESLTYYNAIKDTVWVVCSTNHERYKIVRN |
| | KAGYYESHPVDVTKNLKDIFSQANINYNEGKDIKPIIIESNNAKLLKSIAEQLKLILAMRY |
| | NNGKHGDDEKDYILSPVKNKQGKFFCTLDGNQTLPINADANGAYNIALKGLLLIEKIKK |
| | QQGKIKDLYISNLEWFMFMMSR |
| SEQ ID: | MIKNPSNRHSLPKVIISEVDHEKILEFKIKYEKLARLDRFEVKAMHYEGKEIVFDEVLVN |
| 36 | GGLIEVEYQDDNKTLFVKVGEKSYSIRGKKVGGKQRLLEDRVSKTKVQLELSDGVVDN |
| | KGNLRKSRTERELIVADNIKLYSQIVGREVTTTKEIYLVKRFLAYRSDLLFYYSFVDNFF |
| | KVAGNEKELWKINFDDATSAQFMGYIPFMVNDNLKNDNAYLKDYVRNDVQIKDDLKK |
| | VQTIFSALRHTLLHFNYEFFEKLENGEDVGFDFDIGFLNLLIENIDKLNIDAKKEFIDNEKI |
| | RLFGENLSLAKVYRLYSDICVNRVGFNKFINSMLIKDGVENQVLKAEFNRKFGGNAYTI |
| | DIHSNQEYKRIYNEHKKLVIKVSTLKDGQAIRRGNKKISELKEQMKSMTKKNSLARLEC |
| | KMRLAFGFLYGEYNNYKAFKNNFDTNIKNSQFDVNDVEKSKAYFLSTYERRKPRTREK |
| | LEKVAKDIESLELKTVIANDTLLKFILLMFVFMPQELKGDFLGFVKKYYHDVHSIDDDT |
| | KEQEEDVVEAMSTSLKLKILGRNIRSLTLFKYALSSQVNYNSTDNIFYVEGNRYGKIYKK |
| | LGISHNQEEFDKTLVVPLLRYYSSLFKLMNDFEIYSLAKANPTAVSLQELVDDETSPYKQ |
| | GNYFNFNKMLRDIYGLTSDEIKSGQVVFMRNKIAHFDTEVLLSKPLLGQTKMNLQRKDI |
| | VSFIEARGDIKELLGYDAINDFRMKVIHLRTKMRVYSDKLQTMMDLLRNAKTPNDFYN |
| | VYKVKGVESINKHLLEVLAQTAEERTVEKQIRDGNEKYDL |
| SEQ ID: | LNSIEKIKKPSNRNSIPSIIISDYDENKIKEIKVKYLKLARLDKITIQDMEIRDNIVEFKKILL |
| 37 | NGIEHTIKDNQKIEFDNYEITAYVRASKQRRDGKITQAKYVVTITDKYLRDNEKEKRFKS |
| | TERELPNDTLLMRYKQISGFDTLTSKDIYKIKRYIDFKNEMLFYFQFIEEFFSPLLPKGTNF |
| | YSLNIEQNKDKVVKYIVYRLNDDFKNQSLNQFIKKTDTIKYDFLKIQKILSDFRHALAHF |
| | DFDFIQKFFDDELDKNRFDISTISLIKTMLQEKEEKYYQEKNNYIEDSDTLTLFDEKESNF |
| | SKIHNFYIKISQKKPAFNKLINSFLSKDGVPNEELKSYLATKKIDFFEDIHSNKEYKKIYIK |
| | HKNLVVEKQKEESQEKPNGQKLKNYNDELQKLKDEMNKITKQNSLNRLEVKLRLAFGF |
| | IANEYNYNFKNFNDKFTLDVKKEQKIKVFKNSSNEKLKEYFESTFIEKRFFHFCVKFFNK |
| | KTKKEETKQKNIFNLIENETLEELVKESPLLQIITLLYLFIPKELQGEFVGFILKIYHHTKNI |
| | TNDTKEDEKSIEDTQNSFSLKLKILAKNLRGLQLFNYSLSHNTLYNTKEHFFYEKGNRW |
| | QSVYKSLEISHNQDEFDIHLVIPVIKYYINLNKLIGDFEIYALLTYADKNSITEKLSDITKR |
| | DDLKFRGYYNFSTLLFKTFMINTNYEQNQKSTQYIKQTRNDIAHQNIENMLKAFENNEIF |
| | AQREEIVNYLQKEHKMQEILHYNPINDFTMKTVQYLKSLNIHSQKESKIADIHKKESLVP |
| | NDYYLIYKLKVIELLKQKVIEAIGETKDEEKIKNAIAKEEQIKKGYNK |
| SEQ ID: | MLKHKRKNKNSLARVVLSNYDSNNIYEIKIKYEKLAKLDKINIIEMDYDADNNVMFKK |
| 38 | VLFNNKEIDLSHKDKTKINIELDNKKYNISAKKQIGKTHLVVRNKQTSKISRIKKIQDTYY |
| | RGKDVFILDNNIEILDKKQTKDKFIVTLNDITNNKTTSTEAELIDDTKDIFKKISAKKDLKS |
| | SDIYKIKRFISIRSNFSFYYTFVDNYFKIFHAKKDKNKEELYKIKFKDEINIKPYLENILDN |
| | MKNKNGILYNYANDRKKVLNDLRNIQYVFKEFRHKLAHFDYNFLDNFFSNSVEEKYKQ |
| | KVNEIKLLDILLDNIDSLNVVPKQNYIEDETISVFDAKDIKLKRLYTYYIKLTINYPGFKKL |
| | INSFFIQDGIENQELKEYINNKEKDTQVLKELDNKAYYMDISQYRKYKNIYNKHKELVSE |
| | KELSSDGKKINSLNQKINKLKIDMKNITKPNALNRLIYRLRVAFGFIYKEYATINNENKSF |
| | LQDTKTKRFENISQQDIKSYLDISYQDKGKFFVKSKKTFKNKTTVKYTFEDLDLTLNEIIT |
| | QDDIFVKVIFLFSIFMPKELNGDFFGFINMYYHKMKNISYDTKDIDMLDTISQNMKLKILE |
| | QNIKKTYVFKYYLDLDSSIYSKLVQNIKITEDIDSKKYLYAKIFKYYQHLYKLISDVEIYL |
| | LYKYNSKENLSITIDKDELKHRGYYNFQSLLIKNNINKDDAYWSIVNMRNNLSHQNIDE |
| | LVGHFCKGCLRKSTTDIAELWLRKDILTITNEIINKIESFKDIKITLGYDCVNDFTQKVKQ |
| | YKQKLKASNERLAKKIEEKQNQVVDEKNKEELEKNILNMKNIQKINRYILDIL |
| SEQ ID: | MLKHKRKNKNSLARVVLSNYDSNNIYEIKIKYEKLAKLDKINIIEMDYDADNNVMFKK |
| 39 | VLFNNKEIDLSHKDKTKINIELDNKKYNISAKKQIGKTHLVVRDKQTSKISRIKKIQDTYY |
| | RGKDVFILDNNIEILDKKQTKDKFIVTLNDITNDKTTSTEAELIDDTKDIFKKISAKKDLKS |
| | SDIYKIKRFISIRSNFSFYYTFVDNYFKIFHAKKDKNKEELYKIKFKDEINIKPYLENILDN |
| | MKNKNGILYDYADDREKVLNDLKNIQYVFTEFRHKLAHFDYNFLDNFFSNSVTDQYKQ |
| | KVNEIKLLDILLDNIDSLNVVPKQNYIEDETISVFDAKDIKLKRLYTYYIKLTINYPGFKKL |
| | INSFFIQDGIENQELKEYINNKEKDTQVLKELDNKAYYMDISQYRKYKNIYNKHKELVSE |
| | KELSSDGQKINSLNQKINKLKIEMKNITKPNALNRLIYRLRVAFGFIYKEYATINNENKSF |
| | LQDTKIKRFENISQQDIKNYLDISYQDKGKFFVKSKKTFKNKTTIKYTFEDLDLTLNEIIT |
| | QDDIFVKVIFLFSIFMPKELNGDFFGFINMYYHKMKNISYDTKDIDMLDTISQNMKLKILE |
| | QNIKKTYVFKYYLDLDSSIYSKLVQNIKITEDIDSKKYLYAKIFKYYQHLYKLISDVEIYL |
| | LYKYNSKENLSITIDKDELKHRGYYNFQSLLIKNNINKDDAYWSIVNMRNNLSHQNIDE |
| | LVGHFCKGCLRKSTTDIAELWLRKDILTITNEIINKIESFKDIKITLGYDCVNDFTQKVKQ |
| | YKQKLKASNERLAKKIEEKQNQVVDEKNKEELEKKILNMKNIQKINRYILDIL |
| SEQ ID: | MSQLKNPSNKNSLPRIIISDFNEIKINEIKIKYHKLDRLDKIIVKEMEIINNKIFFKKILENNQ |
| 40 | IKDINSENIELENYILAGEVKPSNTKIILNRDGKEKSFIVYDGFTFKYKPNDKRISETKTNA |
| | KYILTIKDKTRHRESSTQRDILKSSIIETYKQISGFENITSKDIYTIKRYIDFKNEMMFYYTFI |
| | DDFFFPITGKNKQDKKNNFYNYKIKENAKKFISLINYRINDDFKNKNGILYDYLSNKEEIII |
| | NDFIHIQTILKDVRHAIAHFNFDFIQKLFDNEQAFNSKFDGIEILNILFNQKQEKYFEAQTN |
| | YIEEETIKILDEKELSFKKLHSFYSQICQKKPAFNKLINSFIIQDGIENKELKDYISQKYNSK |
| | FDYYLDIHTCKIYKDIYNQHKKFVADKQFLENQKTDGQKIKKLNDQINQLKTKMNNLT |
| | KKNSLKRLEIKFRLAFGFIFTEYQTFKNFNERFIEDIKANKYSTKIELLDYGKIKEYISITHE |
| | EKRFFNYKTFNKKTNKNINKTIFQSLEKETFENLVKNDNLIKMMFLFQLLLPRELKGEFL |
| | GFILKIYHDLKNIDNDTKPDEKSLSELNISTALKLKILVKNIRQINLFNYTISNNTKYEEKE |
| | KRFYEEGNQWKDIYKKLYISHDFDIFDIHLIIPIIKYNINLYKLIGDFEVYLLLKYLERNTN |
| | YKTLDKLIEAEELKYKGYYNFTTLLSKAINIALNDKEYHNITHLRNNTSHQDIQNIISSFK |
| | NNKLLEQRENIIELISKESLKKKLHFDPINDFTMKTLQLLKSLEVHSDKSEKIENLLKKEP |
| | LLPNDVYLLYKLKGIEFIKKELISNIGITKYEEKIQEKIAKGVEK |
| SEQ ID: | MVKNPANRHALPKVIISEVDNNNILEFKIKYEKLARLDKVEVKSMHFDNNKQVVFDEV |
| 41 | VINGGLIEPTYEDKHKKLVVTAGEKSYSIVGQKVGGKPRLLEDRVSKTKVQLELTNYVE |
| | DKEGKKRVSKTERELIVADNIELYSQIVGREVKTTKEIYLIKRFLEYRSDLLFYYGFVDNF |
| | FKVAGNGKELWKIDFTNSDSLHLIEYFKFSINDNLKNDENYLKNYVSDNTKIENDLVKC |
| | QNNFNSLRHALMHFDYDFFEKLFNGEDVGFDFDIEFLNIMIDKVDKLNIDTKKEFIDDEE |
| | VTLFGEALSLKKLYGLFSHIAINRVAFNKLINSFIIEDGIENKELKDFENNKKESQAYEIDI |
| | HSNAEYKALYVQHKKLVMATSAMTDGDEIAKKNQEISDLKEKMKVITKENSLARLEHK |
| | LRLAFGFIYTEYKDYKTFKKHFDQDIKGAKYKGLNVEKLKEYYETTLKNSKPKTDEKLE |
| | DVAKKIDKLSLKELIDDDTLLKFVLLLFIFMPQELKGDFLGFIKKYYHDKKHIDQDTKDK |
| | DTEIEELSTGLKLKVLDKNIRSLSILKHSFSFQVKYNRKDKNFYEDGNLHGKFYKKLSIS |
| | HNQEEFNKSVYAPLFRYYSALYKLINDFEIYALAQHVENHETLADQVNKSQFIQKSYFN |
| | FRKLLDNTDSISQSSSYNTLIVMRNDISHLSYEPLFNYPLDERKSYKKKTQKGVKTFHVE |
| | LLYISRAKIIELISLQTDMKKLLGYDAVNDFNMKVVHLRKRLSVYANKEESIRKMQADA |
| | KTPNDFYNIYKVKGVESINQHLLKVIGVTEAEKSIEKQINEGNKKHNT |
| SEQ ID: | MTKKPSNRNSLPKVIINKVDESSILEFKIKYEKLARLDRFEVRSMRYDGDGRIIFDEVVAN |
| 42 | AGLLDVDYEDDNRTIVVKIENKAYNIYGKKVGGEKRLNGKISKAKVQLILTDSIRKNAN |
| | DTHRHSLTERELINKNEVDLYSKIAEREISTTKDIYLVKRFLAYRSDLLLYYAFINHYVRV |
| | NGNKKEFWKTEIDDKIIDYFIYTINDTLKNKEGYLEKYIVDRDQIKKDLEKIKQIFSHLRH |
| | KLMHYDFRFFTDLFDGKDVDIKVDNSIQKISELLDIEFLNIVIDKLEKLNIDAKKEFIDDE |
| | KITLFGQEIELKKLYSLYAHTSINRVAFNKLINSFLIKDGVENKELKEYFNAHNQGKESY |
| | YIDIHQNQEYKKLYIEHKNLVAKLSATTDGKEIAKINRELADKKEQMKQITKANSLKRL |
| | EYKLRLAFGFIYTEYKDYERFKNSFDTDTKKKKFDAIDNAKIIEYFEATNKAKKIEKLEEI |
| | LKGIDKLSLKTLIQDDILLKFLLLFFTFLPQEIKGEFLGFIKKYYHDITSLDEDTKDKDDEIT |
| | ELPRSLKLKIFSKNIRKLSILKHSLSYQIKYNKKESSYYEAGNVFNKMFKKQAISHNLEEF |
| | GKSIYLPMLKYYSALYKLINDFEIYALYKDMDTSETLSQQVDKQEYKRNEYFNFETLLR |
| | KKFGNDIEKVLVTYRNKIAHLDFNFLYDKPINKFISLYKSREKIVNYIKNHDIQAVLKYD |
| | AVNDFVMKVIQLRTKLKVYADKEQTIESMIQNTQNPNGFYNIYKVKAVENINRHLLKVI |
| | GYTESEKAVEEKIRAGNTSKS |
| SEQ ID: | MIKNPSNRYALPKVIISKIDNQNILEFKIKYKKLSKLDIVKVKSMHYDDRAIIFDEVIVND |
| 43 | GLIDVEYRDNHKTIFVKVGNKSYSISGQKVGGKERLLENRVSKTKVQLELKDKATNRVS |
| | KTERELIVDDNIKIYSQIVGRDVKTTKDIYLIKRFLAYRSDLLFYYGFVNNFFHVANNRSE |
| | FWKIDFNDSNNSKLIEYFKFTINDHLKNDENYLKDYISDNEKLKNDLIKVKNSFEKIRHA |
| | LMHFDYDFFVKLFNGEDVGLELDIEFLDIMIDKLDKLNIDTKKEFIDDEKITIFGEELSLA |
| | KLYRFYAHTAINRVAFNKLINSFIIENGVENQSLKEYFNQQAGGIAYEIDIHQNREYKNL |
| | YNEHKKLVSRVLSISDGQEIAILNQKIAKLKDQMKQITKANSIKRLEYKLRLALGFIYTEY |
| | ENYEEFKNNFDTDIKNGRFTPKDNDGNKRAFDSRELEQLKGYYEATIQTQKPKTDEKIE |
| | EVSKKIDRLSLKSLIADDILLKFILLMFTFMPQELKGEFLGFIKKYYHDTKHIDQDTISDSD |
| | DTIETLSIGLKLKILDKNIRSLSILKHSLSFQTKYNKKDRNYYEDGNIHGKFFKKLGISHN |
| | QEEFNKSVYAPLFRYYSALYKLINDFEIYTLSLHIVGSETLTDQVNKSQFLSGRYFNFRKL |
| | LTQSYHINNNSTHSTIFNAVINMRNDISHLSYEPLFDCPLNGKKSYKRKIRNQFKTINIKPL |
| | VESRKIIIDFITLQTDMQKVLGYDAVNDFTMKIVQLRTRLKAYANKEQTIQKMITEAKTP |
| | NDFYNIYKVQGVEEINKYLLEVIGETQAEKEIREKIERGNIANF |
| SEQ ID: | MKKSIFDQFVNQYALSKTLRFELKPVGETGRMLEEAKVFAKDETIKKKYEATKPFFNKL |
| 44 | HREFVEEALNEVELAGLPEYFEIFKYWKRYKKKFEKDLQKKEKELRKSVVGFFNAQAK |
| | EWAKKYETLGVKKKDVGLLFEENVFAILKERYGNEEGSQIVDESTGKDVSIFDSWKGFT |
| | GYFIKFQETRKNFYKDDGTATALATRIIDQNLKRFCDNLLIFESIRDKIDFSEVEQTMGNS |
| | IDKVFSVIFYSSCLLQEGIDFYNCVLGGETLPNGEKRQGINELINLYRQKTSEKVPFLKLL |
| | DKQILSEKEKFMDEIENDEALLDTLKIFRKSAEEKTTLLKNIFGDFVMNQGKYDLAQIYIS |
| | RESLNTISRKWTSETDIFEDSLYEVLKKSKIVSASVKKKDGGYAFPEFIALIYVKSALEQIP |
| | TEKFWKERYYKNIGDVLNKGFLNGKEGVWLQFLLIFDFEFNSLFEREIIDENGDKKVAG |
| | YNLFAKGFDDLLNNFKYDQKAKVVIKDFADEVLHIYQMGKYFAIEKKRSWLADYDIDS |
| | FYTDPEKGYLKFYENAYEEIIQVYNKLRNYLTKKPYSEDKWKLNFENPTLADGWDKNK |
| | EADNSTVILKKDGRYYLGLMARGRNKLFDDRNLPKILEGVENGKYEKVVYKYFPDQA |
| | KMFPKVCFSTKGLEFFQPSEEVITIYKNSEFKKGYTFNVRSMQRLIDFYKDCLVRYEGW |
| | QCYDFRNLRKTEDYRKNIEEFFSDVAMDGYKISFQDVSESYIKEKNQNGDLYLFEIKNK |
| | DWNEGANGKKNLHTIYFESLFSADNIAMNFPVKLNGQAEIFYRPRTEGLEKERIITKKGN |
| | VLEKGDKAFHKRRYTENKVFFHVPITLNRTKKNPFQFNAKINDFLAKNSDINVIGVDRG |
| | EKQLAYFSVISQRGKILDRGSLNVINGVNYAEKLEEKARGREQARKDWQQIEGIKDLKK |
| | GYISQVVRKLADLAIQYNAIIVFEDLNMRFKQIRGGIEKSVYQQLEKALIDKLTFLVEKEE |
| | KDVEKAGHLLKAYQLAAPFETFQKMGKQTGIVFYTQAAYTSRIDPVTGWRPHLYLKYS |
| | SAEKAKADLLKFKKIKFVDGRFEFTYDIKSFREQKEHPKATVWTVCSCVERFRWNRYL |
| | NSNKGGYDHYSDVTKFLVELFQEYGIDFERGDIVGQIEVLETKGNEKFFKNFVFFFNLIC |
| | QIRNTNASELAKKDGKDDFILSPVEPFFDSRNSEKFGEDLPKNGDDNGAFNIARKGLVIM |
| | DKITKFADENGGCEKMKWGDLYVSNVEWDNFVANK |
| SEQ ID: | MENNFIKKYSLQKTLRFELKPVGETADYIEDFKSEYLKDTVLKDEQRAKDYQEIKTLIDD |
| 45 | YHREYIEECLREPVDKKTGEILDFTQDLEDAFSYYQKLKENPTENRVGWEKEQESLRKK |
| | LVTSFVGNDGLFKKEFITRDLPEWLQKKGLWGEYKDTVENFKKFTTYFSGFHENRKNM |
| | YTAEAQSTAIANRLMNDNLPKFFNNYLAYQTIKEKHPDLVFRLDDALLQAAGVEHLDE |
| | AFQPRYFSRLFAQSGITAFNELIGGRTTENGEKIQGLNEQINLYRQQNPEKAKGFPRFMPL |
| | FKQILSDRETHSFLPDAFENDKELLQALRDYVDAATSEEGMISQLNKAMNQFVTADLKR |
| | VYIKSAALTSLSQELFHFFGVISDAIAWYAEKRLSPKKAQESFLKQEVYAIEELNQAVVG |
| | YIDQLEDQSELQQLLVDLPDPQKPVSSFILTHWQKSQEPLQAVIAKVEPLFELEELSKNK |
| | RAPKHDKDQGGEGFQQVDAIKNMLDAFMEVSHAIKPLYLVKGRKAIDMPDVDTGFYA |
| | DFAEAYSAYEQVTVSLYNKTRNHLSKKPFSKDKIKINFDAPTLLNGWDLNKESDNKSIIL |
| | RKDGNFYLAIMHPKHTKVFDCYSASEAAGKCYEKMNYKLLSGANKMLPKVFFSKKGIE |
| | TFSPPQEILDLYKNNEHKKGATFKLESCHKLIDFFKRNIPKYKVHPTDNFGWDVFGFHFS |
| | PTSSYGDLSGFYREVEAQGYKLWFSDVSEAYINKCVEEGKLFLFQIYNKDFSPNSTGKPN |
| | LHTLYWKGLFEPENLKDVVLKLNGEAEIFYRKHSIKHEDKTIHRAKDPIANKNADNPKK |
| | QSVFDYDIIKDKRYTQDKFFFHVPISLNFKSQGVVRENDKINGLLAAQDDVHVIGIDRGE |
| | RHLLYYTVVNGKGEVVEQGSLNQVATDQGYVVDYQQKLHAKEKERDQARKNWSTIE |
| | NIKELKAGYLSQVVHKLAQLIVKHNAIVCLEDLNFGFKRGRFKVEKQVYQKFEKALIDK |
| | LNYLVFKERGATQAGGYLNAYQLAAPFESFEKLGKQTGILYYVRSDYTSKIDPATGFVD |
| | FLKPKYESMAKSKVFFESFERIQWNQAKGYFEFEFDYKKMCPSRKFGDYRTRWVVCTF |
| | GDTRYQNRRNKSSGQWETETIDVTAQLKALFAAYGITYNQEDNIKDAIAAVKYTKFYK |
| | QLYWLLRLTLSLRHSVTGTDEDFILSPVADENGVFFDSRKATDKQPKDADANGAYHIAL |
| | KGLWNLQQIRQHDWNVEKPKKLNLAMKNEEWFGFAQKKKFRA |
| SEQ ID: | MIKNPSNRYALPKVIISKIDNQNILEFKIKYKKLSKLDIVKVKSMHYDDRAIIFDEVIVND |
| 46 | GLIDVEYRDNHKTIFVKVGNKSYSISGQKVGGKERLLENRVSKTKVQLELKDKATNRVS |
| | KTERELIVDDNIKIYSQIVGRDVKTTKDIYLIKRFLAYRSDLLFYYGFVNNFFHVANNRSE |
| | FWKIDFNDSNNSKLIEYFKFTINDHLKNDENYLKDYISDNEKLKNDLIKVKNSFEKIRHA |
| | LMHFDYDFFVKLFNGEDVGLELDIEFLDIMIDKLDKLNIDTKKEFIDDEKITIFGEELSLA |
| | KLYRFYAHTAINRVAFNKLINSFIIENGVENQSLKEYFNQQAGGIAYEIDIHQNREYKNL |
| | YNEHKKLVSRVLSISDGQEIAILNQKIAKLKDQMKQITKANSIKRLEYKLRLALGFIYTEY |
| | ENYEEFKNNFDTDIKNGRFTPKDNDGNKRAFDSRELEQLKGYYEATIQTQKPKTDEKIE |
| | EVSKKIDRLSLKSLIADDILLKFILLMFTFMPQELKGEFLGFIKKYYHDTKHIDQDTISDSD |
| | DTIETLSIGLKLKILDKNIRSLSILKHSLSFQTKYNKKDRNYYEDGNIHGKFFKKLGISHN |
| | QEEFNKSVYAPLFRYYSALYKLINDFEIYTLSLHIVGSETLTDQVNKSQFLSGRYFNFRKL |
| | LTQSYHINNNSTHSTIFNAVINMRNDISHLSYEPLFDCPLNGKKSYKRKIRNQFKTINIKPL |
| | VESRKIIIDFITLQTDMQKVLGYDAVNDFTMKIVQLRTRLKAYANKEQTIQKMITEAKTP |
| | NDFYNIYKVQGVEEINKYLLEVIGETQAEKEIREKIERGNIANF |
| SEQ ID: | MIKNPSNRHSLPKVIISEVDHEKILEFKIKYEKLARLDRFEVKAMHYEGKEIVFDEVLVN |
| 47 | GGLIEVEYQDDNKTLFVKVGEKSYSIRGKKVGGKQRLLEDRVSKTKVQLELSDGVVDN |
| | KGNLRKSRTERELIVADNIKLYSQIVGREVTTTKEIYLVKRFLAYRSDLLFYYSFVDNFF |
| | KVAGNEKELWKINFDDATSAQFMGYIPFMVNDNLKNDNAYLKDYVRNDVQIKDDLKK |
| | VQTIFSALRHTLLHFNYEFFEKLFNGEDVGFDFDIGFLNLLIENIDKLNIDAKKEFIDNEKI |
| | RLFGENLSLAKVYRLYSDICVNRVGFNKFINSMLIKDGVENQVLKAEFNRKFGGNAYTI |
| | DIHSNQEYKRIYNEHKKLVIKVSTLKDGQAIRRGNKKISELKEQMKSMTKKNSLARLEC |
| | KMRLAFGFLYGEYNNYKAFKNNFDTNIKNSQFDVNDVEKSKAYFLSTYERRKPRTREK |
| | LEKVAKDIESLELKTVIANDTLLKFILLMFVFMPQELKGDFLGFVKKYYHDVHSIDDDT |
| | KEQEEDVVEAMSTSLKLKILGRNIRSLTLFKYALSSQVNYNSTDNIFYVEGNRYGKIYKK |
| | LGISHNQEEFDKTLVVPLLRYYSSLFKLMNDFEIYSLAKANPTAVSLQELVDDETSPYKQ |
| | GNYFNFNKMLRDIYGLTSDEIKSGQVVFMRNKIAHFDTEVLLSKPLLGQTKMNLQRKDI |
| | VSFIEARGDIKELLGYDAINDFRMKVIHLRTKMRVYSDKLQTMMDLLRNAKTPNDFYN |
| | VYKVKGVESINKHLLEVLAQTAEERTVEKQIRDGNEKYDL |
| SEQ ID: | MEEKMLKSYDYFTKLYSLQKTLRFELKPIGKTLEHIKNSGIIESDETLEEQYAIVKNIIDKL |
| 48 | HRKHIDEALSLVDFTKHLDTLKTFQELYLKRGKTDKEKEELEKLSADLRKLIVSYLKGN |
| | VKEKTQHNLNPIKERFEILFGKELFTNEEFFLLAENEKEKKAIQAFKGFTTYFKGFQENR |
| | KNMYSEEGNSTSIAYRIINENLPLFIENIARFQKVMSTIEKTTIKKLEQNLKTELKKHNLPG |
| | IFTIEYFNNVLTQEGISRYNTIIGGKTTHEGVKIQGLNEIINLYNQQSKDVKLPILKPLHKQI |
| | LSEEYSTSFKIKAFENDNEVLKAIDTFWNEHIEKSIHPVTGNKFNILSKIENLCDQLQKYK |
| | DKDLEKLFIERKNLSTVSHQVYGQWNIIRDALRMHLEMNNKNIKEKDIDKYLDNDAFS |
| | WKEIKDSIKIYKEHVEDAKELNENGIIKYFSAMSINEEDDEKEYSISLIKNINEKYNNVKSI |
| | LQEDRTGKSDLHQDKEKVGIIKEFLDSLKQLQWFLRLLYVTVPLDEKDYEFYNELEVYY |
| | EALLPLNSLYNKVRNYMTRKPYSVEKFKLNFNSPTLLDGWDKNKETANLSIILRKNGKY |
| | YLGIMNKENNTIFEYYPGTKSNDYYEKMIYKLLPGPNKMLPKVFFSKKGLEYYNPPKEI |
| | LNIYEKGEFKKDKSGNFKKESLHTLIDFYKEAIAKNEDWEVFNFKFKNTKEYEDISQFYR |
| | DVEEQGYLITFEKVDANYVDKLVKEGKLYLFQIYNKDFSENKKSKGNPNLHTIYWKGL |
| | YDSENLKNVVYKLNGEAEVFYRKKSIDYPEEIYNHGHHKEELLGKFNYPIIKDRRYTQD |
| | KFLFHVPITMNFISKEEKRVNQLACEYLSATKEDVHIIGIDRGERHLLYLSLIDKEGNIKK |
| | QLSLNTIKNENYDKEIDYRVKLDEKEKKRDEARKNWDVIENIKELKEGYMSQVIHIIAK |
| | MMVEEKAILIMEDLNIGFKRGRFKVEKQVYQKFEKMLIDKLNYLVFKNKNPLEPGGSL |
| | NAYQLTSKFDSFKKLGKQSGFIFYVPSAYTSKIDPTTGFYNFIQVDVPNLEKGKEFFSKFE |
| | KIIYNTKEDYFEFHCKYGKFVSEPKNKDNDRKTKESLTYYNAIKDTVWVVCSTNHERY |
| | KIVRNKAGYYESHPVDVTKNLKDIFSQANINYNEGKDIKPIIIESNNAKLLKSIAEQLKLIL |
| | AMRYNNGKHGDDEKDYILSPVKNKQGKFFCTLDGNQTLPINADANGAYNIALKGLLLI |
| | EKIKKQQGKIKDLYISNLEWFMFMMSR |
| SEQ ID: | MEKSLNDFIGLYSVSKTLRFELKPVSETLENIKKFHFLEEDKKKANDYKDVKKIIDNYHK |
| 49 | YFIDDVLKNASFNWKKLEEAIREYNKNKSDDSALVAEQKKLGDAILKLFTSDKRYKALT |
| | AATPKELFESILPDWFGEQCNQDLNKAALKTFQKFTSYFTGFQENRKNVYSAEAIPTAVP |
| | YRIVNDNFPKFLQNVLIFKTIQEKCPQIIDEVEKELSSYLGKEKLAGIFTLESFNKYLGQG |
| | GKENQRGIDFYNQIIGGVVEKEGGINLRGVNQFLNLYWQQHPDFTKEDRRIKMVPLYK |
| | QILSDRSSLSFKIESIENDEELKNALLECADKLELKNDEKKSIFEEVCDLFSSVKNLDLSGI |
| | YINRKDINSVSRILTGDWSWLQSRMNVYAEEKFTTKAEKARWQKSLDDEGENKSKGFY |
| | SLTDLNEVLEYSSENVAETDIRITDYFEHRCRYYVDKETEMFVQGSELVALSLQEMCDDI |
| | LKKRKAMNTVLENLSSENKLREKTDDVAVIKEYLDAVQELLHRIKPLKVNGVGDSTFY |
| | SVYDSIYSALSEVISVYNKTRNYITKKAASPEKYKLNFDNPTLADGWDLNKEQANTSVIL |
| | RKDGMFYLGIMNPKNKPKFAEKYDCGNESCYEKMIYKQFDATKQIPKCSTQKKEVQKY |
| | FLSGATEPYILNDKKSFKSELIITKDIWFMNNHVWDGEKFVPKRDNETRPKKFQIGYFKQ |
| | TGDFDGYKNALSNWISFCKNFLQSYLSATVYDYNFKNSEEYEGLDEFYNYLNATCYKL |
| | NFINIPETEINKMVSEGKLYLFQIYNKDFASGSTGMPNMHTLYWKNLFSDENLKNVCLK |
| | LNGEAELFYRPAGIKEPVIHKEGSYLVNRTTEDGESIPEKIYFEIYKNANGKLEKLSDEAQ |
| | NYISNHEVVIKKAGHEIIKDRHYTEPKFLFHVPLTINFKASGNSYSINENVRKFLKNNPDV |
| | NIIGLDRGERHLIYLSLINQKGEIIKQFTFNEVERNKNGRTIKVNYHEKLDQREKERDAAR |
| | KSWQAIGKIAELKEGYLSAVIHQLTKLMVEYNAVVVMEDLNFGFKRGRFHVEKQVYQ |
| | KFEHILIDKSNYLVFKDRGLNEPGGVLNGYQIAGQFESFQKLGKQSGMLFYVPAGYTSKI |
| | DPKTGFVSMMNFKDLTNVHKKRDFFSKFDNIHYDEANGSFVFTFDYKKFDGKAKEEM |
| | KLTKWSVYSRDKRIVYFAKTKSYEDVLPTEKLQKIFESNGIDYKSGNNIQDSVMAIGAD |
| | LKEGAKPSKEISDFWDGLLSNFKLILQMRNSNARTGEDYIISPVMADDGTFFDSREEFKK |
| | GEDAKLPLDADANGAYHIALKGLSLINKINLSKDEELKKFDMKISNADWFKFAQEKNY |
| | AK |
| SEQ ID: | MNTQKKEFNPKSFKDFTNLYSLNKTLRFSLTPNKKTAEILEFNKQKEVKCFSNDRKIAG |
| 50 | AYQEIKKYLNKLHQEFIQEAMKFFAFSEEELKGFEKEYLNLLNFTDKDNFKKKNKIRNE |
| | YEQERKILTIKIATYFSKFKSEKYQSFNLANITGKKVFSILEQKYKEDKKTLKIIHIFKYKP |
| | TKDEKKEGEAVNFSTYLTGFNENRKNFYKSEDKAGQFATRTIDNLAQFIKNKKLFEDKY |
| | QKNYSKIGILDEQIKIFNLDYFNNLFLQEGLDEYNGILGNNKGEENKSNEGINQKINIFKQ |
| | KEKARLKKEKENFNKSDFPLFKELYKQIGSIRKENDVYVEIKTDKELVEELNNFPKNVEN |
| | YLKDIQSFYKTFFEKLQNEEYELDKIYLPKSVGTYFSYIAFSDWNKLAFIYNKRYKNEKI |
| | KIVEGGDVNVQYRSLEVLKNRIDELKDEDNLNFNKFFIDKLKFNEAKKENNWQNFWFCI |
| | EYYINSQFIGGEKNILNKEKNEYEILPFGSLKELKEKYFEAVKKYKEKMVDTESGLTDDE |
| | EKEIKETLKNYLDRIKEIERIAKYFDLKKSFEEIKQEDLDSNFYGEYQKVVDKTNELKIYQ |
| | YYSEFRNYLTQNNSVEEKIKLNFNSGLLLDGWDLNKEKVKFSIIFQENGKYYLGIINKEK |
| | DKTILDKDKHPEIFTKNSDFRKMEYKLFPSPSKMLPKISFSETAKKGDEDVGWSEEIQKIK |
| | DEFAEFQEYKKKSKDNWKDEFNRGKLNKLIDYYKQVLEKHSEGYMNTYNFELKDSSK |
| | YKNLGEFNDDIARQNYKVKFVGIDKNYIDEKVANGELFLFQIYNKDFSEDKKEGSTNNL |
| | ETIYFKELFSKENLENPVFKLSGGAEMFFRNKIEKKKEKKKLDKDGKPMISKKGEKVVD |
| | KRRFSENKILFHLPIEINYGKGKMPNFNKKINEYISKNPENIKIIGIDRGEKHLLYYSIIDQN |
| | GNNIESMSLNAVDEFGNFVNPEKLEEYEIDNNGKKERRWKYIVNDKEIKVTNYQRKLD |
| | ELEKERQKSRQSWQNINKIKNLKKGYISFVVKKIVDLAIENNAIIILEDLNFGFKSFRQKIE |
| | KNVYQQFEKALIDKLGFVVDKQKQNQRFAPQLSAPFESFQKIGKQTGIVYYVLANNTSK |
| | VCPSCQWIKNFYLKYEKKNTIFNLQKNQKLKVFFEQEKNRFRFEYQMSKEYISVYSDVD |
| | RQRYDKTKNQNKGGYLEYKNSNQKEIIDKDGVIQKQSITLQLKELFKENHIDLEKEILKQ |
| | LDNKKEKNSGYTGVYNKFIYLFNLILQIRNAISFREKDYIQCPSCHFDTRKENYLKINDG |
| | DGNGAYNIALRGLYLLKGKNGIINNLEKIKLIFSNNDYFQWAKKLKNKK |
| SEQ ID: | MQNKQSFADFTNLYSLSKTLRFELKPIGQTQAMLDENKIFEVDENRKKAYDKTKPYFDR |
| 51 | LHREFINESLSNAQLKGISEYFETFKQFRSNQNNKDLKELINKQQKFLRHQIVTLFDENG |
| | KHWATTKYAHLKIKKKNLDILFDEQVFYILKERYGSEKETQLVDKETGAVTSIFDNWKG |
| | FTGYFTKFFETRKNFYKSDGTSTALATRIIDQNLNRFFDNLETFHKIKDKIDVKEVEIFFK |
| | LKADNVFSIDFYNQCLLQNGIDKYNDFLGGQTLENGEKQKGINEIINKYRQDNKDQKLP |
| | FLKKLDKQILSEKDRFINEIESKEEFFQVLTEFYQSATVKVTIIKTLLNDFVHNTDKYKLE |
| | KIYLTKEAFNTIANKWTDETQIFEDNLDLVLKNKKITAKQDFIPLAYIKEALEVIEKDRKF |
| | FKDRYYNDPQIGFFPDQSYWEQFLAILNFEFMTHFQRVAKDKITGKKIELGYFVFEKRIK |
| | ELLDSDPSLNSQSKIIIKEFADEVLHIFQMAKYFALEKKREWKGDYYQLDDQFYNHIDY |
| | GFKDQFYENAYEKIVQPYNKIRNYLTKKPYSDVKWKLNFGNPTLANGWDKNKEADNT |
| | AVILKKDGNYYLGVMKKGKNKIFSDQNKEKYKAYNSAYYEKLVYKLFPDPSKMFPKV |
| | CFSKKGLNFFQPSEEILRIYKNNEFKKGNTFSISSMQKLIAFYIDCLGLYEGWKHYEFKNI |
| | KDVRQYKENIGEFYADVAESGYKLWFEKISEEYITQKNQLGELFLFQIYNKDFAKKTTG |
| | RKNLHTIYFEELFSQTNIDNNFPFKLNGQAELFYRPKSLEKIEEKRNFKRSIVNKKRYTQN |
| | KIFFHVPITLNRTSENIGRFNVRVNNFLANNSNVNIVGVDRGEKNLAYYSIIKQNGEVLK |
| | SGSLNIINGVDYHALLTDRAQRREQERRNWQDVESIKDLKRGYISQVVHELVSLAIKYN |
| | AIIVMEDLNMRFKQIRGGIEKSTYQQLEKALIEKLNFLVNKEETDSNQAGNLLNAYQLT |
| | APFKTFKDMGKQTGIIFYTQASYTSKIDPLTGWRPNIYLRYSNAKQAKADILMFTNIYFS |
| | EKKDRFEFTYDLEKIDDKRKDLPIKTEWTVCSNVERFSWEKSLNNNKGGYVHYPIQDSN |
| | GEESITSKLKKLFMDFGIDLTDIKTQIESLDTNKKDNANFFRKFIFYFQLICQIRNTQVNKS |
| | DDGNDFIFSPVEPFFDSRFADKFRKNLPKNGDENGAYNIARKGLIILHKISDYFVKEGSTD |
| | KISWKDLSISQTEWDNFTTDK |
| SEQ ID: | MKKEKEFKSFGDFTNLYEISKTLRFELKPVENTQTMLDEADVFGKDKVIKDKYTKTKPF |
| 52 | IDKLHREFVDESLKDVSLSGLKKYSEVLENWKKNKKDKDIVKELKKEEERLRKEVVEFF |
| | DNTAKKWANEKYKELGLKKKDIGILFEESVFDLLKEKYGEEQDSFLKEEKGDFLKNEK |
| | GEKVSIFDEWKGFVGYFTKFQETRKNFYKNDGTETALATRIIDQNLKRFCDNIDDFKKIK |
| | NKIDFSEVEKNFNKTADVFSLDFYNQCLLQKGIDSYNEFIGGKTLENGKKLKGVNELVN |
| | EYRQKNKNEKVSFLKLLDKQILSEKEKLSFGIENDEQLLVVLNSFYETAEEKTKILRTLF |
| | GDFVEHNENYDLDKTYISKVAFNTISHKWTNETHKFEELLYGAMKEDKPIGLNYDKKE |
| | DSYKFPDFIALGYLKKCLNNLDCDTKFWKEKYYENNADKKDKDKGFLTGGQNAWDQF |
| | LQIFIFEFNQLFNSEAFDNKGKEIKIGYDNFRKDFEEIINQKDFKNDENLKIAIKNFADSVL |
| | WIYQMAKYFAIEKKRGWDDDFELSEFYTNPSNGYSLFYDRAYEEIVQKYNDLRNYLTK |
| | KPYKEDKWKLNFENPTLANGFDKNKESDNSTVILRKKRKYYLGLMKKGNNKIFEDRNK |
| | AEFIRNIESGAYEKMAYKYLPDVAKMIPKCSTQLNEAKNHFRNSADDLEIKKSFSNPLKI |
| | TKRIFDLNNIQYDKTNVSKKISGDNKGIKIFQKEYYKISGDFDVYKSALNDWIDFCKDFL |
| | SKYDSTKDFDFSILRKTKDYKSLDEFYVDVAKITYKISFTPVSESYIDQKNKNGELYLFEI |
| | YNQDFAKGKMGAKNLHTLYFENVFSPENISKNFPIKLNGNAELFFRPKSIESKKEKRNFV |
| | REIVNKKRYSEDKIFFHCPITLNRETGSIYRFNNYVNNFLSENNINIIGVDRGEKHLAYYS |
| | VIDKNGVKIGGGSFNEINKVDYAKKLEERAGEREQSRKDWQVVEGIKDLKKGYISQVV |
| | RELADLAIKHNAIIVLEDLNMRFKQIRGGIEKSIYQQLEKALIDKLSFLVEKGEKDPNQAG |
| | HILKAYQLAAPFTSFKDMGKQTGIVFYTQASYTSKTCPNCGFRKNNNKFYFENNIGKAQ |
| | DALKKLKTFEYDSENKCFGLSYCLSDFANKEEVEKNKNKKRNNAPYSDIEKKDCFELST |
| | KDAVRYRWHDKNTERGKTFFEGESVYEEKEEKEIGQTKRGLVKEYDISKCLIGLFEKTG |
| | LDYKQNLLDKINSGKFDGTFYKNLFNYLNLLFEIRNSISGTEIDYISCPECQFHTDKSKTIK |
| | NGDDNGSYNIARKGMIILDKIKQFKKENGSLDKMGWGELFIDLEEWDKFAQKKNNNIID |
| | K |
| SEQ ID: | MKYTDFTGIYSVSKTLRFELIPQGSTVENMKREGILNNDMHRADSYKEMKKLIDEYHKA |
| 53 | FIERCLSDFSLKYDDTGKHDSLEEYFFYYEQKRNDKTKKIFEDIQVALRKQISKRFTGDT |
| | AFKRLFKKELIKEDLPSFVKNDPVKTELIKEFSDFTTYFQEFHKNRKNMYTSDAKSTAIA |
| | YRIINENLPKFIDNINAFDIVAKVPEMQEHFKTIADELRSHLQVGNDIDKMFNLQFFNKVL |
| | TQSQLDVYNAVIGGKSEGNKKIQGINEYINLYNQQHKKARLPMLKLLYKQILSDRVAIS |
| | WLQDEFDNDQDMLDTIEAFYNKLNSNETGVLGEGKLKQILMGLDGYNLDGVFMRNDL |
| | QLSEVSQRLCGGWNIIKDAMTSDLKRSVQKKKKETDADFEERVSKLFSAQNSFSIAYIN |
| | QCLGQAGIRCKIQDYFACLGAKEGENEAETTPDIFDQIAEAYHGAAPILNARPSSHNLAQ |
| | DIEKVKAIKALLDALKRLQRFVKPLLGRGDEGDKDNFFYGDFMPIWEVLDQLTPLYNK |
| | VRNRMTRKPYSQEKIKLNFENSTLLNGWDLNKEHDNTSVILRREGLYYLGIMNKNYNKI |
| | FDANNVETIGDCYEKMIYKLLPGPNKMLPKVFFSKSRVQEFSPSKKILEIWESKSFKKGD |
| | NFNLDDCHALIDFYKDSIAKHPDWNKFNFKFSDTQSYTNISDFYRDVNQQGYSLSFTKV |
| | SVDYVNRMVDEGKLYLFQIYNKDFSPQSKGTPNMHTLYWRMLFDERNLHNVIYKLNG |
| | EAEVFYRKASLRCDRPTHPAHQPITCKNENDSKRVCVFDYDIIKNRRYTVDKFMFHVPIT |
| | INYKCTGSDNINQQVCDYLRSAGDDTHIIGIDRGERNLLYLVIIDQHGTIKEQFSLNEIVN |
| | EYKGNTYCTNYHSLLEEKEAGNKKARQDWQTIESIKELKEGYLSQVIHKISMLMQRYH |
| | AIVVLEDLNGSFMRSRQKVEKQVYQKFEHMLINKLNYLVNKQYDATEPGGLLHALQLT |
| | SRMDSFKKLGKQSGFLFYIPAWNTSKIDPVTGFVNLFDTRYCNEAKAKEFFEKFDDISYN |
| | DERDWFEFSFDYRHFTNKPTGTRTQWTLCTQGTRVRTFRNPEKSNHWDNEEFDLTQAF |
| | KDLFNKYGIDIASGLKARIVNGQLTKETSAVKDFYESLLKLLKLTLQMRNSVTGTDIDYL |
| | VSPVADKDGIFFDSRTCGSLLPANADANGAFNIARKGLMLLRQIQQSSIDAEKIQLAPIKN |
| | EDWLEFAQEKPYL |
| SED ID: | MEKEITELTKIRREFPNKKFSSTDMKKAGKLLKAEGPDAVRDFLNSCQEIIGDFKPPVKT |
| 54 | NIVSISRPFEEWPVSMVGRAIQEYYFSLTKEELESVHPGTSSEDHKSFFNITGLSNYNYTS |
| | VQGLNLIFKNAKAIYDGTLVKANNKNKKLEKKFNEINHKRSLEGLPIITPDFEEPFDENG |
| | HLNNPPGINRNIYGYQGCAAKVFVPSKHKMVSLPKEYEGYNRDPNLSLAGFRNRLEIPE |
| | GEPGHVPWFQRMDIPEGQIGHVNKIQRFNFVHGKNSGKVKFSDKTGRVKRYHHSKYKD |
| | ATKPYKFLEESKKVSALDSILAIITIGDDWVVFDIRGLYRNVFYRELAQKGLTAVQLLDL |
| | FTGDPVIDPKKGVVTFSYKEGVVPVFSQKIVPRFKSRDTLEKLTSQGPVALLSVDLGQNE |
| | PVAARVCSLKNINDKITLDNSCRISFLDDYKKQIKDYRDSLDELEIKIRLEAINSLETNQQ |
| | VEIRDLDVFSADRAKANTVDMFDIDPNLISWDSMSDARVSTQISDLYLKNGGDESRVYF |
| | EINNKRIKRSDYNISQLVRPKLSDSTRKNLNDSIWKLKRTSEEYLKLSKRKLELSRAVVN |
| | YTIRQSKLLSGINDIVIILEDLDVKKKFNGRGIRDIGWDNFFSSRKENRWFIPAFHKAFSEL |
| | SSNRGLCVIEVNPAWTSATCPDCGFCSKENRDGINFTCRKCGVSYHADIDVATLNIARV |
| | AVLGKPMSGPADRERLGDTKKPRVARSRKTMKRKDISNSTVEAMVTA |
| SED ID: | MIKPTVSQFLTPGFKLIRNHSRTAGLKLKNEGEEACKKFVRENEIPKDECPNFQGGPAIA |
| 55 | NIIAKSREFTEWEIYQSSLAIQEVIFTLPKDKLPEPILKEEWRAQWLSEHGLDTVPYKEAA |
| | GLNLIIKNAVNTYKGVQVKVDNKNKNNLAKINRKNEIAKLNGEQEISFEEIKAFDDKGY |
| | LLQKPSPNKSIYCYQSVSPKPFITSKYHNVNLPEEYIGYYRKSNEPIVSPYQFDRLRIPIGEP |
| | GYVPKWQYTFLSKKENKRRKLSKRIKNVSPILGIICIKKDWCVFDMRGLLRTNHWKKY |
| | HKPTDSINDLFDYFTGDPVIDTKANVVRFRYKMENGIVNYKPVREKKGKELLENICDQN |
| | GSCKLATVDVGQNNPVAIGLFELKKVNGELTKTLISRHPTPIDFCNKITAYRERYDKLES |
| | SIKLDAIKQLTSEQKIEVDNYNNNFTPQNTKQIVCSKLNINPNDLPWDKMISGTHFISEKA |
| | QVSNKSEIYFTSTDKGKTKDVMKSDYKWFQDYKPKLSKEVRDALSDIEWRLRRESLEF |
| | NKLSKSREQDARQLANWISSMCDVIGIENLVKKNNFFGGSGKREPGWDNFYKPKKENR |
| | WWINAIHKALTELSQNKGKRVILLPAMRTSITCPKCKYCDSKNRNGEKFNCLKCGIELN |
| | ADIDVATENLATVAITAQSMPKPTCERSGDAKKPVRARKAKAPEFHDKLAPSYTVVLRE |
| | AV |
| SED ID: | MEKEITELTKIRREFPNKKFSSTDMKKAGKLLKAEGPDAVRDFLNSCQEIIGDFKPPVKT |
| 56 | NIVSISRPFEEWPVSMVGRAIQEYYFSLTKEELESVHPGTSSEDHKSFFNITGLSNYNYTS |
| | VQGLNLIFKNAKAIYDGTLVKANNKNKKLEKKFNEINHKRSLEGLPIITPDFEEPFDENG |
| | HLNNPPGINRNIYGYQGCAAKVFVPSKHKMVSLPKEYEGYNRDPNLSLAGFRNRLEIPE |
| | GEPGHVPWFQRMDIPEGQIGHVNKIQRFNFVHGKNSGKVKFSDKTGRVKRYHHSKYKD |
| | ATKPYKFLEESKKVSALDSILAIITIGDDWVVFDIRGLYRNVFYRELAQKGLTAVQLLDL |
| | FTGDPVIDPKKGVVTFSYKEGVVPVFSQKIVPRFKSRDTLEKLTSQGPVALLSVDLGQNE |
| | PVAARVCSLKNINDKITLDNSCRISFLDDYKKQIKDYRDSLDELEIKIRLEAINSLETNQQ |
| | VEIRDLDVFSADRAKANTVDMFDIDPNLISWDSMSDARVSTQISDLYLKNGGDESRVYF |
| | EINNKRIKRSDYNISQLVRPKLSDSTRKNLNDSIWKLKRTSEEYLKLSKRKLELSRAVVN |
| | YTIRQSKLLSGINDIVIILEDLDVKKKFNGRGIRDIGWDNFFSSRKENRWFIPAFHKTFSEL |
| | SSNRGLCVIEVNPAWTSATCPDCGFCSKENRDGINFTCRKCGVSYHADIDVATLNIARV |
| | AVLGKPMSGPADRERLGDTKKPRVARSRKTMKRKDISNSTVEAMVTA |
| SEQ ID: | VPDKKETPLVALCKKSFPGLRFKKHDSRQAGRILKSKGEGAAVAFLEGKGGTTQPNFKP |
| 57 | PVKCNIVAMSRPLEEWPIYKASVVIQKYVYAQSYEEFKATDPGKSEAGLRAWLKATRV |
| | DTDGYFNVQGLNLIFQNARATYEGVLKKVENRNSKKVAKIEQRNEHRAERGLPLLTLD |
| | EPETALDETGHLRHRPGINCSVFGYQHMKLKPYVPGSIPGVTGYSRDPSTPIAACGVDRL |
| | EIPEGQPGYVPPWDRENLSVKKHRRKRASWARSRGGAIDDNMLLAVVRVADDWALLD |
| | LRGLLRNTQYRKLLDRSVPVTIESLLNLVTNDPTLSVVKKPGKPVRYTATLIYKQGVVP |
| | VVKAKVVKGSYVSKMLDDTTETFSLVGVDLGVNNLIAANALRIRPGKCVERLQAFTLP |
| | EQTVEDFFRFRKAYDKHQENLRLAAVRSLTAEQQAEVLALDTFGPEQAKMQVCGHLG |
| | LSVDEVPWDKVNSRSSILSDLAKERGVDDTLYMFPFFKGKGKKRKTEIRKRWDVNWA |
| | QHFRPQLTSETRKALNEAKWEAERNSSKYHQLSIRKKELSRHCVNYVIRTAEKRAQCGK |
| | VIVAVEDLHHSFRRGGKGSRKSGWGGFFAAKQEGRWLMDALFGAFCDLAVHRGYRVI |
| | KVDPYNTSRTCPECGHCDKANRDRVNREAFICVCCGYRGNADIDVAAYNIAMVAITGV |
| | SLRKAARASVASTPLESLAAE |
| SEQ ID: | MPKPAVESEFSKVLKKHFPGERFRSSYMKRGGKILAAQGEEAVVAYLQGKSEEEPPNFQ |
| 58 | PPAKCHVVTKSRDFAEWPIMKASEAIQRYIYALSTTERAACKPGKSSESHAAWFAATGV |
| | SNHGYSHVQGLNLIFDHTLGRYDGVLKKVQLRNEKARARLESINASRADEGLPEIKAEE |
| | EEVATNETGHLLQPPGINPSFYVYQTISPQAYRPRDEIVLPPEYAGYVRDPNAPIPLGVVR |
| | NRCDIQKGCPGYIPEWQREAGTAISPKTGKAVTVPGLSPKKNKRMRRYWRSEKEKAQD |
| | ALLVTVRIGTDWVVIDVRGLLRNARWRTIAPKDISLNALLDLFTGDPVIDVRRNIVTFTY |
| | TLDACGTYARKWTLKGKQTKATLDKLTATQTVALVAIDLGQTNPISAGISRVTQENGA |
| | LQ |
| | CEPLDRFTLPDDLLKDISAYRIAWDRNEEELRARSVEALPEAQQAEVRALDGVSKETAR |
| | TQLCADFGLDPKRLPWDKMSSNTTFISEALLSNSVSRDQVFFTPAPKKGAKKKAPVEVM |
| | RKDRTWARAYKPRLSVEAQKLKNEALWALKRTSPEYLKLSRRKEELCRRSINYVIEKTR |
| | RRTQCQIVIPVIEDLNVRFFHGSGKRLPGWDNFFTAKKENRWFIQGLHKAFSDLRTHRSF |
| | YVFEVRPERTSITCPKCGHCEVGNRDGEAFQCLSCGKTCNADLDVATHNLTQVALTGK |
| | TMPKREEPRDAQGTAPARKTKKASKSKAPPAEREDQTPAQEPSQTS |
| SEQ ID: | MSNKTTPPSPLSLLLRAHFPGLKFESQDYKIAGKKLRDGGPEAVISYLTGKGQAKLKDV |
| 59 | KPPAKAFVIAQSRPFIEWDLVRVSRQIQEKIFGIPATKGRPKQDGLSETAFNEAVASLEVD |
| | GKSKLNEETRAAFYEVLGLDAPSLHAQAQNALIKSAISIREGVLKKVENRNEKNLSKTK |
| | RRKEAGEEATFVEEKAHDERGYLIHPPGVNQTIPGYQAVVIKSCPSDFIGLPSGCLAKES |
| | AEALTDYLPHDRMTIPKGQPGYVPEWQHPLLNRRKNRRRRDWYSASLNKPKATCSKRS |
| | GTPNRKNSRTDQIQSGRFKGAIPVLMRFQDEWVIIDIRGLLRNARYRKLLKEKSTIPDLLS |
| | LFTGDPSIDMRQGVCTFIYKAGQACSAKMVKTKNAPEILSELTKSGPVVLVSIDLGQTNP |
| | IAAKVSRVTQLSDGQLSHETLLRELLSNDSSDGKEIARYRVASDRLRDKLANLAVERLSP |
| | EHKSEILRAKNDTPALCKARVCAALGLNPEMIAWDKMTPYTEFLATAYLEKGGDRKVA |
| | TLKPKNRPEMLRRDIKFKGTEGVRIEVSPEAAEAYREAQWDLQRTSPEYLRLSTWKQEL |
| | TKRILNQLRHKAAKSSQCEVVVMAFEDLNIKMMHGNGKWADGGWDAFFIKKRENRW |
| | FMQAFHKSLTELGAHKGVPTIEVTPHRTSITCTKCGHCDKANRDGERFACQKCGFVAH |
| | ADLEIATDNIERVALTGKPMPKPESERSGDAKKSVGARKAAFKPEEDAEAAE |
| SEQ ID: | MSKTKELNDYQEALARRLPGVRHQKSVRRAARLVYDRQGEDAMVAFLDGKEVDEPYT |
| 60 | LQPPAKCHILAVSRPIEEWPIARVTMAVQEHVYALPVHEVEKSRPETTEGSRSAWFKNS |
| | GVSNHGVTHAQTLNAILKNAYNVYNGVIKKVENRNAKKRDSLAAKNKSRERKGLPHF |
| | KADPPELATDEQGYLLQPPSPNSSVYLVQQHLRTPQIDLPSGYTGPVVDPRSPIPSLIPIDR |
| | LAIPPGQPGYVPLHDREKLTSNKHRRMKLPKSLRAQGALPVCFRVFDDWAVVDGRGLL |
| | RHAQYRRLAPKNVSIAELLELYTGDPVIDIKRNLMTFRFAEAVVEVTARKIVEKYHNKY |
| | LLKLTEPKGKPVREIGLVSIDLNVQRLIALAIYRVHQTGESQLALSPCLHREILPAKGLGD |
| | FDKYKSKFNQLTEEILTAAVQTLTSAQQEEYQRYVEESSHEAKADLCLKYSITPHELAW |
| | DKMTSSTQYISRWLRDHGWNASDFTQITKGRKKVERLWSDSRWAQELKPKLSNETRRK |
| | LEDAKHDLQRANPEWQRLAKRKQEYSRHLANTVLSMAREYTACETVVIAIENLPMKG |
| | GFVDGNGSRESGWDNFFTHKKENRWMIKDIHKALSDLAPNRGVHVLEVNPQYTSQTCP |
| | ECGHRDKANRDPIQRERFCCTHCGAQRHADLEVATHNIAMVATTGKSLTGKSLAPQRL |
| | QEAAE |
| SEQ ID: | VAFLDGKEVDEPYTLQPPAKCHILAVSRPIEEWPIARVTMAVQEHVYALPVHEVEKSRP |
| 61 | ETTEGSRSAWFKNSGVSNHGVTHAQTLNAILKNAYNVYNGVIKKVENRNAKKRDSLA |
| | AKNKSRERKGLPHFKADPPELATDEQGYLLQPPSPNSSVYLVQQHLRTPQIDLPSGYTGP |
| | VVDPRSPIPSLIPIDRLAIPPGQPGYVPLHDREKLTSNKHRRMKLPKSLRAQGALPVCFRV |
| | FDDWAVVDGRGLLRHAQYRRLAPKNVSIAELLELYTGDPVIDIKRNLMTFRFAEAVVE |
| | VTARKIVEKYHNKYLLKLTEPKGKPVREIGLVSIDLNVQRLIALAIYRVHQTGESQLALS |
| | PCLHREILPAKGLGDFDKYKSKFNQLTEEILTAAVQTLTSAQQEEYQRYVEESSHEAKA |
| | DLCLKYSITPHELAWDKMTSSTQYISRWLRDHGWNASDFTQITKGRKKVERLWSDSRW |
| | AQELKPKLSNETRRKLEDAKHDLQRANPEWQRLAKRKQEYSRHLANTVLSMAREYTA |
| | CETVVIAIENLPMKGGFVDGNGSRESGWDNFFTHKKENRWMIKDIHKALSDLAPNRGV |
| | HVLEVNPQYTSQTCPECGHRDKANRDPIQRERFCCTHCGAQRHADLEVATHNIAMVAT |
| | TGKSLTGKSLAPQRLQ |
| SEQ ID: | MIKKPSNRHALPKVIISKVDNQNILEFKIKYKKLSRLDRVEIKTMHYDDRAIVFDEVIING |
| 62 | GLIDVEYRDNHKTIFVKVGDKSYSISGQKVGGKERLLENRISQTKVQLELKDEATNRVS |
| | KTERELIVDDNIKLYSQIVGRDVKTTKDIYLIKRFLGYRSDLLFYYGFVNNFFHVANNRP |
| | EFWKIDFNDNRNSKLIEYFIFTINDHLKNDENYLKDYISDRGQIVDDLENIKHIFSALRHG |
| | LMHFDYDFFEALFNGEDIDIKMDNQGNTQPLSSLNIKFLDIMIDKLDKLNIDTKKEFIDAE |
| | KITIFGEELSLAKLYRFYAHTAINRVAFNKLINSFIIENGVENQSLKEYFNQQAGGIAYEID |
| | IHQNREYKNLYNEHKKLVSRVLSISDGQEIATLNQKIVELKEQMKQITKINSIKRLEYKLR |
| | LAFGFIYTEYKNYEEFKNSFDTDIKNGRFTPKDEDGNKRAFDSRELEHLKGYYKATLQT |
| | QKPQTDEKMEEVSKRVDRLSLKSLIGDDTLLKFILLMFTFMPQELKGEFLGFIKKYYHDT |
| | KHIDQDTISDSDDTIEEGLSIGLKLKILDKNIRSLSILKHSLSFQTKYNKKDRSYYEDGNIH |
| | GKFFKKLGISHNQEEFNKSVYAPLFRYYSALYKLINDFEIYTLSLHIVGNETLSDQVNKP |
| | QFLSGRYFNFRKLLTQSYNISNNSTHSVIFNAVINMRNDISHLSYEPLLDCPLNGKKSYKR |
| | KIRNQFRTINIKPLVESRKMIIDFITLQTDMQKVLGCDAVNDFTMKIVQLRTRLKAYANK |
| | EQTIEKMITEAKTPNDFYNIYKVKGVEAINKYLLEVIGETQVEKEIREEIERGNIANS |
| SEQ ID: | MLKKPSNRYALPKVILSTVDHEKILEFKVKYEKLARLDRLVVERMHEDGESVVFDEVIA |
| 63 | NSGDLEIAYQDDHRKLLIQAAGKSYTITGKKVGGKKRKLEERISRAKIQLTLTDGQEDQ |
| | HRRIRATVTEKALLEPKEDRDIYSKISDRKIKTSKEIYLVKRFLSYRSDLLFYYFFVDNFF |
| | KVGNNKQELWKIKFQNQPELIEYFRFIINDRFKNAKNDKFDNYLKNDKAIQEDLEKIQK |
| | VFEKLRHALMHYDYGFFEKLFGGEDQGFDLDIAFLDNFVKKIDKLNIDTKKEFVDDEKI |
| | KIFGEDLNLADLYKLYASISINRVGFNRVVNEMIIKDGIEKSELKRAFEKKLDKTYALDIH |
| | SDPSYKKLYNEHKRLVTEVSTYTDGNKIKEGNQKIAKLKYEMKEITKKNALVRLECKM |
| | RLAFGLIYGRYDTHEAFKNGFDTDLKRGEFAQIGSEEAIGYFNTTFEKSKPKSKEEIKKIA |
| | RQIDNLSLSTLIEDDPLMKFIVLMFLFVPRELKGEFLGFWRKYYHDIHSIDSDAKSDEMP |
| | DEVSLSLKLKILTRNIRRLNLFEYSLSEKIKYSPKNTQFYTDKSPYQKVYKRLKISHNKEE |
| | FDKTLLVPLFRYYSILFKLINDFEIYSLAKANPDASSLSELTKTKHGFRGHYNFTTLMMD |
| | AHKVSQGDSKKHFGIRGEIAHINTKDLIYDPLFRKSKMAQQRNDVIDFVLKYEKEIKAVL |
| | GYDAINDFRMKVVQLRTKLKVYSDKTQTIEKLLNEVEAPDDFYVLYKVKGVEAINKYL |
| | LEIVSVTQAEEEIERKIITGNKRYNT |
| SEQ ID: | MIKSYDDFTKLYSLQKTLRFELKPIGKTLEHIKKSEIIESDETLEEQYAIVKNIIDRFHRKHI |
| 64 | DEALSLVDFTKHLDTFKTIQELYLKRGKTDREKKELEELSADLRKLIVSYLKGNVKQKT |
| | QHNLNPIKERFEILFGKELFTNEEFFTLAENKEEKKAIQAFKGFTTYFKGFQENRKNMYS |
| | EEDKSTAIAYRIINENMPLFIENIARFQKVLDVIEKTKLTELKQNLKTELKGHSVSDIFRIE |
| | YFNNVLTQEGISRYNTIIGGKTTTEGVKIQGLNEIINLHNQQSKDVKLPILKPLHKQILSEE |
| | YSTSFKIKAFENDNEVLKAIDTFWNEHIEKSIHPVTGKRFNILLKIENLCKKLEKYKDKEI |
| | EKLFIERKNLSTVSHQVYGQWNIIRDALRMHLEMNNKNIKEKDIDKYLDNDAFAWKEI |
| | KDSIKIYKEHVEDAKELDENGIVKYFSSMSINEEDDEKEYSISLIKNINEKYNNVKSILEED |
| | RTGKSDLHQDKEKVAIIKEFLDSLKQLQWFLKLLYVTVPLDEKDYEFYNELEVYYEALL |
| | PLNSLYNKVRNYMTRKPYSVEKFKLNFYSPTLLDGWDKNKETANLSIILKKNGKYYLGI |
| | MNKENNTIFENFPKSKSNDYYEKMIYKLLPGPNKMLPKVFFSKKGLEYYKPSKEILRIYE |
| | KGEFKKDKSGNFKKESLHTLIDFYKEAIAKNEDWKIFKFKFKNTREYEDISQFYRDVEEQ |
| | GYLIIFEKVDANYVDKLVEEGELYLFQIYNKDFSENKKSKGNPNLHTIYWESLFDNQNL |
| | KDVVYKLNGEAEVFYRKKSIDYPEEIYNNGHHKEELNGKFNYPIIKDRRYTQDKFLFHV |
| | PITMNFISKEEKRVNQLACEYLSTTKEDVHIIGIDRGERHLLYLSLIDKEGNIKKQLSLNTI |
| | KNENYDKEIDYRVKLDEKEKKRDEARKNWDVIENIKELKEGYMSQVIHIIAKMMAEEK |
| | AILIMEDLNIGFKRGRFKVEKQVYQKFEKMMIDKLNYLVFKNKEPLEPGGSLNAYQLTS |
| | KFDSFKKLGKQSGFIFYVPSAYTSKIDPTTGFYNFIRVDVPNLEKGKEFFSKFEKIIYNTKE |
| | DYFEFHCKYGKFVPEPKNKDNDRKTKESLTYYNAIKDTVWVVCSTHHERYKIVRNKAG |
| | YYESQPVDVTKNLRDIFSEANINYSDGKDIKPIIIESNNAKLLKSIAEQLKLILAMRYNNG |
| | KHDDDEKDYILSPVKNKQGKFFCSLDGDQSLPINADANGAYNIALKGLLLIEKIKKQQG |
| | KAKDLYISNLEWFMFMMSR |
| SEQ ID: | MKSIFDNFTGLYSLSKTLRFELRPVGQTLENIKNGHFLESDKKMADDYQDVKKIIDNYH |
| 65 | KFFIDDVLKGASFDWALLEKELTDFNKNKTDDSKVEAEQKKLREQIAKTLAGDKRFKSL |
| | TASTPNDLFNKDKDFIGWLEQSSVKEIRKDALDTFKKFSSYFKGFQENRKNVYSADDIPT |
| | AVPYRIVNDNFPKFLQNISIFKTIQEKCPQVIADVENELASYLGKEKLADIFTVQAFNKYL |
| | CQGGKENQRGIDFYNQVIGGIAEKEGGVNLRGINQFLNLYWQQHPDFAKENRRIKMVP |
| | LYKQILSDRSSLSFKIETIDTDEELKTAISEYADKLESKSNDEKKSVLDVCVELFDSIKEQN |
| | LQEIYVNRKDINNISRILTGDWSWLQSRMNLYADEVFTTKAEKTRWQKSVDGDEGENK |
| | SKGAYSLAELNRVLEYASENVAETDIRITDYFCHRNRFYYEKESGLFKQGEELVALSIKE |
| | SCEDILSKRKAMNEAFANISESNSLRDNSEDIAKTKTYLDSVQDLLHRIKPLKVNGLGDP |
| | SFYAVFDSIYSALSEVISIYNKTRNYITRKAESPEKYKLNFDNPTLANGWDLNKEKDNTC |
| | VLLRKNGMYYLGIMNPKDKPKFAEKYDCGTESCYEKMIYKLLPGPNKMLPKVFFSTKG |
| | KKQYNPPENILHGYEQGKHKKGVAFDINFCHELIDWFKSAINQHEDWKKFGFKFSDTKS |
| | YKDISDFYREVTEQGYKLTFINIPESEISKMVSEGKLYLFQIYNMDFAPGANGMPNMHTL |
| | YWKNLFSEENLKDVVLKLNGEAELFYRPAGIKEPVVHAKGSYLVNRITKDGEPIPEKIH |
| | DEIYRNANGNLNNLSKEASEYKESHKVVIKQASHEIIKDRHYTEPKFLFHIPLTINFKAPS |
| | MKPTVCTAIQNENVRRFLKNNPDVNIIGLDRGERNLIYLSLINQKGEIIKQFSFNDVEREQ |
| | NGQTVKVNYHEKLDQREKERDAARKSWQAIGKIAELKEGYLSAVIHQLTKLMVEYNAI |
| | VVMEDLNFGFKRGRFHVEKQVYQKFEHMLIDKLNYLVFKDRGLNEPGGALNGYQLAG |
| | QFESFQKLGKQSGMLFYVPAAYTSKIDPKTGFVSMMNFKDLTNVHKKRDFFSKFEDIHF |
| | DEATCSFVFTFDYKNINGKAKEEMKQTKWAVYSREKRIVYFSKTKSSEDIMPTEKLRAL |
| | FESDGIEYKSGNNIHDSVMAVGADLKEGAKPSKEIADFWDGLLYNFKLILQMRNSNAKT |
| | GEDYIISPVMASDGTFFDSRVEAKKGKDAKLPLDADANGAYHIALKGLSLINKINLAGE |
| | DELKKFDMKISNEDWFNFAQEKKYAE |
| SEQ ID: | MTKQNKSVTQNTKRKNFGEFTNLYSLSKTLRFELKPVKATKTILEVERKDRENFKKDRK |
| 66 | IAKNYQKLKGILNELHQEFIQDVMREFSFQEKEIKEFEERYLEALNFKEKDNYKKRTQLK |
| | NAYEKVAKKLAGKIATAFGKYNQEKYGVKFTKKNLTGENVFDILEGKYKGDKKILGIIH |
| | TFKFKPTKEEKKQGKEAVNFSTYLGGFNQNRENFYKGEMKAGQFATRTIENLIQFLKNK |
| | KLFIDKYKDNYQKIGFSQEQVEIFNLNYFNNLFLQEGLDVYNGILGAKKGEKNTENDGL |
| | NQKINLFKQKEKTRCKANGEKFNKSDYPIFKELYKQIGSIKKDNDVYVEIKSDEELVNVL |
| | QSLPEKTANTLREVQKFYENFFDKIFNDEFDLDKIYLPKSVGTHFSHLAFSDWSKLAFVF |
| | NKRWRNEKVKIKEGEDVNVQSRSLADIKKRMEEILEMDGGVSFGKTYCQKVGLEKEAR |
| | TIEDVWSGFWKIIQYHINSQFIGGEKEVFDKEKKDDKTEKIQTIDDLQEEYLQATEMYRE |
| | RMVESEEGLNDGEEKEIKTKLKNYLDRIKDIERIARYFDLRKHFDDIDEASKDGDFYFIY |
| | QELLQDISEAKINDHYNEIRNYLTKANVVDDKFKLNFNDGQTLSGWDLNKETEKFSLIF |
| | KRKVDGGVEYYLGIINKEKNKTIFDKKKHPEIFTENSEFEKMEYKLFPSPSKMLPKIAFTK |
| | NKEGERIKPVFLDENAGKEIAQIKKEFALFQDAKKEDKNKWSDEFDRKKLNKLIDYYKL |
| | VLEKHPEKYMQTFNFVFKSSAKYKNLGEFNDDVARQNYVTKFVSVDKDYIDQKVESGE |
| | LYLFKIHNKDWNLTKAGDTKKQSKKNLHTIYFEELFSEKNIAEPVFKLSGGAEVFFRDAI |
| | EKKKQKKKKDKKGKEILEKFRFTKNKILFHVPITINYGKPSINQGQFNQKINEFIADNSRS |
| | VNILGIDRGEKHLLYYSLVDNNGKIIKSGSLNEINGVDYHEKLDKAEKERQEARKSWQK |
| | INQIKNLKAGYISQVIKKIVDLAIENNAIIVLEDLNFGFKSFRQKIEKNVYQQFEKALIDKL |
| | GFVTDKEKLNHRQAPQLSAPFESFEKMGKQTGIVFYVLATNTSKVCPQCQWKKNIFPHY |
| | STKKSIAENLQKQYKMKMYWRENENRFEFEYKGDGDKEFSSIFSNVDRVRYDKRANN |
| | NQGGYVIYQIDSTTKEKDGRNIKEKSITNLLKELLLEKFEIDNLEGELLVKLSEKSPDVSK |
| | ETIKDFFGLLNSILNIRNSMTDTEEDYIQCPACGFDTRKENKIGIKNGDDNGAYNIALRGR |
| | FLIERIKKAKKEDKKPNLTFSNNDYFQWVREFVK |
| SEQ ID: | MRTTTSLDAFTNRYALSKTLRFELKPIGNTQMMLEQNNVFAKDRAIREKYEKTKPWIDL |
| 67 | LHREFVAESLQNAQLGNLDDYYAALQNVQKITKDTNAEDKKRWKKGFEKQEKRLRKE |
| | VVALFDKAAHIWATQRYPQLKKKTKDFLFEEGVFEHVLFARYGSAPDTTVKIVTSNPET |
| | GEVIDEREESIFKGWKGFTGYFDKFFETRKNFYKDNGTATAIATRAINQNLRRFAENMQ |
| | KLTDIKNNYPELLAHTDFGDFDIAHAQSLDFYARTCLLQEGIDAYNKKFVGVLKSAINE |
| | YQQKNKGVRISYPKTLDNQILGERERRLFDVIEDDRELHDVFRAFVDDGTVFAAEMRQL |
| | AQAFSAQNGTYDYTQIYISKKGFETISRKYTHDTRAWHDALADVFKAKAKKRIATTASG |
| | EKKFPAYIPVAYITQALTLVQESEDTECTWKERYASITENKTLEEGFFAIFADEFERLFVH |
| | MEATVQDTDYVVAEDKAKKLLSDGQITKNEQTTQIIKEYADALLRIYQMAKYFAVEKK |
| | SMWDDAVAIDDTFYETFKEIYGNTHSTIVASYNLLRNYLTKKPWEDVQKWKLNFENPT |
| | LLDGWDKNKEAANFGVILRDGDKFYLGIMRKGHNNIFANQHHSNFEGQGLQKMVYKF |
| | FPDPKKMFPKVCFSAKGMEFFAPSEEIVRIYKNAEFKSGDTFNVESMQKLIDFYKNALQ |
| | KYDGWKIYDFKHLKDTAQYTSNIGEFYDDVAKGGYQLGWQNISKEYVEEKNANGELY |
| | LFQIKNKDWNDGATGRKNLHTLYFEYLFSEKNAAADFVFRLNGGAEVFYRPAAIESKTE |
| | RRGNREVAAKKRYTQDKVFLHVPITLNRTAGDVKTSAFNDAVNRFLAGNPDINIMGIDR |
| | GEKHLAYYSIIDQNGNRIVSGSFNTIGSKDYHALLTERQGAREEARKNWQRVEQIKDLK |
| | KGYISLVVREIADLAIKHNAIIVLENLNMRFKQIRGGIEKSVYQQLEKALIEKLNFLVNKG |
| | EVDATKAGHLLRAYQLAAPFETFEKMGNQTGIIFYTTASYTSQVDPVTGWRPHVYLKY |
| | RNAQKTKEDILRIFDDIVENDEKQRFEFAYRHNGVSWTVCSSVERHRWNRSNNAGKGG |
| | YDVFPVEGEGSITQRLQEACAQRGIDTTRNILAQIDELDESASATVSFLRDLCFYFRLICQI |
| | RNTDDGADDINAQDFLMSPVEPFFDTRNAQEQYPQNGDENGAYNIARKGIIILQKITAW |
| | GRSQDTQRRYPDTFVSQDEWDTFLTQHTT |
| SEQ ID: | MLKEKQFKTFGDFTNLYELSKTLRFELRPTPETKDLLDKNKIIQTDKKIAENYQEIKKYF |
| 68 | DKLHKKFIKEALSNTQIDFSDFCKLWEQNSKDSGKIKDLSRKLRKSIKQAFDKKGADWH |
| | KRYLEKGIKLKKKNLDILFEERVLDILKEEFKDDVDVKLFESFKGFSTYFTNFHESRKNF |
| | YKDDGTASAIATRIIDENLKRFCDNIKVKKHSKKLISELNEREAKIFEADFYNRCLLQQGI |
| | DDYNQVIGDINKKINNLRQNKIENTPTLKILYKQILGDVRRQETEQDAFIEIKNNEEVFDF |
| | LQDFIKHSDENNKYFKNLFYKFIEGKHSLDKIFLAKRFVNTISGKWFASWEVFGAELIKK |
| | FGNKKDLPDFIPFAAVKDVLQNCNIPANELFKEKIKNDEDKNIYDIFINLWKEEFDSNLK |
| | KVEESKKEVENMIAEDKVYSNKKEKRKNDNGEEIEIEIQKEKIKNYADAAMNIFRMMK |
| | YFLLEKNGKTVEGMGEDNNFYNELNIVFKGGEIDGKVYEGVKTYLYYNEFRNYLTKKP |
| | FNEEKTKLNFDCGQILSGWDKNKESEKLGVILRKDNKYYLAIINKKHNKIFDVKKNSYA |
| | YIVGDNFYEKMEYKLFPDAKRMIPKIAFAKNNKEKFGWTDEIQKIKNEYAEFQEGKKN |
| | DKNLWKDKFNKNKMEKLITYYQNCLEKGGYKDIYNFRWKSPDKYSGIGEFNDEIDRQS |
| | YCLKFVKVDFNYVFEKVKSGELYLFQIYNKDFSDKADRAQKENIHTEYFKLLFDQRNLD |
| | NVVLKLSGGAEIFYRPKTEGLPKKKDNKGNEVVRHRRYADDKYFLHLPIQLNFGRGNL |
| | SGGEFNSKINQYLSEQREIKIIGIDRGEKHLAYYSVINQDGKIEEIESLNTVNGIDYRKKLD |
| | ELEKKREQERKSWQSISKIKDLKKGYISHVIKKICDLAIEHNAIIVFEDLSGGFKNSRKKIE |
| | KQIYQNLELALATKLNYLTFKDKNFGESGHYLNAYQLAPKIDNYQDIKMQTGIVFYTPA |
| | GYTSSTCPQCGFRKTLKFDYTATISKAEDLIRGSKLNIVFEKEKNRFKINYLFNPIEKKKK |
| | KIKENELFADAGAKNEFTIYSDVKRIKWHNTGTKRLEEAEGERLLENKNSRGRDKEYDI |
| | NKCLTRLFRENKIDVNGDIIGQITKIKSLKLYQDLFYYLFLATLIRNNVSGSDIDYIQCPSC |
| | HFHSDGGFQKQKFNGDANGAYNIARKGILILKKIKQFAAQDKDMKNFGWKHLTVDINE |
| | WDKFTQK |
| SEQ ID: | MNKNFSNFTELYTLSKTLRFELKPVAQTKENIKKGKFLESDKKKADDYKDVKKIIDNYH |
| 69 | KFFIDDVLKNASFDWTVLEKEMSDFNKSKADDSKVEAEQKKLRDQIAKKLTSDKRFKA |
| | LTASTPSDLFNKDKDFIDWFTQNSTKDINKEALETFKRFSSYFKGFQENRKNVYSAEPIPT |
| | AVPYRLVNDNFPKFLQNIALFKIIQEKCPQVISDVEKELASYLGKEKLADIFTVQAFNKY |
| | LCQGGKENQRGIDFYNNILGGIAEKEGGINLRGINQFLNLYWQQHQDFAKQNKRIKMIP |
| | LFKQILSDRSSLSFKIESINTDQELLTSITEYADKLETKSNDEKKSVLDICSDLFASIKAQNL |
| | QEIYVNRKDINSISRILTGDWSWLQSRMNVYADEVFTTKAEKTRWQKSIDGDEGENKSK |
| | GVFSLAELNSVLEYSSENVSETDVRITDFFDHRNRFYYEKESGLFKQGDELVALSIRESCE |
| | DILAKRKAMDEAFANVSENNSLRDNSEDVAKIKIYLDCVQELLHRIKPLKVNGLGDPAF |
| | YAVFDTVYNSLSEVISLYNKTRNYITKKAANPEKYKLNFDNPTLADGWDLNKEQANTS |
| | VLMRKDGMYYLGIMNPKDKPKFAEKYECGNEACYEKMIYKQFDATKQIPKCSTQVKE |
| | VKKHFQSGATDSIILNDKSKFKLDLVITKEIWFLNNHVWNGEKFVPKRESNETRPKKFQI |
| | GYYKQTGDLGGYKEALNIWISFCKTFLQSYISSSIYDYDFKESSNYDSLDEFYNYLNATC |
| | YKLSFINIPEATISQMVSEGKLYLFQIYNKDFAPGASGMPNMHTLYWKNLFSEENLKNV |
| | VLKLNGEAELFYRPAGIKEPVIHAKGSYLVNRITKDGEPIPEKIHDEIYRNANGKLESLSK |
| | EATEYKASHKVVIKEAKHDIIKDRHYTEPKFLFHVPLTINFKASGNSYINENVRRFLKNN |
| | PDVNVIGLDRGERHLIYLSLINQKGEIIKQFTFNEVERNKNGQVIKVNYHEKLDQREKVR |
| | GAARKSWQAIGKIAELKEGYLSAVIHQLTKLMVEYNAIVVMEDLNFGFKRGRFHVEKQ |
| | VYQKFEHMLIDKLNYLVFKDRGLTEAGGVLNGYQLAGQFESFQKLGKQSGMLFYVPA |
| | GYTSKIDPKTGFASMSNFKDLTNVHKKRAFFSKFDDIHFDDATGSFVFTFDYKNFDGKA |
| | KEEMKRTKWSVYSKDKRIVYLSKTKSYEDVQPTEKIKASLESVGIEYMSGNNLIDSIMVI |
| | GAELKDGAKPSKEIADFWDRLLYNFKLILQMRNSNAKTGEDYIISPVMADDGTFFDSRE |
| | EFKKGENAKMPVDADANGAYHIALKGLSLLKRFDAASENELKKFDMKISNVDWFKFA |
| | QEKSYAE |
| SEQ ID: | MKAKKSFYNQKRKFGKRGYRLHDERIAYSGGIGSMRSIKYELKDSYGIAGLRNRIADAT |
| 70 | ISDNKWLYGNINLNDYLEWRSSKTDKQIEDGDRESSLLGFWLEALRLGFVFSKQSHAPN |
| | DFNETALQDLFETLDDDLKHVLDRKKWCDFIKIGTPKTNDQGRLKKQIKNLLKGNKRE |
| | EIEKTLNESDDELKEKINRIADVFAKNKSDKYTIFKLDKPNTEKYPRINDVQVAFFCHPDF |
| | EEITERDRTKTLDLIINRFNKRYEITENKKDDKTSNRMALYSLNQGYIPRVLNDLFLFVK |
| | DNEDDFSQFLSDLENFFSFSNEQIKIIKERLKKLKKYAEPIPGKPQLADKWDDYASDFGG |
| | KLESWYSNRIEKLKKIPESVSDLRNNLEKIRNVLKKQNNASKILELSQKIIEYIRDYGVSF |
| | EKPEIIKFSWINKTKDGQKKVFYVAKMADREFIEKLDLWMADLRSQLNEYNQDNKVSF |
| | KKKGKKIEELGVLDFALNKAKKNKSTKNENGWQQKLSESIQSAPLFFGEGNRVRNEEV |
| | YNLKDLLFSEIKNVENILMSSEAEDLKNIKIEYKEDGAKKGNYVLNVLARFYARFNEDG |
| | YGGWNKVKTVLENIAREAGTDFSKYGNNNNRNAGRFYLNGRERQVFTLIKFEKSITVE |
| | KILELVKLPSLLDEAYRDLVNENKNHKLRDVIQLSKTIMALVLSHSDKEKQIGGNYIHSK |
| | LSGYNALISKRDFISRYSVQTTNGTQCKLAIGKGKSKKGNEIDRYFYAFQFFKNDDSKIN |
| | LKVIKNNSHKNIDFNDNENKINALQVYSSNYQIQFLDWFFEKHQGKKTSLEVGGSFTIAE |
| | KSLTIDWSGSNPRVGFKRSDTEEKRVFVSQPFTLIPDDEDKERRKERMIKTKNRFIGIDIG |
| | EYGLAWSLIEVDNGDKNNRGIRQLESGFITDNQQQVLKKNVKSWRQNQIRQTFTSPDTK |
| | IARLRESLIGSYKNQLESLMVAKKANLSFEYEVSGFEVGGKRVAKIYDSIKRGSVRKKD |
| | NNSQNDQSWGKKGINEWSFETTAAGTSQFCTHCKRWSSLAIVDIEEYELKDYNDNLFK |
| | VKINDGEVRLLGKKGWRSGEKIKGKELFGPVKDAMRPNVDGLGMKIVKRKYLKLDLR |
| | DWVSRYGNMAIFICPYVDCHHISHADKQAAFNIAV |
| SEQ ID: | MKLTRRRISGNSVDQKITAAFYRDMSQGLLYYDSEDNDCTDKVIESMDFERSWRGRILK |
| 71 | NGEDDKNPFYMFVKGLVGSNDKIVCEPIDVDSDPDNLDILINKNLTGFGRNLKAPDSND |
| | TLENLIRKIQAGIPEEEVLPELKKIKEMIQKDIVNRKEQLLKSIKNNRIPFSLEGSKLVPSTK |
| | KMKWLFKLIDVPNKTFNEKMLEKYWEIYDYDKLKANITNRLDKTDKKARSISRAVSEE |
| | LREYHKNLRTNYNRFVSGDRPAAGLDNGGSAKYNPDKEEFLLFLKEVEQYFKKYFPVK |
| | SKHSNKSKDKSLVDKYKNYCSYKVVKKEVNRSIINQLVAGLIQQGKLLYYFYYNDTWQ |
| | EDFLNSYGLSYIQVEEAFKKSVMTSLSWGINRLTSFFIDDSNTVKFDDITTKKAKEAIESN |
| | YFNKLRTCSRMQDHFKEKLAFFYPVYVKDKKDRPDDDIENLIVLVKNAIESVSYLRNRT |
| | FHFKESSLLELLKELDDKNSGQNKIDYSVAAEFIKRDIENLYDVFREQIRSLGIAEYYKAD |
| | MISDCFKTCGLEFALYSPKNSLMPAFKNVYKRGANLNKAYIRDKGPKETGDQGQNSYK |
| | ALEEYRELTWYIEVKNNDQSYNAYKNLLQLIYYHAFLPEVRENEALITDFINRTKEWNR |
| | KETEERLNTKNNKKHKNFDENDDITVNTYRYESIPDYQGESLDDYLKVLQRKQMARAK |
| | EVNEKEEGNNNYIQFIRDVVVWAFGAYLENKLKNYKNELQPPLSKENIGLNDTLKELFP |
| | EEKVKSPFNIKCRFSISTFIDNKGKSTDNTSAEAVKTDGKEDEKDKKNIKRKDLLCFYLF |
| | LRLLDENEICKLQHQFIKYRCSLKERRFPGNRTKLEKETELLAELEELMELVRFTMPSIPE |
| | ISAKAESGYDTMIKKYFKDFIEKKVFKNPKTSNLYYHSDSKTPVTRKYMALLMRSAPLH |
| | LYKDIFKGYYLITKKECLEYIKLSNIIKDYQNSLNELHEQLERIKLKSEKQNGKDSLYLDK |
| | KDFYKVKEYVENLEQVARYKHLQHKINFESLYRIFRIHVDIAARMVGYTQDWERDMHF |
| | LFKALVYNGVLEERRFEAIFNNNDDNNDGRIVKKIQNNLNNKNRELVSMLCWNKKLNK |
| | NEFGAIIWKRNPIAHLNHFTQTEQNSKSSLESLINSLRILLAYDRKRQNAVTKTINDLLLN |
| | DYHIRIKWEGRVDEGQIYFNIKEKEDIENEPIIHLKHLHKKDCYIYKNSYMFDKQKEWIC |
| | NGIKEEVYDKSILKCIGNLFKFDYEDKNKSSANPKHT |
| SEQ ID: | MLRRDKEVKKLYNVFNQIQVGTKPKKWNNDEKLSPEENERRAQQKNIKMKNYKWRE |
| 72 | ACSKYVESSQRIINDVIFYSYRKAKNKLRYMRKNEDILKKMQEAEKLSKFSGGKLEDFV |
| | AYTLRKSLVVSKYDTQEFDSLAAMVVFLECIGKNNISDHEREIVCKLLELIRKDFSKLDP |
| | NVKGSQGANIVRSVRNQNMIVQPQGDRFLFPQVYAKENETVTNKNVEKEGLNEFLLNY |
| | ANLDDEKRAESLRKLRRILDVYFSAPNHYEKDMDITLSDNIEKEKFNVWEKHECGKKET |
| | GLFVDIPDVLMEAEAENIKLDAVVEKRERKVLNDRVRKQNIICYRYTRAVVEKYNSNEP |
| | LFFENNAINQYWIHHIENAVERILKNCKAGKLFKLRKGYLAEKVWKDAINLISIKYIALG |
| | KAVYNFALDDIWKDKKNKELGIVDERIRNGITSFDYEMIKAHENLQRELAVDIAFSVNN |
| | LARAVCDMSNLGNKESDFLLWKRNDIADKLKNKDDMASVSAVLQFFGGKSSWDINIFK |
| | DAYKGKKKYNYEVRFIDDLRKAIYCARNENFHFKTALVNDEKWNTELFGKIFERETEFC |
| | LNVEKDRFYSNNLYMFYQVSELRNMLDHLYSRSVSRAAQVPSYNSVIVRTAFPEYITNV |
| | LGYQKPSYDADTLGKWYSACYYLLKEIYYNSFLQSDRALQLFEKSVKTLSWDDKKQQR |
| | AVDNFKDHFSDIKSACTSLAQVCQIYMTEYNQQNNQIKKVRSSNDSIFDQPVYQHYKVL |
| | LKKAIANAFADYLKNNKDLFGFIGKPFKANEIREIDKEQFLPDWTSRKYEALCIEVSGSQ |
| | ELQKWYIVGKFLNARSLNLMVGSMRSYIQYVTDIKRRAASIGNELHVSVHDVEKVEKW |
| | VQVIEVCSLLASRTSNQFEDYFNDKDDYARYLKSYVDFSNVDMPSEYSALVDFSNEEQS |
| | DLYVDPKNPKVNRNIVHSKLFAADHILRDIVEPVSKDNIEEFYSQKAEIAYCKIKGKEITA |
| | EEQKAVLKYQKLKNRVELRDIVEYGEIINELLGQLINWSFMRERDLLYFQLGFHYDCLR |
| | NDSKKPEGYKNIKVDENSIKDAILYQIIGMYVNGVTVYAPEKDGDKLKEQCVKGGVGV |
| | KVSAFHRYSKYLGLNEKTLYNAGLEIFEVVAEHEDIINLRNGIDHFKYYLGDYRSMLSIY |
| | SEVFDRFFTYDIKYQKNVLNLLQNILLRHNVIVEPILESGFKTIGEQTKPGAKLSIRSIKSD |
| | TFQYKVKGGTLITDAKDERYLETIRKILYYAENEEDNLKKSVVVTNADKYEKNKESDD |
| | QNKQKEKKNKDNKGKKNEETKSDAEKNNNERLSYNPFANLNFKLSN |
| SEQ ID: | MIKSYDDFINHYAIQKTLRFELQPIGKTREHIQKNGIIEHDEALEQKYQIVKKIIDRFHRKH |
| 89 | IDEALSLVDFTKHLDTFKTIQELYLKRGKTDREKKELEELSADLRKRVVSFLEGKVEGD |
| | ARFAKVQQRYGILFDAKIFKDKEFISTACDDIEKDAIEAFKRFATYFTGFHENRKNMYSA |
| | DEESTAIAYRVINENLPRFLENKARFEKILDVIEKTKLTELKQNLKTELKGHSVSDIFRIEY |
| | FNNVLTQAGITYYNTILGGKTKENGEKVQGLNEIINLFNQKNKDTMLPLLKPLYKQILSE |
| | EYSTSFKIKAFENDNEVLKAIDTFWNEHIEKSIHPVTGKRFNILLKIENLCKKLEKYKDKE |
| | IEKLFIERKNLSTVSHQVYGQWNIIRDALRMHLEMNNKNIKEKDIDKYLDNDAFAWKEI |
| | KDSIKIYKEHVEDAKELDENGIVKYFSSMSINEEDDEKEYSISLIKNINEKYNNVKSILEED |
| | RTGKSDLHQDKEKVAIIKEFLDSLKQLQWFLKLLYVTVPLDEKDYDFYNELEMYHDTL |
| | LPLTTLYNKVRNYMTRKPYSVEKFKLTFEKSTLLDGWDKNKERANLGVILRKGNNYYL |
| | GIMNKKYNDIFDSIPGLTTTDYCEKMNYKLLPGPNKMLPKVFFSKKGVQFYKPSKEILRI |
| | YEKGEFKKDKSGNFKKESLHTLIDFYKEAIAKNEDWKIFKFKFKNTREYADISQFYKDV |
| | ERQGYKISFDKIDWEYILLLVDEGKLFLFKIYNKDFSPYSKGKPNLHTIYWKNIFSHDNL |
| | NNVVYKLNGEAEVFYRKKSIDYPEEIYNNGHHKEELNGKFNYPIIKDKRYAEDKFLFHV |
| | PITMNFISKEEKRVNQLACEYLSTTKEDVHIIGIDRGERHLLYLSLIDKEGNIKKQLSLNTI |
| | KNENYDKEIDYHAKLDEKEKKREEARKNWDVIENIKELKEGYLSQVVHQIAKLMVEYK |
| | AILVMEDLNTGFKRGRFKVEKQVYQKFEKMMIDKLNYLVLKDRQATQPGGSLKAYQL |
| | ASSLESFKKLGKQCGMIFYVPSAYTSKIDPTTGFYNFIRVDVPNLEKGKEFFSKFEKIIYN |
| | TKEDYFEFHCKYGKFVPEPKNKDNDRKTKESLTYYNAIKDTVWVVCSTHHERYKIVRN |
| | KAGYYESQPVDVTKNLRDIFSEANINYSDGKDIKPIIIESNNAKLLKSIAEQLKLILAMRY |
| | NNGKHDDDEKDYILSPVKNKQGKFFCSLDGDQSLPINADANGAYNIALKGLLLIEKIKK |
| | QQGKAKDLYISNLEWFMFMMSR |
| SEQ ID: | MIKSYDDFINHYAIQKTLRFELQPIGKTREHIQKNGIIEHDEALEQKYQIVKKIIDRFHRKH |
| 90 | IDEALSLVDFTKHLDTFKTIQELYLKRGKTDREKKELEELSADLRKLIVSYLKGNVKQKT |
| | QHNLNPIKERFEILFGKELFTNEEFFTLAENKEEKKAIQAFKGFTTYFKGFQENRKNMYS |
| | EEDKSTAIAYRIINENMPLFIENIARFQKVLDVIEKTKLTELKQNLKTELKGHSVSDIFRIE |
| | YFNNVLTQEGISRYNTIIGGKTTTEGVKIQGLNEIINLHNQQSKDVKLPLLKPLYKQILSE |
| | EYSTSFTISAFEKDNDVLQAIGSFCNDCIFYAKNNVNGKAYNLLQTVQAFCNSIDTYNDN |
| | RLDGLHIERKNLATLSHQVYGEWNILRDALQIHYEAYEQKDNGNNNNYLESKTFSWKA |
| | LKDALTTYKSLVEEAQDIDENGFIAYFKDMKFKEEIDGKTTSIDLIENIQTRYKSIETILQE |
| | DRNNKNNLHQEKEKVATIKGFLDSVKYLQWFLNLMYIASPVDDKDYDFYNELEMYHD |
| | TLLPLTTLYNKVRNYMTRKPYSVEKFKLTFEKSTLLDGWDKNKERANLGVILRKGNNY |
| | YLGIMNKKYNDIFDSIPGLTTTDYCEKMNYKLLPGPNKMLPKVFFSKKGVQFYKPSQEII |
| | RLYNNKEFKKGDTFNKNSLHKLINFYKESIAKTEDWSVFQFKFKNTNDYADISQFYKDV |
| | ERQGYKISFDKIDWEYILLLVDEGKLFLFKIYNKDFSPYSKGKPNLHTIYWKNIFSHDNL |
| | NNVVYKLNGEAEVFYRKKSIEYPEEILQKGHHVNELKDKFKYPIIKDKRYAEDKFLFHV |
| | PITMNFLSKEEKRVNQLACEYLSTTKEDVHIIGIDRGERHLLYLSLIDKEGNIKKQLSLNTI |
| | KNENYDKEIDYRVKLDEKEKKRDEARKNWDVIENIKELKEGYMSQVIHIIAKMMAEEK |
| | AILIMEDLNIGFKRGRFKVEKQVYQKFEKMMIDKLNYLVFKNKEPLEPGGSLNAYQLTS |
| | KFDSFKKLGKQSGFIFYVPSAYTSKIDPTTGFYNFIRVDVPNLEKGKEFFSKFEKIIYNTKE |
| | DYFEFHCKYGKFVPEPKNKDNDRKTKESLTYYNAIKDTVWVVCSTHHERYKIVRNKAG |
| | YYESQPVDVTKNLRDIFSEANINYSDGKDIKPIIIESNNAKLLKSIAEQLKLILAMRYNNG |
| | KHDDDEKDYILSPVKNKQGKFFCSLDGDQSLPINADANGAYNIALKGLLLIEKIKKQQG |
| | KAKDLYISNLEWFMFMMSR |
| SEQ ID: | MIKSYDDFINHYAIQKTLRFELQPIGKTREHIQKNGIIEHDEALEQKYQIVKKIIDRFHRKH |
| 91 | IDEALSLVDFTKHLDTFKTIQELYLKRGKTDREKKELEELSADLRKLIVSYLKGNVKQKT |
| | QHNLNPIKERFEILFGKELFTNEEFFTLAENKEEKKAIQAFKGFTTYFKGFQENRKNMYS |
| | EEDKSTAIAYRIINENMPLFIENIARFQKVLDVIEKTKLTELKQNLKTELKGHSVSDIFRIE |
| | YFNNVLTQEGISRYNTIIGGKTTTEGVKIQGLNEIINLHNQQSKDVKLPILKPLHKQILSEE |
| | YSTSFKIKAFENDNEVLKAIDTFWNEHIEKSIHPVTGKRFNILLKIENLCKKLEKYKDKEI |
| | EKLFIERKNLSTVSHQVYGQWNIIRDALRMHLEMNNKNIKEKDIDKYLDNDAFAWKEI |
| | KDSIKIYKEHVEDAKELDENGIVKYFSSMSINEEDDEKEYSISLIKNINEKYNNVKSILEED |
| | RTGKSDLHQDKEKVAIIKEFLDSLKQLQWFLKLLYVTVPLDEKDYEFYNELEVYYEALL |
| | PLNSLYNKVRNYMTRKPYSVEKFKLNFYSPTLLDGWDKNKETANLSIILKKNGKYYLGI |
| | MNKENNTIFENFPKSKSNDYYEKMIYKLLPGPNKMLPKVFFSKKGLEYYKPSKEILRIYE |
| | KGEFKKDKSGNFKKESLHTLIDFYKEAIAKNEDWKIFKFKFKNTREYEDISQFYRDVEEQ |
| | GYLIIFEKVDANYVDKLVEEGELFLFQIYNKDFSENKKSKGNPNLHTIYWESLFDNQNLK |
| | DVVYKLNGEAEVFYRKKSIDYPEEIYNNGHHKEELNGKFNYPIIKDRRYTQDKFLFHVPI |
| | TLNFLAKSDEKVNEMVKNYIAATNEKIHIIGIDRGERNLLYLSLIDSNGNIVKQQSLNIIEL |
| | PKYQKQIDYHAKLNEKEKORLAARQNWDVIENIKELKEGYLSQVIHQIARLMVDYKAIL |
| | VMEDLNFGFKRGRFKVEKQVYQKFEKMLIDKLSYLVFKEKNLCEPGGSLRAYQLSAPF |
| | KSFKALGKQSGMIFYVPAQYTSKIDPTTGFYNFLNIDVSNLARSKETFSKFDKIVYNKKE |
| | DYFEFYCKMINFESANQLTKKSQNKANAELKEFQWILCSTHHDRFKVERKNNQINYCKI |
| | NVNEELKKLLNSKGINYEKSNDLKSEILNIDESKFFKELGYLLKILVSLRYNNGKKGSEE |
| | QDFILSPVKNASGKFFCTLDNNNTLPLDADANGAYNIALKGLMIVQRVKAGGKLDLSIS |
| | KDDWINFLIMNKKLPK |

In some embodiments, target nucleic acid is an RNA and the CRISPR/Cas effector protein is an RNA-targeting CRISPR/Cas effector protein. In some embodiments, the RNA-targeting CRISPR/Cas effector protein comprises an amino acid sequence with at least 90% identity to the amino acid sequence of SEQ ID NO: 21, 62, 43, 41, or 42. In some embodiments, the RNA-targeting CRISPR/Cas effector protein comprises the amino acid sequence of SEQ ID NO: 21, 62, 43, 41, or 42.

In some embodiments, target nucleic acid is a DNA and the CRISPR/Cas effector protein is a DNA-targeting CRISPR/Cas effector protein. In some embodiments, the DNA-targeting CRISPR/Cas effector protein comprises an amino acid sequence with at least 90% identity to the amino acid sequence of SEQ ID NO: 3, 34, 57, 36, 65, 67, 68, 89, 90, 91, or 17. In some embodiments, the DNA-targeting CRISPR/Cas effector protein comprises the amino acid sequence of SEQ ID NO: 3, 34, 57, 36, 65, 67, 68, 89, 90, 91, or 17.

In some embodiments, effector proteins described herein have been modified (also referred to as an engineered protein). In some embodiments, a modification of the effector proteins may include addition of one or more amino acids, deletion of one or more amino acids, substitution of one or more amino acids, or combinations thereof. In some embodiments, effector proteins disclosed herein are engineered proteins. Unless otherwise indicated, reference to effector proteins throughout the present disclosure include engineered proteins thereof.

In some embodiments, effector proteins described herein can be modified with the addition of one or more heterologous peptides or heterologous polypeptides (referred to collectively herein as a heterologous polypeptide). In some embodiments, an effector protein modified with the addition of one or more heterologous peptides or heterologous polypeptides may be referred to herein as a fusion protein.

In some embodiments, a heterologous peptide or heterologous polypeptide comprises a protein tag. In some embodiments, the protein tag is referred to as purification tag or a fluorescent protein. The protein tag may be detectable for use in detection of the effector protein and/or purification of the effector protein. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6XHis tag; a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

A heterologous polypeptide may be located at or near the amino terminus (N-terminus) of the effector protein disclosed herein. A heterologous polypeptide may be located at or near the carboxy terminus (C-terminus) of the effector proteins disclosed herein. In some embodiments, a heterologous polypeptide is located internally in an effector protein described herein (i.e., is not at the N- or C-terminus of an effector protein described herein) at a suitable insertion site.

In some embodiments, an effector protein described herein comprises 1, 2, 3, 4, 5, 6, 7, 8, 9, 10, or more heterologous polypeptides at or near the N-terminus, 1, 2, 3, 4, 5, 6, 7, 8, 9, 10, or more heterologous polypeptides at or near the C-terminus, or a combination of these (e.g., one or more heterologous polypeptides at the amino-terminus and one or more heterologous polypeptides at the carboxy terminus). When more than one heterologous polypeptide is present, each may be selected independently of the others, such that a single heterologous polypeptide may be present in more than one copy and/or in combination with one or more other heterologous polypeptides present in one or more copies. In some embodiments, a heterologous polypeptide is considered near the N- or C-terminus when the nearest amino acid of the heterologous polypeptide is within about 1, 2, 3, 4, 5, 10, 15, 20, 25, 30, 40, 50, or more amino acids along the polypeptide chain from the N- or C-terminus.

In some embodiments of the methods disclosed herein, certain conditions are employed that enhance trans cleavage activity of an effector protein. In some embodiments, under certain conditions, trans cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments of the methods disclosed herein, certain conditions are employed that enhance cis cleavage activity of an effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, or a magnesium salt. In some embodiments, the salt is NaCl, KCl, or MgCl₂. In some embodiments, the salt is KNO₃. In some embodiments, the salt concentration is less than 150 mM, less than 125 mM, less than 100 mM, less than 75 mM, less than 50 mM, less than 25 mM, less than 10 mM, or less than 5 mM.

Certain conditions that may enhance the activity of an effector protein include the pH of a solution in which the activity. For example, increasing pH may enhance trans cleavage activity. For example, the rate of trans cleavage activity may increase with increase in pH up to pH 9 in at least some settings. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 70° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., about 50° C., about 55° C., about 60° C., about 65° C., or about 70° C.

In some embodiments, effector proteins described herein have been modified (also referred to as an engineered protein or engineered polypeptide). In some embodiments, a modification of the effector proteins may include addition of one or more amino acids, deletion of one or more amino acids, substitution of one or more amino acids, or combinations thereof. In some embodiments, effector proteins disclosed herein are engineered proteins. Unless otherwise indicated, reference to effector proteins throughout the present disclosure include engineered proteins thereof.

In some embodiments, engineered effector proteins as described herein comprise one or more amino acid modifications relative to cognate effector protein (e.g., a modification as exemplified when comparing to an effector protein having any one of the amino acid sequences recited in TABLE 1 to the cognate effector protein), and wherein the engineered effector protein exhibits one or more improved characteristics compared to the cognate effector protein (e.g., a naturally occurring effector protein). In some embodiments, a cognate effector protein, as described herein, refers to a naturally occurring effector protein that may be used as the parental effector protein sequence for protein engineering. In some embodiments, the naturally occurring effector protein comprises certain characteristics (e.g., structure and/or activity) that may be of interest for protein engineering.

In some embodiments, the complex comprising the engineered polypeptide and an engineered guide nucleic acid comprises increased stability as compared to a complex comprising the cognate effector protein and an engineered guide nucleic acid. In some embodiments, the one or more improved characteristics of the engineered effector protein compared to the cognate effector protein are selected from: increased catalytic activity at a temperature above 37° C.; decreased catalytic activity at a temperature below 37° C.; increased catalytic activity at a defined salt concentration; increased cleavage rate of target nucleic acid; increased trans cleavage rate; increased formation of a complex comprising the engineered polypeptide and an engineered guide nucleic acid; increased solubility; increased manufacturability (e.g., increased expressibility, solubility, purification, etc.), and increased stability.

Effector proteins of the present disclosure can be engineered, using any suitable protein engineering method known in the art. Examples of suitable protein engineering methods are described herein. Suitable protein engineering methods can include a method of using mutagenesis to generate a novel nucleic acid encoding a novel effector protein or novel polypeptide, which novel effector protein is itself a modified biological molecule and/or contributes to the generation of another modified biological molecule as compared to wild-type equivalents. Protein engineering methods can be geared towards maintaining certain existing protein functions while modifying others (e.g., maintaining binding activity to a guide nucleic acid, while modifying nuclease activity or specificity), increasing existing protein function, gaining a novel protein function, improving the stability of a protein under certain conditions, improving function in different environments, such as, for example, high temperature and/or high salt, or combinations thereof. Suitable protein engineering methods can include, but are not limited to, random mutagenesis, focused mutagenesis, or methods that integrate both random and focused mutagenesis. In some embodiments, effector proteins can be engineered in vitro or in vivo by eukaryotic cells or by prokaryotic cells.

Random mutagenesis engineering methods can generate random point mutations at codons corresponding to specific structurally characterized residues (e.g., protein residues involved in binding or catalysis, such as, for example, catalytic residues in RuvC nuclease active site). Although protein engineering by methods such as directed evolution via repeated random mutagenesis (e.g., random chemical or error prone PCR (epPCR)) and selection can yield engineered proteins with desirable characteristics, some protein engineering efforts require more specificity. For example, protein engineering methods which require mutation of more than one nucleotide relative to a non-modified codon can require focused mutagenesis, which can introduce specific amino acid substitutions at positions corresponding to targeted nucleotide(s) or targeted residue(s). Focused mutagenesis can employ a synthetic nucleic acid, such as a synthetic DNA oligonucleotide comprising one or more modifications, which can also be referred to as a mutagenic oligonucleotide. The mutagenic oligonucleotide, which can be incorporated into a gene library as a mutagenic cassette, can comprise modified/degenerate codons corresponding to targeted residues. Focused mutagenesis can also yield more functional variations, beneficial mutations, or modifications resulting in the desired engineered protein activity while minimizing neutral or deleterious mutations.

Effector proteins can be engineered in vitro or in vivo by focused and/or random mutagenesis methods, such as chemical mutagenesis, combinatorial libraries, computational strategies for high-quality library design, homologous recombination, non-homologous recombination, recombination based methods such as DNA shuffling (e.g., molecular breeding), directed evolution, deletion mutagenesis, error prone PCR (epPCR), insertion mutagenesis, random mutagenesis, scanning mutagenesis, site-directed mutagenesis (SDM) (and similar methods such as: site-specific mutagenesis, oligonucleotide-directed mutagenesis, site-saturation mutagenesis (SSM)), use of mutator strain, assembly PCR, sexual PCR mutagenesis, cassette mutagenesis, recursive ensemble mutagenesis, exponential ensemble mutagenesis, site-specific mutagenesis, gene reassembly, gene site saturation mutagenesis (GSSM), synthetic ligation reassembly (SLR), recombination, replacing codon(s) encoding the same amino acid, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, other mutagenesis methods described herein, or combinations thereof (See, e.g., Packer, M., et al., Nature Reviews Genetics, 16 (7): 379-94 (2015)).

In vivo mutagenesis methods can be focused, random, or combinations thereof. In vivo focused mutagenesis methods can comprise selectively introducing localized DNA damage into a genome, such as, for example, targeting a pathway requiring long-range resection so as to form a single-stranded region during biasing repair and selectively mutating said single-stranded region. In some embodiments, in vivo focused mutagenesis methods comprise delivering a nucleic acid encoding an effector protein and a guide nucleic acid to a cell, and contacting the cell with a mutator compound or mutator enzyme. In some embodiments, in vivo focused mutagenesis methods comprise selectively introducing localized DNA damage in a preselected region of an organism's DNA in vivo, biasing repair of the localized DNA damage by targeting a pathway requiring long-range resectioning of the localized DNA damage, wherein the DNA forms a single-stranded region during the biasing repair, and selectively mutating the single stranded region to cause targeted mutagenesis, optionally wherein the organism is an eukaryotic organism. In some embodiments, localized DNA damage is a double stranded break (DSB). In some embodiments, a DSB is introduced by a DNA mutator enzyme domain (e.g., DNA glycosylase, 3-methyladenine glycosylase (e.g., Maglp), DNA nuclease, Fokl). In some embodiments, biasing repair of the DSB involves contacting the cell with a compound that elicits DNA damage checkpoint activation. In some embodiments, the compound that elicits DNA damage checkpoint activation is a chemical checkpoint activator (e.g., methyl methanesulphonate (MMS)), or an enzymatic checkpoint activator (e.g., Magi). In vivo random mutagenesis methods (i.e., traditional genetic screens) can randomly damage DNA via chemical and/or physical agents such as, for example, alkylating compounds (e.g., ethyl methanesulfonate (EMS)), deaminating compounds (e.g., nitrous acid), base analogues (e.g., 2-aminopurine), radiation (e.g., ultraviolet irradiation), bisulfite, or combinations thereof. In some embodiments, random chemical mutagenesis can facilitate dose-dependent modification or mutation of DNA. In some embodiments, random chemical mutagenesis, which has a broad mutational spectrum, can be used to randomly deactivate genes for a genome-wide screen in vivo or in vitro. In some embodiments, random mutagenesis enhances the error rate during DNA replication, which can lead to off-target mutations and/or deleterious genome mutations.

Random mutator strain mutagenesis, an in vivo random mutagenesis method, can produce randomly mutagenized plasmid libraries upon propagation of the genes cloned in plasmids through a mutator strain, like Escherichia coli XL1-red. In brief, random mutator strain mutagenesis is a method for introducing random point mutations throughout a gene encoding a protein of interest with the use of a plasmid. The method involves transformation and propagation of a plasmid containing the target gene into a mutator strain, isolating the resulting randomly mutagenized plasmid library, transforming the library into a strain comprising the mutant target gene, and screening the mutant target gene phenotype. In some embodiments, the method elicits random mutagenesis via phage-assisted continuous evolution (PACE), a method which harnesses the phage virus bacterial infection cycle to generate multiple rounds of DNA sequence mutations, selecting for DNA mutations in a mutant target gene encoding a protein that result in a desired protein structure or activity. In some embodiments, random mutagenesis involves yeast orthogonal replication. Although the methods generally offer ease of use, host intolerance to a high degree of genomic mutation(s) can place an upper limit on in vivo mutagenesis rates. In vitro random mutagenesis methods generally offer protein engineering methods with higher target mutation rates as compared to most in vivo random mutagenesis methods.

Homologous recombination, a random mutagenesis method which can be carried out in vivo or in vitro, can lead to DNA modification, damage, or repair upon DNA shuffling, family shuffling, staggered extension process (StEP), random chimeragenesis on transient templates (RACHITT), nucleotide exchange and excision technology (NexT), heritable recombination, assembly of designed oligonucleotides (ADO), synthetic shuffling, or combinations thereof. For example, StEP is a modified PCR that uses highly abbreviated annealing and extension steps to generate staggered DNA fragments and promote crossover events along the full length of the template sequence(s), such that most of the resulting polypeptides comprise sequence information from different template sequence(s). RACHITT performs molecular mutagenesis at a high recombination rate by aligning parental gene fragments on a full-length DNA template, which are then stabilized on the template by a single long annealing step at a relatively high ionic strength. RACHITT can yield a considerable number of crossovers per gene in a single annealing step. NexT is also a modified PCR that uses uridine triphosphate (dUTP) as a DNA fragmentation defining exchange nucleotide with thymidine. In NexT, the exchange nucleotides are removed enzymatically, followed by chemical cleavage of the DNA backbone. Finally, the oligonucleotide pool is reassembled into full-length genes by internal primer extension, and the recombined gene library is amplified by standard PCR. Another modified PCR, ADO, is a two-step reaction involving an overlap extension PCR step using synthetic oligonucleotides followed by a PCR amplification step using outer primers, resulting in double-stranded DNA assembled with engineered gene fragments. In some embodiments, homologous recombination (HR) methods can lead to DNA modification comprising mutations generated by knocking out or removing one or more nucleotides. In some embodiments, HR methods repair gene function by identifying sequence homology and replicating the functional version of the target gene. In some embodiments, knock out mutations result in functional modifications to the protein encoded by the modified nucleic acid sequence. HR can prove advantageous in its ability to identify beneficial mutation combinations, eliminate passenger mutations, shuffle functional sequences of orthologous proteins, or combinations thereof.

Error prone PCR (epPCR) mutagenesis, an in vitro random mutagenesis method, can result in the modification/damage of DNA via PCR amplification involving supplemental mixture components such as, for example, proprietary enzyme mixes (e.g., Mutazyme), Taq supplemented with Mg²⁺, Taq supplemented with Mn2+ and/or unequal dNTPs, or combinations thereof. EpPCR involves the modification of DNA or creation of a mutation during PCR amplification of a target gene, a fragment of a target gene, a target sequence, a DNA sequence, or combinations thereof. In some embodiments, the low fidelity of DNA polymerases under certain conditions generates point mutations during PCR amplification of a gene of interest. In some embodiments, the base-pairing fidelity of DNA polymerases can be reduced with increased magnesium concentrations (e.g., Taq supplemented with Mg2+), supplementation with manganese (e.g., Taq supplemented with Mn2+), the use of mutagenic dNTP analogues (e.g., unequal/unbalanced dNTPs), or the use of proprietary enzyme mixes (e.g., Mutazyme) to increase mutation rates (e.g., 10⁻⁴˜10⁻³ per replicated base). Given that each cycle of PCR amplification leads to the accumulation of mutations, high mutation rates (e.g., high number mutations per clone) can be achieved by increasing the number of PCR amplification cycles. EpPCR offers advantages, such as, for example, its tendency for high mutation rates and/or a relatively even mutation spectrum, as well as easy to use commercial formulations. Optionally, a more ideal nucleotide mutational spectrum can be achieved via sequence saturation mutagenesis (SeSaM), a mutagenesis method that randomizes a target sequence at every single nucleotide position. Briefly, SeSaM is a chemo-enzymatic random mutagenesis method which involves the enzymatic insertion of a base, such as the universal base deoxyinosine (2′-deoxyInosine (dI)), throughout the target gene.

Suitable applications of epPCR include, but are not limited to, the generation of neutral drift libraries, which can be used to identify an evolvable starting point for protein engineering (e.g., the directed evolution of a target protein of interest). Generating a neutral drift library can involve exploring accessible sequence space by repeated rounds of mutagenesis and selection for the accumulation of mutations that are largely neutral and compatible with maintaining wild-type function. Mutations that are largely neutral for the wild-type protein function accumulate, while mutations detrimental to the wild-type protein function are purged, yielding a library of high diversity and quality. Specifically, a target gene is mutagenized by epPCR, fused to a reporter nucleic acid (e.g., GFP reporter), and the mutagenized gene variants are then screened for target protein expression. After multiple rounds of mutagenesis and screening, the resulting neutral drift library exhibits sequence diversity that does not destabilize protein structure or protein function. Screening for target protein expression ensures the resulting neutral drift library mostly lacks non-target deleterious mutations.

Another in vitro method for generating high-quality libraries is site-directed saturation mutagenesis (SDSM). SDSM and similar methods such as site-directed mutagenesis (SDM), site-saturation mutagenesis (SSM), site-specific mutagenesis, or oligonucleotide-directed mutagenesis, are in vitro focused mutagenesis methods, capable of fullysampling the amino acid repertoire, focusing on functionally relevant residues, and/or increasing library quality. In some embodiments, SDSM involves NNK and NNS codons (where N can be any of the four nucleotides, K can be G or T, and S can be G or C) on mutagenic primers. SDM, which is commonly applied to study the function of a single amino acid in relation to the rest of the protein, involves the substitution of a single amino acid for another, usually an alanine. In some embodiments, site-directed mutagenesis is performed via means that are synthetic, where the design of the engineered/desirable/target/progeny polynucleotide(s) is derived by analysis of a wild-type/parental set of proteins and/or of the polypeptides correspondingly encoded by the wild-type/parental proteins. SSM, which is a similar method to SDM, involves the substitution of a single amino acid for another, usually for any of the other 19 standard amino acid substituents other than alanine. Thus, the SSM mutagenesis product is a collection of clones, each having a different codon in the targeted position (i.e., saturated), yielding all possible substitutions. Analysis of the SSM mutagenesis product can indicate the relationship between the targeted amino acid positions and protein function. In some embodiments, site-specific protein engineering methods, such as SSM, target the diversification of functionally relevant residues, some of which may not be comprised in the protein's primary structure. In some embodiments, simultaneous SSM of, for example, multiple target residues, can result in combinations of mutations that can exhibit synergistic or epistatic interactions. Combinations of mutations exhibiting epistatic interactions (e.g., sign epistasis, a type of interaction in which mutations can be individually non-desirable/deleterious, but confer gain-of-function in combination) can be selected for with the use of simultaneous SSM. In some embodiments, simultaneous SSM targets combinations of mutations exhibiting synergistic interactions (e.g., a type of interaction in which mutations in combination have a greater effect as compared to the sum of the effects of each individual mutation) with desirable/target effects. Overall, a site-saturation library can result from sequential enrichment of epistatic mutation combinations, sequential enrichment of synergistic mutation combinations, sequential enrichment of functionally relevant mutations, sequential enrichment of functionally relevant residues, or combinations thereof. Site-specific mutagenesis or oligonucleotide-directed mutagenesis involves the modification of DNA or creation of an intentional mutation at a specific location on the oligonucleotide sequence. Modification of DNA or creation of an intentional mutation can involve insertional mutagenesis and/or deletion mutagenesis. Insertional mutagenesis can involve the incorporation of a mutation into a target gene via the incorporation of a few nucleotides (e.g., insertional mutagenesis via conventional PCR, nested PCR, or similar techniques). Deletion mutagenesis can involve the removal of a target gene, a fragment of a target gene, a target sequence, a DNA sequence, a few nucleotides, or combinations thereof (e.g., deletion mutagenesis via inverse PCR, or a similar technique). Site-specific mutagenesis or oligonucleotide-directed mutagenesis can involve amplifying a gene of interest via PCR with the use of a synthetic primer possessing a specific mutation or a target mutation, which can result in a deletion, insertion, or single nucleotide polymorphism (SNP), as confirmed by sequencing. In some embodiments, oligonucleotide-directed mutagenesis involves the replacement of a short sequence with a synthetically mutagenized oligonucleotide. In brief, a synthetically mutagenized oligonucleotide, can comprise one or more modifications, such as, for example, modified codon(s) corresponding to targeted residue(s). Mutagenesis with synthetic oligonucleotides requires sequencing of individual clones after each selection round, grouping individual clones into families, arbitrarily choosing a single family, and reducing the chosen family to a consensus motif. The consensus motif is resynthesized and reinserted into a single gene for additional selection. Oligonucleotide-directed mutagenesis can be best suited for fine-tuning sequence areas of comparatively low information content. Cassette mutagenesis, a type of SDM, uses a short, double-stranded oligonucleotide sequence (e.g., a gene cassette) to replace a fragment of target DNA such that, a sequence block of a single template is typically replaced by a (partially) randomized sequence (e.g., a mutagenic cassette, which can be a mutagenic oligonucleotide).

Computational strategies, an in vitro focused mutagenesis method for high-quality library design, can involve one or more of Rosetta design, computationally guided libraries, incorporating synthetic oligonucleotides via gene reassembly (ISOR), consensus design, reconstructed evolutionary adaptive path (REAP) analysis, and SCHEMA algorithm(s). The method offers an advantage in the form of creating small libraries pre-enriched for functional variation by natural selection and/or in silico filtering. Consensus design (a method which involves the identification of common ancestral mutations (i.e., evolutionary history) by aligning all sequences and identifying the most frequently observed amino acid(s) at each position in the sequence alignment) can lead to the introduction of consensus mutations or significantly distinct/divergent mutations, yielding engineered proteins with improved thermostability, catalytic stability, enzymatic efficiency, or combinations thereof. In contrast, reconstructed evolutionary adaptive path (REAP) analysis provides a method for the identification of significant mutational divergence, which can (i) comprise mutational signatures related to known protein function(s) or protein pathway characteristics, or which can (ii) be used to predict changes in protein function(s) as related to, for example, structural proximity to an active site. In some embodiments, a protein engineering method, incorporating synthetic oligonucleotides via gene reassembly (ISOR), can be used to predict desirable protein engineering outcomes, such as, for example, the introduction of mutations that can improve protein stability and/or protein folding. ISOR, a versatile combinatorial method for the partial diversification of large sets of protein residues or targeted protein positions, offers a method to select target engineered proteins capable of desirable/target activity/properties. As compared to site-specific methods of diversification, ISOR can prove more efficient in identifying target protein positions related to target protein activity, while building a reasonably sized protein library for protein engineering. Briefly, ISOR incorporates synthetic oligonucleotides comprising randomized codons flanked by wild-type sequences to wild-type gene fragments via assembly PCR. The resulting reassembled gene comprises randomized cassettes (e.g., mutagenic cassettes) at target sites. As a factor of oligonucleotide concentration, the resulting reassembled gene comprises semi-randomly introduced mutations, such that resulting variants can comprise a different quantity and/or combination of mutated positions. In some embodiments, randomly introduced mutations can comprise a random subset of the resulting mutations. In some embodiments, ISOR is used to create libraries focused on the randomization of individual positions of interest, on the identification of proteins comprising combinations of mutated residues while maintaining, upregulating, or downregulating wild-type protein function, and/or on the identification of proteins comprising combinations of mutated residues while gaining a desirable protein function. In some embodiments, ISOR is used to create libraries characterizing protein function as related to insertions and/or deletions in sequence positions surrounding an active site of interest.

Computational strategies or computational modelling, as described herein, can facilitate the identification of specific amino acid substitution/modification as related to desired/target engineered protein activity/function. Computational strategies for high-quality library design, can involve, for example, the use of computational algorithms such as SCHEMA and/or Rosetta. Briefly, SCHEMA provides a method for identifying protein fragments and designing novel proteins by recombination of homologous sequences. For example, SCHEMA identifies interacting amino acid residue pairs via structural information, accounting for amino acid residue pair interactions that are broken upon recombination, and predicting which elements in homologous sequences/proteins can be swapped without disturbing the integrity of the protein structure. Briefly, Rosetta is a computational modeling software comprising algorithms which can be used to design methods for protein engineering based on protein structure analysis, such as, for example, protein structure prediction, protein structure refinement, protein conformation, protein docking, functional protein design, and combinations thereof. Rosetta models can be employed to adapt protein engineering methods to specific applications, such as, for example, protein-protein docking interaction/activity of engineered protein(s). Rosetta models can also be employed to consider protein folding, translation, rotation, association, amino acid sequence design, molecular structure interactions, degrees of freedom (DOFs), electrostatic interactions, hydrogen bonding, hydrophobic interactions, or combinations thereof. In some embodiments, Rosetta models can facilitate the design of a protein engineering method to optimize protein sequences (including, for instance, suggesting a single base change) for engineering protein(s) capable of a target protein conformation. In some embodiments, Rosetta models are geared towards maintaining existing protein function, increasing existing protein function, gaining a novel protein function, improving the stability of protein function, improving function in different environments, such as, for example, high temperature and/or high salt, or combinations thereof. In some embodiments, Rosetta's design models can be employed to identify mutations that improve engineered protein stability and binding affinity.

Non-homologous recombination is an in vitro focused mutagenesis method which can lead to DNA modification, damage, or repair upon incremental truncation for the creation of hybrid enzymes (ITCHY), sequence homology-independent protein recombination (SHIPREC), nonhomologous random recombination (NRR), sequence-independent site-directed chimeragenesis (SISDC) and overlap extension PCR. For example, ITCHY is a recombination method capable of generating a single-crossover hybrid library based on generation of N- or C-terminal fragment libraries of two genes by progressive truncation of the coding sequences by an exonuclease followed by ligation. Thus, ITCHY allows the creation of hybrid libraries between fragments of genes without any sequence dependency. SHIPREC is a recombination method capable of generating single-crossover hybrid libraries of unrelated or distantly related proteins by maintaining sequence alignment between the parent sequences and introducing crossovers mainly at structurally related sites distributed over the aligned sequences. NRR is a recombination method that enables nucleic acid or DNA fragments to randomly recombine in a length-controlled manner at sites where there is little or no sequence homology. SISDC is a recombination method that enables the recombination of distantly related (or unrelated) proteins at multiple discrete sites, such as sites related to protein function. In some embodiments, non-homologous recombination (NHR) can lead to the recombination of portions of nucleic acid(s) at sites with low or no sequence homology. Thus, NHR can increase the frequency at which novel modified nucleic acid sequences are generated, yielding a more efficient and/or complete exploration of nucleic acid or protein diversity, as compared to HR. NHR can prove advantageous in its capacity to shuffle distantly related sequences, rearrange gene order, rearrange nucleic acids comprising low information content, or combinations thereof.

In some embodiments, the methods for protein engineering can comprise generating a nucleic acid encoding a polypeptide comprising a mutation or modification (e.g., deleting or adding one or more nucleotides, or a combination thereof) wherein the methods for introducing the mutation or modification comprise any of the protein engineering methods disclosed herein. In some embodiments, the method for protein engineering further comprising expressing nucleic acid comprising a mutation or modification to generate a polypeptide comprising a mutation or modification. In some embodiments, the methods described herein comprise repeating the method for protein engineering until the desired modification or mutation is achieved.

In some embodiments, the methods for protein engineering can further comprise a screening step, an assaying step, an isolation step, a purification step, or combinations thereof. In some embodiments, the engineered effector proteins can be further processed by unfolding (e.g., heat denaturation, dithiothreitol reduction, etc.) and can be further refolded, using any suitable method.

In some embodiments, effector proteins may be engineered to improve thermostability, catalytic stability, enzymatic efficiency, or combinations thereof.

### Guide Nucleic Acids

In the methods disclosed herein, a guide nucleic acid, as well as any components thereof (e.g., spacer sequence, repeat sequence, linker nucleotide sequence, handle sequence, intermediary sequence etc.) may comprise one or more deoxyribonucleotides, ribonucleotides, biochemically or chemically modified nucleotides (e.g., one or more engineered modifications as described herein), or any combinations thereof. Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which can be a nucleotide sequence for use in a guide nucleic acid as described herein.

A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). A guide nucleic acid may be chemically synthesized or recombinantly produced by any suitable methods. Guide nucleic acids and portions thereof may be found in or identified from a CRISPR array present in the genome of a host organism or cell.

In general, a guide nucleic acid comprises a first region that is not complementary to a target nucleic acid (FR) and a second region is complementary to the target nucleic acid (SR). In some embodiments, FR is located 5′ to SR (FR-SR). In some embodiments, SR is located 5′ to FR (SR-FR). In some embodiments, the FR comprises one or more repeat sequences or intermediary sequence. In some embodiments, an effector protein binds to at least a portion of the FR. In some embodiments, the SR comprises a spacer sequence, wherein the spacer sequence can interact in a sequence-specific manner with (e.g., has complementarity with, or can hybridize to a target sequence in) a target nucleic acid.

The guide nucleic acid may also form complexes as described through herein. For example, a guide nucleic acid may hybridize to another nucleic acid, such as target nucleic acid, or a portion thereof. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by hybridizing to different target sequences. In some embodiments, a first guide nucleic acid may hybridize within a location of the target nucleic acid that is different from where a second guide nucleic acid may hybridize the target nucleic acid. In some embodiments, the first loci and the second loci of the target nucleic acid may be located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart.

In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, signal, and the like. In some embodiments, a target sequence is a eukaryotic sequence.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

Guide nucleic acids described herein may comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to at least about 25, or at least about 15 to about 25 linked nucleotides. In some embodiments, the spacer sequence comprises 15-28 linked nucleotides. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, the spacer sequence comprises 10, 11, 12, 13, 14, 15, 16, 17, 18, 19, 20, 21, 22, 23, 24, 25, 26, 27, 28, 29, 30, or more nucleotides.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence is capable of hybridizing to an equal length portion of a target nucleic acid (e.g., a target sequence). In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

It is understood that the spacer sequence of a spacer sequence need not be 100% complementary to that of a target sequence of a target nucleic acid to hybridize or hybridize specifically to the target sequence. For example, the spacer sequence may comprise at least one alteration, such as a substituted or modified nucleotide, that is not complementary to the corresponding nucleotide of the target sequence.

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary RNA sequence is at least 30, 50, 70, 90, 110, 130, 150, 170, 190, or 210 linked nucleotides. In some embodiments, a length of the intermediary RNA sequence is not greater than 30, 50, 70, 90, 110, 130, 150, 170, 190, or 210 linked nucleotides. In some embodiments, the length of the intermediary RNA sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

Guide nucleic acids described herein may comprise one or more handle sequences. In some embodiments, the handle sequence comprises an intermediary sequence. In such instances, at least a portion of an intermediary sequence non-covalently bonds with an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such instances, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided herein. In some embodiments, the linker comprises a sequence of 5′-GAAA-3′.

In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

A handle sequence may comprise or form a secondary structure (e.g., one or more hairpin loops) that facilitates the binding of an effector protein to a guide nucleic acid and/or modification activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). An effector protein may recognize a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) of the guide nucleic acid non-covalently interacts with the one or more polypeptides described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

In some embodiments, a guide nucleic acid comprises a crRNA. In some embodiments, the guide nucleic acid is the crRNA. In general, a crRNA comprises a first region (FR) and a second region (SR), wherein the FR of the crRNA comprises a repeat sequence, and the SR of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary RNA. In some embodiments, a single nucleic acid system comprises a guide nucleic acid comprising a crRNA and an intermediary RNA.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

In some embodiments, a guide nucleic acid comprises a sgRNA. In some embodiments, a guide nucleic acid is a sgRNA. In some embodiments, a sgRNA comprises a first region (FR) and a second region (SR), wherein the FR comprises a handle sequence and the SR comprises a spacer sequence. In some embodiments, the handle sequence and the spacer sequences are directly connected to each other (e.g., covalent bond (phosphodiester bond)). In some embodiments, the handle sequence and the spacer sequence are connected by a linker.

In some embodiments, a sgRNA comprises one or more of one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g, covalently linked, such as through a phosphodiester bond) In some embodiments, a repeat sequence and a spacer sequence are linked in an sgRNA by any suitable linker, examples of which are provided herein.

In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, activity of effector protein requires binding to a tracrRNA molecule.

In some embodiments, a repeat hybridization sequence is at the 3′ end of a tracrRNA. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

Polypeptides (e.g., effector proteins) and nucleic acids (e.g., engineered guide nucleic acids or reporters) can be further modified as described herein. Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues (e.g., phosphotyrosine, phosphoserine, or phosphothreonine).

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g., D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, e.g., cysteines may be used to make thioethers, histidines for linking to a metal ion complex, carboxyl groups for forming amides or esters, amino groups for forming amides, and the like.

Modifications can further include changing of nucleic acids described herein (e.g., engineered guide nucleic acids) to provide the nucleic acid with a new or enhanced feature, such as improved stability. Such modifications of a nucleic acid include a base editing, a base modification, a backbone modification, a sugar modification, or combinations thereof. In some embodiments, the modifications can be of one or more nucleotides, nucleosides, or nucleobases in a nucleic acid.

In some embodiments, nucleic acids (e.g., nucleic acids encoding effector proteins, engineered guide nucleic acids, nucleic acids encoding engineered guide nucleic acids, or reporters) described herein comprise one or more modifications comprising: 2′O-methyl modified nucleotides, 2′ fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2—NH—O-CH₂-, -CH₂—N(CH₃)—O-CH₂-(known as a methylene (methylimino) or MMI backbone), —CH₂-O—N(CH₃)—CH₂—, —CH₂—N(CH₃)—N(CH₃)—CH₂-and-O—N(CH₃)—CH₂-CH₂-(wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH2 component parts; and combinations thereof.

In some embodiments, guide nucleic acids described herein can be selected from a group of non-naturally occurring guide nucleic acids that have been tiled against the nucleic acid sequence of a strain of an infection or genomic locus of interest. Often, guide nucleic acids that are tiled against the nucleic acid of a strain of an infection or genomic locus of interest can be pooled for use in a method described herein. Often, these guide nucleic acids are pooled for detecting a target nucleic acid or segment thereof in a single assay. The pooling of guide nucleic acids that are tiled against a single target nucleic acid or segment thereof can enhance the detection of the target nucleic using the methods described herein. The pooling of guide nucleic acids that are tiled against a single target nucleic acid or segment thereof can ensure broad coverage of the target nucleic acid or segment thereof within a single reaction using the methods described herein. In some embodiments, the tiling is sequential along the target nucleic acid or segment thereof. In some embodiments, the tiling is overlapping along the target nucleic acid or segment thereof. In some embodiments, the tiling comprises gaps between the tiled non-naturally occurring guide nucleic acids along the target nucleic acid or segment thereof. In some embodiments, the tiling of the guide nucleic acids is non-sequential. Often, a method for detecting a target nucleic acid comprises contacting a target nucleic acid to a pool of guide nucleic acids and a programmable nuclease, wherein a guide nucleic acid of the pool of guide nucleic acids has a sequence selected from a group of tiled guide nucleic acid that correspond to nucleic acids of a target nucleic acid or segment thereof; and assaying for a signal produce by cleavage of at least some reporters of a population of reporters. Pooling of guide nucleic acids can ensure broad spectrum identification, or broad coverage, of a target species within a single reaction. In some embodiments, the guide pooling comprises guide nucleic acids that produce the best signal in a DETECTR reaction (e.g., top 10 gRNAs). In some embodiments, there is an increased signal to noise ratio as the number of pooled gRNAs increases (e.g., signal to noise for 1 gRNA <2 pooled gRNAs <3 pooled gRNAs<4 pooled gRNAs<5 pooled gRNAs<6 pooled gRNAs<7 pooled gRNAs<8 pooled gRNAs<9 pooled gRNAs<10 pooled gRNAs).

### Target Nucleic Acids

In some embodiments, the target nucleic acid is a double stranded nucleic acid. In some embodiments, the target nucleic acid is a single stranded nucleic acid. Alternatively, or in combination, the target nucleic acid is a double stranded nucleic acid and is prepared into single stranded nucleic acids before or upon contacting an RNP. In some embodiments, the single stranded nucleic acid comprises a RNA, wherein the RNA comprises a mRNA, a rRNA, a tRNA, a non-coding RNA, a long non-coding RNA, a microRNA (miRNA), and a single-stranded RNA (ssRNA). In some embodiments, the target nucleic acid is a dsRNA or a crRNA. In some embodiments, the target nucleic acid is complementary DNA (cDNA) synthesized from a single-stranded RNA template in a reaction catalyzed by a reverse transcriptase. In some embodiments, the target nucleic acid is or mRNA. In some embodiments, the target nucleic acid is from a virus, a parasite, or a bacterium described herein.

In some embodiments, a target nucleic acid comprising a target sequence comprises a PAM sequence. In some embodiments, the PAM sequence is 3′ to the target sequence. In some embodiments, the PAM sequence is directly 3′ to the target sequence. In some embodiments, the PAM sequence 5′ to the target sequence. In some embodiments, the PAM sequence is directly 5′ to the target sequence. In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, a target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides.

In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides.

In some embodiments, compositions, systems, and methods described herein comprise a target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the sequence of the target nucleic acid. In some embodiments, the change comprises an insertion, deletion, or substitution of one or more nucleotides compared to the target nucleic acid that has not undergone any modification.

In some embodiments, the target nucleic acid comprises a nucleic acid sequence from a pathogen responsible for a disease. Non-limiting examples of pathogens are bacteria, a virus and a fungus. The target nucleic acid, in some embodiments, is a portion of a nucleic acid from a sexually transmitted infection or a contagious disease. In some embodiments, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some embodiments, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target nucleic acid comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. An effector protein of the disclosure may cleave the viral nucleic acid. In some embodiments, the target nucleic acid comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some embodiments, is a portion of a nucleic acid from a virus or a bacterium or other agents responsible for a disease in the plant (e.g., a crop). In some embodiments, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, a target nucleic acid may be a cancer gene or gene associated with a genetic disorder, or an amplicon thereof, as described herein. In some embodiments, the target nucleic acid is a viral nucleic acid or a bacterial nucleic acid. In some embodiments, the target nucleic acid is a viral nucleic acid. In some embodiments, the target nucleic acid is derived from a papovavirus, a human papillomavirus (HPV), a hepadnavirus, a Hepatitis B Virus (HBV), a herpesvirus, a varicella zoster virus (VZV), an Epstein Barr virus (EBY), a Kaposi's sarcoma-associated herpesvirus, an adenovirus, a poxvirus, a parvovirus, an influenza virus, a respiratory syncytial virus, or a coronavirus. In some embodiments, the target nucleic acid is derived from a severe acute respiratory syndrome coronavirus 2 (SARS-CoV-2). In some embodiments, the target nucleic acid is derived from a human cell. In some embodiments, the target nucleic acid is a human fetal nucleic acid or a cancer cell nucleic acid.

The target nucleic acid disclosed herein can be from a genomic locus. The target nucleic acid disclosed herein can be a gene of a monkeypox virus or a segment thereof. In some embodiments, the gene of the monkeypox virus is conserved in monkeypox virus and does not exist in other Orthopoxviruses.

In some embodiments, the gene of the monkeypox virus is OPG123. In some embodiments, the gene of the monkeypox virus is OPG038. In some embodiments, the gene of the monkeypox virus is OPG094. In some embodiments, the gene of the monkeypox virus is OPG037. In some embodiments, the gene of the monkeypox virus is OPG151. In some embodiments, the gene of the monkeypox virus is OPG105. In some embodiments, the gene of the monkeypox virus is OPG199.

In some embodiments, the target nucleic acid is found in the genome of a monkeypox virus belonging to Clade I. In some embodiments, the target nucleic acid is found in the genome of a monkeypox virus belonging to Clade II. In some embodiments, the target nucleic acid is found in the genome of a monkeypox virus belonging to Clade IIa. In some embodiments, the target nucleic acid is found in the genome of a monkeypox virus belonging to Clade IIb.

In some embodiments, at least a portion of at least one target sequence is within about 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, target nucleic acids described herein comprise a mutation. In some embodiments, a composition, system or method described herein can be used to edit a target nucleic acid comprising a mutation such that the mutation is edited to be the wild-type nucleotide or nucleotide sequence. In some embodiments, a composition, system or method described herein can be used to detect a target nucleic acid comprising a mutation. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

Non-limiting examples of mutations are insertion-deletion (indel), a point mutation, single nucleotide polymorphism (SNP), a chromosomal mutation, a copy number mutation or variation, and frameshift mutations. In some embodiments, an indel mutation is an insertion or deletion of one or more nucleotides. In some embodiments, a point mutation comprises a substitution, insertion, or deletion. In some embodiments, a frameshift mutation occurs when the number of nucleotides in the insertion/deletion is not divisible by three, and it occurs in a protein coding region. In some embodiments, a chromosomal mutation can comprise an inversion, a deletion, a duplication, or a translocation of one or more nucleotides. In some embodiments, a copy number variation can comprise a gene amplification or an expanding trinucleotide repeat. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP, or a deletion, may be encoded in the sequence of a target nucleic acid from the germline of an organism or may be encoded in a target nucleic acid from a diseased cell.

In some embodiments, the mutation is associated with a disease, such as a genetic disorder. In some embodiments, the mutation may be encoded in the sequence of a target nucleic acid from the germline of an organism or may be encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

In some embodiments, a target nucleic acid is in a cell. In some embodiments, the cell is a single-cell eukaryotic organism; a plant cell an algal cell; a fungal cell; an animal cell; a cell of an invertebrate animal; a cell of a vertebrate animal such as fish, amphibian, reptile, bird, and mammal; or a cell of a mammal such as a human, a non-human primate, an ungulate, a feline, a bovine, an ovine, and a caprine. In some embodiments, the cell is a eukaryotic cell. In some embodiments, the cell is a mammalian cell, a human cell, or a plant cell. In some embodiments, the cell is a human cell. In some embodiments, the human cell is a: muscle cell, liver cell, lung cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells.

In some embodiments, an effector protein-guide nucleic acid complex may comprise high selectivity for a target sequence. In some embodiments, an RNP comprise a selectivity of at least 200:1, 100:1, 50:1, 20:1, 10:1, or 5:1 for a target nucleic acid over a single nucleotide variant of the target nucleic acid. In some embodiments, an RNP may comprise a selectivity of at least 5:1 for a target nucleic acid over a single nucleotide variant of the target nucleic acid.

By leveraging such effector protein selectivity, some methods described herein may detect a target nucleic acid present in the sample in various concentrations or amounts as a target nucleic acid population. In some embodiments, the method detects at least 2 target nucleic acid populations. In some embodiments, the method detects at least 3, 4, 5, 6, 7, 8, 9, 10, 20, 30, 40, or 50 target nucleic acid populations. In some embodiments, the method detects 3 to 50, 5 to 40, or 10 to 25 target nucleic acid populations. In some embodiments, the method detects at least 2 individual target nucleic acids. In some embodiments, the method detects at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the method detects 1 to 10,000, 100 to 8000, 400 to 6000, 500 to 5000, 1000 to 4000, or 2000 to 3000 individual target nucleic acids. In some embodiments, the method detects target nucleic acid present at least at one copy per 10 non-target nucleic acids, 102 non-target nucleic acids, 103 non-target nucleic acids, 104 non-target nucleic acids, 105 non-target nucleic acids, 106 non-target nucleic acids, 107 non-target nucleic acids, 108 non-target nucleic acids, 109 non-target nucleic acids, or 1010 non-target nucleic acids.

### Reporter Nucleic Acid

As used herein, a reporter comprises a nucleic acid (e.g., RNA and/or DNA). In some embodiments, a reporter is double-stranded. In some embodiments, a reporter is single-stranded. In some embodiments, a reporter comprises a protein that generates a detectable signal or signal. In some embodiments, a reporter is operably linked to the protein that generates a signal. In some embodiments, a signal is a calorimetric, potentiometric, amperometric, optical (e.g., fluorescent, colorimetric, etc.), or piezo-electric signal. In some embodiments, the reporter comprises a detection moiety. In some embodiments, the reporter is configured to release a detection moiety or generate a signal indicative of a presence or absence of the target nucleic acid. For example, the signal can indicate a presence of the target nucleic acid in the sample, and an absence of the signal can indicate an absence of the target nucleic acid in the sample. In some embodiments, suitable detectable labels and/or moieties provide a signal. In some embodiments, non-limiting example of a suitable detectable label and/or moiety comprises an enzyme, a radioisotope, a member of a specific binding pair; a fluorophore; a fluorescent protein; and a quantum dot.

In some embodiments, methods described herein utilize a reporter. By way of non-limiting and illustrative example, a reporter may comprise a nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal.

In some embodiments, the methods described herein utilize the indiscriminate trans-cleavage of the reporter (e.g., comprising a reporter nucleic acid and a detectable moiety) catalyzed by the effector protein (activated upon hybridization of a guide nucleic acid to a target nucleic acid) to cause the release of the detection moiety and hence the generation of the detectable signal, indicating the presence of the target nucleic acid. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded. In some embodiments, the reporter has one or more regions of single strandedness, and/or one or more regions of double strandedness.

In some embodiments, reporters comprise a protein capable of generating a signal.

A signal may be a calorimetric, potentiometric, amperometric, optical (e.g., fluorescent, colorimetric, etc.), or piezo-electric signal. In some embodiments, the reporter comprises a detection moiety. Suitable detectable labels and/or moieties that may provide a signal include, but are not limited to, an enzyme, an enzyme-substrate, a radioisotope, a member of a specific binding pair (e.g., biotin or avidin), a fluorophore, a fluorescent protein, a quantum dot, and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker by sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other embodiments, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

In some embodiments, the reporter comprises a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other embodiments, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed. Table 2 provides exemplary single stranded reporter nucleic acids that may be used in the methods disclosed herein.

TABLE 2
Non-limiting list of exemplary single stranded reporter nucleic acids that may be
used in the methods disclosed herein.

| 5′ Detection Moiety* | Sequence (SEQ ID NO:) | 3′ Quencher* |
|---|---|---|
| /56-FAM/ | rUrUrUrUrU | /3IABKFQ/ |
| /5TYE665/ | rUrUrUrUrU | /3IAbRQSp/ |
| /5Alex594N/ | rUrUrUrUrU | /3IAbRQSp/ |
| /5ATTO633N/ | rUrUrUrUrU | /3IAbRQSp/ |
| /56-FAM/ | rUrUrUrUrUrUrUrU | /3IABKFQ/ |
| /5IRD700/ | rUrUrUrUrUrUrUrU | /3IRQC1N/ |
| /5TYE665/ | rUrUrUrUrUrUrUrU | /3IAbRQSp/ |
| /5Alex594N/ | rUrUrUrUrUrUrUrU | /3IAbRQSp/ |
| /5ATTO633N/ | rUrUrUrUrUrUrUrU | /3IAbRQSp/ |
| /56-FAM/ | rUrUrUrUrUrUrUrUrUrU (SEQ ID NO: | /3IABKFQ/ |
| | 73) | |
| /5IRD700/ | rUrUrUrUrUrUrUrUrUrU (SEQ ID NO: | /3IRQC1N/ |
| | 74) | |
| /5TYE665/ | rUrUrUrUrUrUrUrUrUrU (SEQ ID NO: | /3IAbRQSp/ |
| | 75) | |
| /5Alex594N/ | rUrUrUrUrUrUrUrUrUrU (SEQ ID NO: | /3IAbRQSp/ |
| | 76) | |
| /5ATTO633N/ | rUrUrUrUrUrUrUrUrUrU (SEQ ID NO: | /3IAbRQSp/ |
| | 77) | |
| /56-FAM/ | TTTTrUrUTTTT (SEQ ID NO: 78) | /3IABKFQ/ |
| /5IRD700/ | TTTTrUrUTTTT (SEQ ID NO: 79) | /3IRQC1N/ |
| /5TYE665/ | TTTTrUrUTTTT (SEQ ID NO: 80) | /3IAbRQSp/ |
| /5Alex594N/ | TTTTrUrUTTTT (SEQ ID NO: 81) | /3IAbRQSp/ |
| /5ATTO633N/ | TTTTrUrUTTTT (SEQ ID NO: 82) | /3IAbRQSp/ |
| /56-FAM/ | TTrUrUTT | /3IABKFQ/ |
| /5IRD700/ | TTrUrUTT | /3IRQC1N/ |
| /5TYE665/ | TTrUrUTT | /3IAbRQSp/ |
| /5Alex594N/ | TTrUrUTT | /3IAbRQSp/ |
| /5ATTO633N/ | TTrUrUTT | /3IAbRQSp/ |
| /56-FAM/ | TArArUGC | /3IABkFQ/ |
| /5IRD700/ | TArArUGC | /3IRQC1N/ |
| /5TYE665/ | TArArUGC | /3IAbRQSp/ |
| /5Alex594N/ | TArArUGC | /3IAbRQSp/ |
| /5ATTO633N/ | TArArUGC | /3IAbRQSp/ |
| /56-FAM/ | TArUrGGC | /3IABKFQ/ |
| /5IRD700/ | TArUrGGC | /3IRQC1N/ |
| /5TYE665/ | TArUrGGC | /3IAbRQSp/ |
| /5Alex594N/ | TArUrGGC | /3IAbRQSp/ |
| /5ATTO633N/ | TArUrGGC | /3IAbRQSp/ |
| /56-FAM/ | rUrUrUrUrU | /3IABKFQ/ |
| /5IRD700/ | rUrUrUrUrU | /3IRQC1N/ |
| /5TYE665/ | rUrUrUrUrU | /3IAbRQSp/ |
| /5Alex594N/ | rUrUrUrUrU | /3IAbRQSp/ |
| /5ATTO633N/ | rUrUrUrUrU | /3IAbRQSp/ |
| /56-FAM/ | TTATTATT | /3IABKFQ/ |
| /56-FAM/ | TTATTATT | /3IABKFQ/ |
| /5IRD700/ | TTATTATT | /3IRQC1N/ |
| /5TYE665/ | TTATTATT | /3IAbRQSp/ |
| /5Alex594N/ | TTATTATT | /3IAbRQSp/ |
| /5ATTO633N/ | TTATTATT | /3IAbRQSp/ |
| /56-FAM/ | TTTTTT | /3IABKFQ/ |
| /56-FAM/ | TTTTTTTT | /3IABKFQ/ |
| /56-FAM/ | TTTTTTTTTT (SEQ ID NO: 83) | /3IABKFQ/ |
| /56-FAM/ | TTTTTTTTTTTT (SEQ ID NO: 84) | /3IABKFQ/ |
| /56-FAM/ | TTTTTTTTTTTTTT (SEQ ID NO: 85) | /3IABKFQ/ |
| /56-FAM/ | AAAAAA | /3IABKFQ/ |
| /56-FAM/ | CCCCCC | /3IABKFQ/ |
| /56-FAM/ | GGGGGG | /3IABKFQ/ |
| /56-FAM/ | TTATTATT | /3IABKFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5Alex488N/: 5′ Alexa Fluor 488 (NHS Ester) (Integrated DNA Technologies)
/5ATTO633N/: 5′ ATTO TM 633 (NHS Ester) (Integrated DNA Technologies)
/3IRQCIN/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

The generation of the detectable signal from the release of the detection moiety may indicate that cleavage by the effector protein has occurred and that the sample contains the target nucleic acid. In some embodiments, the detection moiety comprises a fluorescent dye.

Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

In some embodiments, a detection moiety comprises any moiety that generates a detectable product or detectable signal upon cleavage of the reporter by the effector protein. In some embodiments, the detectable product comprises a detectable unit generated from the detectable moiety and that emits a detectable signal as described herein. In some embodiments, the detectable product further comprises a detectable label, a fluorophore, a reporter, or a combination thereof. In some embodiments, the detectable product comprises RNA, DNA, or both. In some embodiments, the detectable product is configured to generate a signal indicative of the presence or absence of the target nucleic acid in, for instance, a cell or a sample.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by interaction of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only DNA residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified DNAs.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, the methods disclosed herein utilize a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, the methods disclosed herein utilize an effector protein and a reporter nucleic acid configured to undergo trans cleavage by the effector protein. Trans cleavage of the reporter may generate a signal from the reporter or alter a signal from the reporter. In some embodiments, the signal is an optical signal, such as a fluorescence signal or absorbance band. Trans cleavage of the reporter may alter the wavelength, intensity, or polarization of the optical signal. For example, the reporter may comprise a fluorophore and a quencher, such that trans cleavage of the reporter separates the fluorophore and the quencher thereby increasing a fluorescence signal from the fluorophore. Herein, detection of reporter cleavage to determine the presence of a target nucleic acid may be referred to as ‘DETECTR’. Further, the quantitation based on the number of positive nanovolumes as detected by reporter cleavage to determine the amount or quantity of a target nucleic acid using the methods disclosed herein may be referred to as ‘digital DETECTR’.

In the presence of a large amount of non-target nucleic acids, an activity of an effector protein (e.g., an effector protein as disclosed herein) may be inhibited. This is because the activated effector proteins collaterally cleave any nucleic acid. If total nucleic acids are present in large amounts, they may outcompete reporters for the effector proteins. In some embodiments, the methods disclosed herein utilize an excess of reporter(s), such that the concentration of the reporter in the reaction mixture is greater than the concentration of the target nucleic acid.

In some embodiments, the concentration of the reporter is greater than the concentration of target nucleic acids and non-target nucleic acids. The non-target nucleic acids may be from the original sample, either lysed or unlysed. In some embodiments, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids.

In some embodiments, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

Further description of editing or detecting a target nucleic acid in a gene of interest can be found in more detail in Kim et al., “Enhancement of target specificity of CRISPR-Cas12a by using a chimeric DNA-RNA guide”, Nucleic Acids Res. 2020 Sep. 4; 48 (15): 8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus a-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep. 2015 Jul. 28; 12 (4): 673-83, which are hereby incorporated by reference in their entirety.

In some embodiments, the reporter nucleic acid comprises ribonucleotides. In some embodiments, the reporter nucleic acid comprises deoxyribonucleotides. In some embodiments, the reporter nucleic acid comprises both ribonucleotides and deoxyribonucleotides. In some embodiments, the reporter nucleic acid comprises a single stranded nucleic acid. In some embodiments, the reporter nucleic acid comprises a double stranded nucleic acid. In some embodiments, the reporter nucleic acid comprises one or more regions of single stranded nucleic acid and one or more regions of double stranded nucleic acid. In some embodiments, the reporter nucleic acid comprises a modified nucleobase, a modified sugar moiety, and/or a modified nucleic acid linkage.

In some embodiments, the reporter nucleic acid comprises the nucleic acid sequence of rUrUrUrUrU. In some embodiments, the reporter nucleic acid comprises the nucleic acid sequence of TTATTATT. In some embodiments, the reporter nucleic acid comprises the nucleic acid sequence of TTTTTTTTTTTT (SEQ ID NO: 86). In some embodiments, the 5′ end of the reporter nucleic acid is bound to a fluorescent dye, such as, Alexa Fluor 647. Non-limiting examples of fluorescent dyes include Alexa Fluor 350, Alexa Fluor 405, Alexa Fluor 488, Alexa Fluor 532, Alexa Fluor 546, Alexa Fluor 555, Alexa Fluor 561, Alexa Fluor 568, Alexa Fluor 594, Alexa Fluor 647, Alexa Fluor 660, Alexa Fluor 680, Alexa Fluor 700, and Alexa Fluor 750. In some embodiments, the 3′ end of the reporter nucleic acid is bound to a dark quencher, such as, for example, 3′ Iowa Black® RQ (“3IAbRQSp”).

In some embodiments, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some embodiments, the reporter nucleic acid is a homopolymeric reporter nucleic acid comprising 5 to 20 consecutive adenines, 5 to 20 consecutive thymines, 5 to 20 consecutive cytosines, or 5 to 20 consecutive guanines. In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, methods of detecting target nucleic acids in a sample have a threshold, wherein the threshold corresponds to a minimal amount of target nucleic acid that must be present in the sample, prior to partitioning into nanovolumes, in order for detection to occur. For example, in some embodiments, when a threshold of detection is 10 nM, then a signal can be detected when a target nucleic acid is present in the sample at a concentration of 10 nM or more. In such embodiments, the methods are not capable of detecting target nucleic acids that are present in a sample at a concentration less than 10 nM. In some embodiments, the threshold is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some embodiments, the threshold is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some embodiments, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some embodiments, the threshold is in a range of from 2 aM to 100 pM, from 20 aM to 50 pM, from 50 aM to 20 pM, from 200 aM to 5 pM, or from 500 aM to 2 pM.

In some embodiments, a minimum concentration at which the methods detect a target nucleic acid a sample is in a range of from 1 zeptomolar (zM) to 1 nM, 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some embodiments, a minimum concentration at which the methods detect in a sample is in a range of from 2 aM to 100 pM, from 20 aM to 50 pM, from 50 aM to 20 pM, from 200 aM to 5 pM, or from 500 aM to 2 pM. In some embodiments, a minimum concentration at which the methods detect a single stranded target nucleic acid in a sample is in a range of from 1 zM to 100 pM. In some embodiments, a minimum concentration at which the methods detect a target nucleic acid in a sample is in a range of from 1 fM to 100 pM. In some embodiments, a minimum concentration at which the methods detect a single stranded target nucleic acid in a sample is in a range of from 10 fM to 100 pM. In some embodiments, a minimum concentration at which the methods detect a single stranded target nucleic acid in a sample is in a range of from 800 fM to 100 pM. In some embodiments, a minimum concentration at which the methods detect a single stranded target nucleic acid in a sample is in a range of from 1 pM to 10 pM. In some embodiments, the devices, systems, fluidic devices, kits, and methods described herein detect a single stranded target nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some embodiments, a minimum concentration at which the methods detect a target nucleic acid at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, a minimum concentration at which the methods detect a target nucleic acid at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, a minimum concentration at which the methods detect a target nucleic acid at a concentration of from 20 nM to 50 μM, from 50 nM to 20 μM, or from 200 nM to 5 μM.

In some embodiments, the methods disclosed herein detect a target nucleic acid in less than 60 minutes. In some embodiments, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some embodiments, the detectable signal is detectable in a time period in the range of about 10 min to about 24 hours, for example, about 15 min, about 30 min, about 45 min, about 1 hour, about 90 min, about 2 hours, about 5 hours, about 10 hours, about 15 hours, about 20 hours, or about 24 hours, including all values and subranges that lie therebetween. In some embodiments, the detectable signal is detectable in less than 90 minutes. In some embodiments, the detectable signal is detectable in less than 30 minutes. In some embodiments, the detectable signal is detectable in a time period in the range of about 15 min to about 24 hours.

In some embodiments, the detectable signal is detectable in at least about 120 minutes, at least about 110 minutes, at least about 100 minutes, at least about 90 minutes, at least about 80 minutes, at least about 70 minutes, at least about 60 minutes, at least about 55 minutes, at least about 50 minutes, at least about 45 minutes, at least about 40 minutes, at least about 35 minutes, at least about 30 minutes, at least about 25 minutes, at least about 20 minutes, at least about 15 minutes, at least about 10 minutes, or at least about 5 minutes. In some embodiments, the sample is contacted with the reagents for from 5 minutes to 120 minutes, from 5 minutes to 100 minutes, from 10 minutes to 90 minutes, from 15 minutes to 45 minutes, or from 20 minutes to 35 minutes.

In some embodiments, methods of detecting are performed in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some embodiments, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

In some embodiments, the detection occurs within 5 minutes of contacting a sample and/or a target nucleic acid with a composition described herein. In some embodiments, the detection occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, the detection occurs within 1 to 120, 5 to 100, 10 to 90, 15 to 80, 20 to 60, or 30 to 45 minutes of contacting the target nucleic acid.

### Test Samples

Various sample types comprising a target nucleic acid of interest are consistent with the present disclosure. These samples may comprise a target nucleic acid for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, cancer, genetic disorder, or any mutation of interest.

In some embodiments, the test sample comprises DNA from a cell lysate. In some embodiments, the test sample comprises cells. In some embodiments, the test sample is a blood, serum, plasma, urine, aspirate, fecal or biopsy sample. In some embodiments, the test sample comprises, or derived from, wastewater.

In some embodiments, a sample comprises a target nucleic acid from 0.001% to 20% of total nucleic acids in the sample. In some embodiments, a sample comprises a target nucleic acid from 0.005% to 20% of total nucleic acids in the sample. In some embodiments, a sample comprises a target nucleic acid from 0.05% to 20% of total nucleic acids in the sample. In some embodiments, the target nucleic acid is 0.1% to 10% of the total nucleic acids in the sample. In some embodiments, the target nucleic acid is 0.1% to 5% of the total nucleic acids in the sample. In some embodiments, the target nucleic acid is 0.1% to 1% of the total nucleic acids in the sample. In some embodiments, the target nucleic acid is in any amount less than 100% of the total nucleic acids in the sample. In some embodiments, the target nucleic acid is 100% of the total nucleic acids in the sample. In some embodiments, the sample comprises a portion of the target nucleic acid and at least one nucleic acid comprising less than 100% sequence identity to the portion of the target nucleic acid but no less than 50% sequence identity to the portion of the target nucleic acid. For example, the portion of the target nucleic acid comprises a mutation as compared to at least one nucleic acid comprising less than 100% sequence identity to the portion of the target nucleic acid but no less than 50% sequence identity to the portion of the target nucleic acid. In some embodiments, the portion of the target nucleic acid comprises a single nucleotide mutation as compared to at least one nucleic acid comprising less than 100% sequence identity to the portion of the target nucleic acid but no less than 50% sequence identity to the portion of the target nucleic acid.

In some embodiments, a sample comprises target nucleic acid populations at different concentrations or amounts. In some embodiments, the sample has at least 2 target nucleic acid populations. In some embodiments, the sample has at least 3, 4, 5, 6, 7, 8, 9, 10, 20, 30, 40, or 50 target nucleic acid populations. In some embodiments, the sample has 3 to 50, 5 to 40, or 10 to 25 target nucleic acid populations.

In some embodiments, a sample has at least 2 individual target nucleic acids. In some embodiments, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the sample comprises 1 to 10,000, 100 to 8000, 400 to 6000, 500 to 5000, 1000 to 4000, or 2000 to 3000 individual target nucleic acids.

In some embodiments, the test sample comprises about 10,000 molecules to about 100,000 molecules of the target nucleic acid. In some embodiments, the test sample comprises about 50,000 molecules of the target nucleic acid.

In some embodiments, a sample comprises one copy of target nucleic acid per 10 non-target nucleic acids, 102 non-target nucleic acids, 103 non-target nucleic acids, 104 non-target nucleic acids, 105 non-target nucleic acids, 106 non-target nucleic acids, 107 non-target nucleic acids, 108 non-target nucleic acids, 109 non-target nucleic acids, or 1010 non-target nucleic acids.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 M, or less than 1 mM. In some embodiments, the sample comprises a target nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). In some embodiments, a biological sample is saliva. In some embodiments, the biological sample is taken as a swab from a wound or a lesion. A tissue sample from a subject may be dissociated or liquified prior to application to detection system of the present disclosure. Non-limiting examples of environmental samples are soil, air, or water. In some embodiments, an environmental sample is taken as a swab from a surface of interest or taken directly from the surface of interest. In some embodiments, a sample is a wastewater sample. Wastewater surveillance for a pathogen (e.g., SARS-CoV-2, monkeypox virus) can serve as an early indicator that a pathogen is spreading in a community.

In some embodiments, the sample is a raw (unprocessed, unedited, unmodified) sample. Raw samples may be applied to a system for detecting or editing a target nucleic acid, such as those described herein. In some embodiments, the sample is diluted with a buffer or a fluid or concentrated prior to its application to the system or be applied neat to the detection system. Sometimes, the sample contains no more 20 μl of buffer or fluid. The sample, in some embodiments, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl.

In some embodiments, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine.

In some embodiments, the sample is taken from nematodes, protozoans, helminths, or malarial parasites. In some embodiments, the sample comprises nucleic acids from a cell lysate from a eukaryotic cell, a mammalian cell, a human cell, a prokaryotic cell, or a plant cell. In some embodiments, the sample comprises nucleic acids expressed from a cell.

In some embodiments, samples are used for diagnosing a disease. In some embodiments the disease is cancer. The sample used for cancer testing may comprise at least one target nucleic acid that may hybridize to a guide nucleic acid of the reagents described herein. The target nucleic acid, in some embodiments, comprises a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker. In some embodiments, the assay may be used to detect “hotspots” in target nucleic acids that may be predictive of a cancer. In some embodiments, the target nucleic acid comprises a portion of a nucleic acid that is associated with a cancer. In some embodiments, the target nucleic acid is a portion of a nucleic acid from a genomic locus, any DNA amplicon of, a reverse transcribed mRNA, or a cDNA from a locus of at least one of a gene. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

The methods disclosed herein can be used as a companion diagnostic with medicaments used to treat a disease or an infection, or can be used in reagent kits, point-of-care diagnostics, or over-the-counter diagnostics. The methods may be used as a point of care diagnostic or as a lab test for detection of a target nucleic acid and, thereby, detection of a condition in a subject from which the sample was taken. The methods may be used in various sites or locations, such as in laboratories, in hospitals, in physician offices/laboratories (POLs), in clinics, at remotes sites, or at home.

In some embodiments, samples are used to diagnose a genetic disorder, also referred to as genetic disorder testing. The sample used for genetic disorder testing may comprise at least one target nucleic acid that may hybridize to a guide nucleic acid of the reagents described herein. The target nucleic acid, in some embodiments, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some embodiments, the target nucleic acid is a nucleic acid from a genomic locus, a transcribed mRNA, or a reverse transcribed mRNA, a DNA amplicon of or a cDNA from a locus of a gene.

A sample used for phenotyping testing may comprise at least one target nucleic acid that may hybridize to a guide nucleic acid of the reagents described herein. The target nucleic acid, in some embodiments, is a nucleic acid encoding a sequence associated with a phenotypic trait. A sample used for genotyping testing may comprise at least one target nucleic acid that may hybridize to a guide nucleic acid of the reagents described herein. A target nucleic acid, in some embodiments, is a nucleic acid encoding a sequence associated with a genotype of interest. A sample used for ancestral testing may comprise at least one target nucleic acid that may hybridize to a guide nucleic acid of the reagents described herein. A target nucleic acid, in some embodiments, is a nucleic acid encoding a sequence associated with a geographic region of origin or ethnic group. A sample may be used for identifying a disease status. For example, a sample is any sample described herein, and is obtained from a subject for use in identifying a disease status (e.g., infected with monkeypox virus or uninfected) of a subject. Sometimes, a method comprises obtaining a saliva sample or a wound swab sample from a subject; and identifying a disease status of the subject. In some embodiments, the disease is cancer. In some embodiments, the disease is a genetic disorder. In some embodiments, a method comprises obtaining a serum sample from a subject; and identifying a disease status of the subject.

### Reaction Mixtures

In some embodiments, the reaction mixtures described herein comprise a solution or buffer; a reagent; a support medium; other components or appurtenances as described herein; or combinations thereof.

In general, the reaction mixtures comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, the reaction mixtures comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, the reaction mixtures comprise a solution, wherein the solution comprises at least one salt. In some embodiments, the at least one salt is selected from potassium acetate, magnesium acetate, sodium chloride, potassium chloride, magnesium chloride, calcium chloride, and any combination thereof. In some embodiments, the concentration of the at least one salt in the solution is 5 mM to 100 mM, 5 mM to 10 mM, 1 mM to 60 mM, or 1 mM to 10 mM. In some embodiments, the concentration of the at least one salt is about 105 mM. In some embodiments, the concentration of the at least one salt is about 55 mM. In some embodiments, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some embodiments, the salt is a magnesium salt and the concentration of magnesium in the solution is at least 5 mM, 7 mM, at least 9 mM, at least 11 mM, at least 13 mM, or at least 15 mM. In some embodiments, the concentration of magnesium is less than 20 mM, less than 18 mM, or less than 16 mM.

In some embodiments, the reaction mixtures comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, the reaction mixtures comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, the reaction mixtures comprise a solution, wherein the solution comprises at least one reducing agent. Exemplary reducing agents comprise dithiothreitol (DTT), β-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, the reaction mixtures comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, the reaction mixtures comprise a solution, wherein the solution comprises a co-factor. In some embodiments, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21 (13): 3728-3739). In some embodiments, an effector or a multimeric complex thereof forms a complex with a co-factor. In some embodiments, the co-factor is a divalent metal ion. In some embodiments, the divalent metal ion is selected from Mg²⁺, Mn²⁺, Zn²⁺, Ca²⁺, Cu²⁺. In some embodiments, the divalent metal ion is Mg²⁺. In some embodiments, the co-factor is Mg²⁺.

In some embodiments, the reaction mixture comprises a buffer, wherein the buffer comprises tricine, MgOAc, BSA, TCEP, imidazole, KCl, MgCl₂, BSA, Igepal Ca-630, glycerol, HEPES, KOAc, Triton-X 100, Tris-HCl, (NH4)2SO4, Tween-20, TMAO, or any combination thereof.

### Multiplexing

The methods, systems, compositions, and kits described herein can be multiplexed in a number of ways. These methods of multiplexing are, for example, consistent with methods, systems, compositions, and kits disclosed herein for detection of a target nucleic acid within the sample.

Multiplexing can be spatial multiplexing wherein multiple different target nucleic acids are detected at the same time, but the reactions are spatially separated. In some embodiments, the multiple target nucleic acids or segments thereof are detected using the same programmable nuclease, but different non-naturally occurring guide nucleic acids. In some embodiments, the multiple target nucleic acids are detected using the different programmable nucleases. Alternatively, multiplexing can be single reaction multiplexing wherein multiple different target acids are detected in a single reaction volume (e.g., in a single nanovolume). In some embodiments, a single population of programmable nucleases is used in single reaction multiplexing. In some embodiments, at least two different populations of programmable nucleases are used in single reaction multiplexing.

Furthermore, signals from multiplexing can be quantified. For example, a method of quantification for a disease panel comprises assaying for a plurality of unique target nucleic acids in a plurality of aliquots from a sample, assaying for a control nucleic acid in a second aliquot of the sample, and quantifying a plurality of signals of the plurality of unique target nucleic acids by measuring signals produced by cleavage of reporters compared to the signal produced in the second aliquot. In some embodiments, the plurality of unique target nucleic acids or segments thereof are from a plurality of viruses in the sample. In some embodiments, the quantification of a signal of the plurality correlates with a concentration of a unique target nucleic acid or segment thereof of the plurality.

In some embodiments, the reagents for multiplexed assays comprise multiple non-naturally occurring guide nucleic acids, multiple programmable nucleases, and multiple single stranded reporters, where a combination of one of the non-naturally occurring guide nucleic acids, one of the programmable nucleases, and one of the single stranded reporters detects one target nucleic acid or segment thereof and can provide a detection spot on the detection region. In some embodiments, the combination of a non-naturally occurring guide nucleic acid, a programmable nuclease, and a single stranded reporter configured to detect one target nucleic acid or segment thereof is mixed with at least one other combination in a single reagent chamber. In some embodiments, the combination of a non-naturally occurring guide nucleic acid, a programmable nuclease, and a single stranded reporter configured to detect one target nucleic acid or segment thereof is mixed with at least one other combination on a single support medium. When these combinations of reagents are contacted with the sample, the reaction for the multiple target nucleic acids or segments thereof occurs simultaneously in the same medium or reagent chamber (e.g., in the same nanovolume).

In some embodiments, the combination of a non-naturally occurring guide nucleic acid, a programmable nuclease, and a single stranded reporter configured to detect one target nucleic acid or segment thereof is provided in its own reagent chamber or its own support medium (e.g., in its own nanovolume). In this case, multiple reagent chambers or support mediums are provided in the device, kit, or system, where one reagent chamber is designed to detect one target nucleic acid or segment thereof. In this case, multiple support mediums are used to detect the panel of viral infections, or other diseases of interest.

Multiplexing of a DNA-activated programmable DNA nuclease, such as a Type V CRISPR-Cas protein, with a DNA-activated programmable RNA nuclease, such as a Type VI protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing.

In some embodiments, multiplexing enables detections of different segments of the same gene at the same time. In some embodiments, multiplexing enables detections of at least 2, 3, 4, 5, 6, 7, 8, 9, 10, 15, 20, 25, 30, 35, 40, 45, 50, 60, 70, 80, 90, 100 or more different segments of the same gene at the same time. In some embodiments, multiplexing enables detections of 2-500, 2-400, 2-300, 2-200, 2-100, 2-90, 2-80, 2-70, 2-60, 2-50, 2-45, 2-40, 2-35, 2-30, 2-25, 2-20, 2-15, 2-10, or 2-5 different segments of the same gene at the same time. In some embodiments, the gene is a monkeypox gene.

In some embodiments, multiplexing enables detections of different genes at the same time. In some embodiments, multiplexing enables detections of at least 2, 3, 4, 5, 6, 7, 8, 9, 10, 15, 20, 25, 30, 35, 40, 45, 50, 60, 70, 80, 90, 100 or more different genes at the same time. In some embodiments, multiplexing enables detections of 2-500, 2-400, 2-300, 2-200, 2-100, 2-90, 2-80, 2-70, 2-60, 2-50, 2-45, 2-40, 2-35, 2-30, 2-25, 2-20, 2-15, 2-10, or 2-5 different genes at the same time. In some embodiments, at least of the genes is a monkeypox gene.

In some embodiments, multiplexing enables detections of both different segments of the same gene and different genes at the same time. In some embodiments, there are at least 2, 3, 4, 5, 6, 7, 8, 9, 10, 15, 20, 25, 30, 35, 40, 45, 50, 60, 70, 80, 90, 100 or more different segments of the same gene. In some embodiments, there are 2-500, 2-400, 2-300, 2-200, 2-100, 2-90, 2-80, 2-70, 2-60, 2-50, 2-45, 2-40, 2-35, 2-30, 2-25, 2-20, 2-15, 2-10, or 2-5 different segments of the same gene. In some embodiments, there are at least 2, 3, 4, 5, 6, 7, 8, 9, 10, 15, 20, 25, 30, 35, 40, 45, 50, 60, 70, 80, 90, 100 or more different genes at the same time. In some embodiments, there are 2-500, 2-400, 2-300, 2-200, 2-100, 2-90, 2-80, 2-70, 2-60, 2-50, 2-45, 2-40, 2-35, 2-30, 2-25, 2-20, 2-15, 2-10, or 2-5 different genes at the same time. In some embodiments, at least of the genes is a monkeypox gene.

### Methods For Detecting and Assaying for Monkeypox Virus

Provided herein are compositions, systems, kits, and methods of detecting and assaying for monkeypox virus. The detection assays disclosed herein may provide low cost, portable, and accurate detection of monkeypox virus and may be performed using commercially available reagents. Such assays may be referred to herein as monkeypox virus DNA Endonuclease-Targeted CRISPR (clustered regularly interspaced short palindromic repeats) Trans Reporter (DETECTR) assays.

In particular, the various methods, compositions, and kits disclosed herein use a programmable nuclease complexed with a non-naturally occurring guide nucleic acid that hybridizes to a target sequence of a target nucleic acid from monkeypox virus. The complex can be contacted to a sample from a subject. The subject may or may not be infected with monkeypox virus. The target nucleic acid in the sample can be amplified by thermal amplification (e.g., PCR) or isothermal amplification (e.g., LAMP). If the subject is infected with monkeypox virus, the non-naturally occurring guide nucleic acid hybridizes to the target nucleic acid leading to activation of programmable nuclease. Upon activation, the programmable nuclease can cleave a reporter, wherein the reporter optionally comprises a detectable label attached to a polynucleotide (e.g., polydeoxyribonucleotide or polyribonucleotide). Upon cleavage of the polynucleotide, the detectable label emits a detectable signal, which is then detected and quantified (e.g., the detectable label may be a fluorophore and the detectable signal may be fluorescence). Upon detection of a detectable label, it can be determined that the sample from the subject contained target nucleic acids from monkeypox virus. A patient may be diagnosed with monkey pox if the presence of monkeypox virus is detected in a sample from the patient. In some embodiments, a DETECTR assay may detect multiple target nucleic acids or amplicons. In some embodiments, a DETECTR assay may detect multiple target nucleic acids that are specific to monkeypox virus. In some embodiments, a DETECTR assay may detect a combination of one or more target nucleic acid(s) specific to monkeypox virus and one or more target nucleic acid(s) present in other Orthopoxviruses to distinguish or discriminate between monkeypox virus and other Orthopoxviruses in patient samples.

A number of reagents are consistent with the methods, compositions, and kits disclosed herein. The reagents described herein may be used in methods of assaying for a target nucleic acid in a sample, the method comprising a) amplifying the target nucleic acid using at least one amplification primer; b) contacting the sample to a reporter and a composition comprising a programmable nuclease and a guide nucleic acid that hybridizes to the target nucleic acid or an amplified product thereof, wherein the programmable nuclease cleaves the reporter upon hybridization of the guide nucleic acid to the target nucleic acid or the amplification product thereof; and c) assaying for a change in a signal, wherein the change in the signal is produced by cleavage of the reporter; wherein the target nucleic acid is a gene of a monkeypox virus or a segment thereof; and optionally wherein the at least one amplification primer comprises a nucleotide sequence at least 85%, at least 87%, at least 90%, at least 92%, at least 94%, at least 95%, at least 96%, at least 98%, or 100% identical to any one of SEQ ID 5 NOs: 92-235.

### Amplification Primers

The reagents described herein may comprise amplification primers.

In some embodiments, an amplification primer comprises a nucleotide sequence at least 80%, at least 85%, at least 87%, at least 90%, at least 92%, at least 94%, at least 95 10%, at least 96%, at least 98%, or 100% to any one of SEQ ID NOs: 92-235, as provided in Table 3 below.

TABLE 3
Exemplary Amplification Primer Sequences

| SEQ | | | | |
|---|---|---|---|---|
| ID NO | Primer Name | Sequence | Primer Set | Associated sgRNA |
| 92. | M44982 monkeypox- | TTTTTCATCGACTAG | MPXV-set1 | MPXV-1 or MPXV-3 |
| | set1-F3 | ACGT | | |
| 93. | M44983 monkeypox- | CATTGATGAATGTCA | MPXV-set1 | MPXV-1 or MPXV-3 |
| | set1-B3 | TAACTTCA | | |
| 94. | M44984 monkeypox- | GCAAGAATTCACCAT | MPXV-set1 | MPXV-1 or MPXV-3 |
| | set1-FIP | GTTGGTTAAATAGCG | | |
| | | ATTGGTGTTGT | | |
| 95. | M44985 monkeypox- | CTTATGGTTTTTTAA | MPXV-set1 | MPXV-1 or MPXV-3 |
| | set1-BIP | CGCAATGGTCGAAG | | |
| | | ATGGTAAGATCCGTC | | |
| 96. | M44986 monkeypox- | ACTTACTGCGACCAG | MPXV-set1 | MPXV-1 or MPXV-3 |
| | set1-LF | G | | |
| 97. | M44987 monkeypox- | ATATACTGACCGAGT | MPXV-set1 | MPXV-1 or MPXV-3 |
| | set1-LB | AGG | | |
| 98. | M44988 monkeypox- | GTTTATTCTCAAATA | MPXV-set2 | MPXV-1 or MPXV-3 |
| | set2-F3 | GCGATTG | | |
| 99. | M44989 monkeypox- | CATTGATGAATGTCA | MPXV-set2 | MPXV-1 or MPXV-3 |
| | set2-B3 | TAACTTCA | | |
| 100. | M44990 monkeypox- | ATCGTTAATAGTGTG | MPXV-set2 | MPXV-1 or MPXV-3 |
| | set2-FIP | CAAGAATTCAGTGTT | | |
| | | GTAAGGATCCTGG | | |
| 101. | M44991 monkeypox- | CTTATGGTTTTTTAA | MPXV-set2 | MPXV-1 or MPXV-3 |
| | set2-BIP | CGCAATGGTCGAAG | | |
| | | ATGGTAAGATCCGTC | | |
| 102. | M44992 monkeypox- | TACTGCGACCAGGAT | MPXV-set2 | MPXV-1 or MPXV-3 |
| | set2-LF | C | | |
| 103. | M44993 monkeypox- | TATACTGACCGAGTA | MPXV-set2 | MPXV-1 or MPXV-3 |
| | set2-LB | G | | |
| 104. | M44994 monkeypox- | TTTTTCATCGACTAG | MPXV-set3 | MPXV-1 or MPXV-3 |
| | set3-F3 | ACGT | | |
| 105. | M44995 monkeypox- | CATTGATGAATGTCA | MPXV-set3 | MPXV-1 or MPXV-3 |
| | set3-B3 | TAACTTCA | | |
| 106. | M44996 monkeypox- | GCAAGAATTCACCAT | MPXV-set3 | MPXV-1 or MPXV-3 |
| | set3-FIP | GTTGGAATAGCGATT | | |
| | | GGTGTTGT | | |
| 107. | M44997 monkeypox- | TGGTTTTTTAACGCA | MPXV-set3 | MPXV-1 or MPXV-3 |
| | set3-BIP | ATGGTCGAAGATGGT | | |
| | | AAGATCCGTC | | |
| 108. | M44998 monkeypox- | ACTTACTGCGACCAG | MPXV-set3 | MPXV-1 or MPXV-3 |
| | set3-LF | G | | |
| 109. | M44999 monkeypox- | ATATACTGACCGAGT | MPXV-set3 | MPXV-1 or MPXV-3 |
| | set3-LB | AGG | | |
| 110. | M45000 monkeypox- | GTTTATTCTCAAATA | MPXV-set4 | MPXV-1 or MPXV-3 |
| | set4-F3 | GCGATTG | | |
| 111. | M45001 monkeypox- | CATTGATGAATGTCA | MPXV-set4 | MPXV-1 or MPXV-3 |
| | set4-B3 | TAACTTCA | | |
| 112. | M45002 monkeypox- | ATCGTTAATAGTGTG | MPXV-set4 | MPXV-1 or MPXV-3 |
| | set4-FIP | CAAGAATTCAGTGTT | | |
| | | GTAAGGATCCTGG | | |
| 113. | M45003 monkeypox- | CTTATGGTTTTTTAA | MPXV-set4 | MPXV-1 or MPXV-3 |
| | set4-BIP | CGCAATGGTCGAAG | | |
| | | ATGGTAAGATCCGTC | | |
| 114. | M45004 monkeypox- | CATGTTGGTTAACTT | MPXV-set4 | MPXV-1 or MPXV-3 |
| | set4-LF | ACTGC | | |
| 115. | M45005 monkeypox- | ATATACTGACCGAGT | MPXV-set4 | MPXV-1 or MPXV-3 |
| | set4-LB | AGG | | |
| 116. | M45006 monkeypox- | GTTTATTCTCAAATA | MPXV-set5 | MPXV-1 or MPXV-3 |
| | set5-F3 | GCGATTG | | |
| 117. | M45007 monkeypox- | CATTGATGAATGTCA | MPXV-set5 | MPXV-1 or MPXV-3 |
| | set5-B3 | TAACTTCA | | |
| 118. | M45008 monkeypox- | ATCGTTAATAGTGTG | MPXV-set5 | MPXV-1 or MPXV-3 |
| | set5-FIP | CAAGAATTCAGTGTT | | |
| | | GTAAGGATCCTGG | | |
| 119. | M45009 monkeypox- | CTTATGGTTTTTTAA | MPXV-set5 | MPXV-1 or MPXV-3 |
| | set5-BIP | CGCAATGGTCGAAG | | |
| | | ATGGTAAGATCCGTC | | |
| 120. | M45010 monkeypox- | GTTGGTTAACTTACT | MPXV-set5 | MPXV-1 or MPXV-3 |
| | set5-LF | GCGA | | |
| 121. | M45011 monkeypox- | TATACTGACCGAGTA | MPXV-set5 | MPXV-1 or MPXV-3 |
| | set5-LB | G | | |
| 122. | M45012 monkeypox- | GTTTATTCTCAAATA | MPXV-set6 | MPXV-1 or MPXV-3 |
| | set6-F3 | GCGATTG | | |
| 123. | M45013 monkeypox- | CATTGATGAATGTCA | MPXV-set6 | MPXV-1 or MPXV-3 |
| | set6-B3 | TAACTTCA | | |
| 124. | M45014 monkeypox- | CGTTAATAGTGTGCA | MPXV-set6 | MPXV-1 or MPXV-3 |
| | set6-FIP | AGAATTCAGTGTTGT | | |
| | | AAGGATCCTGG | | |
| 125. | M45015 monkeypox- | TGGTTTTTTAACGCA | MPXV-set6 | MPXV-1 or MPXV-3 |
| | set6-BIP | ATGGTCGAAGATGGT | | |
| | | AAGATCCGTC | | |
| 126. | M45016 monkeypox- | CATGTTGGTTAACTT | MPXV-set6 | MPXV-1 or MPXV-3 |
| | set6-LF | ACTGC | | |
| 127. | M45017 monkeypox- | ATATACTGACCGAGT | MPXV-set6 | MPXV-1 or MPXV-3 |
| | set6-LB | AGG | | |
| 128. | M45018 monkeypox- | GTTTACTTTTAGTCC | MPXV-set7 | MPXV-2 |
| | set7-F3 | GTATC | | |
| 129. | M45019 monkeypox- | TTACTCTTTTGTATC | MPXV-set7 | MPXV-2 |
| | set7-B3 | GCA | | |
| 130. | M45020 monkeypox- | ACAATTATTGGTGAG | MPXV-set7 | MPXV-2 |
| | set7-FIP | TGTCATATGACAGTC | | |
| | | AACACTATGTTAGC | | |
| 131. | M45021 monkeypox- | CGGTAATCTTGTCGA | MPXV-set7 | MPXV-2 |
| | set7-BIP | TGAGTCTCTCGGGTA | | |
| | | TTCGGTAG | | |
| 132. | M45022 monkeypox- | GTGAAAGCTATATCG | MPXV-set7 | MPXV-2 |
| | set7-LF | ACAG | | |
| 133. | M45023 monkeypox- | GACATATAGTATTCT | MPXV-set7 | MPXV-2 |
| | set7-LB | TGTATTC | | |
| 134. | M45024 monkeypox- | TCCGTATCCAGTCAA | MPXV-set8 | MPXV-2 |
| | set8-F3 | CAC | | |
| 135. | M45025 monkeypox- | TCCGTATCCAGTCAA | MPXV-set8 | MPXV-2 |
| | set8-B3 | CAC | | |
| 136. | M45026 monkeypox- | TCCGTATCCAGTCAA | MPXV-set8 | MPXV-2 |
| | set8-FIP | CAC | | |
| 137. | M45027 monkeypox- | TCCGTATCCAGTCAA | MPXV-set8 | MPXV-2 |
| | set8-BIP | CAC | | |
| 138. | M45028 monkeypox- | GTGAAAGCTATATCG | MPXV-set8 | MPXV-2 |
| | set8-LF | ACAG | | |
| 139. | M45029 monkeypox- | GACATATAGTATTCT | MPXV-set8 | MPXV-2 |
| | set8-LB | TGTATTC | | |
| 140. | M45030 monkeypox- | GTTTACTTTTAGTCC | MPXV-set9 | MPXV-2 |
| | set9-F3 | GTATC | | |
| 141. | M45031 monkeypox- | TTACTCTTTTGTATC | MPXV-set9 | MPXV-2 |
| | set9-B3 | GCA | | |
| 142. | M45032 monkeypox- | ATTGGTGAGTGTCAT | MPXV-set9 | MPXV-2 |
| | set9-FIP | ATGACAGTCAACACT | | |
| | | ATGTTAGC | | |
| 143. | M45033 monkeypox- | CGGTAATCTTGTCGA | MPXV-set9 | MPXV-2 |
| | set9-BIP | TGAGTCTCTCGGGTA | | |
| | | TTCGGTAG | | |
| 144. | M45034 monkeypox- | GTGAAAGCTATATCG | MPXV-set9 | MPXV-2 |
| | set9-LF | ACAG | | |
| 145. | M45035 monkeypox- | GACATATAGTATTCT | MPXV-set9 | MPXV-2 |
| | set9-LB | TGTATTC | | |
| 146. | M45036 monkeypox- | TCCGTATCCAGTCAA | MPXV-set10 | MPXV-2 |
| | set10-F3 | CAC | | |
| 147. | M45037 monkeypox- | TTACTCTTTTGTATC | MPXV-set10 | MPXV-2 |
| | set10-B3 | GCA | | |
| 148. | M45038 monkeypox- | AATTCTACAATTATT | MPXV-set10 | MPXV-2 |
| | set10-FIP | GGTGAGTGTATGTTA | | |
| | | GCATTTCTGTCG | | |
| 149 | M45039 monkeypox- | CGGTAATCTTGTCGA | MPXV-set10 | MPXV-2 |
| | set10-BIP | TGAGTCTCTCGGGTA | | |
| | | TTCGGTAG | | |
| 150. | M45040 monkeypox- | GTGAAAGCTATATCG | MPXV-set10 | MPXV-2 |
| | set10-LF | ACAG | | |
| 151. | M45041 monkeypox- | GACATATAGTATTCT | MPXV-set10 | MPXV-2 |
| | set10-LB | TGTATTC | | |
| 152. | M45042 monkeypox- | TCCGTATCCAGTCAA | MPXV-set11 | MPXV-2 |
| | set11-F3 | CAC | | |
| 153. | M45043 monkeypox- | TTACTCTTTTGTATC | MPXV-set11 | MPXV-2 |
| | set11-B3 | GCA | | |
| 154. | M45044 monkeypox- | AATTCTACAATTATT | MPXV-set11 | MPXV-2 |
| | set11-FIP | GGTGAGTGTATGTTA | | |
| | | GCATTTCTGTCG | | |
| 155. | M45045 monkeypox- | CGGTAATCTTGTCGA | MPXV-set11 | MPXV-2 |
| | set11-BIP | TGAGTCTCTCGGGTA | | |
| | | TTCGGTAG | | |
| 156. | M45046 monkeypox- | GTGAAAGCTATATCG | MPXV-set11 | MPXV-2 |
| | set11-LF | ACAG | | |
| 157. | M45047 monkeypox- | GACATATAGTATTCT | MPXV-set11 | MPXV-2 |
| | set11-LB | TGTATTC | | |
| 158. | M45048 monkeypox- | TCCGTATCCAGTCAA | MPXV-set12 | MPXV-2 |
| | set12-F3 | CAC | | |
| 159. | M45049 monkeypox- | TTACTCTTTTGTATC | MPXV-set12 | MPXV-2 |
| | set12-B3 | GCA | | |
| 160. | M45050 monkeypox- | CTACAATTATTGGTG | MPXV-set12 | MPXV-2 |
| | set12-FIP | AGTGTATGTTAGCAT | | |
| | | TTCTGTCG | | |
| 161. | M45051 monkeypox- | CGGTAATCTTGTCGA | MPXV-set12 | MPXV-2 |
| | set12-BIP | TGAGTCTCTCGGGTA | | |
| | | TTCGGTAG | | |
| 162. | M45052 monkeypox- | GTGAAAGCTATATCG | MPXV-set12 | MPXV-2 |
| | set12-LF | ACAG | | |
| 163. | M45053 monkeypox- | GACATATAGTATTCT | MPXV-set12 | MPXV-2 |
| | set12-LB | TGTATTC | | |
| 164. | M45054 monkeypox- | TATTCGACTGGTGTC | MPXV-set13 | MPXV-4 |
| | set13-F3 | AGG | | |
| 165. | M45055 monkeypox- | TGTATTGTGTGCGCG | MPXV-set13 | MPXV-4 |
| | set13-B3 | TAA | | |
| 166. | M45056 monkeypox- | CTATCACTCCTATTA | MPXV-set13 | MPXV-4 |
| | set13-FIP | AAGGCTGGATACGTT | | |
| | | CGATATAAACATATG | | |
| | | C | | |
| 167. | M45057 monkeypox- | TCTGTATAATAAGAT | MPXV-set13 | MPXV-4 |
| | set13-BIP | GCAAAGGCAAAAAT | | |
| | | AATTCACATATTGG | | |
| 168. | M45058 monkeypox- | TGAACCGATCCACTG | MPXV-set13 | MPXV-4 |
| | set13-LF | ATG | | |
| 169. | M45059 monkeypox- | GTAGTAAAGATGCTA | MPXV-set13 | MPXV-4 |
| | set13-LB | GTG | | |
| 170. | M45060 monkeypox- | TATTCGACTGGTGTC | MPXV-set14 | MPXV-4 |
| | set14-F3 | AGG | | |
| 171. | M45061 monkeypox- | TGTATTGTGTGCGCG | MPXV-set14 | MPXV-4 |
| | set14-B3 | TAA | | |
| 172. | M45062 monkeypox- | TCACTCCTATTAAAG | MPXV-set14 | MPXV-4 |
| | set14-FIP | GCTGCGTTCGATATA | | |
| | | AACATATGC | | |
| 173. | M45063 monkeypox- | TCTGTATAATAAGAT | MPXV-set14 | MPXV-4 |
| | set14-BIP | GCAAAGGCAAAAAT | | |
| | | AATTCACATATTGG | | |
| 174. | M45064 monkeypox- | TGAACCGATCCACTG | MPXV-set14 | MPXV-4 |
| | set14-LF | ATG | | |
| 175. | M45065 monkeypox- | GTAGTAAAGATGCTA | MPXV-set14 | MPXV-4 |
| | set14-LB | GTG | | |
| 176. | M45066 monkeypox- | AATACATGTCTTAGA | MPXV-set15 | MPXV-6 |
| | set15-F3 | TGTTC | | |
| 177. | M45067 monkeypox- | TCTTGATAATCTTGA | MPXV-set15 | MPXV-6 |
| | set15-B3 | TGAGT | | |
| 178. | M45068 monkeypox- | AGAAATACTTTAGAT | MPXV-set15 | MPXV-6 |
| | set15-FIP | ACGTGCGTAAAACTT | | |
| | | AATCTCTCTCCT | | |
| 179. | M45069 monkeypox- | AGTTAAATTAGATTT | MPXV-set15 | MPXV-6 |
| | set15-BIP | CGAACGAAGGTATA | | |
| | | CCAGAAAAGACGGT | | |
| 180. | M45070 monkeypox- | GGTATCAATTTTTGT | MPXV-set15 | MPXV-6 |
| | set15-LF | TAAGAG | | |
| 181. | M45071 monkeypox- | TCAAATGAACGCCA | MPXV-set15 | MPXV-6 |
| | set15-LB | GAGGCGT | | |
| 182. | M45072 monkeypox- | CAATACATGTCTTAG | MPXV-set16 | MPXV-6 |
| | set16-F3 | ATGTTC | | |
| 183. | M45073 monkeypox- | TCTTGATAATCTTGA | MPXV-set16 | MPXV-6 |
| | set16-B3 | TGAGT | | |
| 184. | M45074 monkeypox- | GAAATACTTTAGATA | MPXV-set16 | MPXV-6 |
| | set16-FIP | CGTGCCAAACTTAAT | | |
| | | CTCTCTCC | | |
| 185. | M45075 monkeypox- | GTTAAATTAGATTTC | MPXV-set16 | MPXV-6 |
| | set16-BIP | GAACGAAGGTATAC | | |
| | | CAGAAAAGACGGT | | |
| 186. | M45076 monkeypox- | GGTATCAATTTTTGT | MPXV-set16 | MPXV-6 |
| | set16-LF | TAAGAG | | |
| 187. | M45077 monkeypox- | TCAAATGAACGCCA | MPXV-set16 | MPXV-6 |
| | set16-LB | GAGGCGT | | |
| 188. | M45078 monkeypox- | TTCAGGGAAATCGCA | MPXV-set17 | MPXV-8 |
| | set17-F3 | TCT | | |
| 189. | M45079 monkeypox- | CCCATATACTTTATT | MPXV-set17 | MPXV-8 |
| | set17-B3 | CATGG | | |
| 190. | M45080 monkeypox- | CCATTAGCTCCATAA | MPXV-set17 | MPXV-8 |
| | set17-FIP | TACAGTTCTATGAAA | | |
| | | GGAAAGAATGT | | |
| 191. | M45081 monkeypox- | CAAAATATGTAGAA | MPXV-set17 | MPXV-8 |
| | set17-BIP | AAGGAGGTTTGAAT | | |
| | | GAGATATTCTGAG | | |
| 192. | M45082 monkeypox- | TTGACGCTGGAGAA | MPXV-set17 | MPXV-8 |
| | set17-LF | ATGA | | |
| 193. | M45083 monkeypox- | AGAACATGGATAAG | MPXV-set17 | MPXV-8 |
| | set17-LB | GTTAGC | | |
| 194. | M45084 monkeypox- | TTCAGGGAAATCGCA | MPXV-set18 | MPXV-8 |
| | set18-F3 | TCT | | |
| 195. | M45085 monkeypox- | CCCATATACTTTATT | MPXV-set18 | MPXV-8 |
| | set18-B3 | CATGG | | |
| 196. | M45086 monkeypox- | CCATTAGCTCCATAA | MPXV-set18 | MPXV-8 |
| | set18-FIP | TACAGTCTATGAAAG | | |
| | | GAAAGAATG | | |
| 197. | M45087 monkeypox- | CAAAATATGTAGAA | MPXV-set18 | MPXV-8 |
| | set18-BIP | AAGGAGGTGAATGA | | |
| | | GATATTCTGAG | | |
| 198. | M45088 monkeypox- | TGACGCTGGAGAAA | MPXV-set18 | MPXV-8 |
| | set18-LF | TGA | | |
| 199. | M45089 monkeypox- | AGAACATGGATAAG | MPXV-set18 | MPXV-8 |
| | set18-LB | GTTAGC | | |
| 200. | M45090 monkeypox- | TCGCATCTTCTATGA | MPXV-set19 | MPXV-8 |
| | set19-F3 | AAG | | |
| 201. | M45091 monkeypox- | CCCATATACTTTATT | MPXV-set19 | MPXV-8 |
| | set19-B3 | CATGG | | |
| 202. | M45092 monkeypox- | CCATTAGCTCCATAA | MPXV-set19 | MPXV-8 |
| | set19-FIP | TACAGTGAAAGAAT | | |
| | | GTATTCATTTCTCCA | | |
| 203. | M45093 monkeypox- | CAAAATATGTAGAA | MPXV-set19 | MPXV-8 |
| | set19-BIP | AAGGAGGTTTGAAT | | |
| | | GAGATATTCTGAG | | |
| 204. | M45094 monkeypox- | ACTGACGAGATTGAC | MPXV-set19 | MPXV-8 |
| | set19-LF | GC | | |
| 205. | M45095 monkeypox- | AGAACATGGATAAG | MPXV-set19 | MPXV-8 |
| | set19-LB | GTTAGC | | |
| 206. | M45096 monkeypox- | TCGCATCTTCTATGA | MPXV-set20 | MPXV-8 |
| | set20-F3 | AAG | | |
| 207. | M45097 monkeypox- | CCCATATACTTTATT | MPXV-set20 | MPXV-8 |
| | set20-B3 | CATGG | | |
| 208. | M45098 monkeypox- | CATTAGCTCCATAAT | MPXV-set20 | MPXV-8 |
| | set20-FIP | ACAGAGAATGTATTC | | |
| | | ATTTCTCC | | |
| 209. | M45099 monkeypox- | TATGTAGAAAAGGA | MPXV-set20 | MPXV-8 |
| | set20-BIP | GGTGAATGAGATATT | | |
| | | CTGAG | | |
| 210. | M45100 monkeypox- | ACTGACGAGATTGAC | MPXV-set20 | MPXV-8 |
| | set20-LF | GC | | |
| 211. | M45101 monkeypox- | GAACATGGATAAGG | MPXV-set20 | MPXV-8 |
| | set20-LB | TTAGC | | |
| 212. | M45102 monkeypox- | TGTTATCCAACTTGA | MPXV-set21 | MPXV-7 |
| | set21-F3 | CAA | | |
| 213. | M45103 monkeypox- | ATACTCGAGTCTCTG | MPXV-set21 | MPXV-7 |
| | set21-B3 | CTG | | |
| 214. | M45104 monkeypox- | AGGTTATTATCTGGA | MPXV-set21 | MPXV-7 |
| | set21-FIP | TCATCTATCACTTAA | | |
| | | TATCAGAGAGATAG | | |
| | | AAGA | | |
| 215. | M45105 monkeypox- | GTAAATCCCACAGA | MPXV-set21 | MPXV-7 |
| | set21-BIP | ACTAATGGTTCGCCA | | |
| | | TCTATCCTCT | | |
| 216. | M45106 monkeypox- | GCGTTTGTCTCATAT | MPXV-set21 | MPXV-7 |
| | set21-LF | GT | | |
| 217. | M45107 monkeypox- | TCTAGGTACTTATGG | MPXV-set21 | MPXV-7 |
| | set21-LB | ACAAC | | |
| 218. | M45108 monkeypox- | AGTTCCATTATCTAA | MPXV-set22 | MPXV-7 |
| | set22-F3 | AGCT | | |
| 219. | M45109 monkeypox- | ATACTCGAGTCTCTG | MPXV-set22 | MPXV-7 |
| | set22-B3 | CTG | | |
| 220. | M45110 monkeypox- | GTTTGTCTCATATGT | MPXV-set22 | MPXV-7 |
| | set22-FIP | TCTTCTATATCCATG | | |
| | | TTATCCAACTT | | |
| 221. | M45111 monkeypox- | GTAAATCCCACAGA | MPXV-set22 | MPXV-7 |
| | set22-BIP | ACTAATGTTCGCCAT | | |
| | | CTATCCTCTG | | |
| 222. | M45112 monkeypox- | CTCTGATATTAAGAT | MPXV-set22 | MPXV-7 |
| | set22-LF | TTGTC | | |
| 223. | M45113 monkeypox- | TCTAGGTACTTATGG | MPXV-set22 | MPXV-7 |
| | set22-LB | ACAAC | | |
| 224. | M45114 monkeypox- | GGAATCCATGTTATC | MPXV-set23 | MPXV-7 |
| | set23-F3 | CAA | | |
| 225. | M45115 monkeypox- | CCAATACTCGAGTCT | MPXV-set23 | MPXV-7 |
| | set23-B3 | CTG | | |
| 226. | M45116 monkeypox- | TCATCTATCAGCGTT | MPXV-set23 | MPXV-7 |
| | set23-FIP | TGTCTCTTGACAAAT | | |
| | | CTTAATATCAGAG | | |
| 227. | M45117 monkeypox- | GTAAATCCCACAGA | MPXV-set23 | MPXV-7 |
| | set23-BIP | ACTAATGTTCGCCAT | | |
| | | CTATCCTCTG | | |
| 228. | M45118 monkeypox- | CATATGTTCTTCTAT | MPXV-set23 | MPXV-7 |
| | set23-LF | CT | | |
| 229. | M45119 monkeypox- | TCTAGGTACTTATGG | MPXV-set23 | MPXV-7 |
| | set23-LB | ACAAC | | |
| 230. | M45120 monkeypox- | TGTTATCCAACTTGA | MPXV-set24 | MPXV-7 |
| | set24-F3 | CAA | | |
| 231. | M45121 monkeypox- | AATACTCGAGTCTCT | MPXV-set24 | MPXV-7 |
| | set24-B3 | GCT | | |
| 232. | M45122 monkeypox- | TCAGGAGGTTATTAT | MPXV-set24 | MPXV-7 |
| | set24-FIP | CTGGAGAGAGATAG | | |
| | | AAGAACATATGAGA | | |
| 233. | M45123 monkeypox- | GTAAATCCCACAGAC | MPXV-set24 | MPXV-7 |
| | set24-BIP | TAATGTTCGCCATCT | | |
| | | ATCCTCTG | | |
| 234. | M45124 monkeypox- | TCATCTATCAGCGTT | MPXV-set24 | MPXV-7 |
| | set24-LF | TG | | |
| 235. | M45125 monkeypox- | TCTAGGTACTTATGG | MPXV-set24 | MPXV-7 |
| | set24-LB | ACAAC | | |

In some embodiments, amplifying a target nucleic acid comprises at least one amplification primer. In some embodiments, amplifying a target nucleic acid comprises at least two amplification primers. In some embodiments, amplifying a target nucleic acid comprises at least three amplification primers. In some embodiments, amplifying a target nucleic acid comprises at least four amplification primers. In some embodiments, amplifying a target nucleic acid comprises at least five amplification primers. In some embodiments, amplifying a target nucleic acid comprises at least six amplification primers. In some embodiments, amplifying a target nucleic acid comprises three amplification primers. In some embodiments, amplifying a target nucleic acid comprises six amplification primers.

In some embodiments, an amplification primer is a FIP primer, a BIP primer, a B3 primer, a F3 primer, a LB primer, or a LF primer. In some embodiments, amplifying a target nucleic acid comprises at least one primer selected from the group consisting of a FIP primer, a BIP primer, a B3 primer, a F3 primer, a LB primer, and a LF primer. In some embodiments, amplifying a target nucleic acid comprises at least two primers selected from the group consisting of a FIP primer, a BIP primer, a B3 primer, a F3 primer, a LB primer, and a LF primer. In some embodiments, amplifying a target nucleic acid comprises at least three primers selected from the group consisting of a FIP primer, a BIP primer, a B3 primer, a F3 primer, a LB primer, and a LF primer. In some embodiments, amplifying a target nucleic acid comprises at least four primers selected from the group consisting of a FIP primer, a BIP primer, a B3 primer, a F3 primer, a LB primer, and a LF primer. In some embodiments, amplifying a target nucleic acid comprises at least five primers selected from the group consisting of a FIP primer, a BIP primer, a B3 primer, a F3 primer, a LB primer, and a LF primer. In some embodiments, amplifying a target nucleic acid comprises at least six primers selected from the group consisting of a FIP primer, a BIP primer, a B3 primer, a F3 primer, a LB primer, and a LF primer. In some embodiments, amplifying a target nucleic acid comprises a FIP primer, a BIP primer, a B3 primer, a F3 primer, a LB primer, and a LF primer. In some embodiments, amplifying a target nucleic acid comprises a FIP primer, a F3 primer, and a LF primer. In some embodiments, amplifying a target nucleic acid comprises a BIP primer, a B3 primer, and a LB primer.

In some embodiments, a FIP primer comprises a nucleotide sequence at least 80%, at least 85%, at least 87%, at least 90%, at least 92%, at least 94%, at least 95%, at least 96%, at least 98%, or 100% identical to any one of SEQ ID NOs 94, 100, 106, 112, 118, 124, 130, 136, 142, 148, 154, 160, 166, 171, 178, 184, 190, 196, 202, 208, 214, 220, 226, or 232. In some embodiments, a BIP primer comprises a nucleotide sequence at least 80%, at least 85%, at least 87%, at least 90%, at least 92%, at least 94%, at least 95%, at least 96%, at least 98%, or 100% identical to any one of SEQ ID NOs 4, 10, 16, 22, 28, 34, 40, 46, 52, 58, 64, 70, 76, 82, 88, 94, 100, 106, 112, 118, 124, 130, 136, or 14295, 101, 107, 113, 119, 125, 131, 137, 143, 149, 155, 161, 167, 172, 179, 185, 191, 197, 203, 209, 215, 221, 227, or 233. In some embodiments, a B3 primer comprises a nucleotide sequence at least 80%, at least 85%, at least 87%, at least 90%, at least 92%, at least 94%, at least 95%, at least 96%, at least 98%, or 100% identical to any one of SEQ ID NOs 93, 99, 105, 111, 117, 123, 129, 135, 141, 147, 153, 159, 165, 170, 177, 183, 189, 195, 201, 207, 213, 219, 225, or 231. In some embodiments, a F3 primer comprises a nucleotide sequence at least 80%, at least 85%, at least 87%, at least 90%, at least 92%, at least 94%, at least 95%, at least 96%, at least 98%, or 100% identical to any one of SEQ ID NOs 92, 98, 104, 110, 116, 122, 128, 134, 140, 146, 152, 158, 164, 170, 176, 182, 188, 194, 200, 206, 212, 218, 224, or 230. In some embodiments, a LF primer comprises a nucleotide sequence at least 80%, at least 85%, at least 87%, at least 90%, at least 92%, at least 94%, at least 95%, at least 96%, at least 98%, or 100% identical to any one of SEQ ID NOs 96, 102, 108, 114, 120, 126, 132, 138, 144, 150, 156, 162, 168, 173, 180, 186, 192, 198, 204, 210, 216, 222, 228, or 234. In some embodiments, a LB primer comprises a nucleotide sequence at least 80%, at least 85%, at least 87%, at least 90%, at least 92%, at least 94%, at least 95%, at least 96%, at least 98%, or 100% identical to any one of SEQ ID NOs 97, 103, 109, 115, 121, 127, 133, 139,145,151, 157, 163, 169, 174, 181, 187, 193, 199, 205, 211, 217, 223, 229, or 235.

In some embodiments, the amplifying comprises isothermal amplification. In some embodiments, the amplifying comprises helicase dependent amplification (HDA), circular helicase dependent amplification (cHDA), strand displacement amplification (SDA), loop mediated amplification (LAMP), exponential amplification reaction (EXPAR), rolling circle amplification (RCA), ligase chain reaction (LCR), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). In some embodiments, the amplifying comprises loop mediated amplification (LAMP).

In some embodiments, the amplifying comprises a thermal cycling amplification. In some embodiments, the amplifying comprises polymerase chain reaction (PCR).

The amplifying can improve at least one of sensitivity, specificity, or accuracy of the detection the target nucleic acid. The reagents for nucleic acid amplification can comprise a recombinase, an oligonucleotide primer, a single-stranded DNA binding (SSB) protein, and a polymerase. The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. In some embodiments, the nucleic acid amplification reaction is performed at a temperature of around 20-65° C. In some embodiments, the nucleic acid amplification reaction is performed at a temperature no greater than 20° C., 25° C., 30° C., 35° C., 37° C., 40° C., 45° C., 50° C., 55° C., 60° C., 65° C. In some embodiments, the nucleic acid amplification reaction is performed at a temperature of at least 20° C., 25° C., 30° C., 35° C., 37° C., 40° C., 45° C., 50° C., 55° C., 60° C., of 65° C.

In some embodiments, the amplifying comprises contacting the sample to reagents for amplification. In some embodiments, the contacting the sample to reagents for amplification occurs concurrent to the contacting the sample to the reporter and the composition comprising the programmable nuclease and the guide nucleic acid. In some embodiments, the contacting the sample to reagents for amplification occurs prior to the contacting the sample to the reporter and the composition comprising the programmable nuclease and the guide nucleic acid. In some embodiments, the reagents for amplification comprise a polymerase and dNTPs.

### Guide Nucleic Acids

The reagents described herein may comprise guide nucleic acids, as described herein, designed to target at least a segment of a target nucleic acid of monkeypox virus. The reagents described herein comprise multiple guide nucleic acids. Each guide nucleic acid comprises a sequence (e.g., a spacer sequence) that is reverse complementary to a segment of a target nucleic acid of monkeypox virus. Each guide nucleic acid specifically binds to the segment of the target nucleic acid of monkeypox virus. In some embodiments, each guide nucleic acid is able to distinguish between two target nucleic acids, wherein the two target nucleic acids comprise a difference in the nucleotide sequences between each other. In some embodiments, the guide nucleic acid is non-naturally occurring and made by artificial combination of otherwise separate segments of sequence. In some embodiments, the artificial combination is performed by chemical synthesis, by genetic engineering techniques, or by the artificial manipulation of isolated segments of nucleic acids. The non-naturally occurring guide nucleic acid or segment thereof can be designed and made to provide desired functions.

In some embodiments, a guide nucleic acid comprises a nucleotide sequence at least 80%, at least 85%, at least 87%, at least 90%, at least 92%, at least 94%, at least 95%, at least 96%, at least 98%, or 100% identical to any one of SEQ ID NOs 236-247, as provided in Table below.

TABLE 4
Exemplary Non-Naturally Occurring Guide Nucleic Acid Sequences

| SEQ ID | | |
|---|---|---|
| NO | Name | Guide Nucleic Acid Sequence* |
| 236. | R13932 | gACCGCUUCACCAAGUGCUGUCCCUUAGGGGAUUAG |
| | MPXV-1 | CACUUGAGUGAAGGUGGGCUGCUUGCAUCAGCCUAA |
| | | UGUCGAGAAGUGCUUUCUUCGGAAAGUAACCCUCGA |
| | | AACAAAUUCAUUgaaaGAAUGAAGGAAUGCAACUCGG |
| | | CUACACCUAUCGUUAA |
| 237. | R13933 | gACCGCUUCACCAAGUGCUGUCCCUUAGGGGAUUAG |
| | MPXV-2 | CACUUGAGUGAAGGUGGGCUGCUUGCAUCAGCCUAA |
| | | UGUCGAGAAGUGCUUUCUUCGGAAAGUAACCCUCGA |
| | | AACAAAUUCAUUgaaaGAAUGAAGGAAUGCAACACCA |
| | | AUAGUGAGUUCGGCGA |
| 238. | R13934 | gACCGCUUCACCAAGUGCUGUCCCUUAGGGGAUUAG |
| | MPXV-3 | CACUUGAGUGAAGGUGGGCUGCUUGCAUCAGCCUAA |
| | | UGUCGAGAAGUGCUUUCUUCGGAAAGUAACCCUCGA |
| | | AACAAAUUCAUUgaaaGAAUGAAGGAAUGCAACCGAU |
| | | AGGUGUAGCCGAUAAA |
| 239. | R13935 | gACCGCUUCACCAAGUGCUGUCCCUUAGGGGAUUAG |
| | MPXV-4 | CACUUGAGUGAAGGUGGGCUGCUUGCAUCAGCCUAA |
| | | UGUCGAGAAGUGCUUUCUUCGGAAAGUAACCCUCGA |
| | | AACAAAUUCAUUgaaaGAAUGAAGGAAUGCAACUUAA |
| | | CGAUUGUCGACCCUCU |
| 240. | R13936 | gACCGCUUCACCAAGUGCUGUCCCUUAGGGGAUUAG |
| | MPXV-5 | CACUUGAGUGAAGGUGGGCUGCUUGCAUCAGCCUAA |
| | | UGUCGAGAAGUGCUUUCUUCGGAAAGUAACCCUCGA |
| | | AACAAAUUCAUUgaaaGAAUGAAGGAAUGCAACUGAA |
| | | GAUGCCAUGUACUACG |
| 241. | R13937 | gACCGCUUCACCAAGUGCUGUCCCUUAGGGGAUUAG |
| | MPXV-6 | CACUUGAGUGAAGGUGGGCUGCUUGCAUCAGCCUAA |
| | | UGUCGAGAAGUGCUUUCUUCGGAAAGUAACCCUCGA |
| | | AACAAAUUCAUUgaaaGAAUGAAGGAAUGCAACACUU |
| | | UUACGCCUCUGGCGUU |
| 242. | R13938 | gACCGCUUCACCAAGUGCUGUCCCUUAGGGGAUUAG |
| | MPXV-7 | CACUUGAGUGAAGGUGGGCUGCUUGCAUCAGCCUAA |
| | | UGUCGAGAAGUGCUUUCUUCGGAAAGUAACCCUCGA |
| | | AACAAAUUCAUUgaaaGAAUGAAGGAAUGCAACAAAA |
| | | UGGCCAAAGCGGGUUA |
| 243. | R13939 | gACCGCUUCACCAAGUGCUGUCCCUUAGGGGAUUAG |
| | MPXV-8 | CACUUGAGUGAAGGUGGGCUGCUUGCAUCAGCCUAA |
| | | UGUCGAGAAGUGCUUUCUUCGGAAAGUAACCCUCGA |
| | | AACAAAUUCAUUgaaaGAAUGAAGGAAUGCAACGAUC |
| | | CACUGCUGAACAGCUA |
| 244. | R13977 | gACCGCUUCACCAAGUGCUGUCCCUUAGGGGAUUAG |
| | MPXV-3 | CACUUGAGUGAAGGUGGGCUGCUUGCAUCAGCCUAA |
| | (fixed) | UGUCGAGAAGUGCUUUCUUCGGAAAGUAACCCUCGA |
| | | AACAAAUUCAUUgaaaGAAUGAAGGAAUGCAACUUUA |
| | | UCGGCUACACCUAUCG |
| 245. | R13978 | gACCGCUUCACCAAGUGCUGUCCCUUAGGGGAUUAG |
| | MPXV-7 | CACUUGAGUGAAGGUGGGCUGCUUGCAUCAGCCUAA |
| | (fixed) | UGUCGAGAAGUGCUUUCUUCGGAAAGUAACCCUCGA |
| | | AACAAAUUCAUUgaaaGAAUGAAGGAAUGCAACUAAC |
| | | CCGCUUUGGCCAUUUU |
| 246. | R13979 | gACCGCUUCACCAAGUGCUGUCCCUUAGGGGAUUAG |
| | MPXV-8 | CACUUGAGUGAAGGUGGGCUGCUUGCAUCAGCCUAA |
| | (fixed) | UGUCGAGAAGUGCUUUCUUCGGAAAGUAACCCUCGA |
| | | AACAAAUUCAUUgaaaGAAUGAAGGAAUGCAACAUAG |
| | | CUGUUCAGCAGUGGAU |
| 247. | R13980 | gACCGCUUCACCAAGUGCUGUCCCUUAGGGGAUUAG |
| | MPXV-2 | CACUUGAGUGAAGGUGGGCUGCUUGCAUCAGCCUAA |
| | (fixed) | UGUCGAGAAGUGCUUUCUUCGGAAAGUAACCCUCGA |
| | | AACAAAUUCAUUgaaaGAAUGAAGGAAUGCAACUCGC |
| | | CGAACUCACUAUUGGU |

**The first lowercase base (g) is the starting nucleotide from T7. The middle lowercase bases (gaaa) are a linker between (i) the portion of the handle that comprises at least a portion of a tracrRNA sequence and (ii) the portion of the handle that contains at least a portion of a repeat sequence of the sgRNA backbone.

The target site sequence of each non-naturally occurring guide nucleic acid is disclosed in Table below.

TABLE 5
Exemplary Target Site Sequences of Guide
Nucleic Acids

| SEQ ID | | |
|---|---|---|
| NO | Name | Target Site Sequence |
| 248. | R13932 MPXV-1 | TCGGCTACACCTATCGTTAA |
| 249. | R13933 MPXV-2 | ACCAATAGTGAGTTCGGCGA |
| 250. | R13934 MPXV-3 | CGATAGGTGTAGCCGATAAA |
| 251. | R13935 MPXV-4 | TTAACGATTGTCGACCCTCT |
| 252. | R13936 MPXV-5 | TGAAGATGCCATGTACTACG |
| 253. | R13937 MPXV-6 | ACTTTTACGCCTCTGGCGTT |
| 254. | R13938 MPXV-7 | AAAATGGCCAAAGCGGGTTA |
| 255. | R13939 MPXV-8 | GATCCACTGCTGAACAGCTA |
| 256. | R13977 MPXV-3 | TTTATCGGCTACACCTATCG |
| | (fixed) | |
| 257. | R13978 MPXV-7 | TAACCCGCTTTGGCCATTTT |
| | (fixed) | |
| 258. | R13979 MPXV-8 | ATAGCTGTTCAGCAGTGGAT |
| | (fixed) | |
| 259. | R13980 MPXV-2 | TCGCCGAACTCACTATTGGT |
| | (fixed) | |

### Effector Proteins

The reagents described herein comprise a programmable nuclease (also referred to, interchangeably, as an effector protein) capable of being activated when complexed with the guide nucleic acid and the target nucleic acid or segment thereof segment, as described herein. The reagents for monkeypox detection may comprise any of the effector proteins described herein. For example, in some embodiments, the Type V CRISPR/Cas enzyme is a programmable Cas14 nuclease. In some embodiments, the Cas14 nuclease is Cas14a.1.

### Reporter Nucleic Acids

The reagents described herein comprise one or more reporters as described herein. The one or more reporters may comprise any of the detection moieties described herein. In some embodiments, the one or more reporters may be in solution. In some embodiments, the one or more reporters may be immobilized on a solid support or other support medium.

In some embodiments, the reporter comprises a nucleic acid conjugated to an affinity molecule and the affinity molecule conjugated to the fluorophore (e.g., nucleic acid-affinity molecule-fluorophore) or the nucleic acid conjugated to the fluorophore and the fluorophore conjugated to the affinity molecule (e.g., nucleic acid-fluorophore-affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the reporter can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicated that no intervening molecules, polypeptides, proteins, or other moieties are present between the two moieties directly conjugated to each other. For example, if a reporter comprises a nucleic acid directly conjugated to an affinity molecule and an affinity molecule directly conjugated to a fluorophore-no intervening moiety is present between the nucleic acid and the affinity molecule and no intervening moiety is present between the affinity molecule and the fluorophore. The affinity molecule can be biotin, avidin, streptavidin, or any similar molecule.

In some embodiments, the reporter comprises a substrate-nucleic acid. The substrate may be sequestered from its cognate enzyme when present as in the substrate-nucleic acid, but then is released from the nucleic acid upon cleavage, wherein the released substrate can contact the cognate enzyme to produce a detectable signal. In some embodiments, the reporter comprises an enzyme-nucleic acid. The enzyme may be sterically hindered when present as in the enzyme-nucleic acid, but then functional upon cleavage from the nucleic acid.

In some embodiments, an enzyme is invertase. In some embodiments, the substrate of invertase is sucrose or DNS reagent.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid support and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

In some embodiments, the signal is present prior to reporter cleavage and changes upon reporter cleavage. In some embodiments, the signal is absent prior to reporter cleavage and is present upon reporter cleavage. In some embodiments, the detectable signal is generated directly by the cleavage event. Alternatively, or in combination, the detectable signal is generated indirectly by the signal event. In some embodiments, the detected target nucleic acid or segment thereof is identified based on its spatial location on the detection region of the support medium. In some embodiments, the second detectable signal is generated in a spatially distinct location than the first generated signal. In some embodiments, the detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

### Buffers

The reagents described herein comprise buffers. These buffers are compatible with the other reagents and samples as described herein for detection of a target nucleic acid.

In some embodiments, a buffer comprises 20 mM HEPES pH 6.8, 50 mM KCl, 5 mM MgCl₂, and 5% glycerol. In some embodiments, the buffer comprises from 0 to 100, 0 to 75, 0 to 50, 0 to 25, 0 to 20, 0 to 10, 0 to 5, 5 to 10,5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. In some embodiments, the buffer comprises 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In some embodiments, the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. In some embodiments, the buffer can comprise 0 to 25, 0 to 20, 0 to 10, 0 to 5, 5 to 10, 5 to 15, 5 to 20, 5 to 25, 5 to 30% glycerol.

In some embodiments, a buffer comprises 100 mM Imidazole pH 7.5; 250 mM KCl, 25 mM MgCl₂, 50 μg/mL BSA, 0.05% Igepal Ca-630, and 25% Glycerol. In some embodiments, the buffer comprises 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Imidazole pH 7.5. In some embodiments, the buffer comprises 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In some embodiments, the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. In some embodiments, the buffer comprises 0 to 100, 0 to 75, 0 to 50, 0 to 25, 0 to 20, 0 to 10, 0 to 5, 5 to 50, 5 to 75, 5 to 100, 10 to 20, 10 to 50, 10 to 75, 10 to 100, 25 to 50, 25 to 75 25 to 100, 50 to 75, or 50 to 100 μg/mL BSA. In some embodiments, the buffer comprises 0 to 1, 0 to 0.5, 0 to 0.25, 0 to 0.01, 0 to 0.05, 0 to 0.025, 0 to 0.01, 0.01 to 0.025, 0.01 to 0.05, 0.01 to 0.1, 0.01 to 0.25, 0.01, to 0.5, 0.01 to 1, 0.025 to 0.05, 0.025 to 0.1, 0.025, to 0.5, 0.025 to 1, 0.05 to 0.1, 0.05 to 0.25, 0.05 to 0.5, 0.05 to 0.75, 0.05 to 1, 0.1 to 0.25, 0.1 to 0.5, or 0.1 to 1% Igepal Ca-630. The buffer can comprise 0 to 25, 0 to 20, 0 to 10, 0 to 5, 5 to 10, 5 to 15, 5 to 20, 5 to 25, 5 to 30% glycerol.

A buffer of the present disclosure may comprise a viral lysis buffer. In some embodiments, the methods provided herein further comprising lysing the sample. In some 20 embodiments, the lysing comprises contacting the sample to a lysis buffer. In some embodiments, a viral lysis buffer lyses a monkeypox virus capsid in a viral sample (e.g., a sample collected from an individual suspected of having a monkeypox virus infection), releasing a viral genome. In some embodiments, the viral lysis buffer is compatible with amplification (e.g., RT-LAMP amplification) of a target region of the viral genome. In some embodiments, the viral lysis buffer is compatible with detection (e.g., a DETECTR reaction disclosed herein). In some embodiments, a sample is prepared in a one-step sample preparation method comprising suspending the sample in a viral lysis buffer compatible with amplification, detection (e.g., a DETECTR reaction), or both. A viral lysis buffer compatible with amplification (e.g., LAMP amplification), detection (e.g., DETECTR), or both, may comprise a buffer (e.g., Tris-HCl, phosphate, or HEPES), a reducing agent (e.g., N-Acetyl Cysteine (NAC), Dithiothreitol (DTT), β-mercaptoethanol (BME), or tris(2-carboxyethyl) phosphine (TCEP)), a chelating agent (e.g., EDTA or EGTA), a detergent (e.g., deoxycholate, NP-40 (Ipgal), Triton X-100, or Tween 20), a salt (e.g., ammonium acetate, magnesium acetate, manganese acetate, potassium acetate, sodium acetate, ammonium chloride, potassium chloride, magnesium chloride, manganese chloride, sodium chloride, ammonium sulfate, magnesium sulfate, manganese sulfate, potassium sulfate, or sodium sulfate), or a combination thereof. For example, a viral lysis buffer may comprise a buffer and a reducing agent, or a viral lysis buffer may comprise a buffer and a chelating agent. The viral lysis buffer may be formulated at a low pH. For example, the viral lysis buffer may be formulated at a pH of from about pH 4 to about pH 5. In some embodiments, the viral lysis buffer is formulated at a pH of from about pH 4 to about pH 9. The viral lysis buffer may further comprise a preservative (e.g., ProClin 150). In some embodiments, the viral lysis buffer comprises an activator of the amplification reaction. For example, the buffer may comprise primers, dNTPs, or magnesium (e.g., MgSO₄, MgCl₂ or MgOAc), or a combination thereof, to activate the amplification reaction. In some embodiments, an activator (e.g., primers, dNTPs, or magnesium) may be added to the buffer following lysis of the monkeypox virus to initiate the amplification reaction.

A viral lysis buffer may comprise a pH of about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 6, about 6.5, about 7, about 7.5, about 8, about 8.5, or about 9. In some embodiments, a viral lysis buffer may comprise a pH of from 3.5 to 4.5, from 4 to 5, from 4.5 to 5.5, from 3.5 to 4, from 4 to 4.5, from 4.5 to 5, from 5 to 5.5, from 5 to 6, from 6 to 7, from 7 to 8, or from 8 to 9.

A viral lysis buffer may comprise a magnesium concentration of about 0 mM, about 2 mM, about 4 mM, about 5 mM, about 6 mM, about 8 mM, about 10 mM, about 12 mM, about 13 mM, about 14 mM, about 15 mM, about 20 mM, about 25 mM, about 30 mM, about 35 mM, about 40 mM, about 45 mM, about 50 mM, about 55 mM, or about 60 mM of magnesium (e.g., MgSO₄, MgCl₂ or MgOAc). A viral lysis buffer may comprise a magnesium concentration of from 0 mM to 5 mM, from 5 mM to 10 mM, from 10 mM to 15 mM, from 15 mM to 20 mM, from 20 mM to 25 mM, from 25 mM to 30 mM, from 30 mM to 40 mM, from 40 mM to 50 mM, or from 50 mM to 60 mM of magnesium (e.g., MgSO₄, MgCl₂ or MgOAc). In some embodiments, the magnesium may be added after viral lysis to activate an amplification reaction.

A viral lysis buffer may comprise a reducing agent (e.g., NAC, DTT, BME, or TCEP) at a concentration of about 1 mM, about 2 mM, about 3 mM, about 4 mM, about 5 mM, about 6 mM, about 7 mM, about 8 mM, about 10 mM, about 12 mM, about 15 mM, about 20 mM, about 25 mM, about 30 mM, about 40 mM, about 50 mM, about 60 mM, about 7 mM, about 80 mM, about 90 mM, about 100 mM, or about 120 mM. A viral lysis buffer may comprise a reducing agent (e.g., NAC, DTT, BME, or TCEP) at a concentration of from 1 mM to 5 mM, from 5 mM to 10 mM, from 10 mM to 15 mM, from 15 mM to 20 mM, from 20 mM to 25 mM, from 25 mM to 30 mM, from 30 mM to 40 mM, from 40 mM to 50 mM, from 50 mM to 60 mM, from 60 mM to 70 mM, from 70 mM to 80 mM, or from 80 mM to 90 mM, from 90 mM to 100 mM, or from 100 mM to 120 mM. A viral lysis buffer may comprise a chelator (e.g., EDTA or EGTA) at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 10 mM, about 12 mM, about 15 mM, about 20 mM, about 25 mM, or about 30 mM. A viral lysis buffer may comprise a chelator (e.g., EDTA or EGTA) at a concentration of from 0.1 mM to 0.5 mM, from 0.25 mM to 0.5 mM, from 0.4 mM to 0.6 mM, from 0.5 mM to 1 mM, from 1 mM to 5 mM, from 5 mM to 10 mM, from 10 mM to 15 mM, from 15 mM to 20 mM, from 20 mM to 25 mM, or from 25 mM to 30 mM.

A viral lysis buffer may comprise a salt (e.g., ammonium acetate ((NH₄)₂OAc), magnesium acetate (MgOAc), manganese acetate (MnOAc), potassium acetate (K₂OAc), sodium acetate (Na₂OAc), ammonium chloride (NH₄Cl), potassium chloride (KCl), magnesium chloride (MgCl₂), manganese chloride (MnCl₂), sodium chloride (NaCl), ammonium sulfate ((NH₄)₂SO₄), magnesium sulfate (MgSO₄), manganese sulfate (MnSO₄), potassium sulfate (K₂SO₄), or sodium sulfate (Na₂SO₄)) at a concentration of about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 90 mM, or about 100 mM. A viral lysis buffer may comprise a salt (e.g., (NH₄)₂OAc, MgOAc, MnOAc, K₂OAc, Na₂OAc, NH₄Cl, KCl, MgCl₂, MnCl₂, NaCl, (NH₄)₂SO₄, MgSO₄, MnSO₄, K₂SO₄, or Na₂SO₄) at a concentration of from 1 mM to 5 mM, from 1 mM to 10 mM, from 5 mM to 10 mM, from 10 mM to 15 mM, from 15 mM to 20 mM, from 20 mM to 25 mM, from 25 mM to 30 mM, from 30 mM to 35 mM, from 35 mM to 40 mM, from 40 mM to 45 mM, from 45 mM to 50 mM, from 50 mM to 55 mM, from 55 mM to 60 mM, from 60 mM to 70 mM, from 70 mM to 80 mM, from 80 mM to 90 mM, or from 90 mM to 100 mM.

A viral lysis buffer may comprise a detergent (e.g., deoxycholate, NP-40 (Ipgal), Triton X-100, or Tween 20) at a concentration of about 0.01%, about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.00%, about 1.10%, about 1.20%, about 1.30%, about 1.40%, about 1.50%, about 2.00%, about 2.50%, about 3.00%, about 3.50%, about 4.00%, about 4.50%, or about 5.00%. A viral lysis buffer may comprise a detergent (e.g., deoxycholate, NP-40 (Ipgal), Triton X-100, or Tween 20) at a concentration of from 0.01% to 0.10%, from 0.05% to 0.15%, from 0.10% to 0.20%, from 0.15% to 0.25%, from 0.20% to 0.30%, from 0.25% to 0.35%, from 0.30% to 0.40%, from 0.35% to 0.45%, from 0.40% to 0.50%, from 0.45% to 0.55%, from 0.50% to 0.60%, from 0.55% to 0.65%, from 0.60% to 0.70%, from 0.65% to 0.75%, from 0.70% to 0.80%, from 0.75% to 0.85%, from 0.80% to 0.90%, from 0.85% to 0.95%, from 0.90% to 1.00%, from 0.95% to 1.10%, from 1.00% to 1.20%, from 1.10% to 1.30%, from 1.20% to 1.40%, from 1.30% to 1.50%, from 1.40% to 1.60%, from 1.50% to 2.00%, from 2.00% to 2.50%, from 2.50% to 3.00%, from 3.00% to 3.50%, from 3.50% to 4.00%, from 4.00% to 4.50%, or from 4.50% to 5.00%.

A lysis reaction may be performed at a range of temperatures. In some embodiments, a lysis reaction may be performed at about room temperature. In some embodiments, a lysis reaction may be performed at about 95° C. In some embodiments, a lysis reaction may be performed at from 1° C. to 10° C., from 4° C. to 8° C., from 10° C. to 20° C., from 15° C. to 25° C., from 15° C. to 20° C., from 18° C. to 25° C., from 18° C. to 95° C., from 20° C. to 37° C., from 25° C. to 40° C., from 35° C. to 45° C., from 40° C. to 60° C., from 50° C. to 70° C., from 60° C. to 80° C., from 70° C. to 90° C., from 80° C. to 95° C., or from 90° C. to 99° C. In some embodiments, a lysis reaction may be performed for about 5 minutes, about 15 minutes, or about 30 minutes. In some embodiments, a lysis reaction may be performed for from 2 minutes to 5 minutes, from 3 minutes to 8 minutes, from 5 minutes to 15 minutes, from 10 minutes to 20 minutes, from 15 minutes to 25 minutes, from 20 minutes to 30 minutes, from 25 minutes to 35 minutes, from 30 minutes to 40 minutes, from 35 minutes to 45 minutes, from 40 minutes to 50 minutes, from 45 minutes to 55 minutes, from 50 minutes to 60 minutes, from 55 minutes to 65 minutes, from 60 minutes to 70 minutes, from 65 minutes to 75 minutes, from 70 minutes to 80 minutes, from 75 minutes to 85 minutes, or from 80 minutes to 90 minutes.

In some embodiments, the methods, compositions, and kits described herein detect a target nucleic acid (e.g., a nucleic acid from a monkeypox virus) in a sample where the sample is contacted with the reagents for a predetermined length of time sufficient for the trans cleavage to occur or cleavage reaction to reach completion. In some embodiments, the methods, compositions, and kits described herein detect a target nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some embodiments, the methods, compositions, and kits described herein can detect a target nucleic acid or segment thereof in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes.

In some embodiments, sample lysis, amplification, detection, or any combination thereof is carried out in a single volume. In some embodiments, sample lysis, amplification, detection, or any combination thereof is carried out in separate volumes.

### Devices

A number of devices are consistent with the methods, compositions, and kits disclosed herein.

The method described herein can be carried out in various ways. In some embodiments, the method is carried out on a lateral flow strip. In some embodiments, the method is carried out on a high throughput workflow. In some embodiments, the method is carried out on a handheld device. In some embodiments, the method is carried out on a cartridge-based workflow.

The results from a completed assay can be detected and analyzed in various ways. In some embodiments, the device measures or detects a calorimetric, potentiometric, amperometric, optical (e.g., fluorescent, colorimetric, etc.), piezo-electric, piezo-electric, chemical, electrochemical, or magnetic signal. In some embodiments, the results are visible to the eye and can be read directly by the user without the use of another measurement or detection device. In some embodiments, a positive control spot and a detection spot in the detection region of a lateral flow strip is visible by eye, and the results can be read by the user. In some embodiments, the positive control spot and the detection spot in the detection region is visualized by an imaging device or other device depending on the type of signal. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively, or in combination, the imaging device can capture fluorescence, ultraviolet (UV), infrared (IR), or visible wavelength signals. The imaging device may have an excitation source to provide the excitation energy and captures the emitted signals. In some embodiments, the excitation source can be a camera flash and optionally a filter. In some embodiments, the imaging device is used together with an imaging box that is placed over the support medium to create a dark room to improve imaging. The imaging box can be a cardboard box that the imaging device can fit into before imaging. In some embodiments, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid or segment thereof, which indicates the presence of the gene, virus, or the agent responsible for the disease. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, cleanup of an environment.

### Multiplexing

The methods, compositions, and kits described herein can be multiplexed in a number of ways, as described herein.

In some embodiments, multiplexing can be enabled by immobilization of multiple categories of reporters within a fluidic system, to enable detection of multiple target nucleic acids or segments thereof within a single sample.

The methods, compositions, and kits described herein can be multiplexed by various configurations of the reagents and the support medium. In some embodiments, the kit or system is designed to have multiple support mediums encased in a single housing. In some embodiments, the multiple support mediums housed in a single housing share a single sample pad. The single sample pad may be connected to the support mediums in various designs such as a branching or a radial formation. In some embodiments, each of the multiple support mediums has its own sample pad. In some embodiments, the kit or system is designed to have a single support medium encased in a housing, where the support medium comprises multiple detection spots for detecting multiple target nucleic acids or segments thereof.

In some embodiments, this reacted sample is applied to the multiplexed support medium described herein. In some embodiments, the methods, compositions, and kits described herein can be multiplexed in a configuration lacking a support medium.

In some embodiments, multiplexing enables detections of different segments of the same gene of the monkeypox virus at the same time. In some embodiments, multiplexing enables detections of different genes of the monkeypox virus at the same time. In some embodiments, multiplexing enables distinguish between a gene of the monkeypox virus or a segment thereof and a gene of another Orthopoxvirus or a segment thereof. In some embodiments, the other Orthopoxvirus is abatino macacapox virus. In some embodiments, the other Orthopoxvirus is akhmeta virus. In some embodiments, the other Orthopoxvirus is alaskapox virus. In some embodiments, the other Orthopoxvirus is camelpox virus. In some embodiments, the other Orthopoxvirus is cowpox virus. In some embodiments, the other Orthopoxvirus is ectromelia virus. In some embodiments, the other Orthopoxvirus is raccoonpox virus. In some embodiments, the other Orthopoxvirus is skunkpox virus. In some embodiments, the other Orthopoxvirus is taterapox virus. In some embodiments, the other Orthopoxvirus is vaccinia virus. In some embodiments, the other Orthopoxvirus is variola virus. In some embodiments, the other Orthopoxvirus is volepox virus.

### Detection of Disease as a Research Tool, Point-of-Care, or Over-the-Counter

Disclosed herein are methods of assaying for one or more target nucleic acid(s) that can be used for disease detection. The disease can be monkeypox. The various methods, compositions, and kits disclosed herein can be used as a companion diagnostic with medicaments used to treat monkeypox. In some embodiments, the methods, compositions, and kits disclosed herein specifically target and assay for a target nucleic acid of monkeypox virus.

Also disclosed herein are methods of assaying for one or more target nucleic acid(s) that can be used as a research tool, point-of-care, or over-the-counter. In some embodiments, one or more target nucleic acid(s) are from monkeypox virus.

For example, a method of assaying for a target nucleic acid (e.g., from a monkeypox virus) in a sample comprises contacting the sample to a complex comprising a non-naturally occurring guide nucleic acid comprising a segment that is reverse complementary to a segment of the target nucleic acid and a programmable nuclease that exhibits sequence independent cleavage upon forming a complex comprising the segment of the non-naturally occurring guide nucleic acid binding to the segment of the target nucleic acid; and assaying for a signal indicating cleavage of at least some protein-nucleic acids of a population of protein-nucleic acids, wherein the signal indicates a presence of the target nucleic acid or segment thereof in the sample and wherein absence of the signal indicates an absence of the target nucleic acid or segment thereof in the sample.

In yet another example, a method of assaying for a plurality of target nucleic acid in a sample comprise amplifying the target nucleic acid of monkeypox virus using at least one amplification primer; contacting the sample to a plurality of complexes comprising a non-naturally occurring guide nucleic acid comprising a segment that is reverse complementary to a segment of a target nucleic acid of the plurality of target nucleic acids and a programmable nuclease that exhibits sequence independent cleavage upon forming a complex comprising the segment of the non-naturally occurring guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The plurality of complexes may comprise programmable nucleases complexes with non-naturally occurring guide nucleic acids directed to different target nucleic acids.

In some embodiments, a method of assaying for a target nucleic acid in a sample comprising a) amplifying the target nucleic acid using at least one amplification primer; b) contacting the sample to a reporter and a composition comprising a programmable nuclease and a guide nucleic acid that hybridizes to the target nucleic acid or an amplified product thereof, wherein the programmable nuclease cleaves the reporter upon hybridization of the guide nucleic acid to the target nucleic acid or the amplification product thereof; and c) assaying for a change in a signal, wherein the change in the signal is produced by cleavage of the reporter; wherein the target nucleic acid is a gene of a monkeypox virus or a segment thereof; and optionally wherein the at least one amplification primer comprises a nucleotide sequence at least 85%, at least 87%, at least 90%, at least 92%, at least 94%, at least 95%, at least 96%, at least 98%, or 100% identical to any one of SEQ ID NOs: 92-235.

In some embodiments, any of the reagents (e.g., amplification primers, guide nucleic acids, programmable nucleases, reporters), target nucleic acids, devices, assay formats, or other items disclosed herein can be used as research tools. The research tools can be used to detect any number of target nucleic acids or segments thereof, mutations, or other indications disclosed herein in a laboratory setting. Reagent kits can be provided as reagent packs for open box instrumentation. The high sensitivity lab tests can be performed in a single assay.

In some embodiments, any of the reagents (e.g., amplification primers, guide nucleic acids, programmable nucleases, reporters), target nucleic acids, devices, assay formats, or other items disclosed herein can be used in a point-of-care (POC) test, which can be carried out at a decentralized location such as a hospital, POL, or clinic. These point-of-care tests can be used to diagnose any of the indications disclosed herein, such as monkeypox. POC tests can be provided as small instruments with a consumable test card, wherein the test card is any of the assay formats (e.g., a lateral flow assay) disclosed herein. This may be valuable in detecting diseases in a developing country and as a global healthcare tool to detect the spread of a disease or efficacy of a treatment or provide early detection of a disease.

In some embodiments, any of the reagents (e.g., amplification primers, guide nucleic acids, programmable nucleases, reporters), target nucleic acids, devices, assay formats, or other items disclosed herein can be used in an over-the-counter (OTC), readerless format, which can be used at remote sites or at home to diagnose a range of indications, such as monkeypox. OTC products can include a consumable test card, wherein the test card is any of the assay formats (e.g., a lateral flow assay) disclosed herein. In an OTC product, the test card can be interpreted visually or using a mobile phone.

Assays that deliver results in under an hour, for example, in 15 to 60 minutes, are particularly desirable for at home testing for many reasons. For example, antivirals can be most effective when administered within the first 48 hours after disease exposure. Thus, the methods disclosed herein, which are capable of delivering results in under an hour, may allow for the delivery of anti-viral therapy during the first 48 hours after infection. Additionally, the systems and assays provided herein, which are capable of delivering quick diagnoses and results, can help keep or send a patient at home, improve comprehensive disease surveillance, and prevent the spread of an infection.

### Compositions

Disclosed herein are compositions for use to detect a target nucleic acid. The target nucleic acid can be from a monkeypox virus.

In some embodiments, a composition comprises a non-naturally occurring guide nucleic acid comprising a nucleotide sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 236-247. In some embodiments, a composition comprises an amplification primer comprising a nucleotide sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 92-235.

In some embodiments, the composition further comprises one or more programmable nuclease(s) described herein. In some embodiments, the composition further comprises one or more reporter(s) described herein. In some embodiments, the composition further comprises one or more reagent(s) for amplification described herein. In some embodiments, the composition further comprises the lysis buffer described herein. In some embodiments, the composition further comprises the negative control nucleic acid described herein.

### Kits

Disclosed herein are kits, reagents, and systems for use to detect a target nucleic acid. The target nucleic acid can be from a monkeypox virus.

In some embodiments, the kit comprises the reagents and a support medium. The reagent may be provided in a reagent chamber or on the support medium. Alternatively, the reagent may be placed into the reagent chamber or the support medium by the individual using the kit. Optionally, the kit further comprises a buffer and a dropper. The reagent chamber can be a test well or container. The opening of the reagent chamber may be large enough to accommodate the support medium. The buffer may be provided in a dropper bottle for ease of dispensing. The dropper can be disposable and transfer a fixed volume. The dropper can be used to place a sample into the reagent chamber or on the support medium.

In some embodiments, a kit for detecting a target nucleic acid comprising a support medium; a non-naturally occurring guide nucleic acid targeting a segment of the target nucleic acid; a programmable nuclease capable of being activated when complexed with the non-naturally occurring guide nucleic acid and the target nucleic acid segment; and a single stranded reporter comprising a detection moiety, wherein the reporter is capable of being cleaved by the activated nuclease, thereby generating a detectable signal.

In some embodiments, a kit for detecting a target nucleic acid or segment thereof comprising a PCR plate; a non-naturally occurring guide nucleic acid targeting a segment of the target nucleic acid; a programmable nuclease capable of being activated when complexed with the non-naturally occurring guide nucleic acid and the target nucleic acid segment; and a single stranded reporter comprising a detection moiety, wherein the reporter is capable of being cleaved by the activated nuclease, thereby generating a detectable signal. The wells of the PCR plate can be pre-aliquoted with the non-naturally occurring guide nucleic acid targeting a segment of the target nucleic acid, a programmable nuclease capable of being activated when complexed with the non-naturally occurring guide nucleic acid and the target sequence, and at least one population of a single stranded reporter comprising a detection moiety. A user can thus add the biological sample of interest to a well of the pre-aliquoted PCR plate and measure for the detectable signal with a fluorescent light reader or a visible light reader.

In some embodiments, such kits may include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Examples

The following examples, which are included herein for illustration purposes only, are not intended to be limiting.

### Example 1: Detection of a Target Nucleic Acid Using Different RNA-Targeting CRISPR/Cas Effector Proteins

To evaluate the use of different RNA-targeting CRISPR/Cas proteins in a method of quantitating a target nucleic acid, the following experiment was performed. A reagent mixture was generated that comprised the following components: 10 nM of a Cas protein (comprising the amino acid sequence of SEQ ID NO: 21, 62 or 43), 10 nM of a SARS-CoV-2 N gene guide RNA (R4684) or an off-target guide RNA, 1 μM reporter (5Alex647N/rUrUrUrUrU/3IAbRQSp), and buffer (20 mM imidazole pH 7.5, 50 mM KCl, 5 mM MgCl₂, 10 μg/mL BSA, 0.01% Igepal Ca-630, 5% glycerol). The reagent mixture was mixed with different amounts of the target nucleic acid (synthetic SARS-CoV-2 N gene)—6000 copies/chamber, 2400 copies/chamber, 960 copies/chamber, 384 copies/chamber, 154 copies/chamber, 61 copies/chamber, and 0 copies/chamber. 10 μL of each of the resulting master reaction mixtures was loaded on a partitioning chamber of the digital PCR chip, which comprised about 20,000 nanovolumes. The master reaction mixture was distributed into 20,000 nanovolumes during the partitioning step at 4° C. for 15 min. Subsequently, the nanovolumes were incubated at 37° C. for 30 min, followed by depressurization at ambient temperature for 40 min. Thereafter, the partitioning chambers were scanned to detect fluorescent signals from the cleaved reporter at 150 millisecond exposure using a red laser.

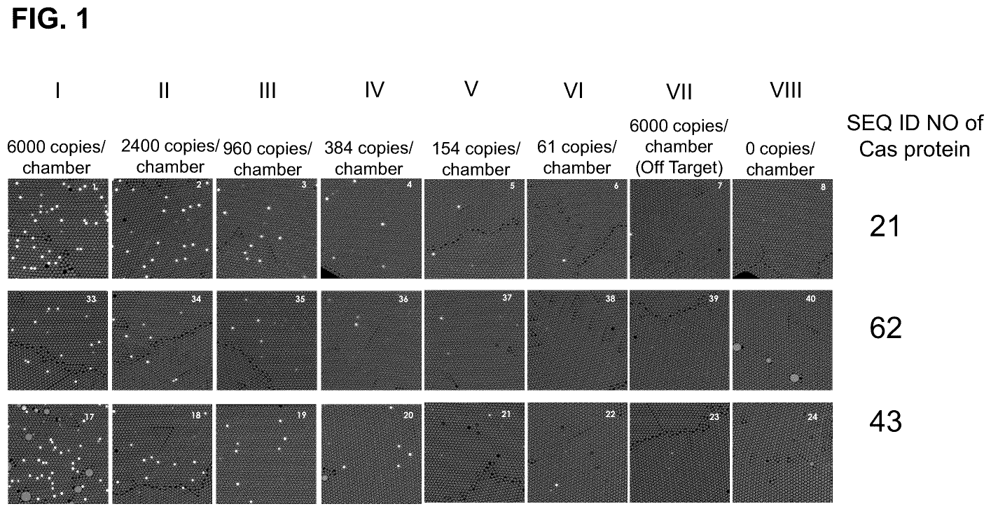

As shown in FIG. 1, the presence of the target nucleic acid in a particular nanovolume in the chamber was detected by the presence of a positive signal in that nanovolume. Furthermore, the number of positive nanovolumes (that is, the nanovolumes in which a positive signal reflected the presence of the target nucleic acid) was proportional to the number of copies of target nucleic acid added to the chamber, indicating the quantitative nature of this assay. Moreover, when the chamber was loaded with a reaction mixture comprising an off-target guide nucleic acid (“Off Target”), no nanovolumes in that chamber were seen to be positive, indicating the specificity of this assay in detecting the presence of the target nucleic acid only when all components of the reaction mixture were present. Finally, FIG. 1 also demonstrates that this quantitation assay may be used with multiple different RNA targeting CRISPR/Cas protein enzymes, such as a Cas protein comprising the amino acid sequence of SEQ ID NO: 21, 62 or 43.

### Example 2: Detection of a Target Nucleic Acid Using Varying Concentrations of an RNA-Targeting CRISPR/Cas Effector Protein

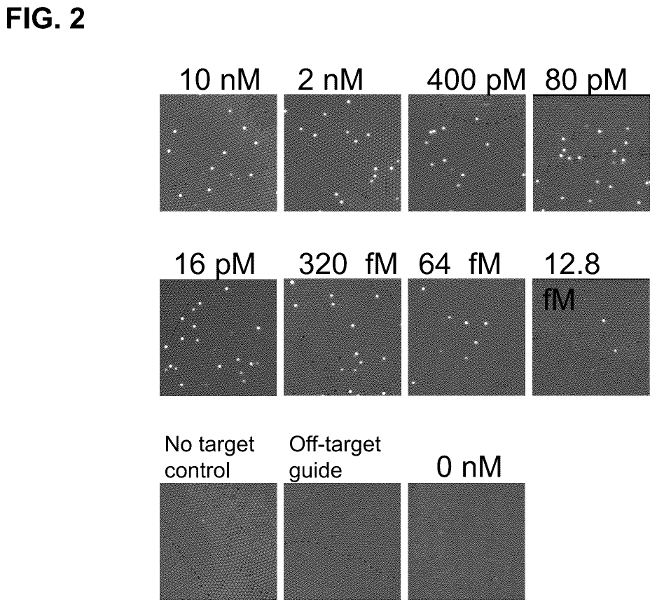

To try to understand the effects of reducing the CRISPR/Cas effector protein concentration on this quantitation assay, similar reaction mixtures as described in Example 1 were generated using different concentrations of CRISPR/Cas effector protein (0 fM, 12.8 fM, 64 fM, 320 fM, 16 pM, 80 pM, 400 pM, 2 nM, or 10 nM), 10 nM SARS-CoV-2 N-gene targeting guide RNA or an off-target guide RNA (OTG), and 600 or 0 copies per chamber of the target nucleic acid (Twist Synthetic SARS-CoV-2 Synthetic RNA Control) and loaded on the partitioning chamber as described in Example 1. Subsequently, as described in Example 1, the nanovolumes were incubated at 37° C. for 30 min, followed by depressurization at ambient temperature for 40 min and scanning to detect signals from the cleaved reporter. As shown in FIG. 2, while the negative control chambers (no-target control, NTC (0 copies/chamber), with 10 nM CRISPR/Cas protein; off-target guide, OTG, with 10 nM CRISPR/Cas protein; and “0 nM”, the reaction mixture with no CRISPR/Cas protein) did not show any fluorescence as expected, assay sensitivity was maintained even at extremely low concentrations of the CRISPR/Cas protein, e.g., in the range of fM.

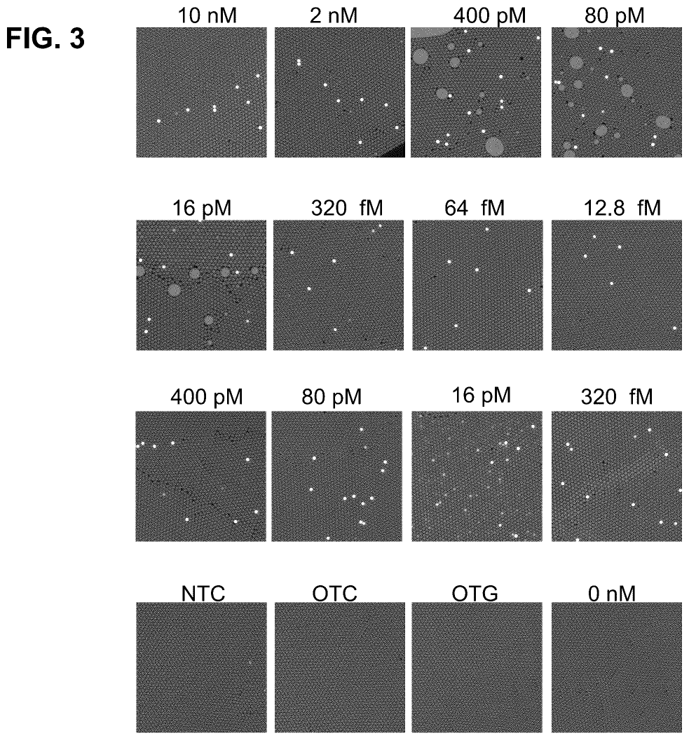

A similar experiment was performed with 600 copies per chamber of a different source of SARS-CoV-2 target nucleic acid (ATCC® synthetic SARS-CoV-2 RNA), which further confirmed that extremely low concentrations of the CRISPR/Cas protein may be used for quantitation in the assay disclosed here with a sensitivity of detection that is comparable to using higher concentrations of the CRISPR/Cas protein (FIG. 3).

### Example 3: Detection of a Target Nucleic Acid in the Presence of Other Reagents

To evaluate whether low pH crude lysis buffer has any inhibitory effects on the quantitation assays described herein, different volumes (0%, 5%, 10%, or 20%) of pH lysis buffer were added to the reaction mixture prepared as described in Example 1, using 10 nM Cas protein comprising the amino acid sequence of SEQ ID No: 21 and 5000 copies/chamber of Twist Synthetic SARS-CoV-2 Synthetic RNA Control target nucleic acid before distributing the reaction mixture into nanovolumes within the chamber. Each mixture also comprised a SARS-CoV-2 N-gene targeting guide RNA (R4684) or an off-target guide (OTG, R5882) as a negative control.

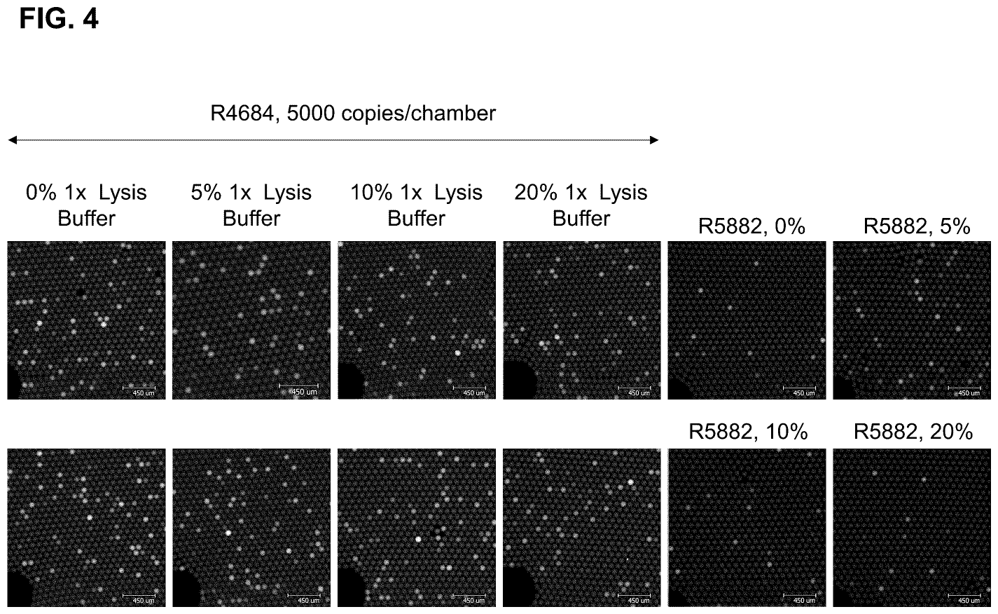

As shown in FIG. 4, positive nanovolumes were reliably obtained even when 10% lysis buffer was used, indicating that the Cas protein comprising the amino acid sequence of SEQ ID No: 21 is tolerant of crude lysis buffer up to 10% of the total assay volume. 20% lysis buffer started to show some dropout of positive nanovolumes under the conditions tested, though additional optimization could be done to improve performance with higher concentrations of lysis buffer if desired.

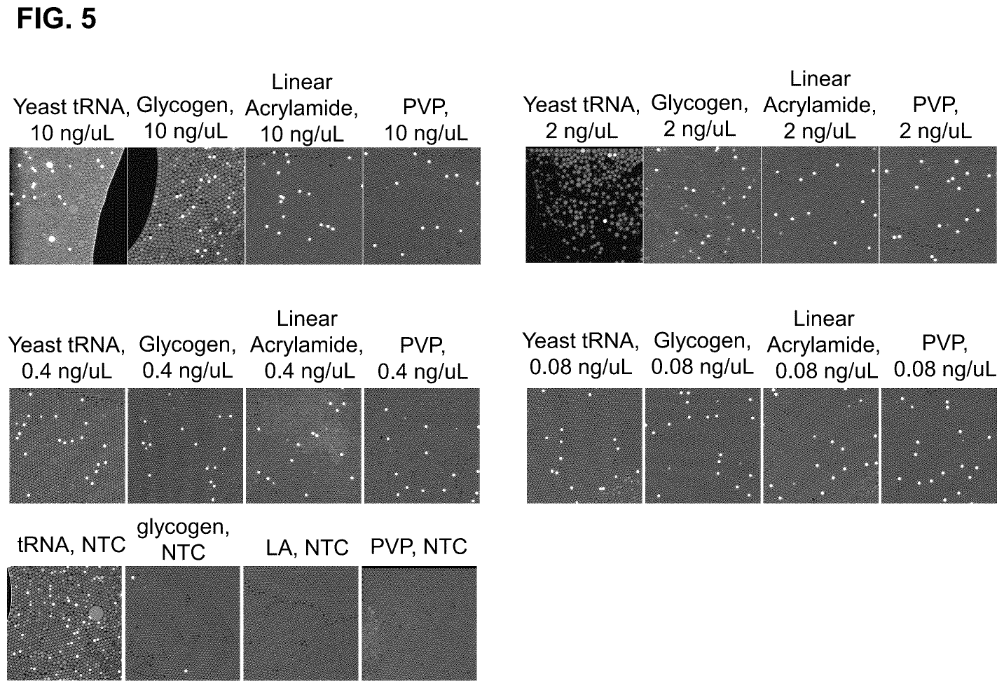

To further evaluate the effect of other reagents on the quantitative assays described herein, carrier molecules (e.g., yeast tRNA, glycogen, polyvinylpyrrolidone, PVP) were tested at different concentrations for their ability to help stabilize RNA during the distribution of the reaction mixture into nanovolumes. Reaction mixtures containing the Cas protein comprising the amino acid sequence of SEQ ID NO: 21 and Twist Synthetic SARS-CoV-2 Synthetic RNA Control target nucleic acid were prepared as described above, and different concentrations of carrier molecules (yeast tRNA, glycogen, linear acrylamide, and PVP) were added before distribution into nanovolumes. The chambers were processed and imaged as described in Example 1. FIG. 5 shows that the addition of tRNA resulted in high background signal under the conditions tested even in the presence of the negative non-targeting control (NTC) nucleic acid. However, the use of the other carrier molecules tested, such as, glycogen, linear acrylamide and PVP did not interfere with the assay reactions and signal detection under the conditions tested.

### Example 4: Specific Detection of a Target Nucleic Acid in a Mixture of Viral RNAs

To evaluate the use of the quantitative assays disclosed herein to specifically detect a viral target nucleic acid in a mixture of RNAs derived from three different viruses, the following experiment was performed.

A reagent mixture was generated that comprises the following components: 10 nM Cas protein comprising the amino acid sequence of SEQ ID NO: 21, R4684 guide RNA (GCCACCCCAAAAAUGAAGGGGACUAAAACAAGAAGAAUUCAGAUUUUUAA (SEQ ID NO: 87)), 1 μM reporter (5Alex647N/rUrUrUrUrU/3IAbRQSp), and buffer (20 mM imidazole pH 7.5, 50 mM KCl, 5 mM MgCl₂, 10 μg/mL BSA, 0.01% Igepal Ca-630, 5% glycerol). The reagent mixture was mixed with different amounts of the synthetic SARS-CoV-2 target nucleic acid and two off-target nucleic acids from Influenza A and Influenza B, as listed in Table A. 10 μL of each of the resulting master reaction mixtures was loaded on a partitioning chamber of the digital PCR chip, which comprised about 20,000 nanovolumes. The master reaction mixture was processed, and the signals were detected as described in Example 1.

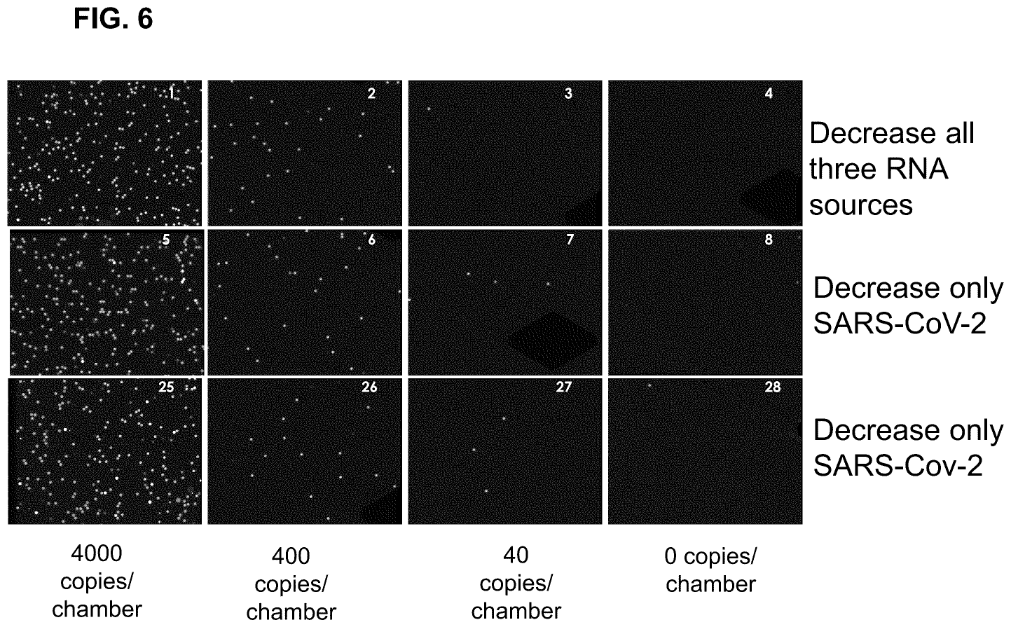

TABLE A
Amounts of the synthetic SARS-CoV-2 target nucleic acid and two off-
target nucleic acids from Influenza A and Influenza B used in the study

| Chamber | | Amount of SARS-CoV2 | Influenza A Virus | Influenza B Virus |
|---|---|---|---|---|
| number | | target nucleic acid | nucleic acid | nucleic acid |
| in FIG. 6 | Guide | (copies/chamber) | (copies/chamber) | (copies/chamber) |
| 1 | R4684 | 4000 | 4000 | 4000 |
| 2 | R4684 | 400 | 400 | 400 |
| 3 | R4684 | 40 | 40 | 40 |
| 4 | R4684 | 0 | 0 | 0 |
| 5 | R4684 | 4000 | 4000 | 4000 |
| 6 | R4684 | 400 | 4000 | 4000 |
| 7 | R4684 | 40 | 4000 | 4000 |
| 8 | R4684 | 0 | 4000 | 4000 |
| 25 | R4684 | 4000 | 4000 | 4000 |
| 26 | R4684 | 400 | 4000 | 4000 |
| 27 | R4684 | 40 | 4000 | 4000 |
| 28 | R4684 | 0 | 4000 | 4000 |

As shown in FIG. 6, when the concentration of all the three RNAs (the target SARS-CoV-2 RNA and the off-target Influenza A and B RNAs) was decreased, a corresponding decrease in the number of positive nanovolumes was seen (top row). Notably, a similar decrease in the number of positive nanovolumes was seen when decreasing the concentration of just the SARS-CoV-2 target nucleic acid (duplicates shown in middle and bottom rows). These results demonstrate that the positive nanovolumes were a result of specific detection of the target SARS-CoV-2 RNA. Accordingly, the quantitative assays described herein are capable of specifically detecting and quantitating a target nucleic acid in a mixture of multiple different nucleic acids, suggesting that the assays described herein may be effectively used to quantitate nucleic acids in complex samples, such as clinical patient samples, for diagnostic purposes. Furthermore, given the specificity of the detection indicated by these data, the quantitative assays described herein may be multiplexed to detect multiple different target nucleic acids in the sample.

### Example 5: Detection of a Target Nucleic Acid Using Different DNA-Targeting CRISPR/Cas Effector Proteins

To evaluate the use of different DNA-targeting CRISPR/Cas proteins in the quantitation assays disclosed herein, the following experiment was performed. A reagent mixture was generated that comprised the following components: 40 nM of the Cas protein comprising the amino acid sequence of SEQ ID No: 34, 62.5 nM of a SARS-CoV-2 N gene crRNA (GCCACCCCAAAAAUGAAGGGGACUAAAACAAGAAGAAUUCAGAUUUUUAA (SEQ ID NO: 87)), 1.5 μM of a ssDNA reporter (5Alex647N/TTATTATT/3IAbRQSp, or 5Alex647N/TTTTTTTTTTTT/3IAbRQSp (SEQ ID NO: 88)), and buffer (20 mM Tris-HCl, pH 8.8, 2 mM KOAc, 0.02 mg/mL BSA, 15 mM MgOAc).

The reagent mixture was mixed, either with different amounts of the target nucleic acid (a synthetic SARS-CoV-2 N gene dsDNA), for example, 25,000 copies/chamber, 18,750 copies/chamber, 12,500 copies/chamber/chamber, or 6,250 copies/chamber; or with a no-target control (NTC; 0 copies/reaction) or an off-target control (OTC). 25 μL of each of the resulting master reaction mixtures was loaded on a partitioning chamber of the digital PCR chip, which comprised about 30,000 nanovolumes. The master reaction mixture was distributed into nanovolumes during the partitioning step at 4° C. for 15 min. Subsequently, the nanovolumes were incubated at 60° C. for 90 min, followed by depressurization at ambient temperature for 40 min. Thereafter, the chambers were scanned to detect signals from the cleaved reporter at 50 millisecond exposure using a red laser.

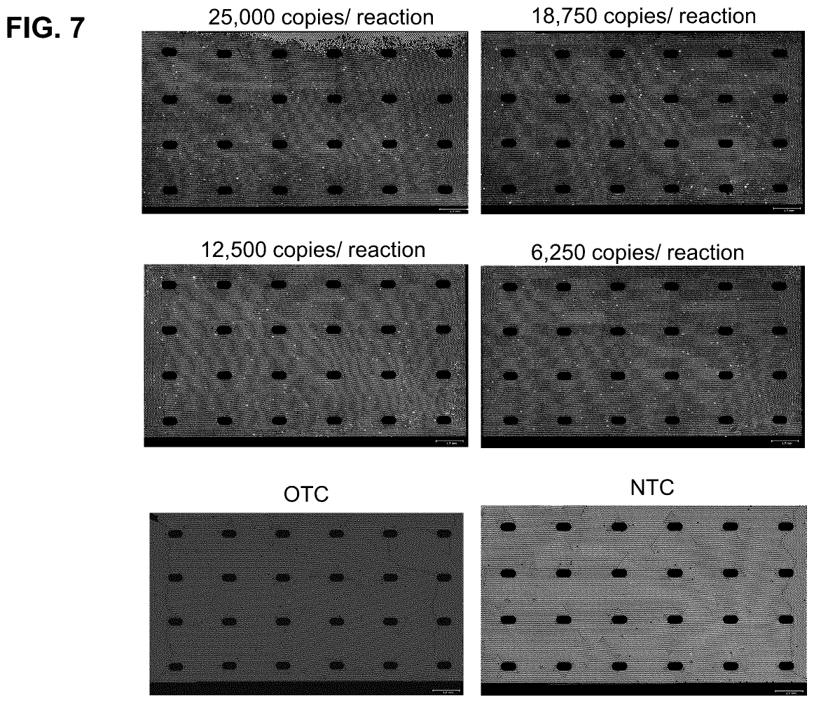

As shown in FIG. 7, the presence of the target DNA in a particular nanovolume in the chamber was detected by the presence of a positive signal in that nanovolume. Notably, when the chamber was loaded with a reaction mixture comprising an off-target DNA or no-target DNA, no nanovolumes in that chamber were seen to be positive, indicating the specificity of this assay in detecting the presence of only the target nucleic acid. FIG. 7 further demonstrates that the quantitation assays described herein may be used not only with RNA-targeting Cas proteins, but also with DNA-targeting Cas proteins, highlighting the versatility of this assay.

The ability of the assays described herein to detect and quantitate target DNA was further investigated using another DNA-targeting Cas protein comprising the amino acid sequence of SEQ ID NO: 3. A similar reagent mixture was generated as described above, and mixed with either the target nucleic acid (a synthetic SARS-CoV-2 N gene dsDNA) or a no-target control (NTC). 25 μL of each of the resulting master reaction mixtures was loaded on a chamber of the digital PCR chip in a QIAcuity Digital PCR System. The master reaction mixture was distributed into nanovolumes during the partitioning step at 23° C. for 15 min. Subsequently, the nanovolumes were incubated at 55° C. for 45 min. Thereafter, the chambers were scanned to detect signals from the cleaved reporter.

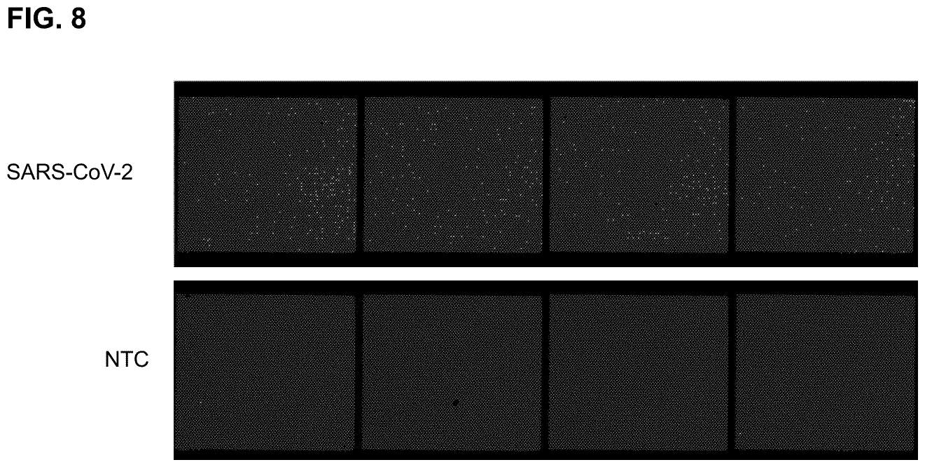

As shown in FIG. 8, the presence of the target DNA in a particular nanovolume in the chamber was detected by the presence of a positive signal in that nanovolume. Notably, when the chamber was loaded with a reaction mixture comprising a non-targeting DNA, no nanovolumes in that chamber were seen to be positive, indicating the specificity of this assay in detecting the presence of only the target nucleic acid. FIG. 8 further establishes that the quantitation assays described herein may be used with multiple different DNA targeting Cas proteins.

### Example 6: Detection of Synthetic Standards Representing Wastewater Targets

To evaluate whether the quantitative assays described herein may be used to detect and quantitate target nucleic acids in wastewater samples, the following experiments were performed. Nucleic acids derived from SARS-CoV2 and PMMOV can be used as targets for their detection in wastewater.

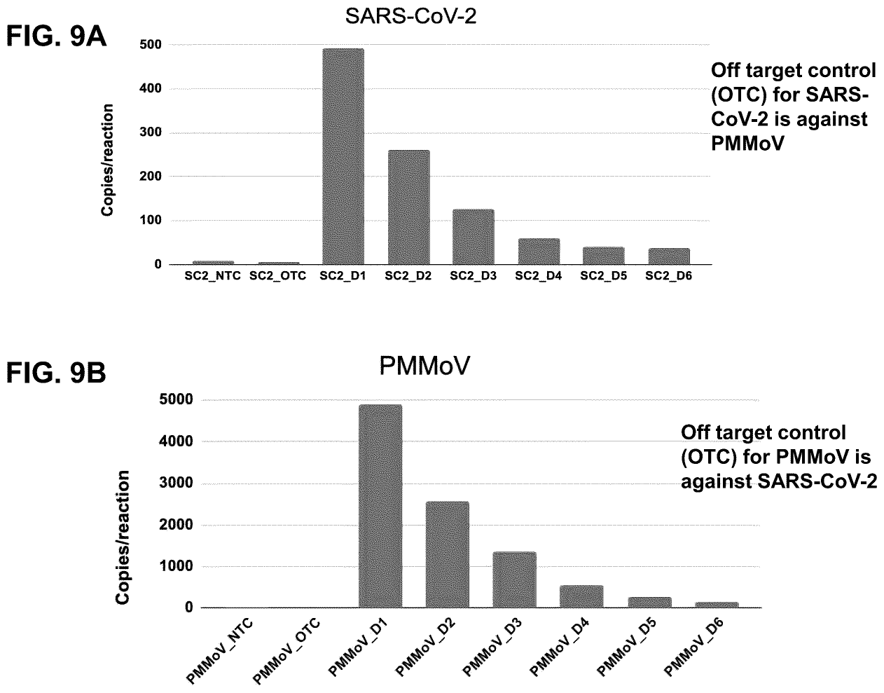

A reagent mixture was generated that comprised the following components: Cas protein, SARS-CoV-2 N gene crRNA or PMMoV crRNA, and 1.5 μM reporter. The reagent mixture was mixed with: (i) different concentrations of the target nucleic acid from dilution D1 down to dilution D6 (a synthetic SARS-CoV-2 N gene or a synthetic nucleic acid derived from pepper mild mottle virus (PMMoV)); or (ii) a non-targeting control (NTC); or (iii) an off-targeting control (OTC), as indicated in FIGS. 9A and 9B. The reaction mixtures were processed and signals detected as described in Example 1.

FIG. 9A shows that the quantitative assays described herein employing the SARS-CoV-2 guide RNA specifically detected SARS-Co-2 target nucleic acid, while emitting minimal or no signal upon exposure to PMMoV nucleic acid (OTC in FIG. 9A). FIG. 9B, on the other hand, shows that the quantitative assays described herein employing the PMMoV guide RNA specifically detected PMMoV target nucleic acid, while emitting minimal or no signal upon exposure to SARS-CoV-2 nucleic acid (OTC in FIG. 9B). These results illustrate not only the specificity of the quantitative assays described herein, but also that these assays can be used to effectively detect and quantitate target nucleic acids that are found in a complex, real-world sample, such as, wastewater.

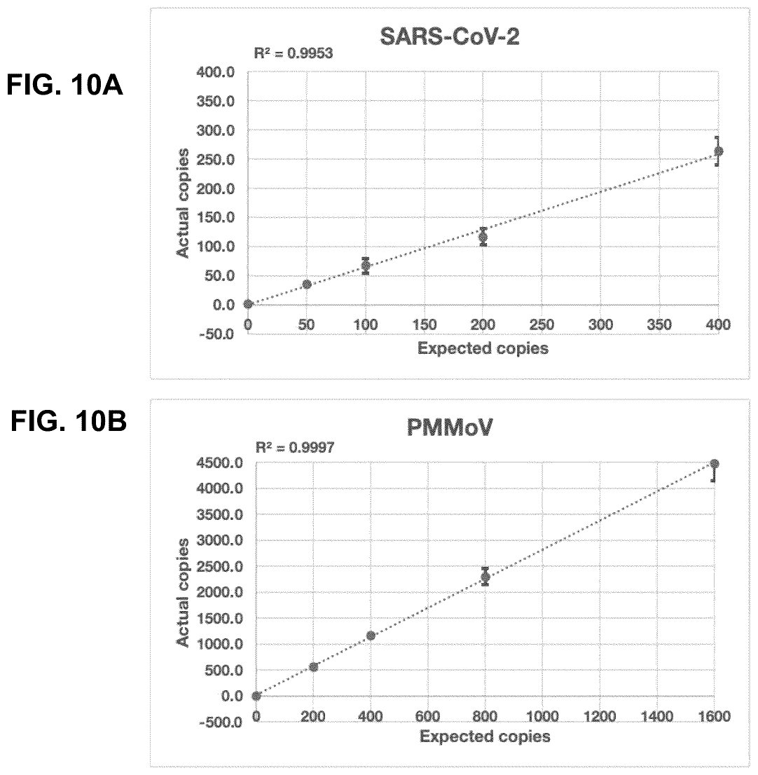

Moreover, FIGS. 10A and 10B show standard curves generated using the detection of SARS-CoV-2 target nucleic acid or PMMoV target nucleic acid, which show a relatively linear relationship between the number of positive nanovolumes detected (y-axis) and the number of copies added per chamber (x-axis). The curves highlight that the quantitative assays described herein may be used for absolute or relative quantitation of a target nucleic acid in a wastewater sample, as compared to control samples comprising defined amounts of the target nucleic acid.

In sum, the experiments described herein demonstrate the wide range of applications for the quantitative assays described herein, including in quantitating target RNA using RNA-targeting CRISPR-Cas proteins, quantitating target DNA using DNA-targeting CRISPR-Cas proteins, quantitating a single target nucleic acid specifically among a mixture of different nucleic acids, and quantitating a target nucleic acid in a complex sample, such as, wastewater.

### Example 7: Detection of Different Concentrations of Target Nucleic Acid Using an RNA-Targeting CRISPR/Cas Effector Protein

To evaluate the threshold of detection of an RNA-targeting CRISPR/Cas protein comprising the amino acid sequence of SEQ ID NO: 21 in a method of quantitating a target nucleic acid, the following experiment was performed. A reagent mixture was generated that comprised the following components: 10 nM of a Cas protein (comprising the amino acid sequence of SEQ ID NO: 21), 10 nM of a SARS-CoV-2 N gene guide RNA (R4684) or an off-target guide RNA, and buffer (20 mM imidazole pH 7.5, 50 mM KCl, 5 mM MgCl₂, 10 μg/mL BSA, 0.01% Igepal Ca-630, 5% glycerol). The reagent mixture was incubated at 37° C. for 30 minutes to pre-complex the Cas protein and guide RNA, then 1 μM reporter (5Alex647N/rUrUrUrUrU/3IAbRQSp) was added to the mix. The reagent mixture was mixed with different amounts of the target nucleic acid (synthetic SARS-CoV-2 N gene)-10,000 copies/chamber (3,333 copies/μL), 5,000 copies/chamber (1,667 copies/μL), 2,500 copies/chamber (833 copies/μL), 1,250 copies/chamber (417 copies/μL), 625 copies/chamber (208 copies/μL), 313 copies/chamber (104 copies/μL), 157 copies/chamber (52 copies/μL), 78 copies/chamber (26 copies/μL), 39 copies/chamber (13 copies/μL), 20 copies/chamber (7 copies/μL), 10 copies/chamber (3 copies/μL), and 0 copies/chamber (10 copies/μL). 10 μL of each of the resulting master reaction mixtures was loaded on a partitioning chamber of the digital PCR chip, which comprised about 20,000 nanovolumes. The master reaction mixture was distributed into 20,000 nanovolumes during the partitioning step at 4° C. for 15 min. Subsequently, the nanovolumes were incubated at 37° C. for 30 min, followed by depressurization at ambient temperature for 40 min. Thereafter, the partitioning chambers were scanned to detect fluorescent signals from the cleaved reporter at 150 millisecond exposure using a red laser.

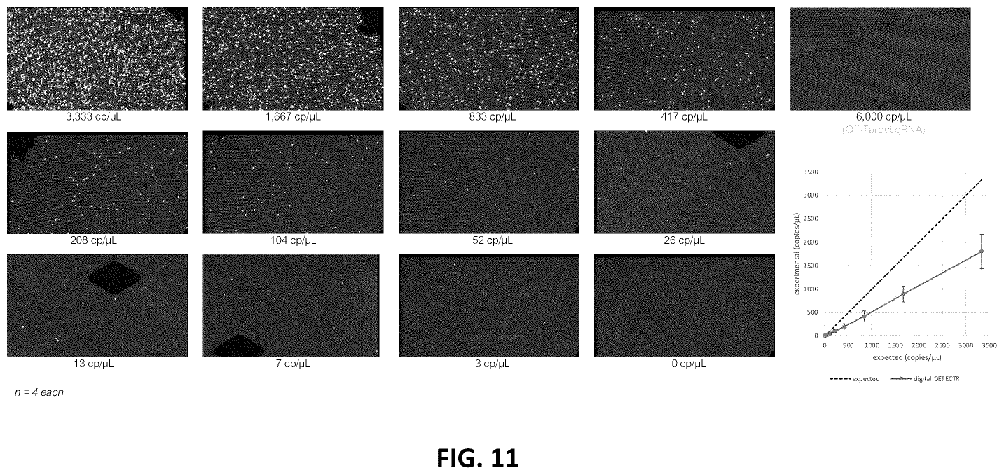

As shown in FIG. 11, the presence of the target nucleic acid in a particular nanovolume in the chamber was detected by the presence of a positive signal in that nanovolume. Furthermore, the number of positive nanovolumes (that is, the nanovolumes in which a positive signal reflected the presence of the target nucleic acid) was proportional to the number of copies of target nucleic acid added to the chamber, indicating the quantitative nature of this assay. Moreover, when the chamber was loaded with a reaction mixture comprising an off-target guide nucleic acid (“Off Target”), no nanovolumes in that chamber were seen to be positive, indicating the specificity of this assay in detecting the presence of the target nucleic acid only when all components of the reaction mixture were present. Finally, FIG. 11 also demonstrates that this quantitation assay is relatively linear over a range of at least 0 copies/μL to 3,333 copies/μL under the conditions tested. Interestingly, the slope of the experimental digital DETECTR copies detected to expected copies detected was not 1:1, indicating that the system was not operating at 100% efficiency under the conditions tested. Without being bound by any particular theory, it is hypothesized that one possible reason for this observed inefficiency is due to the efficiency of the gRNA (and/or CRISPR/Cas enzyme and/or complex) in binding to the target nucleic acid. Such guide efficiency can be adjusted for as described herein in order to better correlate the observed number of positive nanovolumes with the actual copies per μL in a sample of interest.

### Example 8: Detection of a Target Nucleic Acid in Clinical Samples

To evaluate the use of an RNA-targeting CRISPR/Cas protein comprising the amino acid sequence of SEQ ID NO: 21 in a method of quantitating a target nucleic acid extracted from a clinical sample, the following experiment was performed. A digital DETECTR reagent mixture was generated that comprised the following components: 10 nM of a Cas protein (comprising the amino acid sequence of SEQ ID NO: 21), 10 nM of a SARS-CoV-2 N gene guide RNA (R4684) or an off-target guide RNA, 1 μM reporter (5Alex647N/rUrUrUrUrU/3IAbRQSp), and buffer (20 mM imidazole pH 7.5, 50 mM KCl, 5 mM MgCl₂, 10 μg/mL BSA, 0.01% Igepal Ca-630, 5% glycerol). A digital droplet PCR (ddPCR) reagent mixture was generated using the 3-Color Crystal Digital PCR™ kit for detection of CoVID-19 from ApexBio. Viral nucleic acids were purified from SARS-CoV-2-positive clinical respiratory samples in saline using the EZ1&2 Virus Mini Kit v2.0 from Qiagen. The digital DETECTR reagent mixture or ddPCR reagent mixture was mixed with a 1:100 dilution, 1:10 dilution, or undiluted sample of the extracted viral nucleic acids from each clinical sample or water (non-template control, NTC). 25 μL of each of the resulting ddPCR or digital DETECTR reaction mixtures was loaded on a partitioning chamber of the digital PCR chip, which comprised about 20,000 nanovolumes. The ddPCR reaction mixture was distributed into 20,000 nanovolumes during the partitioning step at 40° C. for 12 min. Subsequently, the ddPCR nanovolumes were incubated at 50° C. for 10 min to allow for cDNA synthesis to occur before an initial denaturation step at 95° C. for 1 min, and then thermocycled for 45 cycles (denaturation 95° C. for 10 seconds, annealing/extension at 55° C. for 30 seconds). The partitioning chamber was then depressurized at ambient temperature for 33 min. Thereafter, the ddPCR partitioning chambers were scanned to detect fluorescent signals from the ddPCR nanovolumes. The digital DETECTR reaction mixture was distributed into 20,000 nanovolumes during the partitioning step at 4° C. for 15 min. Subsequently, the digital DETECTR nanovolumes were incubated at 37° C. for 30 min, followed by depressurization at ambient temperature for 40 min. Thereafter, the digital DETECTR partitioning chambers were scanned to detect fluorescent signals from the cleaved reporter at 150 millisecond exposure using a red laser.

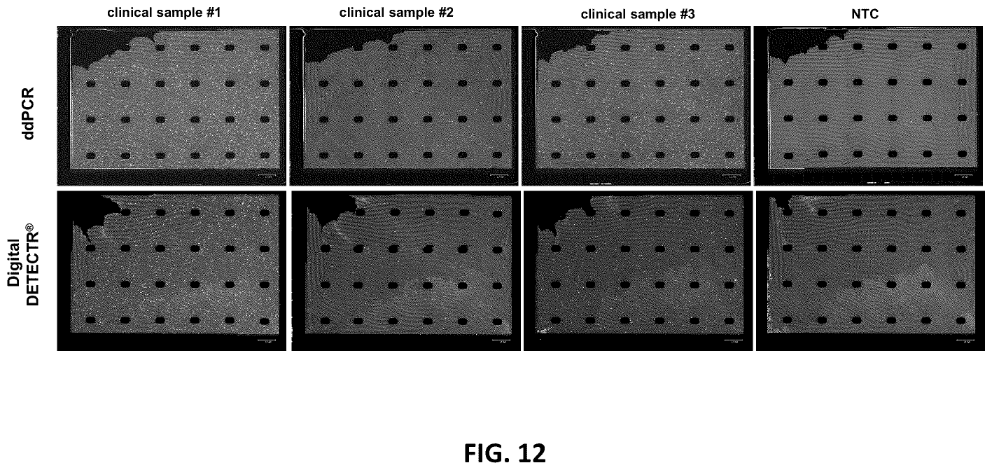

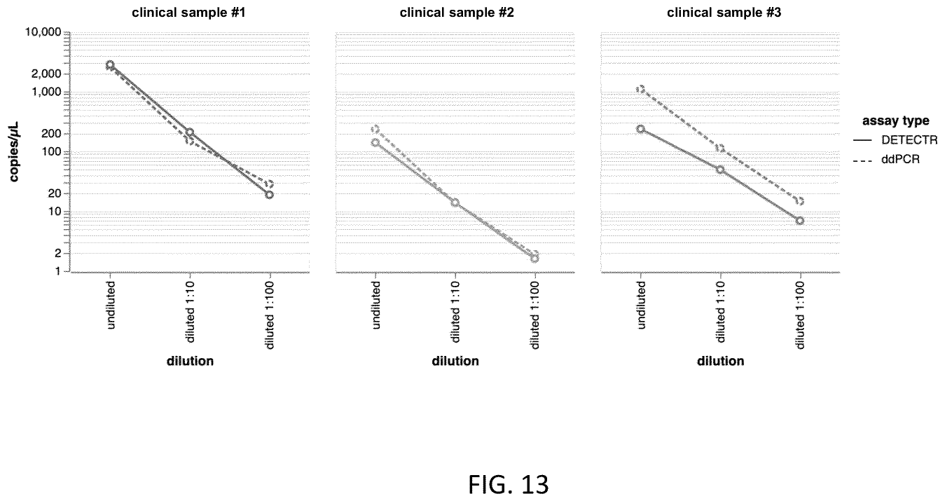

As shown in FIG. 12, the presence of the target nucleic acid in a particular nanovolume in the chamber was detected by the presence of a positive signal in that nanovolume. Furthermore, the number of positive nanovolumes (that is, the nanovolumes in which a positive signal reflected the presence of the target nucleic acid) was proportional to the number of copies of target nucleic acid added to the chamber for both ddPCR and digital DETECTR assayed clinical samples. FIG. 13 and TABLE B below show side-by-side comparisons of the number of copies detected by ddPCR or digital DETECTR for three clinical samples, indicating the quantitative, linear nature of this assay under the conditions tested.

TABLE B
SARS-CoV-2 Clinical Sample Comparison of ddPCR and digital DETECTR

| | Undiluted | 1/10 Dilution | 1/100 Dilution |
|---|---|---|---|
| | (copies/uL) | (copies/uL) | (copies/uL) |
| clinical sample #1 (ddPCR) | 2543.0 | 147.7 | 28.2 |
| clinical sample #1 (DETECTR) | 2809.8 | 208.5 | 18.7 |
| clinical sample #2 (ddPCR) | 235.2 | 13.7 | 1.9 |
| clinical sample #2 (DETECTR) | 139.4 | 13.9 | 1.6 |
| clinical sample #3 (ddPCR) | 1092.2 | 111.7 | 14.6 |
| clinical sample #3 (DETECTR) | 235.5 | 49.1 | 6.9 |

### Example 9: Detection of a Target Nucleic Acid in Wastewater Samples

To evaluate whether the quantitative assays described herein may be used to detect and quantitate target nucleic acids in wastewater samples, the following experiments were performed. Wastewater samples were collected over a period of two months and viral RNA was extracted therefrom. Digital DETECTR reagent mixtures for SARS-CoV-2 N-gene or PMMoV were generated and processed, and signals detected, as described in Example 6. Digital PCR reagent mixtures for SARS-CoV-2 N-gene were generated and processed, and signals detected, as described in Example 8. Digital PCR reagent mixtures for PMMoV generated using the UltraPlex 1-Step ToughMix® qPCR kit from Quanta BioSciences and PMMoV primers obtained from the SARS-CoV-2 RT-qPCR Kit for Wastewater from Promega. Digital PCR reagent mixtures for PMMoV were processed, and signals detected, as described in Example 8 with respect to SARS-CoV-2. Standard curves for SARS-CoV-2 and PMMoV were generated as described in Example 6 and used to adjust the digital DETECTR copy number values based on gRNA efficiency. Briefly, standard curves generated with actual vs. expected copies as described in Example 6, and the slope of the curve used as an “efficiency coefficient” to transform measured DETECTR copy numbers into actual copy numbers for comparison against ddPCR.

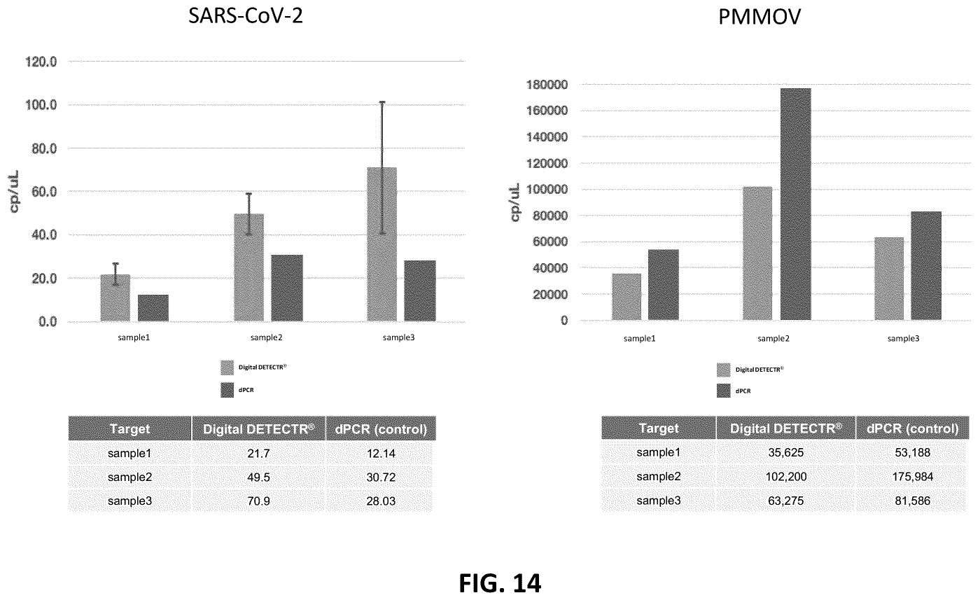

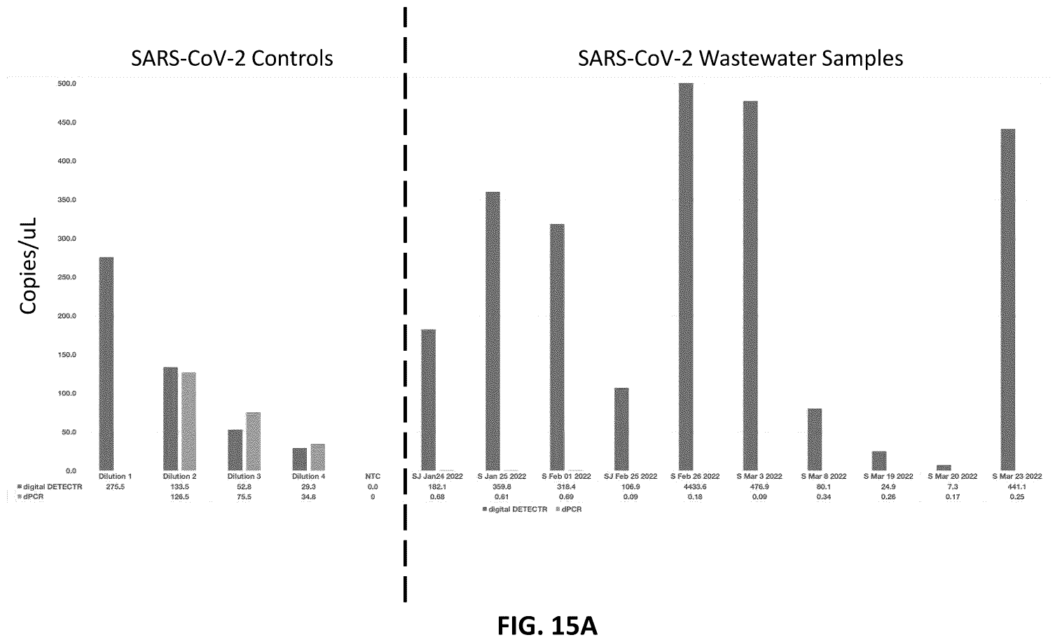

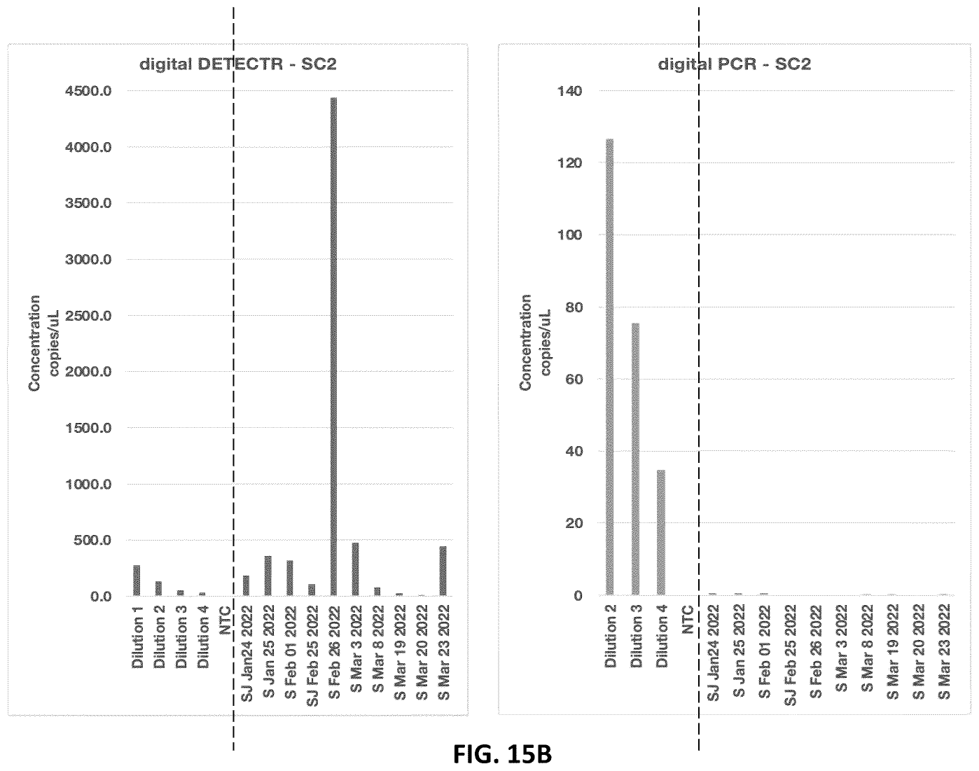

FIG. 14 shows a comparison of the number of copies per microliter (cp/μL) determined for three wastewater samples using digital DETECTR and ddPCR. These data show that digital DETECTR has a roughly equivalent dynamic range to digital PCR for viral quantification under the conditions tested for both SARS-CoV-2 and PMMOV RNA in wastewater samples. FIGS. 15A and 15B show the difference in signal obtained for digital DETECTR-assayed wastewater samples compared to dPCR-assayed wastewater samples.

While the number of copies/μL obtained using positive control samples with known concentrations (dilutions 1˜4 or NTC) for standard curve generation was relatively similar for both dPCR and digital DETECTR, the signal generated by dPCR was significantly lower than that of the digital DETECTR signal for corresponding wastewater samples. FIG. 15A shows a head-to-head comparison of the digital DETECTR and dPCR signals graphed with the same scaling along the Y-axis while FIG. 15B shows the digital DETECTR and dPCR signals graphed with different Y-axis scaling, in order to highlight the stark differences in signals obtained by each method. One possible reason for this observed difference in signal strength is that dPCR may be more sensitive to inhibitors (e.g., salts) left in the wastewater samples following sample prep and viral RNA extraction than digital DETECTR. These results illustrate not only the specificity of the digital DETECTR quantitative assays described herein, but also that these assays can be used to effectively detect and quantitate target nucleic acids that are found in a complex, real-world sample, such as, wastewater.

### Example 10: Detection of a Target Nucleic Acid Using Different DNA-Targeting CRISPR/Cas Effector Proteins at Different Reaction Temperatures

To evaluate the use of different DNA-targeting CRISPR/Cas proteins at different reaction temperatures in the quantitation assays disclosed herein, the following experiment was performed. A reagent mixture was generated that comprised the following components: 40 nM of the Cas protein comprising the amino acid sequence of SEQ ID No: 65, 67, 68, 34, 17, 89, 90, or 91, 40 nM of a SARS-CoV-2 N gene crRNA, 1.5 μM of a ssDNA reporter (5Alex647N/TTATTATT/3IAbRQSp, or 5Alex647N/TTTTTTTTTTTT/3IAbRQSp (SEQ ID NO: 88)), and buffer (20 mM Tris-HCl, pH 8.8, 2 mM KOAc, 0.02 mg/mL BSA, 15 mM MgOAc). The reagent mixture was incubated at 37° C. for 30 minutes to pre-complex the Cas protein and guide RNA. The reagent mixture was then mixed with 1000 copies/microliter of the target nucleic acid (a synthetic SARS-CoV-2 N gene dsDNA) or with a no-target control (NTC; 0 copies/reaction) or an off-target control (OTC). 40 μL of each of the resulting master reaction mixtures was loaded on a partitioning chamber of the digital PCR chip, which comprised about 30,000 nanovolumes. The master reaction mixture was distributed into nanovolumes during the partitioning step at room temperature for 15 min. Subsequently, the nanovolumes were incubated at 37° C. or 50° C. for 45 minutes, followed by depressurization at ambient temperature for 40 min. Thereafter, the chambers were scanned to detect signals from the cleaved reporter at 50 millisecond exposure using a red laser.

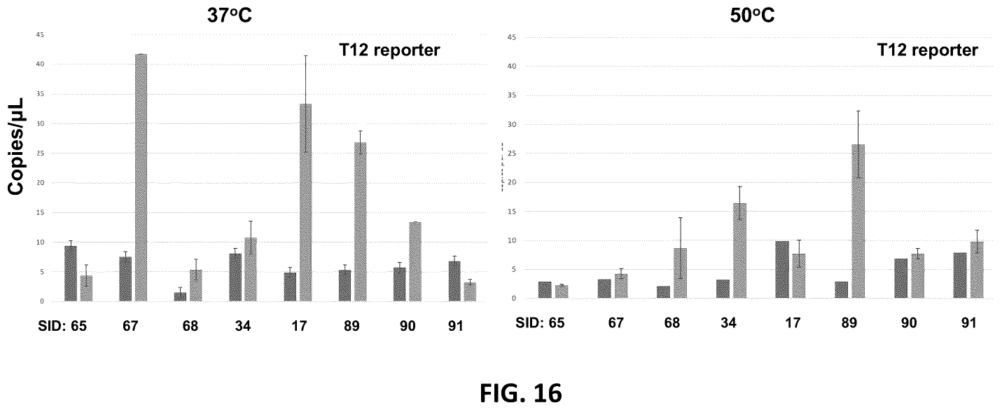

As shown in FIG. 16, the presence of the target DNA in a particular nanovolume in the chamber was detected by the presence of a positive signal in that nanovolume. FIG. 16 further establishes that the quantitation assays described herein may be used with multiple different DNA-targeting Cas proteins and that different DNA-targeting Cas proteins may be operative at different temperatures, which may be particularly useful for warm-start methods such as those described in Example 11.

### Example 11: Warm-Start Detection of a Target Nucleic Acid

To evaluate the use of delaying tactics to improve signal quality and quantitative range, a warm-start strategy is employed to reduce and/or prevent activation of the CRISPR/Cas complex during partitioning until the complex has reached a predetermined reaction temperature. A reagent mixture is generated at room temperature that comprises the following components: 40 nM of a Cas protein comprising the amino acid sequence of SEQ ID No: 34, 62.5 of nM a SARS-CoV-2 N gene crRNA (GCCACCCCAAAAAUGAAGGGGACUAAAACAAGAAGAAUUCAGAUUUUUAA (SEQ ID NO: 87)), 1.5 μM of a ssDNA reporter (5Alex647N/TTATTATT/3IAbRQSp, or 5Alex647N/TTTTTTTTTTTT/3IAbRQSp (SEQ ID NO: 88)), and buffer (20 mM Tris-HCl, pH 8.8, 2 mM KOAc, 0.02 mg/mL BSA, 15 mM MgOAc). The Cas protein is associated with a blocking thermosensitive aptamer which prevents catalytic activity of the Cas protein at or below room temperature. The blocking thermosensitive aptamer is configured to denature at temperatures about 50° C.

The reagent mixture is mixed, either with different amounts of the target nucleic acid (a synthetic SARS-CoV-2 N gene dsDNA), for example, 25,000 copies/chamber, 18,750 copies/chamber, 12,500 copies/chamber/chamber, or 6,250 copies/chamber; or with a no-target control (NTC; 0 copies/reaction) or an off-target control (OTC). 25 μL of each of the resulting master reaction mixtures is loaded on a partitioning chamber of the digital PCR chip, which comprises about 30,000 nanovolumes. The master reaction mixture was distributed into nanovolumes during the partitioning step at 4° C. for 15 min. Subsequently, the nanovolumes are incubated at 60° C. for 90 min (at which point the aptamer is denatured and the Cas protein is activated), followed by depressurization at ambient temperature for 40 min. Thereafter, the chambers are scanned to detect signals from the cleaved reporter at 50 millisecond exposure using a red laser. The presence of the target DNA in a particular nanovolume in the chamber is detected by the presence of a positive signal in that nanovolume. When the chamber is loaded with a reaction mixture comprising an off-target DNA or no-target DNA, no nanovolumes in that chamber will be seen to be positive.

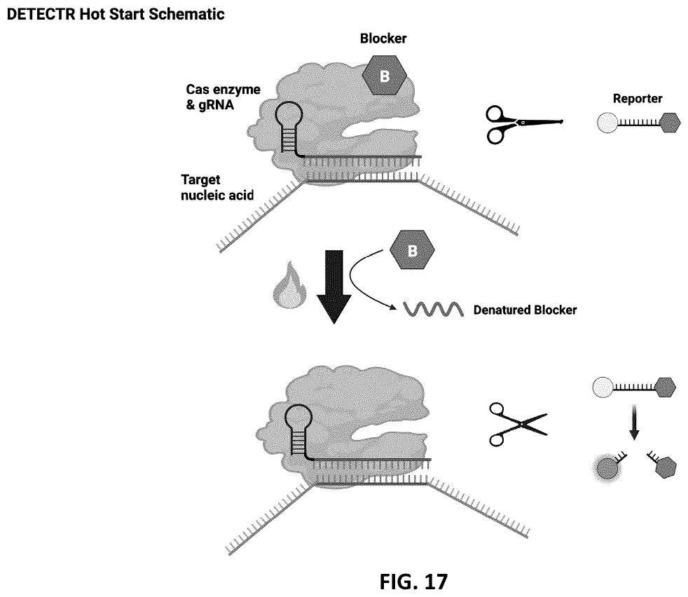

FIG. 17 shows a schematic of the exemplary warm-start strategy which may be employed to delay activation of the CRISPR/Cas complex until the complex has reached a predetermined reaction temperature.

### Example 12: Two-Pot LAMP DETECTR Reactions for Detection of Monkeypox Virus

This example describes LAMP DETECTR reactions for the detection of monkeypox virus in a two-step, two-pot reaction.

SgRNA-Cas14a.1 complexes are prepared by pre-incubating Cas 14a.1 with sgRNA MPXV-1 (SEQ ID NO: 236) for 30 minutes at 37° C. After formation of the sgRNA-Cas14a.1 complexes, a labeled ssDNA reporter is added to the reaction.

Samples of patients either positive or negative for monkeypox virus are collected. Viral DNA targets in the samples are amplified by loop mediated amplification (LAMP) using six amplification primers: M44982 monkeypox-set1-F3 (SEQ ID NO: 92), M44983 monkeypox-set1-B3 (SEQ ID NO: 93), M44984 monkeypox-set1-FIP (SEQ ID NO: 94), M44985 monkeypox-set1-BIP (SEQ ID NO: 95), M44986 monkeypox-set1-LF (SEQ ID NO: 96), M44987 monkeypox-set1-LB (SEQ ID NO: 97).

After completion of the pre-amplification step, 2 μL of amplicon is combined with 18 μL of sgRNA-Cas14a.1 complexes and 80 μL of 1X reaction buffer. The 100 μL Cas14a.1 trans-cleavage assay is allowed to proceed for 10 minutes at 37° C.

A lateral flow strip (Milenia HybriDetect 1, TwistDx) is then added to the reaction tube and a result is visualized after approximately 2-3 minutes. A single band, close to the sample application pad indicates a negative result, whereas a single band close to the top of the strip or two bands indicates a positive result.

### Example 13: One-Pot LAMP DETECTR Reactions for Detection of Monkeypox Virus

This example describes LAMP DETECTR reactions for the detection of monkeypox virus in a one-step, one-pot reaction.

SgRNA-Cas14a.1 complexes are prepared by pre-incubating Cas 14a.1 with sgRNA MPXV-1 (SEQ ID NO: 236) for 30 minutes at 37° C. After formation of the sgRNA-Cas14a.1 complexes, a fluorophore-quencher labeled ssDNA reporter, amplification primers, dNTPs, and a polymerase are added to the reaction in a 1X reaction buffer.

Samples of patients either positive or negative for monkeypox virus are collected and added to the reaction and the one-pot amplification/trans-cleavage assay is allowed to proceed for 30 minutes at 37° C. Viral DNA targets in the samples are amplified by loop mediated amplification (LAMP) using six amplification primers: M44982 monkeypox-set1-F3 (SEQ ID NO: 92), M44983 monkeypox-set1-B3 (SEQ ID NO: 93), M44984 monkeypox-set1-FIP (SEQ ID NO: 94), M44985 monkeypox-set1-BIP (SEQ ID NO: 95), M44986 monkeypox-set1-LF (SEQ ID NO: 96), M44987 monkeypox-set1-LB (SEQ ID NO: 97). Amplification occurs concurrently with detection of the viral DNA targets and/or amplicons. Presence of the target DNA, or an amplicon thereof, within the sample activates the sgRNA-Cas14a.1 complex to trans-cleave the reporter, thereby separating the fluorophore and the quencher from one another and resulting in a detectable fluorescent signal. Absence of the target DNA, or an amplicon thereof, within the sample results in no trans-cleavage the reporter, and thus no separation of the fluorophore and the quencher from one another and no detectable fluorescent signal.

The foregoing is illustrative of the present disclosure, and is not to be construed as limiting thereof. The invention is defined by the following claims, with equivalents of the claims to be included therein.

What is claimed is:

1. A method of quantitating a target nucleic acid in a test sample, the method comprising:
(a) contacting the test sample with the following components (i) through (iii), resulting in at least two nanovolumes of reaction mixture:
(i) a CRISPR/Cas effector protein,
(ii) a guide RNA, comprising a region that is capable of binding to the CRISPR/Cas effector protein, and a guide sequence that is capable of hybridizing with the target nucleic acid,
(iii) a reporter nucleic acid that is single stranded and does not hybridize with the guide sequence of the guide RNA;
(b) measuring detectable signals detected from the at least two nanovolumes and generated by cleavage of the reporter nucleic acid by the CRISPR/Cas effector protein, and
(c) quantitating the target nucleic acid in the test sample based on the measured signals from the at least two nanovolumes.

2. The method of claim 1, wherein the contacting step comprises sequentially adding each of the components (i) through (iii) to the test sample in the at least two nanovolumes.

3. The method of claim 1, wherein the contacting step comprises: (a) adding each of the components (i) through (iii) to the test sample to generate a master reaction mixture, wherein the master reaction mixture has a volume of more than 1 nL, and (b) distributing the master reaction mixture into the at least two nanovolumes.

4. The method of any one of claims 1-3, wherein each of the at least two nanovolumes comprises no more than 1 molecule of the target nucleic acid.

5. The method of any one of claims 1-4, wherein the CRISPR/Cas effector protein and the guide RNA are incubated with each other prior to step (a).

6. The method of any one of claims 1-4, wherein the CRISPR/Cas effector protein and the guide RNA are not incubated with each other prior to step (a).

7. The method of any one of claims 1-6, further comprising quantitating multiple target nucleic acids in the test sample, wherein the at least two nanovolumes of reaction mixture comprises one or more guide RNAs, wherein at least one of the one or more guide RNAs comprises a guide sequence that is capable of hybridizing with each of the multiple target nucleic acids.

8. The method of claim 7, wherein each of the at least two nanovolumes of reaction mixture comprises at least one of the one or more guide RNAs comprising a guide sequence that is capable of hybridizing with each of the multiple target nucleic acids.

9. The method of claim 7, wherein each of the at least two nanovolumes of reaction mixture comprises at least one of the one or more guide RNAs comprising a guide sequence that is capable of hybridizing with no more than one of the multiple target nucleic acids.

10. The method of any one of claims 1-9, wherein the method does not comprise amplifying the target nucleic acid in the test sample.

11. The method of any one of claims 1-10, wherein the test sample comprises about 10,000 molecules to about 100,000 molecules of the target nucleic acid.

12. The method of claim 11, wherein the test sample comprises about 50,000 molecules of the target nucleic acid.

13. The method of any one of claims 1-12, wherein the contacting step results in a number of nanovolumes in the range of about 5000 nanovolumes to about 100,000 nanovolumes.

14. The method of any one of claims 1-13, wherein step (b) comprises measuring a binary signal from each of the at least two nanovolumes.

15. The method of any one of claims 1-14, comprising contacting the test sample with a precursor guide RNA array, wherein the CRISPR/Cas effector protein cleaves the precursor guide RNA array to produce the guide RNA.

16. The method of any one of claims 1-15, wherein the target nucleic acid is DNA or RNA.

17. The method of any one of claims 1-16, wherein the target nucleic acid is a viral nucleic acid or a bacterial nucleic acid.

18. The method of claim 17, wherein the target nucleic acid is a viral nucleic acid.

19. The method of claim 18, wherein the target nucleic acid is derived from a papovavirus, a human papillomavirus (HPV), a hepadnavirus, a Hepatitis B Virus (HBV), a herpesvirus, a varicella zoster virus (VZV), an Epstein Barr virus (EBY), a Kaposi's sarcoma-associated herpesvirus, an adenovirus, a poxvirus, a parvovirus, an influenza virus, a respiratory syncytial virus, an orthopoxvirus, or a coronavirus.

20. The method of claim 19, wherein the target nucleic acid is derived from a severe acute respiratory syndrome coronavirus 2 (SARS-CoV-2).

21. The method of any one of claims 1-20, wherein the target nucleic acid is derived from a human cell.

22. The method of any one of claims 1-21, wherein the target nucleic acid is a human fetal nucleic acid or a cancer cell nucleic acid.

23. The method of any one of claims 1-22, wherein the target nucleic acid is single stranded.

24. The method of any one of claims 1-22, wherein the target nucleic acid is double stranded.

25. The method of any one of claims 1-24, wherein the test sample comprises DNA or RNA from a cell lysate.

26. The method of any one of claims 1-25, wherein the test sample comprises cells.

27. The method of any one of claims 1-26, wherein the test sample is a blood, serum, plasma, urine, aspirate, fecal, wastewater, or biopsy sample.

28. The method of any one of claims 1-27, further comprising quantitating a positive control target nucleic acid in a positive control sample, the method comprising:
(a) contacting the positive control sample with the following components (i) through (iii) resulting in at least two nanovolumes of reaction mixture:
(i) a CRISPR/Cas effector protein;
(ii) a positive control guide RNA, comprising a region that is capable of binding to the CRISPR/Cas effector protein, and a guide sequence that is capable of hybridizing with the positive control target nucleic acid; and
(iii) a reporter nucleic acid that is single stranded and does not hybridize with the guide sequence of the positive control guide RNA;
(b) measuring detectable signals detected from the at least two nanovolumes and produced by cleavage of the reporter nucleic acid by the CRISPR/Cas effector protein, and
(c) quantitating the amount of positive control target nucleic acid in the positive control sample based on the measured signals from the at least two nanovolumes.

29. The method of claim 28, wherein the at least two nanovolumes comprises more than one type of CRISPR/Cas effector protein, and one or more positive control guide RNAs capable of binding to each of the more than one type of CRISPR/Cas effector protein.

30. The method of claim 29, wherein the at least two nanovolumes comprises a Cas12 protein and a Cas13 protein, a positive control guide RNA comprising a region that is capable of binding to Cas12 protein and a positive control guide RNA comprising a region that is capable of binding to a Cas13 protein.

31. The method of any one of claims 1-30, further comprising quantitating the target nucleic acid in a positive control sample, the method comprising:
(a) contacting the positive control sample with the following components (i) through (iii) resulting in at least two nanovolumes of reaction mixture:
(i) a CRISPR/Cas effector protein;
(ii) the guide RNA, comprising a region that is capable of binding to the CRISPR/Cas effector protein, and a guide sequence that is capable of hybridizing with the target nucleic acid; and
(iii) a reporter nucleic acid that is single stranded and does not hybridize with the guide sequence of the guide RNA;
(b) measuring detectable signals detected from the at least two nanovolumes produced by cleavage of the reporter nucleic acid by the CRISPR/Cas effector protein, and
(c) quantitating the amount of target nucleic acid in the positive control sample based on the measured signals from the at least two nanovolumes.

32. The method of claim 31, wherein the method comprises generating a standard curve for the target nucleic acid in the positive control sample, and obtaining an absolute quantitation of the target nucleic acid in the test sample based on the standard curve.

33. The method of claim 31, wherein the method comprises obtaining a relative quantitation of the target nucleic acid in the test sample based on the quantitation of the target nucleic acid in a positive control sample.

34. The method of any one of claims 1-33, wherein the detectable signal is detectable in less than 90 minutes.

35. The method of any one of claims 1-34, wherein the detectable signal is detectable in less than 30 minutes.

36. The method of any one of claims 1-35, wherein the CRISPR/Cas effector protein comprises an amino acid sequence with at least 90% identity to the amino acid sequence of any one of SEQ ID NOs: 1-72.

37. The method of any one of claims 1-36, wherein the CRISPR/Cas effector protein comprises the amino acid sequence of any one of SEQ ID NOs: 1-72.

38. The method of any one of claims 1-37, wherein target nucleic acid is an RNA.

39. The method of claim 38, wherein the CRISPR/Cas effector protein is an RNA-targeting CRISPR/Cas effector protein.

40. The method of claim 39, wherein the RNA-targeting CRISPR/Cas effector protein comprises an amino acid sequence with at least 90% identity to the amino acid sequence of SEQ ID NO: 21, 62, 43, 41, or 42.

41. The method of claim 40, wherein the RNA-targeting CRISPR/Cas effector protein comprises the amino acid sequence of SEQ ID NO: 21, 62, 43, 41, or 42.

42. The method of any one of claims 1-37, wherein target nucleic acid is a DNA.

43. The method of claim 42, wherein the CRISPR/Cas effector protein is a DNA-targeting CRISPR/Cas effector protein.

44. The method of claim 43, wherein the DNA-targeting CRISPR/Cas effector protein comprises an amino acid sequence with at least 90% identity to the amino acid sequence of SEQ ID NO: 3, 34, 57, 36, 65, 67, 68, 89, 90, 91, or 17.

45. The method of claim 44, wherein the DNA-targeting CRISPR/Cas effector protein comprises the amino acid sequence of SEQ ID NO: 3, 34, 57, 36, 65, 67, 68, 89, 90, 91, or 17.

46. The method of any one of claims 1-45, wherein the reaction mixture comprises a buffer, wherein the buffer comprises tricine, MgOAc, BSA, TCEP, imidazole, KCl, MgCl₂, BSA, Igepal Ca-630, glycerol, HEPES, KOAc, Triton-X 100, Tris-HCl, (NH₄)₂SO₄, Tween-20, TMAO, or any combination thereof.

47. The method of any one of claims 1-46, wherein the reporter nuclear acid is a RNA.

48. The method of any one of claims 1-46, wherein the reporter nuclear acid is a DNA.

49. The method of any one of claims 1-48, wherein the reporter nucleic acid comprises a modified nucleobase, a modified sugar moiety, and/or a modified nucleic acid linkage.

50. A method of assaying for a target nucleic acid in a sample, the method comprising:
a) amplifying the target nucleic acid using at least one amplification primer;
b) contacting the sample to:
i. a reporter; and
ii. a composition comprising a programmable nuclease and a guide nucleic acid that hybridizes to the target nucleic acid or an amplified product thereof, wherein the programmable nuclease cleaves the reporter upon hybridization of the guide nucleic acid to the target nucleic acid or the amplification product thereof; and
c) assaying for a change in a signal, wherein the change in the signal is produced by cleavage of the reporter;
wherein the target nucleic acid is a gene of a monkeypox virus or a segment thereof; and
optionally wherein the at least one amplification primer comprises a nucleotide sequence at least 85%, at least 87%, at least 90%, at least 92%, at least 94%, at least 95%, at least 96%, at least 98%, or 100% identical to any one of SEQ ID NOs: 92-235.
